(19) United States
(12) Patent Application Publication
(10) Pub. No.: US 2025/0011449 A1
(43) Pub. Date: Jan. 9, 2025

(54) CIRCULARIZED ANTIBODY MOLECULES

(71) Applicant: Regeneron Pharmaceuticals, Inc., Tarrytown, NY (US)

(72) Inventors: Tri NGUYEN, Dobbs Ferry, NY (US); Samarthaben Jayeshkumar PATEL, Tarrytown, NY (US); Yang SHEN, Scarsdale, NY (US); Chia-Yang LIN, Scarsdale, NY (US)

(73) Assignee: Regeneron Pharmaceuticals, Inc., Tarrytown, NY (US)

(21) Appl. No.: 18/738,719

(22) Filed: Jun. 10, 2024

Related U.S. Application Data
(60) Provisional application No. 63/472,312, filed on Jun. 11, 2023.

(51) Int. Cl. C07K 16/28

(52) U.S. Cl. CPC C07K 16/2878; C07K 2317/31; C07K 2317/35; C07K 2317/40; C07K 2317/64; C07K 2319/30

(57) ABSTRACT

The present disclosure provides circularized antibody molecules and methods of their production and use.

### 1. CROSS-REFERENCE TO RELATED APPLICATIONS

This application claims the priority benefit of U.S. provisional application No. 63/472,312, filed on Jun. 11, 2023, the contents of which are incorporated herein in their entirety by reference thereto.

### 2. SEQUENCE LISTING

The instant application contains a Sequence Listing which has been submitted electronically and is hereby incorporated by reference in its entirety. Said copy, created on Jun. 5, 2024, is named RGN-037US_SL.xml and is 773, 199 bytes in size.

### 3. BACKGROUND

Antibodies are the key effector molecules of the immune system. An essential part of the adaptive immune response is the production of a diverse set of antibodies that are capable of neutralizing invading pathogens or disease-causing molecules.

Avidity of an antibody molecule represents the overall strength of the antibody-antigen interaction and is influenced by the binding affinity of its individual Fab domains, the antibody valency, and the structural arrangement of the antibody molecule. Another important factor for the functionality of an antibody is the specificity of its Fab domains. For instance, a bispecific antibody can recognize two antigens or two binding sites on the same antigen, simultaneously.

Antibody engineering has been used to enhance properties of antibody biotherapeutics. One approach is generating relatively bulky multivalent antibodies with multiple Fab domains attached to multimerization domains. Smaller formats, such as diabodies or triabodies have also been generated, but these formats are derived from scFv domains and have reduced affinity to their target molecules.

There remains a need for antibody molecules suitable for binding target molecules with both high affinity and high avidity.

### 4. SUMMARY

The present disclosure relates to circularized antibody molecules, their precursor polypeptides, and methods and production and use thereof.

The circularized antibody molecules generally comprise three antibody fragments (such as Fab domains or Fc regions) and may optionally comprise fusion partners. The antibody fragments are each generally composed to two associated polypeptide chains, one contained within a cyclized polypeptide chain (sometimes referred to herein as a “cyclizable domain”) and another on a separate polypeptide chain (sometimes referred to herein as a “counterpart domain”). In some embodiments, the cyclizable domain is an Fd domain and the counterpart domain is a light chain (LC). In other embodiments, the cyclizable domain is a light chain (LC) and the counterpart domain is an Fd domain. In yet other embodiments, the cyclizable domain and the counterpart domain are both Fc domains. Exemplary circularized antibody molecules are described in Section 6.2 and numbered embodiments 124 to 269 and 287.

Each pair of antibody fragments in the circularized antibody molecules is typically at an angle ranging between 55° and 65°, e.g., as set forth in Section 6.2 and numbered embodiments 2 to 7. Achieving the correct angle is facilitated by separating each pair of antibody fragments by an appropriate linker. Exemplary linkers separating the pairs of antibody fragments, sometimes referred to herein as L-1, L-2 and L-3, are disclosed in Section 6.6 and numbered embodiments 20 to 34 and 56.

The polypeptide chains comprising the counterpart domains can comprise fusion partners, which may include single chain antibodies and antibody fragments as well as receptor fragments. Exemplary fusion partners are disclosed in Section 6.9 and numbered embodiments 58 and 59. The fusion partners can be separated from the counterpart domains by linkers. Exemplary linkers separating the fusion partners and the counterpart domains, sometimes referred to as L-4, L-5 and L-6, are disclosed in Section 6.7 and numbered embodiment 56.

In some embodiments, the antibody fragments and/or fusion partners bind to a coronavirus spike protein, for example comprise anti-spike protein binding sequences (e.g., CDRs or VH/VL sequences as set forth in Section 6.5.1 in the case of Fab, scFv or sdAb antibody fragments) or comprise ACE2 receptor sequences (e.g., ACE2 receptor sequences as set forth in Section 6.9.2.1 in the case of receptor-based fusion partners).

The circularized antibody molecules of the disclosure are typically produced by circularization of precursor polypeptides comprising split inteins at their termini. Suitable precursor polypeptides and methods of producing the circularized antibody molecules are described in Section 6.2 and numbered embodiments 1 to 123. Suitable split inteins are described in Section 6.8 and numbered embodiments 122 to 123. Suitable methods of producing the circularized antibody molecules and the resulting populations of circularized antibody molecules are described in Section 6.11 and numbered embodiments 270 to 275 and 287.

The present disclosure further provides nucleic acids encoding the precursors of the circularized antibody molecules proteins of the disclosure, host cells engineered to express the precursors, and recombinant methods for the production of the precursors and the circularized antibody molecules. Such nucleic acids, host cells and production methods are described in Section 6.10 and numbered embodiments 276 to 278.

The present disclosure further provides pharmaceutical compositions comprising the circularized antibody molecules and populations of the disclosure as well as methods of their use in therapy. Pharmaceutical compositions are described in Section 6.12 and numbered embodiments 279 and 287. Method of use of the circularized antibody molecules and pharmaceutical compositions are described in Section 6.13 and numbered embodiments 280 to 286.

Other features and advantages of aspects of the fusion proteins of the present disclosure will become apparent from the following more detailed description, taken in conjunction with the accompanying drawings.

### 5. BRIEF DESCRIPTION OF THE FIGURES

FIGS. 1A-1D display the components that can be used to produce circularized antibody molecules of the disclosure. FIG. 1A shows a polypeptide comprising three cyclizable domains connected to one another via linkers and located between two split intein fragments. From N- to C-terminal, the polypeptide comprises: a first split intein (I-A), a first portion of a first linker (the linker referred to as linker 1 or L-1, the first portion referred to as L-1A)—first cyclizable domain (“CycD-1”)—second linker (linker 2 or L-2)—second cyclizable domain (CycD-2)—third linker (linker 3 or L-3)—third cyclizable domain (CycD-3)—a second portion of the first linker (L-1B)—second split intein (I-B). FIG. 1B shows a counterpart domain (COD) that can pair with CycD-1 (CoD-1), CycD-2 (CoD-2) and/or CycD-3 (CoD-3). FIG. 1C shows a CoD that can pair with CycD-1 (CoD-1), CycD-2 (CoD-2) and/or CycD-3 (CoD-3), with a fusion partner (FP-1, FP-2, or FP-3, respectively) at its N-terminus. FIG. 1D shows a CoD that can pair with CycD-1 (CoD-1), CycD-2 (CoD-2) and/or CycD-3 (CoD-3), with a fusion partner (FP-1, FP-2, or FP-3, respectively) at its C-terminus.

FIGS. 2A-2O show the components and production from linear precursors of exemplary circularized antibody molecules. FIG. 2A shows a polypeptide comprising three Fd heavy chains connected to one another via linkers and located between two split intein fragments. From N- to C-terminal, the polypeptide comprises: a first split intein (I-A)—a first portion of a first linker (the linker referred to as linker 1 or L-1, the first portion referred to as L-1A)—first Fd domain (“Fd-1”)—second linker (linker 2 or L-2)—second Fd domain (Fd-2)—third linker (linker 3 or L-3)—third Fd domain (Fd-3)—a second portion of the first linker (L-1B)—second split intein (I-B). FIG. 2B shows a light chain (LC) that can pair with Fd-1 (light chain 1 or LC-1), Fd-2 (light chain 2 or LC-2) and/or Fd-3 (light chain 3 or LC-3). The association of Fd-1 and LC-1 forms a first Fab domain (Fab-1), the association of Fd-2 and LC-2 forms a second Fab domain (Fab-2), and the association of Fd-3 and LC-3 forms a third Fab domain (Fab-3). FIG. 2C shows a polypeptide comprising three light chains connected to one another via linkers and located between two split intein fragments. From N- to C-terminal, the polypeptide comprises: I-A-L-1A—first light chain (light chain 1 or LC-1)-L-2—second light chain (light chain 2 or LC-2)-L-3—third light chain (light chain 3 or LC-3)-L-1B-I-B. FIG. 2D shows an Fd domain that can pair with LC-1 (Fd-1), LC-2 (Fd-2) and/or LC-3 (Fd-3). The association of Fd-1 and LC-1 forms a first Fab domain (Fab-1), the association of Fd-2 and LC-2 forms a second Fab domain (Fab-2), and the association of Fd-3 and LC-3 forms a third Fab domain (Fab-3). FIG. 2E shows a polypeptide comprising three Fc (Fc) domains (e.g., Fc (knob) or Fc (hole) domains) connected to one another via linkers and located between two split intein fragments. From N- to C-terminal, the polypeptide comprises: I-A-L-1A—first Fc domain (Fc domain 1 or Fc-1)-L-2—second Fc domain (Fc domain 2 or Fc-2)-L-3—third Fc domain (Fc domain 3 or Fc-3)-L-1B-I-B. FIG. 2F shows a counterpart Fc domain (e.g., an Fc (hole) domain if the Fc-1/2/3 are Fc (knob) domains or an Fc (knob) domain if the Fc-1/2/3 are Fc (hole) domains) that can pair with Fc-1 (Fc-4), Fc-2 (Fc-5) and/or Fc-3 (Fc-6). The association of Fc-1 and Fc-4 forms a first Fc region (Fc-R1), the association of Fc-2 and Fc-5 forms a second Fc region (Fc-R2), and the association of Fc-3 and Fc-6 forms a third Fc region (Fc-R3). FIG. 2G shows a polypeptide comprising both Fd and Fc domains connected to one another via linkers and located between two split intein fragments. From N- to C-terminal, the polypeptide comprises: I-A-L-1A-Fd-1-L-2-Fc-1-L-3-Fd-2-L-1B-I-B. FIG. 2H shows a counterpart LC and a counterpart Fc domain. FIG. 2I shows a polypeptide comprising both LC and Fc domains connected to one another via linkers and located between two split intein fragments. From N- to C-terminal, the polypeptide comprises: I-A-L-1A-LC-1-L-2-Fc-1-L-3-LC-2-L-1B-I-B. FIG. 2J shows a counterpart Fd domain and a counterpart Fc domain FIG. 2K illustrates the circularization steps of a linear precursor comprising the Fd-containing polypeptide depicted in FIG. 2A and the light chain polypeptide(s) depicted in FIG. 2B. When co-expressed in mammalian cells, each Fd pairs with its respective LC to form a linear construct (referred to as a “precursor”) comprising three Fab domains (Fab-1, Fab-2, and Fab-3) flanked by the first and second split intein fragments. When subjected to appropriate conditions, e.g., as described in Section 6.11, the split inteins located at each end of the construct form an intein complex, producing a cyclic intermediate from the linear construct. Next, the intein complex undergoes trans-splicing, which generates a circularized antibody molecule upon the excision of the intein complex. FIG. 2L illustrates the circularization steps of a linear precursor comprising the light chain-containing polypeptide depicted in FIG. 2C and the Fd-containing polypeptide(s) depicted in FIG. 2D. FIG. 2M illustrates the circularization steps of a linear precursor comprising the Fc-containing polypeptide depicted in FIG. 2E and the counterpart Fc-containing polypeptide(s) depicted in FIG. 2F. FIG. 2N illustrates the circularization steps of a linear precursor comprising the Fd and Fc-containing polypeptide depicted in FIG. 2G, and the LC- and Fc-containing polypeptides depicted in FIG. 2H. FIG. 2O illustrates the circularization steps of a linear precursor comprising the LC and Fc-containing polypeptide depicted in FIG. 2I, and the Fd- and counterpart Fc-containing polypeptides depicted in FIG. 2J.

FIGS. 3A-3E show the components and production from linear precursors of an exemplary circularized antibody molecule according to FIG. 2K, which further comprises fusion partners. FIG. 3A shows an Fd-containing polypeptide comprising from N- to C-terminal: I-A-L-1A-Fd-1-L-2-Fd-2-L-3-Fd-3-L-1B-I-B. FIG. 3B shows a polypeptide chain comprising a light chain (LC) that can pair with Fd-1 (LC-1), Fd-2 (LC-2) and/or Fd-3 (LC-3), with a fusion partner (FP-1, FP-2, or FP-3, respectively) at its N-terminus. FIG. 3C shows a polypeptide chain comprising an LC that can pair with Fd-1 (LC-1), Fd-2 (LC-2) and/or Fd-3 (LC-3), with a fusion partner (FP-1, FP-2, or FP-3, respectively) at its C-terminus. As shown, each LC-FP pair is separated by a linker (L-4 separating FP-1 and LC-1, L-5 separating FP-2 and LC-2, or L-6 separating FP-3 and LC-3). FIG. 3D illustrates the circularization steps of a linear precursor comprising the Fd-containing polypeptide depicted in FIG. 3A and the light chain polypeptide(s) depicted in FIG. 3B. FIG. 3E illustrates the circularization steps of a linear precursor comprising the Fd-containing polypeptide depicted in FIG. 3A and the light chain polypeptide(s) depicted in FIG. 3C.

FIGS. 4A-4E show the components and production from linear precursors of an exemplary circularized antibody molecule according to FIG. 2L, which further comprises fusion partners. FIG. 4A shows an LC-containing polypeptide comprising from N- to C-terminal: I-A-L-1A-LC-1-L-2-LC-2-L-3-LC-3-L-1B-I-B. FIG. 4B shows a polypeptide chain comprising an Fd domain (Fd) that can pair with LC-1 (Fd-1), LC-2 (Fd-2) and/or LC-3 (Fd-3), with a fusion partner (FP-1, FP-2, or FP-3, respectively) at its N-terminus. FIG. 4C shows a polypeptide chain comprising an Fd domain that can pair with LC-1 (Fd-1), LC-2 (Fd-2) and/or LC-3 (Fd-3), with a fusion partner (FP-1, FP-2, or FP-3, respectively) at its C-terminus. As shown, each Fd-FP pair is separated by a linker (L-4 separating FP-1 and Fd-1, L-5 separating FP-2 and Fd-2, or L-6 separating FP-3 and Fd-3). FIG. 4D illustrates the circularization steps of a linear precursor comprising the light chain-containing polypeptide depicted in FIG. 4A and the Fd-containing polypeptide(s) depicted in FIG. 4B. FIG. 4E illustrates the circularization steps of a linear precursor comprising the light chain-containing polypeptide depicted in FIG. 4A and the Fd-containing polypeptide(s) depicted in FIG. 4C.

FIGS. 5A-5E show the components and production from linear precursors of an exemplary circularized antibody molecule according to FIG. 2M, which further comprises fusion partners. FIG. 5A shows an Fc-containing polypeptide (e.g., an Fc (knob) domain) comprising from N- to C-terminal: I-A-L-1A-LC-1-L-2-LC-2-L-3-LC-3-L-1B-I-B. FIG. 5B shows an Fc domain (e.g., an Fc (hole) domain) that can pair with Fc-1 (Fc-4), Fc-2 (Fc-5) and/or Fc-3 (Fc-6), with a fusion partner (FP-1, FP-2, or FP-3, respectively) at its N-terminus. FIG. 5C shows an Fc domain (e.g., an Fc (hole) domain) that can pair with Fc-1 (Fc-4), Fc-2 (Fc-5) and/or Fc-3 (Fc-6), with a fusion partner (FP-1, FP-2, or FP-3, respectively) at its C-terminus. As shown, each Fd-FP pair is separated by a linker (L-4 separating FP-1 and Fd-1, L-5 separating FP-2 and Fd-2, or L-6 separating FP-3 and Fd-3). FIG. 5D illustrates the circularization steps of a linear precursor comprising the Fc-containing polypeptides depicted in FIGS. 5A and 5B. FIG. 5E illustrates the circularization steps of a linear precursor comprising the Fc-containing polypeptides depicted in FIGS. 5A and 5C.

FIGS. 6A-6E show metadynamics simulations of a circularized antibody molecule comprising three Fab domains (sometimes referred to herein as “tri-Fab” or “tri-Fab construct” for convenience) comprising linkers with different lengths separating the Fd domains. FIG. 6A is a cartoon depiction of a desired angle between two adjacent Fab domains. In order for three Fab domains to form an equilateral triangle, each Fab-Fab angle would be approximately 60° to maintain a low energy state. FIG. 6B shows heat maps for each of the three angles of a tri-Fab construct comprising a seven amino-acid (7aa) linker, depicting the free energy (kcal/mol) associated with adjacent collective variable (CV) distances. Darker areas of the heat maps represent lower energy states. FIG. 6C shows heat maps for each of the three angles of a tri-Fab construct comprising a 5aa linker. FIG. 6D shows heat maps for each of the three angles of a tri-Fab construct comprising a 3aa linker. FIG. 6E shows heat maps for the same angle of a tri-Fab construct comprising GS-linkers of different lengths.

FIGS. 7A-7B show the results of an SDS-PAGE analysis of linear and circularized (cyclic) tri-Fab constructs. FIG. 7A shows a representative non-reducing SDS-PAGE, which shows bands that correspond to full length tri-Fab constructs and excess LC polypeptides. FIG. 7B shows a representative reducing SDS-PAGE, which shows bands that correspond to linear and cyclic tri-Fd polypeptides, LC polypeptide, and cyclic tri-Fd with an LC. Neither reducing nor non-reducing gels showed any intein-comprising constructs, suggesting complete removal of the intein complex upon circularization.

FIGS. 8A-8B display shape analysis results of tri-Fab constructs. FIG. 8A shows the relation between molecular structure and shape factor ρ, which is the ratio between radius of gyration (R_{g}) and hydrodynamic radius (Rₕ). FIG. 8B shows representative R_{g} and Rₕ values and the resulting p ratios for three tri-Fab constructs.

FIGS. 9A-9E show negative stain electron microscopy (EM) imaging results for cyclic tri-Fab constructs. FIG. 9A shows the top populated classes of a 2D classification assessment. FIG. 9B shows an exemplary image of a putative cyclic class representing 2.3% of the particles analyzed. FIG. 9C shows an exemplary image of a putative cyclic class representing 2.1% of the particles analyzed. FIG. 9D shows an exemplary image of a putative cyclic class representing 1.8% of the particles analyzed. FIG. 9E shows an exemplary image of a putative cyclic class representing 1.6% of the particles analyzed.

### 6. DETAILED DESCRIPTION

### 6.1. Definitions

As used herein, the following terms are intended to have the following meanings:

About, Approximately: The terms “about”, “approximately” and the like are used throughout the specification in front of a number to show that the number is not necessarily exact (e.g., to account for fractions, variations in measurement accuracy and/or precision, timing, etc.). It should be understood that a disclosure of “about X” or “approximately X” where X is a number is also a disclosure of “X.” Thus, for example, a disclosure of an embodiment in which one sequence has “about X % sequence identity” to another sequence is also a disclosure of an embodiment in which the sequence has “X % sequence identity” to the other sequence.

ACE2 Moiety: The term “ACE2 moiety” refers to a moiety comprising an amino acid sequence that has at least 70% sequence identity to an extracellular portion of human ACE2 that is capable of binding the RBD of SARS-CoV or SARS-CoV-2 RBD, for example an amino acid sequence having at least 70% sequence identity to the peptidase domain (PD) of human ACE2. In some embodiments, an ACE2 moiety comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to the peptidase domain of human ACE2. In further embodiments, the ACE2 moiety comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to the peptidase and neck domains of human ACE2. Typically, the ACE2 moiety lacks a transmembrane domain.

And, or: Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

Antibody: The term “antibody” as used herein refers to a polypeptide (or set of polypeptides) of the immunoglobulin family that is capable of binding an antigen non-covalently, reversibly and specifically. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes, but is not limited to, monoclonal antibodies, human antibodies, humanized antibodies, camelized antibodies, chimeric antibodies, bispecific or multispecific antibodies and anti-idiotypic (anti-id) antibodies. The antibodies can be of any isotype/class (e.g., IgG, IgE, IgM, IgD, IgA and IgY) or subclass (e.g., IgG1, IgG2, IgG3, IgG4, IgA1 and IgA2). Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding domain or amino-terminus of the antibody. The N-terminus is a variable region and at the C-terminus is a constant region; the CH3 and CL domains represent the carboxy-terminus of the heavy and light chain, respectively, of natural antibodies. For convenience, and unless the context dictates otherwise, the reference to an antibody also refers to antibody fragments as well as engineered antibodies that include non-naturally occurring antigen-binding domains and/or antigen-binding domains having non-native configurations. Thus, the circularized molecules of the disclosure, as well as their non-circularized precursors, fall within the definition of “antibody” unless the context dictates otherwise.

Antibody Fragment: The term “antibody fragment” refers to a fragment of an antibody, e.g., structural and/or functional component of a naturally occurring or engineered antibody, on one or more polypeptide chains. In some embodiments, the antibody fragment is formed by association of two polypeptide chains. In some embodiments, an antibody fragment does not have the ability to bind to an antigen. Thus, an antibody fragment can comprise or consist of an Fc region or a fragment thereof, e.g., a fragment comprising CH2 and/or CH3 domains (with or without a hinge domain). In other embodiments, an antibody fragment retains the ability to bind to a target molecule. Thus, an antibody fragment can comprise or consist of a Fab domain.

Antigen-Binding Domain: The term “antigen-binding domain” or “ABD” as used herein refers to a portion of an antibody or antibody fragment that has the ability to bind to an antigen non-covalently, reversibly and specifically. Examples of an antibody fragment that can comprise an ABD include, but are not limited to, a single-chain Fv (scFv), a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., 1989, Nature 341:544-546), which consists of a VH domain; and an isolated complementarity determining region (CDR). Thus, the term “antibody fragment” encompasses both proteolytic fragments of antibodies (e.g., Fab and F(ab)₂ fragments) and engineered proteins comprising one or more portions of an antibody (e.g., an scFv). Antibody fragments can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology 23:1126-1136).

Associated: The term “associated” in the context of an antibody (e.g., a circularized antibody molecule of the disclosure or a precursor thereof) refers to a functional relationship between two or more polypeptide chains. In particular, the term “associated” means that two or more polypeptides are associated with one another, e.g., non-covalently through molecular interactions or covalently through one or more disulfide bridges or chemical cross-linkages, so as to produce a functional protein. Examples of associations that might be present in a circularized antibody molecule of the disclosure include (but are not limited to) associations between VH and VL regions in a Fab or Fv and associations between CH1 and CL in a Fab.

Bispecific: The term “bispecific” as used herein in relation to a circularized antibody molecule refers to a circularized antibody molecule whose antigen binding domains have two different binding specificities. For instance, the antigen binding domains in a bispecific circularized antibody molecule can bind to two different portions of the same target antigen (or, in the case of a viral protein, different variants of the same target antigen) or the antigen binding domains can bind to two different target antigens. Bispecific circularized antibody molecules may be bivalent or have additional valencies (e.g., may be trivalent, tetravalent, pentavalent or hexavalent).

Bivalent: The term “bivalent” as used herein in relation to a circularized antibody molecule refers to a circularized antibody molecule that has two antigen-binding domains, e.g., two Fab domains, one Fab domain and one non-Fab antigen-binding domain such as an scFv or a sdAb, or two non-Fab antigen-binding domains such as scFvs, sdAbs, or a combination thereof. In some embodiments, the Fab domains represent the antibody fragments of the circularized antibody molecules and the scFvs and/or sdAbs represent fusion partners. The antigen binding domains in a bivalent circularized antibody molecule can have the same or different binding specificities. Accordingly, a bivalent circularized antibody molecule can be monospecific or bispecific.

Complementarity Determining Region: The terms “complementarity determining region” or “CDR,” as used herein, refer to the sequences of amino acids within antibody variable regions which confer antigen specificity and binding affinity. For example, in general, there are three CDRs in each heavy chain variable region (e.g., CDR-H1, CDR-H2, and CDR-H3) and three CDRs in each light chain variable region (CDR-L1, CDR-L2, and CDR-L3). The precise amino acid sequence boundaries of a given CDR can be determined using any of a number of well-known schemes, including those described by Kabat et al., 1991, “Sequences of Proteins of Immunological Interest,” 5th Ed. Public Health Service, National Institutes of Health, Bethesda, MD (“Kabat” numbering scheme), Al-Lazikani et al., 1997, JMB 273:927-948 (“Chothia” numbering scheme) and ImmunoGenTics (IMGT) numbering (Lefranc, 1999, The Immunologist 7:132-136; Lefranc et al., 2003, Dev. Comp. Immunol. 27:55-77 (“IMGT” numbering scheme). For example, for classic formats, under Kabat, the CDR amino acid residues in the heavy chain variable domain (VH) are numbered 31-35 (CDR-H1), 50-65 (CDR-H2), and 95-102 (CDR-H3); and the CDR amino acid residues in the light chain variable domain (VL) are numbered 24-34 (CDR-L1), 50-56 (CDR-L2), and 89-97 (CDR-L3). Under Chothia, the CDR amino acids in the VH are numbered 26-32 (CDR-H1), 52-56 (CDR-H2), and 95-102 (CDR-H3); and the amino acid residues in VL are numbered 26-32 (CDR-L1), 50-52 (CDR-L2), and 91-96 (CDR-L3). By combining the CDR definitions of both Kabat and Chothia, the CDRs consist of amino acid residues 26-35 (CDR-H1), 50-65 (CDR-H2), and 95-102 (CDR-H3) in human VH and amino acid residues 24-34 (CDR-L1), 50-56 (CDR-L2), and 89-97 (CDR-L3) in human VL. Under IMGT the CDR amino acid residues in the VH are numbered approximately 26-35 (CDR-H1), 51-57 (CDR-H2) and 93-102 (CDR-H3), and the CDR amino acid residues in the VL are numbered approximately 27-32 (CDR-L1), 50-52 (CDR-L2), and 89-97 (CDR-L3) (numbering according to “Kabat”). Under IMGT, the CDR regions of an antibody can be determined using the program IMGT/DomainGap Align.

Counterpart Domain: The term “counterpart domain” as used herein refers to a polypeptide comprising an amino acid sequence capable of pairing with a cyclizable domain, wherein the pairing of the counterpart domain and the cyclizable domain generates a structural and/or functional component of an antibody molecule, e.g., a Fab domain or an Fc region comprising two associated homodimeric or heterodimeric Fc domains. For instance, a counterpart domain comprising or consisting of an Fd domain can associate with a cyclizable domain comprising or consisting of a light chain (LC), upon which the Fd domain and the LC form a Fab. Alternatively, a counterpart domain comprising or consisting of an Fc domain can associate with a cyclizable Fc domain to form an Fc region.

COVID-19: The term “COVID-19” (sometimes referred to herein as “COVID” for convenience) is the abbreviation of “Coronavirus disease 2019” and refers to the infectious disease caused by SARS-CoV-2 infection. Patients with COVID-19 may experience a wide range of symptoms ranging from mild to severe, which may include but are not limited to, fever, chills, cough, shortness of breath, difficulty breathing, fatigue, muscle aches, body aches, headache, loss of smell, loss of taste, sore throat, congestion, runny nose, nausea, and diarrhea.

Cyclizable Domain: The term “cyclizable domain” as used herein refers to one of three amino acid sequences present in a polypeptide chain representing one portion of an antibody fragment and which can associate with another portion of antibody fragment on a separate polypeptide chain to form the complete antibody fragment, e.g., a Fab domain or an Fc region comprising two associated homodimeric or heterodimeric Fc domains. For instance, a cyclizable domain comprising or consisting of an Fd domain can associate with a light chain (LC), upon which the Fd domain and the LC form a Fab domain. Alternatively, a cyclizable domain comprising or consisting of an Fc domain can associate with another Fc domain on a separate polypeptide chain to form an Fc region. Thus, for convenience, the term “cyclizable domain” encompasses the amino acid sequence when present in a polypeptide chain that is either circularized or capable of being circularized, e.g., by splicing of N- and C-terminal inteins.

Epitope: An epitope, or antigenic determinant, is a portion of an antigen recognized by an antibody or other antigen-binding moiety as described herein. An epitope can be linear or conformational.

Fab: The term “Fab” refers to a pair of polypeptide chains, the first comprising a variable heavy (VH) domain of an antibody operably linked (typically N-terminal to) to a first constant domain (referred to herein as C1), and the second comprising variable light (VL) domain of an antibody N-terminal operably linked (typically N-terminal) to a second constant domain (referred to herein as C2) capable of pairing with the first constant domain. In a native antibody, the VH is N-terminal to the first constant domain (CH1) of the heavy chain and the VL is N-terminal to the constant domain of the light chain (CL). The Fabs of the disclosure can be arranged according to the native orientation or include domain substitutions or swaps that facilitate correct VH and VL pairings. For example, it is possible to replace the CH1 and CL domain pair in a Fab with a CH3-domain pair to facilitate correct modified Fab-chain pairing in heterodimeric molecules. It is also possible to reverse CH1 and CL, so that the CH1 is attached to VL and CL is attached to the VH, a configuration generally known as Crossmab. The term “Fab” encompasses single chain Fabs.

Fc Domain and Fc Region: The term “Fc domain” refers to a portion of the heavy chain that pairs with the corresponding portion of another heavy chain. The term “Fc region” refers to the region formed by association of two heavy chain Fc domains. The two Fc domains within the Fc region may be the same or different from one another. In a native antibody the Fc domains are typically identical, but one or both Fc domains might be modified to allow for heterodimerization, e.g., via a knob-in-hole interaction. Fc domains may or may not include a hinge domain. In some embodiments, when used as a cyclizable domain, the Fc domains do not include a hinge domain. Exemplary Fc domains are described in Section 6.4.

Fd: As used herein, the term “Fd” or “Fd domain” refers to a portion of the heavy chain comprising the VH and CH1 and which pairs with a light chain to form a Fab, or an engineered version suitable for use in a domain-reversed Fab in which a CL instead of a CH1 is attached to the VH, which can pair with a light chain comprising a VL domain and a CH1 domain.

Fv: The term “Fv” refers to the minimum antibody fragment derivable from an immunoglobulin that contains a complete target recognition and binding site. This region consists of a dimer of one heavy and one light chain variable domain in a tight, noncovalent association (VH-VL dimer). It is in this configuration that the three CDRs of each variable domain interact to define a target binding site on the surface of the VH-VL dimer. Often, the six CDRs confer target binding specificity to the antibody. However, in some instances even a single variable domain (or half of an Fv comprising only three CDRs specific for a target) can have the ability to recognize and bind a target. The reference to a VH-VL dimer herein is not intended to convey any particular configuration. When present on a single polypeptide chain (e.g., a scFv), the VH and be N-terminal or C-terminal to the VL.

Hexavalent: The term “hexavalent” as used herein in relation to a circularized antibody molecule refers to a circularized antibody molecule that has six antigen-binding domains, e.g., three Fab domains and three non-Fab antigen-binding domains such as scFvs, sdAbs, or a combination thereof, two Fab domains and four non-Fab antigen-binding domains such as scFvs, sdAbs, or a combination thereof, one Fab domain and five non-Fab antigen-binding domains such as scFvs, sdAbs, or a combination thereof, or six non-Fab antigen-binding domains such as scFvs, sdAbs, or a combination thereof. In some embodiments, the Fab domains represent the antibody fragments of the circularized antibody molecules and the scFvs and/or sdAbs represent fusion partners. The antigen binding domains in a hexavalent circularized antibody molecule can have the same or different binding specificities. Accordingly, a hexavalent circularized antibody molecule can be monospecific or multispecific (e.g., bispecific, trispecific, etc.).

Hinge: The term “hinge”, as used herein, is intended to include the region of consecutive amino acid residues that connect the C-terminus of the CH1 to the N-terminus of the CH2 domain of an immunoglobulin. Several amino acids of the N-terminus of the CH2 domain, which are coded by the CH2 exon, are also considered part of the “lower hinge”. Without being bound by any one theory, amino acids of the hinge region of IgG1, IgG2 and IgG4 have been characterized as comprising 12-15 consecutive amino acids encoded by a distinct hinge exon, and several N-terminal amino acids of the CH2 domain (encoded by the CH2 exon) (Brekke et al., 1995, Immunology Today 16 (2): 85-90). On the other hand, IgG3 comprises a hinge region consisting of four segments: one upper segment resembling the hinge region of IgG1, and 3 segments that are identical amino acid repeats unique to IgG3.

Host Cell or Recombinant Host Cell: The terms “host cell” or “recombinant host cell” refer to a cell that has been genetically-engineered, e.g., through introduction of a heterologous nucleic acid. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A host cell may carry the heterologous nucleic acid transiently, e.g., on an extrachromosomal heterologous expression vector, or stably, e.g., through integration of the heterologous nucleic acid into the host cell genome. For purposes of expressing a precursor of a circularized antibody molecule of the disclosure, a host cell is preferably a cell line of mammalian origin or mammalian-like characteristics, such as monkey kidney cells (COS, e.g., COS-1, COS-7), HEK293), baby hamster kidney (BHK, e.g., BHK21), Chinese hamster ovary (CHO), NSO, PerC6, BSC-1, human hepatocellular carcinoma cells (e.g., Hep G2), SP2/0, HeLa, Madin-Darby bovine kidney (MDBK), myeloma and lymphoma cells, or derivatives and/or engineered variants thereof. The engineered variants include, e.g., derivatives that grow at higher density than the original cell lines and/or glycan profile modified derivatives and and/or site-specific integration site derivatives.

Light Chain: As used herein, the term “light chain” refers to an immunoglobulin light chain comprising the VL and CL domains of any antibody and which pairs with an Fd domain to form a Fab, or an engineered version suitable for use in a domain-reversed Fab in which a CH1 instead of a CL is attached to the VL, which can pair with an Fd domain comprising a VH domain and a CL domain.

Monospecific: The term “monospecific” as used herein refers to circularized antibody molecule that has two or more antigen binding domains that bind to the same portion of the same antigen.

Multispecific: The term “multispecific” as used herein in relation to a circularized antibody molecule refers to a circularized antibody molecule whose antigen binding domains have two or more different binding specificities.

Operably Linked: The term “operably linked” refers to a functional relationship between two or more peptide or polypeptide domains or nucleic acid (e.g., DNA) segments. In the context of a fusion protein or other polypeptide, the term “operably linked” means that two or more amino acid segments are linked so as to produce a functional polypeptide. For example, in the context of a circularized antibody molecule of the disclosure or precursor thereof, separate components (e.g., two Fd domains, or a light chain and a fusion partner) can be operably linked directly or through peptide linker sequences. In the context of a nucleic acid encoding a fusion protein, “operably linked” means that the two nucleic acids are joined such that the amino acid sequences encoded by the two nucleic acids remain in-frame. In the context of transcriptional regulation, the term refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system.

Pentavalent: The term “pentavalent” as used herein in relation to a circularized antibody molecule refers to a circularized antibody molecule that has five antigen-binding domains, e.g., three Fab domain and two non-Fab antigen-binding domains such as scFvs, sdAbs, or a combination thereof, two Fab domain and three non-Fab antigen-binding domains such as scFvs, sdAbs, or a combination thereof, one Fab domain and four non-Fab antigen-binding domains such as scFvs, sdAbs, or a combination thereof, or five non-Fab antigen-binding domains such as scFvs, sdAbs, or a combination thereof. In some embodiments, the Fab domains represent the antibody fragments of the circularized antibody molecules and the scFvs and/or sdAbs represent fusion partners. The antigen binding domains in a pentavalent circularized antibody molecule can have the same or different binding specificities. Accordingly, a pentavalent circularized antibody molecule can be monospecific or multispecific (e.g., bispecific, trispecific, etc.).

Polypeptide, Peptide and Protein: The terms “polypeptide”, “peptide” and “protein” are used interchangeably herein to refer to a polymer of amino acid residues. The polymer may be composed of a single polypeptide chain or multiple (e.g., two, three, four or more polypeptide chains).

Precursor: As used herein in relation to a circularized antibody molecule, a precursor is a polypeptide comprising one or more polypeptide chains, in which one polypeptide chain comprises a split intein at its N- and C-termini. Upon splicing of the split intein, a circularized antibody molecule of the disclosure is formed. The precursor may further comprise a signal sequence at its N-terminus, the processing of which results in the polypeptide chain with the split intein at its N- and C-termini.

Primary Target Molecule: As used herein, the term “primary target molecule” refers to a target molecule recognized by the Fab domains in the circularized portion of the circularized antibody molecules of the disclosure, e.g., by any of Fab-1, Fab-2, and Fab-3.

Recognize: The term “recognize” as used herein refers to an antibody or antibody fragment (e.g., a Fab fragment) that finds and interacts (e.g., binds) with its epitope.

Secondary Target Molecule: As used herein, the term “secondary target molecule” refers to a target molecule recognized by a fusion partner present in the circularized antibody molecules of the disclosure, e.g., by any of FP-1, FP-2 and FP-3.

Single Chain Fab or scFab: The term “single chain Fab” or “scFab” as used herein refers an ABD comprising a VH domain, a CH1 domain, a VL domain, a CL domain and a linker. In some embodiments, the foregoing domains and linker are arranged in one of the following orders in a N-terminal to C-terminal orientation: (a) VH-CH1-linker-VL-CL, (b) VL-CL-linker-VH-CH1, (c) VH-CL-linker-VL-CH1 or (d) VL-CH1-linker-VH-CL. Linkers are suitably peptide linkers of at least 30 amino acids, preferably between 32 and 50 amino acids. Single chain Fab fragments are typically stabilized via the natural disulfide bond between the CL domain and the CH1 domain. In addition, these single chain Fab molecules might be further stabilized by generation of interchain disulfide bonds via insertion of cysteine residues (e.g., at position 44 in the VH domain and position 100 in the VL domain according to Kabat numbering).

Single Chain Fv or scFv: The term “single-chain Fv” or “scFv” as used herein refers to ABDs comprising the VH and VL domains of an antibody, wherein these domains are present in a single polypeptide chain. Preferably, the Fv polypeptide further comprises a polypeptide linker between the VH and VL domains which enables the scFv to form the desired structure for antigen-binding. For a review of scFv see Pluckthun in The Pharmacology of Monoclonal Antibodies, vol. 113, Rosenburg and Moore eds. (1994), Springer-Verlag, New York, pp. 269-315. The VH and VL and be arranged in the N- to C-terminal order VH-VL or VL-VH, typically separated by a linker, for example a linker as set forth in Section 6.7.

Single Domain Antibody or sdAb: The term “single-domain antibody” or “sdAb” as used herein refers to ABDs comprising one variable domain of a heavy chain. Single domain antibodies are also referred to as nanobodies in the art.

Specifically (or Selectively) Binds: The term “specifically (or selectively) binds” to an antigen or an epitope refers to a binding reaction that is determinative of the presence of a cognate antigen or an epitope in a heterogeneous population of proteins and other molecules. The binding reaction can be but need not be mediated by an antibody or antibody fragment. The term “specifically binds” does not exclude cross-species reactivity. For example, an antigen-binding domain (e.g., an antigen-binding fragment of an antibody) that “specifically binds” to an antigen from one species may also “specifically bind” to that antigen in one or more other species. Thus, such cross-species reactivity does not itself alter the classification of an antigen-binding domain as a “specific” binder. In certain embodiments, an antigen-binding domain of the disclosure that specifically binds to a human antigen has cross-species reactivity with one or more non-human mammalian species, e.g., a primate species (including but not limited to one or more of Macaca fascicularis, Macaca mulatta, and Macaca nemestrina) or a rodent species, e.g., Mus musculus.

Subject: The term “subject” includes human and non-human animals. Non-human animals include all vertebrates, e.g., mammals and non-mammals, such as non-human primates, sheep, dog, cow, chickens, amphibians, and reptiles. In preferred embodiments, the subject is human.

Target Molecule: The term “target molecule” as used herein refers to any biological molecule (e.g., protein, carbohydrate, lipid or combination thereof), e.g., a molecule expressed on a cell surface, that can be specifically bound by an antibody.

Tetravalent: The term “tetravalent” as used herein in relation to a circularized antibody molecule refers to a circularized antibody molecule that has four antigen-binding domains, e.g., three Fab domains and one non-Fab antigen binding domain such as an scFv or sdAb, two Fab domain and two non-Fab antigen-binding domains such as scFvs, sdAbs, or a combination thereof, one Fab domain and three non-Fab antigen-binding domains such as scFvs, sdAbs, or a combination thereof, or four non-Fab antigen-binding domains such as scFvs, sdAbs, or a combination thereof. In some embodiments, the Fab domains represent the antibody fragments of the circularized antibody molecules and the scFvs and/or sdAbs represent fusion partners. The antigen binding domains in a tetravalent circularized antibody molecule can have the same or different binding specificities. Accordingly, a trivalent circularized antibody molecule can be monospecific or multispecific (e.g., bispecific, trispecific, etc.).

Treat, Treatment, Treating: As used herein, the terms “treat”, “treatment” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more circularized antibody molecules of the disclosure. In specific embodiments, the terms “treat”, “treatment” and “treating” refer to the amelioration of at least one measurable physical parameter of a disorder, not necessarily discernible by the patient. In some embodiments, the disorder is a proliferative disorder. In relation to a proliferative disorder, the terms “treat”, “treatment” and “treating” can refer to the amelioration of at least one measurable physical parameter of a proliferative disorder, such as growth of a tumor, not necessarily discernible by the patient. In other embodiments the terms “treat”, “treatment” and “treating” refer to the inhibition of the progression of the proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count. In other embodiments, the disorder is an infectious disease, e.g., a viral disease. In some embodiments, the disease or condition is caused by a coronavirus infection, for example SARS-CoV or SARS-CoV-2, for example COVID-19. In some embodiments, the disease or condition is any other ailment associated with SARS-CoV or SARS-CoV-2 infection, or similar infections. With reference to these diseases and conditions, the terms “treat”, “treatment” and “treating” refer to the reduction or amelioration of the progression, severity and/or duration of the disease or the amelioration of one or more symptoms (preferably, one or more discernible symptoms) of a disease resulting from the administration of one or more circularized antibody molecules of the disclosure. In specific embodiments, the terms “treat”, “treatment” and “treating” refer to the amelioration of at least one measurable physical parameter of COVID-19, such as blood oxygen saturation levels, not necessarily discernible by the patient. In other embodiments the terms “treat”, “treatment” and “treating” refer to the inhibition of the progression of COVID-19, either physically by, e.g., stabilization of a discernible symptom, physiologically by, e.g., stabilization of a physical parameter, or both. In other embodiments the terms “treat”, “treatment” and “treating” refer to the reduction or elimination of infection.

Trispecific: The term “trispecific” as used herein in relation to a circularized antibody molecule refers to a circularized antibody molecule whose antigen binding domains have three different binding specificities. For instance, the antigen binding domains in a trispecific circularized antibody molecule can bind to different portions of the same target antigen (or, in the case of a viral protein, different variants of the same target antigen), the antigen binding domains can bind to different target antigens, or a combination thereof. Trispecific circularized antibody molecules may be trivalent or have additional valencies (e.g., may be tetravalent, pentavalent or hexavalent).

Trivalent: The term “trivalent” as used herein in relation to a circularized antibody molecule refers to a circularized antibody molecule that has three antigen-binding domains, e.g., three Fab domains, two Fab domains and one non-Fab antigen binding domain such as an scFv or sdAb, one Fab domain and two non-Fab antigen-binding domains such as scFvs, sdAbs, or a combination thereof, or three non-Fab antigen-binding domains such as scFvs, sdAbs, or a combination thereof. In some embodiments, the Fab domains represent the antibody fragments of the circularized antibody molecules and the scFvs and/or sdAbs represent fusion partners. The antigen binding domains in a trivalent circularized antibody molecule can have the same or different binding specificities. Accordingly, a trivalent circularized antibody molecule can be monospecific or multispecific (e.g., bispecific or trispecific).

Universal Light Chain, ULC: The term “universal light chain” or “ULC” as used herein refers to a light chain variable region (VL) that can pair with more than one heavy chain variable region (VL). In the context of a circularized antibody molecule, the term “universal light chain” or “ULC” refers to a light chain polypeptide capable of pairing with the corresponding Fd domain and also capable of pairing with other Fd domains. ULCs can also include constant domains, e.g., a CL domain of an antibody. Universal light chains are also known as “common light chains.”

VH: The term “VH” refers to the variable region of an immunoglobulin heavy chain of an antibody, including the heavy chain of an Fv, scFv, dsFv or Fab.

VL: The term “VL” refers to the variable region of an immunoglobulin light chain, including the light chain of an Fv, scFv, dsFv or Fab.

### 6.2. Circularized Antibody Molecules

The present disclosure provides circularized antibody molecules comprising three antibody fragments, e.g., Fab domains and/or Fc regions. The three antibody fragments can be of the same type; for instance, a circularized antibody molecule can comprise three Fab domains or three Fc regions. A circularized antibody molecule can also comprise a combination of both Fab domains and Fc regions, e.g., two Fab domains and one Fc region or one Fab domain and two Fc regions.

The Fab domains and/or Fc regions of the circularized antibody molecules are at angles close to 60° relative to one another, e.g., angles ranging between 55° and 65°, to permit efficient circularization. In some embodiments, the angle between each pair of Fab domains and/or Fc regions ranges between 58° and 62°. In further embodiments, the angle between each pair of Fab domains and/or Fc regions is 62° or less or 61° or less. In yet further embodiments, the wherein the angle between each pair of Fab domains and/or Fc regions is 60°+1° or 60°+0.5°.

Achieving the appropriate angle between the Fab domains and/or Fc regions is facilitated by the use of the appropriate linkers, referred to herein as the first linker (L-1), the second linker (L-2) and the third linker (L-3). Suitable linker sequences for L-1 through L-3 are described in Section 6.6.

The circularized antibody molecules are generated by the splicing of a precursor molecule comprising a first split intein and a second split intein at its N- and C-termini, respectively. Suitable split inteins are described in Section 6.8.

In general, a precursor molecule that can be used to generate a circularized antibody molecule of the disclosure comprises a first polypeptide chain comprising three cyclizable domains (CycDs) flanked by split inteins, and a second, third, and fourth polypeptide chain comprising counterpart domains (CoDs) that can pair with each of the CycDs of the first polypeptide chain, thereby generating the three antibody fragments of the antibody molecule. From N- to C-terminal, the first polypeptide comprises: a first split intein (I-A), a first portion of a first linker (L-1A), a first cyclizable domain (CycD-1), a second linker (L-2), a second cyclizable domain (CycD-2), a third linker (L-3), a third cyclizable domain (CycD-3), a second portion of the first linker (L-1B), and a second split intein (I-B). Upon splicing of the first split intein (I-A) and the second split intein (I-B), a circularized polypeptide chain is produced in which the first linker becomes contiguous.

In some embodiments, the precursor polypeptide comprises a first polypeptide chain comprising, in N- to C-terminal orientation: (a) a first split intein (I-A), a first portion of the first linker (L-1A), a first Fd domain (Fd-1), the second linker (L-2), a second Fd domain (Fd-2), the third linker (L-3), a third Fd domain (Fd-3), a second portion of the first linker (L-1B), and a second split intein (I-B), wherein upon splicing of the first split intein (I-A) and the second split intein (I-B), a circularized polypeptide chain is produced in which the first linker becomes contiguous. Each Fd domain in the first polypeptide chain may be associated with a polypeptide chain (e.g., the precursor polypeptide comprises a second polypeptide chain, a third polypeptide chain and a fourth polypeptide chain) comprising a counterpart light chain.

In other embodiments, the precursor polypeptide comprises a first polypeptide chain comprising, in N- to C-terminal orientation: (a) a first split intein (I-A), a first portion of the first linker (L-1A), a first light chain (LC-1), the second linker (L-2), a second light chain (LC-2), the third linker (L-3), a third light chain (LC-3), a second portion of the first linker (L-1B), and a second split intein (I-B), wherein upon splicing of the first split intein (I-A) and the second split intein (I-B), a circularized polypeptide chain is produced in which the first linker becomes contiguous. Each light chain in the first polypeptide chain may be associated with a polypeptide chain (e.g., the precursor polypeptide comprises a second polypeptide chain, a third polypeptide chain and a fourth polypeptide chain) comprising a counterpart Fd domain.

In some other embodiments, the precursor polypeptide comprises a first polypeptide chain comprising, in N- to C-terminal orientation: (a) a first split intein (I-A), a first portion of the first linker (L-1A), a first Fc domain (Fc-1), the second linker (L-2), a second Fc domain (Fc-2), the third linker (L-3), a third Fc domain (Fc-3), a second portion of the first linker (L-1B), and a second split intein (I-B), wherein upon splicing of the first split intein (I-A) and the second split intein (I-B), a circularized polypeptide chain is produced in which the first linker becomes contiguous. Each Fc domain in the first polypeptide chain may be associated with a polypeptide chain (e.g., the precursor polypeptide comprises a second polypeptide chain, a third polypeptide chain and a fourth polypeptide chain) comprising a counterpart Fc domain. In some embodiments, the Fc regions formed by association of the Fc domains on the first polypeptide chain and the counterpart Fc domains are homodimeric. In other embodiments, the Fc regions formed by association of the Fc domains on the first polypeptide chain and the counterpart Fc domains are heterodimeric. For example, in some embodiments, the Fc domains on the first polypeptide are Fc (knob) domains and the counterpart Fc domains are Fc (hole) domains. In other embodiments, the Fc domains on the first polypeptide are Fc (hole) domains and the counterpart Fc domains are Fc (knob) domains.

In some embodiments, the precursor polypeptide comprises a first polypeptide chain comprising, in N- to C-terminal orientation: (a) a first split intein (I-A), a first portion of the first linker (L-1A), a first Fd domain (Fd-1), the second linker (L-2), an Fc domain (Fc-1), the third linker (L-3), a second Fd domain (Fd-2), a second portion of the first linker (L-1B), and a second split intein (I-B), wherein upon splicing of the first split intein (I-A) and the second split intein (I-B), a circularized polypeptide chain is produced in which the first linker becomes contiguous. Each Fd and Fc domain in the first polypeptide chain may be associated with a polypeptide chain (e.g., the precursor polypeptide comprises a second polypeptide chain, a third polypeptide chain and a fourth polypeptide chain) comprising a counterpart LC or Fc domain. In some embodiments, the Fc region formed by association of the Fc domain on the first polypeptide chain and the counterpart Fc domain are homodimeric. In other embodiments, the Fc region formed by association of the Fc domain on the first polypeptide chain and the counterpart Fc domain are heterodimeric. For example, in some embodiments, the Fc domain on the first polypeptide is an Fc (knob) domain and the counterpart Fc domain is an Fc (hole) domain. In other embodiments, the Fc domain on the first polypeptide is an Fc (hole) domain and the counterpart Fc domain is an Fc (knob) domain.

In some embodiments, the precursor polypeptide comprises a first polypeptide chain comprising, in N- to C-terminal orientation: (a) a first split intein (I-A), a first portion of the first linker (L-1A), a first Fd domain (Fd-1), the second linker (L-2), a second Fd domain (Fd-2), the third linker (L-3), an Fc domain (Fc-1), a second portion of the first linker (L-1B), and a second split intein (I-B), wherein upon splicing of the first split intein (I-A) and the second split intein (I-B), a circularized polypeptide chain is produced in which the first linker becomes contiguous. Each Fd and Fc domain in the first polypeptide chain may be associated with a polypeptide chain (e.g., the precursor polypeptide comprises a second polypeptide chain, a third polypeptide chain and a fourth polypeptide chain) comprising a counterpart LC or Fc domain. In some embodiments, the Fc region formed by association of the Fc domain on the first polypeptide chain and the counterpart Fc domain are homodimeric. In other embodiments, the Fc region formed by association of the Fc domain on the first polypeptide chain and the counterpart Fc domain are heterodimeric. For example, in some embodiments, the Fc domain on the first polypeptide is an Fc (knob) domain and the counterpart Fc domain is an Fc (hole) domain. In other embodiments, the Fc domain on the first polypeptide is an Fc (hole) domain and the counterpart Fc domain is an Fc (knob) domain.

In some embodiments, the precursor polypeptide comprises a first polypeptide chain comprising, in N- to C-terminal orientation: (a) a first split intein (I-A), a first portion of the first linker (L-1A), an Fc domain (Fc-1), the second linker (L-2), a first Fd domain (Fd-1), the third linker (L-3), a second Fd domain (Fd-2), a second portion of the first linker (L-1B), and a second split intein (I-B), wherein upon splicing of the first split intein (I-A) and the second split intein (I-B), a circularized polypeptide chain is produced in which the first linker becomes contiguous. Each Fd and Fc domain in the first polypeptide chain may be associated with a polypeptide chain (e.g., the precursor polypeptide comprises a second polypeptide chain, a third polypeptide chain and a fourth polypeptide chain) comprising a counterpart LC or Fc domain. In some embodiments, the Fc region formed by association of the Fc domain on the first polypeptide chain and the counterpart Fc domain are homodimeric. In other embodiments, the Fc region formed by association of the Fc domain on the first polypeptide chain and the counterpart Fc domain are heterodimeric. For example, in some embodiments, the Fc domain on the first polypeptide is an Fc (knob) domain and the counterpart Fc domain is an Fc (hole) domain. In other embodiments, the Fc domain on the first polypeptide is an Fc (hole) domain and the counterpart Fc domain is an Fc (knob) domain.

In other embodiments, the precursor polypeptide comprises a first polypeptide chain comprising, in N- to C-terminal orientation: (a) a first split intein (I-A), a first portion of the first linker (L-1A), a first light chain (LC-1), the second linker (L-2), an Fc domain (Fc-1), the third linker (L-3), a second light chain (LC-2), a second portion of the first linker (L-1B), and a second split intein (I-B), wherein upon splicing of the first split intein (I-A) and the second split intein (I-B), a circularized polypeptide chain is produced in which the first linker becomes contiguous. Each LC and Fc domain in the first polypeptide chain may be associated with a polypeptide chain (e.g., the precursor polypeptide comprises a second polypeptide chain, a third polypeptide chain and a fourth polypeptide chain) comprising a counterpart Fd or Fc domain. In some embodiments, the Fc region formed by association of the Fc domain on the first polypeptide chain and the counterpart Fc domain are homodimeric. In other embodiments, the Fc region formed by association of the Fc domain on the first polypeptide chain and the counterpart Fc domain are heterodimeric. For example, in some embodiments, the Fc domain on the first polypeptide is an Fc (knob) domain and the counterpart Fc domain is an Fc (hole) domain. In other embodiments, the Fc domain on the first polypeptide is an Fc (hole) domain and the counterpart Fc domain is an Fc (knob) domain.

In some embodiments, the precursor polypeptide comprises a first polypeptide chain comprising, in N- to C-terminal orientation: (a) a first split intein (I-A), a first portion of the first linker (L-1A), a first light chain (LC-1), the second linker (L-2), a second light chain (LC-2), the third linker (L-3), an Fc domain (Fc-1), a second portion of the first linker (L-1B), and a second split intein (I-B), wherein upon splicing of the first split intein (I-A) and the second split intein (I-B), a circularized polypeptide chain is produced in which the first linker becomes contiguous. Each light chain and Fc domain in the first polypeptide chain may be associated with a polypeptide chain (e.g., the precursor polypeptide comprises a second polypeptide chain, a third polypeptide chain and a fourth polypeptide chain) comprising a counterpart Fd or Fc domain. In some embodiments, the Fc region formed by association of the Fc domain on the first polypeptide chain and the counterpart Fc domain are homodimeric. In other embodiments, the Fc region formed by association of the Fc domain on the first polypeptide chain and the counterpart Fc domain are heterodimeric. For example, in some embodiments, the Fc domain on the first polypeptide is an Fc (knob) domain and the counterpart Fc domain is an Fc (hole) domain. In other embodiments, the Fc domain on the first polypeptide is an Fc (hole) domain and the counterpart Fc domain is an Fc (knob) domain.

In some embodiments, the precursor polypeptide comprises a first polypeptide chain comprising, in N- to C-terminal orientation: (a) a first split intein (I-A), a first portion of the first linker (L-1A), an Fc domain (Fc-1), the second linker (L-2), a first light chain (LC-1), the third linker (L-3), a second light chain (LC-2), a second portion of the first linker (L-1B), and a second split intein (I-B), wherein upon splicing of the first split intein (I-A) and the second split intein (I-B), a circularized polypeptide chain is produced in which the first linker becomes contiguous. Each light chain and Fc domain in the first polypeptide chain may be associated with a polypeptide chain (e.g., the precursor polypeptide comprises a second polypeptide chain, a third polypeptide chain and a fourth polypeptide chain) comprising a counterpart Fd or Fc domain. In some embodiments, the Fc region formed by association of the Fc domain on the first polypeptide chain and the counterpart Fc domain are homodimeric. In other embodiments, the Fc region formed by association of the Fc domain on the first polypeptide chain and the counterpart Fc domain are heterodimeric. For example, in some embodiments, the Fc domain on the first polypeptide is an Fc (knob) domain and the counterpart Fc domain is an Fc (hole) domain. In other embodiments, the Fc domain on the first polypeptide is an Fc (hole) domain and the counterpart Fc domain is an Fc (knob) domain.

In some embodiments, the precursor polypeptide comprises a first polypeptide chain comprising, in N- to C-terminal orientation: (a) a first split intein (I-A), a first portion of the first linker (L-1A), a first Fc domain (Fc-1), the second linker (L-2), a second Fc domain (Fc-2), the third linker (L-3), an Fd domain (Fd-1), a second portion of the first linker (L-1B), and a second split intein (I-B), wherein upon splicing of the first split intein (I-A) and the second split intein (I-B), a circularized polypeptide chain is produced in which the first linker becomes contiguous. Each Fd and Fc domain in the first polypeptide chain may be associated with a polypeptide chain (e.g., the precursor polypeptide comprises a second polypeptide chain, a third polypeptide chain and a fourth polypeptide chain) comprising a counterpart LC or Fc domain. In some embodiments, the Fc regions formed by association of the Fc domains on the first polypeptide chain and the counterpart Fc domains are homodimeric. In other embodiments, the Fc regions formed by association of the Fc domains on the first polypeptide chain and the counterpart Fc domains are heterodimeric. For example, in some embodiments, the Fc domains on the first polypeptide are Fc (knob) domains and the counterpart Fc domain are Fc (hole) domains. In other embodiments, the Fc domains on the first polypeptide are Fc (hole) domains and the counterpart Fc domains are Fc (knob) domains.

In some embodiments, the precursor polypeptide comprises a first polypeptide chain comprising, in N- to C-terminal orientation: (a) a first split intein (I-A), a first portion of the first linker (L-1A), a first Fc domain (Fc-1), the second linker (L-2), an Fd domain (Fd-1), the third linker (L-3), a second Fc domain (Fc-2), a second portion of the first linker (L-1B), and a second split intein (I-B), wherein upon splicing of the first split intein (I-A) and the second split intein (I-B), a circularized polypeptide chain is produced in which the first linker becomes contiguous. Each Fd and Fc domain in the first polypeptide chain may be associated with a polypeptide chain (e.g., the precursor polypeptide comprises a second polypeptide chain, a third polypeptide chain and a fourth polypeptide chain) comprising a counterpart LC or Fc domain. In some embodiments, the Fc regions formed by association of the Fc domains on the first polypeptide chain and the counterpart Fc domains are homodimeric. In other embodiments, the Fc regions formed by association of the Fc domains on the first polypeptide chain and the counterpart Fc domains are heterodimeric. For example, in some embodiments, the Fc domains on the first polypeptide are Fc (knob) domains and the counterpart Fc domain are Fc (hole) domains. In other embodiments, the Fc domains on the first polypeptide are Fc (hole) domains and the counterpart Fc domains are Fc (knob) domains.

In some embodiments, the precursor polypeptide comprises a first polypeptide chain comprising, in N- to C-terminal orientation: (a) a first split intein (I-A), a first portion of the first linker (L-1A), an Fd domain (Fd-1), the second linker (L-2), a first Fc domain (Fc-1), the third linker (L-3), a second Fc domain (Fc-2), a second portion of the first linker (L-1B), and a second split intein (I-B), wherein upon splicing of the first split intein (I-A) and the second split intein (I-B), a circularized polypeptide chain is produced in which the first linker becomes contiguous. Each Fd and Fc domain in the first polypeptide chain may be associated with a polypeptide chain (e.g., the precursor polypeptide comprises a second polypeptide chain, a third polypeptide chain and a fourth polypeptide chain) comprising a counterpart LC or Fc domain. In some embodiments, the Fc regions formed by association of the Fc domains on the first polypeptide chain and the counterpart Fc domains are homodimeric. In other embodiments, the Fc regions formed by association of the Fc domains on the first polypeptide chain and the counterpart Fc domains are heterodimeric. For example, in some embodiments, the Fc domains on the first polypeptide are Fc (knob) domains and the counterpart Fc domain are Fc (hole) domains. In other embodiments, the Fc domains on the first polypeptide are Fc (hole) domains and the counterpart Fc domains are Fc (knob) domains.

In some embodiments, the precursor polypeptide comprises a first polypeptide chain comprising, in N- to C-terminal orientation: (a) a first split intein (I-A), a first portion of the first linker (L-1A), a first Fc domain (Fc-1), the second linker (L-2), a second Fc domain (Fc-2), the third linker (L-3), a light chain (LC-1), a second portion of the first linker (L-1B), and a second split intein (I-B), wherein upon splicing of the first split intein (I-A) and the second split intein (I-B), a circularized polypeptide chain is produced in which the first linker becomes contiguous. Each light chain and Fc domain in the first polypeptide chain may be associated with a polypeptide chain (e.g., the precursor polypeptide comprises a second polypeptide chain, a third polypeptide chain and a fourth polypeptide chain) comprising a counterpart Fd or Fc domain. In some embodiments, the Fc regions formed by association of the Fc domains on the first polypeptide chain and the counterpart Fc domains are homodimeric. In other embodiments, the Fc regions formed by association of the Fc domains on the first polypeptide chain and the counterpart Fc domains are heterodimeric. For example, in some embodiments, the Fc domains on the first polypeptide are Fc (knob) domains and the counterpart Fc domain are Fc (hole) domains. In other embodiments, the Fc domains on the first polypeptide are Fc (hole) domains and the counterpart Fc domains are Fc (knob) domains.

In some embodiments, the precursor polypeptide comprises a first polypeptide chain comprising, in N- to C-terminal orientation: (a) a first split intein (I-A), a first portion of the first linker (L-1A), a first Fc domain (Fc-1), the second linker (L-2), a light chain (LC-1), the third linker (L-3), a second Fc domain (Fc-2), a second portion of the first linker (L-1B), and a second split intein (I-B), wherein upon splicing of the first split intein (I-A) and the second split intein (I-B), a circularized polypeptide chain is produced in which the first linker becomes contiguous. Each light chain and Fc domain in the first polypeptide chain may be associated with a polypeptide chain (e.g., the precursor polypeptide comprises a second polypeptide chain, a third polypeptide chain and a fourth polypeptide chain) comprising a counterpart Fd or Fc domain. In some embodiments, the Fc regions formed by association of the Fc domains on the first polypeptide chain and the counterpart Fc domains are homodimeric. In other embodiments, the Fc regions formed by association of the Fc domains on the first polypeptide chain and the counterpart Fc domains are heterodimeric. For example, in some embodiments, the Fc domains on the first polypeptide are Fc (knob) domains and the counterpart Fc domain are Fc (hole) domains. In other embodiments, the Fc domains on the first polypeptide are Fc (hole) domains and the counterpart Fc domains are Fc (knob) domains.

In some embodiments, the precursor polypeptide comprises a first polypeptide chain comprising, in N- to C-terminal orientation: (a) a first split intein (I-A), a first portion of the first linker (L-1A), a light chain (LC-1), the second linker (L-2), a first Fc domain (Fc-1), the third linker (L-3), a second Fc domain (Fc-2), a second portion of the first linker (L-1B), and a second split intein (I-B), wherein upon splicing of the first split intein (I-A) and the second split intein (I-B), a circularized polypeptide chain is produced in which the first linker becomes contiguous. Each light chain and Fc domain in the first polypeptide chain may be associated with a polypeptide chain (e.g., the precursor polypeptide comprises a second polypeptide chain, a third polypeptide chain and a fourth polypeptide chain) comprising a counterpart Fd or Fc domain. In some embodiments, the Fc regions formed by association of the Fc domains on the first polypeptide chain and the counterpart Fc domains are homodimeric. In other embodiments, the Fc regions formed by association of the Fc domains on the first polypeptide chain and the counterpart Fc domains are heterodimeric. For example, in some embodiments, the Fc domains on the first polypeptide are Fc (knob) domains and the counterpart Fc domain are Fc (hole) domains. In other embodiments, the Fc domains on the first polypeptide are Fc (hole) domains and the counterpart Fc domains are Fc (knob) domains.

In some embodiments, the polypeptide chains associated with the first polypeptide chain comprise first fusion partners, e.g., the second polypeptide chain comprises a first fusion partner (FP-1), the third polypeptide chain comprises a second fusion partner (FP-2), and the fourth polypeptide chain comprises a third fusion partner (FP-3). In various embodiments, the fusion partners are at the N-termini of the second, third and fourth polypeptide chains. In other embodiments, the fusion partners are the C-termini of the second, third and fourth polypeptide chains. Suitable fusion partners are described in Section 6.9.

The fusion partner may be separated from the Fd, LC, or Fc on their polypeptide chain by a linker. Thus, the second, third and fourth polypeptide chains may comprise a fourth linker (L-4), a fifth linker (L-5), and a sixth linker (L-6). Suitable linkers for use as L-4, L-5 and L-6 are described in Section 6.7.

Methods of circularizing the precursor molecules are described in Section 6.11.

The present disclosure further provides circularized antibody molecules which are produced by the circularization of the precursor molecules described herein. Thus, the present disclosure provides circularized antibody molecules comprising three antibody fragment domains, such as Fab domains and/or Fc regions, separated by a contiguous L-1 linker, an L-2 linker and an L-3 linker.

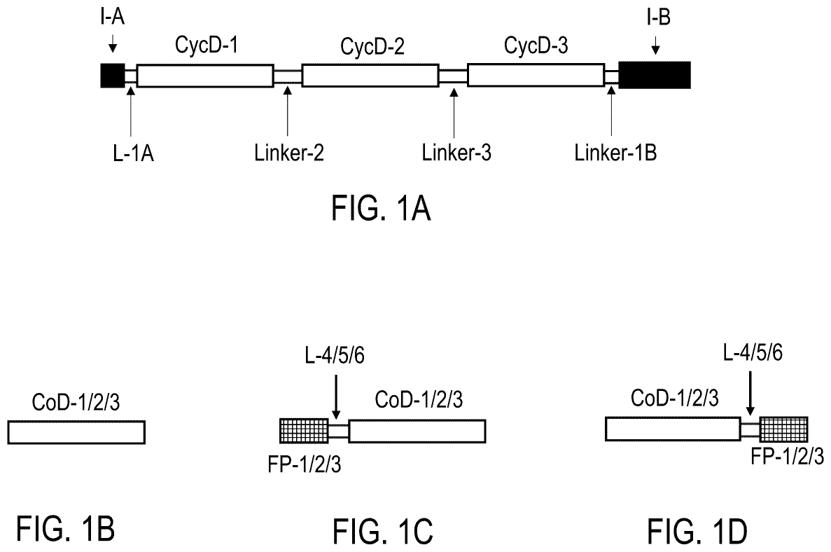

Various permutations of the circularized antibody molecules and their precursor molecules are depicted in FIGS. 1-5.

### 6.3. Fab Domains

Fab domains were traditionally produced by proteolytic cleavage of immunoglobulin molecules using enzymes such as papain. The Fab domains can comprise constant domain and variable region sequences from any suitable species, and thus can be murine, chimeric, human or humanized.

Fab domains typically comprise a CH1 domain attached to a VH domain which pairs with a CL domain attached to a VL domain. In a wild-type immunoglobulin, the VH domain is paired with the VL domain to constitute the Fv region, and the CH1 domain is paired with the CL domain to further stabilize the binding site. A disulfide bond between the two constant domains can further stabilize the Fab domain.

In some embodiments, it is advantageous to use Fab heterodimerization strategies to permit the correct association of Fab domains belonging to the same antigen-binding domain and minimize aberrant pairing of Fab domains belonging to different antigen-binding domains. For example, the Fab heterodimerization strategies shown in Table 1 below can be used:

TABLE 1
Fab Heterodimerization Strategies

| STRATEGY | VH | CH1 | VL | CL | REFERENCE |
|---|---|---|---|---|---|
| CrossMabCH1-CL | WT | CL domain | WT | CH1 domain | Schaefer et al., 2011, |
| | | | | | Cancer Cell 2011; |
| | | | | | 20: 472-86; |
| | | | | | PMID: 22014573. |
| orthogonal Fab | 39K, | H172A, | 1R, 38D, | L135Y, | Lewis et al., 2014, Nat |
| VHVRD1CH1CRD2 - | 62E | F174G | (36F) | S176W | Biotechnol 32: 191-8 |
| VLVRD1CλCRD2 | | | | | |
| orthogonal Fab | 39Y | WT | 38R | WT | Lewis et al., 2014, Nat |
| VHVRD2CH1wt - | | | | | Biotechnol 32: 191-8 |
| VLVRD2Cλwt | | | | | |
| TCR CαCβ | 39K | TCR Cα | 38D | TCR Cβ | Wu et al., 2015, |
| | | | | | MABD 7: 364-76 |
| CR3 | WT | T192E | WT | N137K, | Golay at al., 2016, J |
| | | | | S114A | Immunol 196: 3199-211. |
| MUT4 | WT | L143Q, | WT | V133T, | Golay at al., 2016, J |
| | | S188V | | S176V | Immunol 196: 3199-211. |
| DuetMab | WT | F126C | WT | S121C | Mazor et al., 2015, |
| | | | | | MABD 7: 377-89; Mazor |
| | | | | | et al., 2015, MABD |
| | | | | | 7: 461-669. |
| Domain | WT | CH3 + knob | WT | CH3 + hole | Wozniak-Knopp et al., |
| exchanged | | or hole | | or knob | 2018, PloS ONE13(4): |
| | | mutation | | mutation | e0195442 |

Accordingly, in certain embodiments, correct association between the two polypeptides of a Fab is promoted by exchanging the VL and VH domains of the Fab for each other or exchanging the CH1 and CL domains for each other, e.g., as described in WO 2009/080251.

Correct Fab pairing can also be promoted by introducing one or more amino acid modifications in the CH1 domain and one or more amino acid modifications in the CL domain of the Fab and/or one or more amino acid modifications in the VH domain and one or more amino acid modifications in the VL domain. The amino acids that are modified are typically part of the VH: VL and CH1: CL interface such that the Fab components preferentially pair with each other rather than with components of other Fabs.

In one embodiment, the one or more amino acid modifications are limited to the conserved framework residues of the variable (VH, VL) and constant (CH1, CL) domains as indicated by the Kabat numbering of residues. Almagro, 2008, Frontiers In Bioscience 13:1619-1633 provides a definition of the framework residues on the basis of Kabat, Chothia, and IMGT numbering schemes.

In one embodiment, the modifications introduced in the VH and CH1 and/or VL and CL domains are complementary to each other. Complementarity at the heavy and light chain interface can be achieved on the basis of steric and hydrophobic contacts, electrostatic/charge interactions or a combination of the variety of interactions. The complementarity between protein surfaces is broadly described in the literature in terms of lock and key fit, knob into hole, protrusion and cavity, donor and acceptor, etc., all implying the nature of structural and chemical match between the two interacting surfaces.

In one embodiment, the one or more introduced modifications introduce a new hydrogen bond across the interface of the Fab components. In one embodiment, the one or more introduced modifications introduce a new salt bridge across the interface of the Fab components. Exemplary substitutions are described in WO 2014/150973 and WO 2014/082179, the contents of which are hereby incorporated by reference.

In some embodiments, the Fab domain comprises a 192E substitution in the CH1 domain and 114A and 137K substitutions in the CL domain, which introduces a salt-bridge between the CH1 and CL domains (see, e.g., Golay et al., 2016, J Immunol 196:3199-211).

In some embodiments, the Fab domain comprises a 143Q and 188V substitutions in the CH1 domain and 113T and 176V substitutions in the CL domain, which serves to swap hydrophobic and polar regions of contact between the CH1 and CL domain (see, e.g., Golay et al., 2016, J Immunol 196:3199-211).

In some embodiments, the Fab domain can comprise modifications in some or all of the VH, CH1, VL, CL domains to introduce orthogonal Fab interfaces which promote correct assembly of Fab domains (Lewis et al., 2014, Nature Biotechnology 32:191-198). In an embodiment, 39K, 62E modifications are introduced in the VH domain, H172A, F174G modifications are introduced in the CH1 domain, 1 R, 38D, (36F) modifications are introduced in the VL domain, and L135Y, S176W modifications are introduced in the CL domain. In another embodiment, a 39Y modification is introduced in the VH domain and a 38R modification is introduced in the VL domain.

Fab domains can also be modified to replace the native CH1: CL disulfide bond with an engineered disulfide bond, thereby increasing the efficiency of Fab component pairing. For example, an engineered disulfide bond can be introduced by introducing a 126C in the CH1 domain and a 121 C in the CL domain (see, e.g., Mazor et al., 2015, MABD 7:377-89).

Fab domains can also be modified by replacing the CH1 domain and CL domain with alternative domains that promote correct assembly. For example, Wu et al., 2015, MABD 7:364-76, describes substituting the CH1 domain with the constant domain of the T cell receptor and substituting the CL domain with the b domain of the T cell receptor, and pairing these domain replacements with an additional charge-charge interaction between the VL and VH domains by introducing a 38D modification in the VL domain and a 39K modification in the VH domain.

### 6.4. Fc Regions

The circularized antibody molecules of the disclosure can include pairs of Fc domains that form Fc regions, derived from any suitable species. In some embodiments the Fc domains are operably linked to fusion partners. In some embodiments, the Fc domain is derived from a human Fc domain. In further embodiments, an IgG Fc domain is fused to a fusion partner.

The Fc domains that can be incorporated into circularized antibody molecules can be derived from any suitable class of antibody, including IgA (including subclasses IgA1 and IgA2), IgD, IgE, IgG (including subclasses IgG1, IgG2, IgG3 and IgG4), and IgM. In one embodiment, the Fc domain is derived from IgG1, IgG2, IgG3 or IgG4. In one embodiment, the Fc domain is derived from IgG1. In another embodiment, the Fc domain is derived from IgG4.

In native antibodies, the heavy chain Fc domain of IgA, IgD and IgG is composed of two heavy chain constant domains (CH2 and CH3) and that of IgE and IgM is composed of three heavy chain constant domains (CH2, CH3 and CH4). These dimerize to create an Fc region.

In the circularized antibody molecules of the present disclosure, the Fc region, and/or the Fc domains within it, can comprise heavy chain constant domains from one or more different classes of antibody, for example one, two, or three different classes.

In one embodiment, the Fc region comprises CH2 and CH3 domains derived from IgG1.

In one embodiment, the Fc region comprises CH2 and CH3 domains derived from IgG2.

In one embodiment, the Fc region comprises CH2 and CH3 domains derived from IgG3.

In one embodiment, the Fc region comprises CH2 and CH3 domains derived from IgG4.

In one embodiment the Fc region comprises a CH4 domain from IgM. The IgM CH4 domain is typically located at the C-terminus of the CH3 domain.

In one embodiment the Fc region comprises CH2 and CH3 domains derived from IgG and a CH4 domain derived from IgM.

In some embodiments, the Fc domain includes a CH2 domain and/or a CH3 domain set forth in Table 2 below, and/or a CH2 domain and/or a CH3 domain having at least 90%, at least 95%, at least 97%, at least 98%, or at least 99% sequence identity thereto, e.g., a variant CH2 domain and/or variant CH3 domain at least 90%, at least 95%, at least 97%, at least 98%, or at least 99% sequence identity to a CH2 domain and/or CH3 domain as set forth in Table 2 and one or more substitutions that alter effector function, e.g., as described in Section 6.4.1.1, or permit heterodimerization and/or facilitate purification, e.g., as described in Section 6.4.1.2.

TABLE 2

| Fc Domain | Sequence |
|---|---|
| CH2 | CH2: |
| SEQ ID NO: 10 of | GPSVFLFPPK PKDTLMISRT PEVTCVVVDV SQEDPEVQFN |
| WO2014/121087 | WYVDGVEVHN AKTKPREEQF NSTYRVVSVL TVLHQDWLNG |
| CH3 | KEYKCKVSNK GLPSSIEKTI SKAK (SEQ ID NO: 4) |
| SEQ ID NO: 11 of | CH3: |
| WO2014/121087 | GQPREPQVYT LPPSRDELTK NQVSLTCLVK GFYPSDIAVE |
| | WESNGQPENN YKTTPPVLDS DGSFFLYSKL TVDKSRWQQG |
| | NVFSCSVMHE ALHNHYTQKS LSLSPGK (SEQ ID NO: 5) |
| CH2 | CH2: |
| SEQ ID NO: 10 of | GPSVFLFPPK PKDTLMISRT PEVTCVVVDV SQEDPEVQFN |
| WO2014/121087 | WYVDGVEVHN AKTKPREEQF NSTYRVVSVL TVLHQDWLNG |
| CH3 | KEYKCKVSNK GLPSSIEKTI SKAK (SEQ ID NO: 4) |
| SEQ ID NO: 12 of | CH3: |
| WO2014/121087 | GQPREPQVYT LPPSQEEMTK NQVSLTCLVK GFYPSDIAVE |
| | WESNGQPENN YKTTPPVLDS DGSFFLYSRL TVDKSRWQEG |
| | NVFSCSVMHE ALHNHYTQKS LSLSLGK (SEQ ID NO: 6) |
| CH2 | CH2: |
| SEQ ID NO: 10 of | GPSVFLFPPK PKDTLMISRT PEVTCVVVDV SQEDPEVQFN |
| WO2014/121087 | WYVDGVEVHN AKTKPREEQF NSTYRVVSVL TVLHQDWLNG |
| CH3 | KEYKCKVSNK GLPSSIEKTI SKAK (SEQ ID NO: 4) |
| SEQ ID NO: 41 of | CH3: |
| WO2014/121087 | GQPREPQVYT LPPSRDELTK NQVSLTCLVK GFYPSDIAVE |
| | WESNGQPENN YKTTPPVLDS DGSFFLYSKL TVDKSRWQQG |
| | NVFSCSVMHE ALHNRFTQKS LSLSPGK (SEQ ID NO: 7) |
| CH2 | CH2: |
| SEQ ID NO: 10 of | GPSVFLFPPK PKDTLMISRT PEVTCVVVDV SQEDPEVQFN |
| WO2014/121087 | WYVDGVEVHN AKTKPREEQF NSTYRVVSVL TVLHQDWLNG |
| CH3 | KEYKCKVSNK GLPSSIEKTI SKAK (SEQ ID NO: 4) |
| SEQ ID NO: 42 of | CH3: |
| WO2014/121087 | GQPREPQVYT LPPSQEEMTK NQVSLTCLVK GFYPSDIAVE |
| | WESNGQPENN YKTTPPVLDS DGSFFLYSRL TVDKSRWQEG |
| | NVFSCSVMHE ALHNRFTQKS LSLSLGK (SEQ ID NO: 8) |
| CH2 | CH2: |
| SEQ ID NO: 10 of | GPSVFLFPPK PKDTLMISRT PEVTCVVVDV SQEDPEVQFN |
| WO2014/121087 | WYVDGVEVHN AKTKPREEQF NSTYRVVSVL TVLHQDWLNG |
| CH3 ″knob″ | KEYKCKVSNK GLPSSIEKTI SKAK (SEQ ID NO: 4) |
| | CH3: |
| | GQPREPQVYT LPPSQEEMTK NQVSLWCLVK GFYPSDIAVE |
| | WESNGQPENN YKTTPPVLDS DGSFFLYSRL TVDKSRWQEG |
| | NVFSCSVMHE ALHNHYTQKS LSLSLGK (SEQ ID NO: 9) |
| CH2 | CH2: |
| SEQ ID NO: 10 of | GPSVFLFPPK PKDTLMISRT PEVTCVVVDV SQEDPEVQFN |
| WO2014/121087 | WYVDGVEVHN AKTKPREEQF NSTYRVVSVL TVLHQDWLNG |
| CH3 ″hole″ with | KEYKCKVSNK GLPSSIEKTI SKAK (SEQ ID NO: 4) |
| star mutations | CH3: |
| | GQPREPQVYT LPPSQEEMTK NQVSLSCAVK GFYPSDIAVE |
| | WESNGQPENN YKTTPPVLDS DGSFFLVSRL TVDKSRWQEG |
| | NVFSCSVMHE ALHNRFTQKS LSLSPGK (SEQ ID NO: 10) |
| WT IgG1 CH2 | CH2: |
| WT IgG1 CH3 | GPSVFLFPPK PKDTLMISRT PEVTCVVVDV SHEDPEVKFN |
| | WYVDGVEVHN AKTKPREEQY NSTYRVVSVL TVLHQDWLNG |
| | KEYKCKVSNK ALPAPIEKTI SKAK (SEQ ID NO: 11) |
| | CH3: |
| | GQPREPQVYT LPPSRDELTK NQVSLTCLVK GFYPSDIAVE |
| | WESNGQPENN YKTTPPVLDS DGSFFLYSKL TVDKSRWQQG |
| | NVFSCSVMHE ALHNHYTQKS LSLSPGK (SEQ ID NO: 5) |

The heavy chain constant domains for use in producing an Fc region for the circularized antibody molecules of the present disclosure may include variants of the naturally occurring constant domains described above. Such variants may comprise one or more amino acid variations compared to wildtype constant domains. In one example, the Fc region of the present disclosure comprises at least one constant domain that varies in sequence from the wildtype constant domain. It will be appreciated that the variant constant domains may be longer or shorter than the wildtype constant domain.

Preferably, the variant constant domains are at least 60% identical or similar to a wildtype constant domain. In another example the variant constant domains are at least 70% identical or similar. In another example the variant constant domains are at least 80% identical or similar. In another example the variant constant domains are at least 90% identical or similar. In another example the variant constant domains are at least 95% identical or similar.

The Fc domains that are incorporated into the circularized antibody molecules of the present disclosure may comprise one or more modifications that alter the functional properties of the proteins, for example, binding to Fc-receptors such as FcRn or leukocyte receptors, binding to complement, modified disulfide bond architecture, or altered glycosylation patterns. Exemplary Fc modifications that alter effector function are described in Section 6.4.1.1.

The Fc domains can also be altered to include modifications that improve manufacturability of asymmetric circularized antibody molecules, for example by allowing heterodimerization, which is the preferential pairing of non-identical Fc domains over identical Fc domains. Heterodimerization permits the production of circularized antibody molecules in which different polypeptide components (e.g., different fusion partners) are connected to Fc domains that differ in sequence. Examples of heterodimerization strategies are exemplified in Section 6.4.1.2.

It will be appreciated that any of the modifications mentioned above can be combined in any suitable manner to achieve the desired functional properties and/or combined with other modifications to alter the properties of the circularized antibody molecules.

6.4.1.1. Fc Domains with Altered Effector Function

In some embodiments, the Fc domain comprises one or more amino acid substitutions that reduces binding to an Fc receptor and/or effector function.

In a particular embodiment, the Fc receptor is an Fcγ receptor. In one embodiment, the Fc receptor is a human Fc receptor. In one embodiment, the Fc receptor is an activating Fc receptor. In a specific embodiment, the Fc receptor is an activating human Fcγ receptor, more specifically human FcγRIIIa, FcγRI or FcγRIIa, most specifically human FcγRIIIa. In one embodiment, the effector function is one or more selected from the group of complement dependent cytotoxicity (CDC), antibody-dependent cell-mediated cytotoxicity (ADCC), antibody-dependent cellular phagocytosis (ADCP), and cytokine secretion. In a particular embodiment, the effector function is ADCC.

In one embodiment, the Fc domain (e.g., an Fc domain of a circularized antibody molecule or an Fc region (e.g., a pair of Fc domains of a circularized antibody molecule that form an Fc region) comprises an amino acid substitution at a position selected from the group of E233, L234, L235, N297, P331 and P329 (numberings according to Kabat EU index). In a more specific embodiment, the Fc domain or the Fc region comprises an amino acid substitution at a position selected from the group of L234, L235 and P329 (numberings according to Kabat EU index). In some embodiments, the Fc domain or the Fc region comprises the amino acid substitutions L234A and L235A (numberings according to Kabat EU index). In one such embodiment, the Fc domain or region is an IgD Fc domain or region, particularly a human IgD Fc domain or region. In one embodiment, the Fc domain or the Fc region comprises an amino acid substitution at position P329. In a more specific embodiment, the amino acid substitution is P329A or P329G, particularly P329G (numberings according to Kabat EU index). In one embodiment, the Fc domain or the Fc region comprises an amino acid substitution at position P329 and a further amino acid substitution at a position selected from E233, L234, L235, N297 and P331 (numberings according to Kabat EU index). In a more specific embodiment, the further amino acid substitution is E233P, L234A, L235A, L235E, N297A, N297D or P331S. In particular embodiments, the Fc domain or the Fc region comprises amino acid substitutions at positions P329, L234 and L235 (numberings according to Kabat EU index). In more particular embodiments, the Fc domain comprises the amino acid mutations L234A, L235A and P329G (“P329G LALA”, “PGLALA” or “LALAPG”).

Typically, the same one or more amino acid substitution is present in each of the two Fc domains of an Fc region. Thus, in a particular embodiment, each Fc domain of the Fc region comprises the amino acid substitutions L234A, L235A and P329G (Kabat EU index numbering), i.e. in each of the first and the second Fc domains in the Fc region the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index).

In one embodiment, the Fc domain is an IgG1 Fc domain, particularly a human IgG1 Fc domain. In some embodiments, the IgG1 Fc domain is a variant IgG1 comprising D265A, N297A mutations (EU numbering) to reduce effector function.

In another embodiment, the Fc domain is an IgG4 Fc domain with reduced binding to Fc receptors. In a particular embodiment, the IgG4 with reduced effector function comprises the amino acid sequence of SEQ ID NO:31 of WO2014/121087, sometimes referred to herein as IgG4s or hIgG4s, or the CH2 and/or CH3 portion thereof.

For heterodimeric Fc regions, it is possible to incorporate a combination of the variant IgG4 Fc sequences set forth above, for example an Fc region comprising an Fc domain comprising the amino acid sequence of SEQ ID NO:30 of WO2014/121087 or the CH2 and/or CH3 portion thereof, an Fc domain comprising the amino acid sequence of SEQ ID NO: 37 of WO2014/121087 or the CH2 and/or CH3 portion thereof, an Fc region comprising an Fc domain comprising the amino acid sequence of SEQ ID NO:31 of WO2014/121087 or the CH2 and/or CH3 portion thereof, or an Fc domain comprising the amino acid sequence of SEQ ID NO:38 of WO2014/121087 or the CH2 and/or CH3 portion thereof.

### 6.4.1.2. Fc Heterodimerization Variants

Certain circularized antibody molecules entail dimerization between two Fc domains that, unlike a native immunoglobulin, are operably linked to non-identical N-terminal or C-terminal regions. Inadequate heterodimerization of two Fc domains to form an Fc region can be an obstacle for increasing the yield of desired heterodimeric Fc comprising molecules and represents challenges for purification. A variety of approaches available in the art can be used for enhancing dimerization of Fc domains that might be present in the circularized antibody molecules of the disclosure, for example as disclosed in EP 1870459A1; U.S. Pat. Nos. 5,582,996; 5,731,168; 5,910,573; 5,932,448; 6,833,441; 7,183,076; U.S. Patent Application Publication No. 2006204493A1; and PCT Publication No. WO 2009/089004A1.

In some embodiments, the present disclosure provides circularized antibody molecules comprising Fc heterodimers, i.e., Fc regions comprising heterologous, non-identical Fc domains. Typically, each Fc domain in the Fc heterodimer comprises a CH3 domain of an antibody. The CH3 domains are derived from the constant region of an antibody of any isotype, class, or subclass, and preferably of IgG (IgG1, IgG2, IgG3 and IgG4) class, as described in the preceding section.

Heterodimerization of the two different heavy chains at CH3 domains give rise to the desired circularized antibody molecule, while homodimerization of identical heavy chains will reduce yield of the circularized antibody molecule. Thus, in a preferred embodiment, the polypeptides that associate to form a circularized antibody molecule of the disclosure will contain CH3 domains with modifications that favor heterodimeric association relative to unmodified Fc domains.

In a specific embodiment, said modification promoting the formation of Fc heterodimers is a so-called “knob-into-hole” or “knob-in-hole” modification, comprising a “knob” modification in one of the Fc domains and a “hole” modification in the other Fc domain. The knob-into-hole technology is described e.g., in U.S. Pat. Nos. 5,731,168; 7,695,936; Ridgway et al., 1996, Prot Eng 9:617-621, and Carter, 2001, Immunol Meth 248:7-15. Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine).

Accordingly, in some embodiments, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable. Preferably said amino acid residue having a larger side chain volume is selected from the group consisting of arginine (R), phenylalanine (F), tyrosine (Y), and tryptophan (W). Preferably said amino acid residue having a smaller side chain volume is selected from the group consisting of alanine (A), serine(S), threonine (T), and valine (V). The protuberance and cavity can be made by altering the nucleic acid encoding the polypeptides, e.g., by site-specific mutagenesis, or by peptide synthesis. An exemplary substitution is Y470T.

In a specific such embodiment, in the first Fc domain the threonine residue at position 366 is replaced with a tryptophan residue (T366W), and in the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numbering according to Kabat EU index). In a further embodiment, in the first Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second Fc domain additionally the tyrosine residue at position 349 is replaced by a cysteine residue (Y349C) (numbering according to Kabat EU index). In a particular embodiment, the first Fc domain comprises the amino acid substitutions S354C and T366W, and the second Fc domain comprises the amino acid substitutions Y349C, T366S, L368A and Y407V (numbering according to Kabat EU index).

In some embodiments, electrostatic steering (e.g., as described in Gunasekaran et al., 2010, J Biol Chem 285 (25): 19637-46) can be used to promote the association of the first and the second Fc domains of the Fc region.

As an alternative, or in addition, to the use of Fc domains that are modified to promote heterodimerization, an Fc domain can be modified to allow a purification strategy that enables selections of Fc heterodimers. In one such embodiment, one polypeptide comprises a modified Fc domain that abrogates its binding to Protein A, thus enabling a purification method that yields a heterodimeric protein. See, for example, U.S. Pat. No. 8,586,713. As such, the circularized antibody molecule can comprise a first CH3 domain and a second CH3 domain, wherein the first and second CH3 domains differ from one another by at least one amino acid, and wherein at least one amino acid difference reduces binding of the circularized antibody molecule to Protein A as compared to a corresponding circularized antibody molecule lacking the amino acid difference. In one embodiment, the first CH3 domain binds Protein A and the second CH3 domain contains a mutation/modification that reduces or abolishes Protein A binding such as an H95R modification (by IMGT exon numbering; H435R by EU numbering). The second CH3 may further comprise a Y96F modification (by IMGT; Y436F by EU). This class of modifications is referred to herein as “star” mutations.

In some embodiments, the Fc region can contain one or more mutations (e.g., knob and hole mutations) to facilitate heterodimerization as well as star mutations to facilitate purification.

### 6.5. Primary Target Molecules

The circularized antibody molecules of the disclosure can bind to target molecules. Non-limiting examples of primary target molecules that can be targeted with circularized antibody molecules include coronavirus spike proteins (e.g., SARS-CoV, SARS-CoV2, MERS-CoV, 229E, NL63, OC43, HKU1, etc.), hemagglutinin (HA), and TNF superfamily receptors (e.g., 4-1BB, OX40, CD40, trail receptor, etc.).

In some embodiments, the circularized antibody molecules of the disclosure bind to target molecules via Fab domains. In other embodiments, the circularized antibody molecules bind to target molecules via fusion partners, which are described in Section 6.9. In yet other embodiments, the circularized antibody molecules bind to target molecules via both their Fab domains and their fusion partners.

### 6.5.1. Coronavirus Spike Proteins

In some embodiments, the circularized antibody molecules comprise one, two or three Fab domains that binds to a spike protein of a coronavirus, e.g., SARS-CoV2. In some embodiments, all three Fab domains are identical or comprise identical CDR sequences.

In some embodiments, the Fab domain competes with an antibody having the sequence set forth in Table 3 below for binding to spike protein and/or comprises binding portions of an exemplary antibody or an antibody having an antibody sequence set forth in Table 3. In some aspects, the Fab competes with an antibody set forth in Table 1 for binding to a spike protein. In further aspects, the Fab comprises CDRs having CDR sequences of an antibody set forth in Table 3. In some embodiments, the Fab comprises all 6 CDR sequences of the antibody set forth in Table 3. In other embodiments, the Fab comprises at least the heavy chain CDR sequences (CDR-H1, CDR-H2, CDR-H3 and the light chain CDR sequences of a universal light chain. In further aspects, a Fab comprises a VH comprising the amino acid sequence of the VH of an antibody set forth in Table 3. In some embodiments, the Fab further comprises a VL comprising the amino acid sequence of the VL of the antibody set forth in Table 3. In other embodiments, the Fab further comprises a universal light chain VL sequence.

TABLE 3
Exemplary Spike Protein Antibodies
and Antigen-Binding Domains

| | | Blocking/ |
|---|---|---|
| | | Non- |
| | | blocking |
| | | ACE2 |
| Target/Epitope | Antibody Name and/or Binding Sequences | binding |
| SARS-CoV-2 | P5-22 | Blocking |
| Spike protein | VH: SEQ ID NO: 2 of PCT Publication | |
| | No: WO 2022/052968 A1 | |
| | VL: SEQ ID NO: 4 of PCT Publication | |
| | No: WO 2022/052968 A1 | |
| SARS-CoV-2 | P14-44 | Blocking |
| Spike protein | VH: SEQ ID NO: 6 of PCT Publication | |
| | No: WO 2022/052968 A1 | |
| | VL: SEQ ID NO: 8 of PCT Publication | |
| | No: WO 2022/052968 A1 | |
| SARS-CoV-2 | P15-16 | Blocking |
| Spike protein | VH: SEQ ID NO: 10 of PCT Publication | |
| | No: WO 2022/052968 A1 | |
| | VL: SEQ ID NO: 12 of PCT Publication | |
| | No: WO 2022/052968 A1 | |
| SARS-CoV-2 | P10-20 | Blocking |
| Spike protein | VH: SEQ ID NO: 14 of PCT Publication | |
| | No: WO 2022/052968 A1 | |
| | VL: SEQ ID NO: 16 of PCT Publication | |
| | No: WO 2022/052968 A1 | |
| SARS-CoV-2 | P14-37 | Blocking |
| Spike protein | VH: SEQ ID NO: 18 of PCT Publication | |
| | No: WO 2022/052968 A1 | |
| | VL: SEQ ID NO: 20 of PCT Publication | |
| | No: WO 2022/052968 A1 | |
| SARS-CoV-2 | P23-29 | Blocking |
| Spike protein | VH: SEQ ID NO: 22 of PCT Publication | |
| | No: WO 2022/052968 A1 | |
| | VL: SEQ ID NO: 24 of PCT Publication | |
| | No: WO 2022/052968 A1 | |
| SARS-CoV-2 | P3-11 | Blocking |
| Spike protein | VH: SEQ ID NO: 26 of PCT Publication | |
| | No: WO 2022/052968 A1 | |
| | VL: SEQ ID NO: 28 of PCT Publication | |
| | No: WO 2022/052968 A1 | |
| aa 319-532 | P16-A3 | Blocking |
| of S-protein | VH: SEQ ID NO: 17 of PCT Publication | |
| RBD domain | No: WO 2021/196268 A1 | |
| | VL: SEQ ID NO: 18 of PCT Publication | |
| | No: WO 2021/196268 A1 | |
| aa 319-532 | P17-A11 | Blocking |
| of S-protein | VH: SEQ ID NO: 19 of PCT Publication | |
| RBD domain | No: WO 2021/196268 A1 | |
| | VL: SEQ ID NO: 20 of PCT Publication | |
| | No: WO 2021/196268 A1 | |
| aa 319-532 | R15-F7 | Blocking |
| of S-protein | VH: SEQ ID NO: 1 of PCT Publication | |
| RBD domain | No: WO 2022/104918 A1 | |
| | VL: SEQ ID NO: 2 of PCT Publication | |
| | No: WO 2022/104918 A1 | |
| S-protein | DH1041 | Blocking |
| RBD domain | VH: SEQ ID NO: 1100 of PCT Publication | |
| | No: WO 2022/060906 A1 | |
| | VL: SEQ ID NO: 1102 of PCT Publication | |
| | No: WO 2022/060906 A1 | |
| aa 481-491 | DH1042 | Blocking |
| of S-protein | VH: SEQ ID NO: 1104 of PCT Publication | |
| RBD domain | No: WO 2022/060906 A1 | |
| | VL: SEQ ID NO: 1106 of PCT Publication | |
| | No: WO 2022/060906 A1 | |
| S-protein | DH1043 | Blocking |
| RBD domain | VH: SEQ ID NO: 1108 of PCT Publication | |
| | No: WO 2022/060906 A1 | |
| | VL: SEQ ID NO: 1110 of PCT Publication | |
| | No: WO 2022/060906 A1 | |
| S-protein | DH1044 | Non- |
| RBD domain | VH: SEQ ID NO: 1112 of PCT Publication | blocking |
| | No: WO 2022/060906 A1 | |
| | VL: SEQ ID NO: 1114 of PCT Publication | |
| | No: WO 2022/060906 A1 | |
| S-protein | DH1221 | Blocking |
| RBD domain | VH: SEQ ID NO: 1214 of PCT Publication | |
| | No: WO 2022/060906 A1 | |
| | VL: SEQ ID NO: 1226 of PCT Publication | |
| | No: WO 2022/060906 A1 | |
| S-protein | DH1222 | Blocking |
| RBD domain | VH: SEQ ID NO: 1216 of PCT Publication | |
| | No: WO 2022/060906 A1 | |
| | VL: SEQ ID NO: 1228 of PCT Publication | |
| | No: WO 2022/060906 A1 | |
| S-protein | DH1223 | Blocking |
| RBD domain | VH: SEQ ID NO: 1218 of PCT Publication | |
| | No: WO 2022/060906 A1 | |
| | VL: SEQ ID NO: 1230 of PCT Publication | |
| | No: WO 2022/060906 A1 | |
| S-protein | DH1224 | Blocking |
| RBD domain | VH: SEQ ID NO: 1220 of PCT Publication | |
| | No: WO 2022/060906 A1 | |
| | VL: SEQ ID NO: 1232 of PCT Publication | |
| | No: WO 2022/060906 A1 | |
| S-protein | DH1225 | Blocking |
| RBD domain | VH: SEQ ID NO: 1222 of PCT Publication | |
| | No: WO 2022/060906 A1 | |
| | VL: SEQ ID NO: 1234 of PCT Publication | |
| | No: WO 2022/060906 A1 | |
| S-protein | DH1226 | Blocking |
| RBD domain | VH: SEQ ID NO: 1224 of PCT Publication | |
| | No: WO 2022/060906 A1 | |
| | VL: SEQ ID NO: 1236 of PCT Publication | |
| | No: WO 2022/060906 A1 | |
| S-protein | mAb S1D2 | Blocking |
| RBD domain | VH: SEQ ID NO: 6 of PCT Publication | |
| SEQ ID NO: | No: WO 2022/032139 A1 | |
| 5 of PCT | VL: SEQ ID NO: 7 of PCT Publication | |
| WO 2022/ | No: WO 2022/032139 A1 | |
| 032139 A1 | | |
| S-protein | mAb S1D7270 | Blocking |
| RBD domain | VH: SEQ ID NO: 28 of PCT Publication | |
| SEQ ID NO: | No: WO 2022/032139 A1 | |
| 5 of PCT | VL: SEQ ID NO: 29 of PCT Publication | |
| WO 2022/ | No: WO 2022/032139 A1 | |
| aa 331-524 | 413-2 | |
| of S-protein | VH: SEQ ID NO: 15 of PCT Publication | |
| RBD domain | No: WO 2021/248279 A1 | |
| SEQ ID | VL: SEQ ID NO: 43 of PCT Publication | |
| NO: 61 | No: WO 2021/248279 A1 | |
| aa 331-524 | 413-3 | |
| of S-protein | VH: SEQ ID NO: 16 of PCT Publication | |
| RBD domain | No: WO 2021/248279 A1 | |
| SEQ ID | VL: SEQ ID NO: 44 of PCT Publication | |
| NO: 61 | No: WO 2021/248279 A1 | |
| aa 331-524 | 505-1 | |
| of S-protein | VH: SEQ ID NO: 17 of PCT Publication | |
| RBD domain | No: WO 2021/248279 A1 | |
| SEQ ID | VL: SEQ ID NO: 45 of PCT Publication | |
| NO: 61 | No: WO 2021/248279 A1 | |
| aa 331-524 | 505-3 | |
| of S-protein | VH: SEQ ID NO: 18 of PCT Publication | |
| RBD domain | No: WO 2021/248279 A1 | |
| SEQ ID | VL: SEQ ID NO: 46 of PCT Publication | |
| NO: 61 | No: WO 2021/248279 A1 | |
| aa 331-524 | 505-5 | |
| of S-protein | VH: SEQ ID NO: 19 of PCT Publication | |
| RBD domain | No: WO 2021/248279 A1 | |
| SEQ ID | VL: SEQ ID NO: 47 of PCT Publication | |
| NO: 61 | No: WO 2021/248279 A1 | |
| aa 331-524 | 515-5 | |
| of S-protein | VH: SEQ ID NO: 20 of PCT Publication | |
| RBD domain | No: WO 2021/248279 A1 | |
| SEQ ID | VL: SEQ ID NO: 48 of PCT Publication | |
| NO: 61 | No: WO 2021/248279 A1 | |
| aa 331-524 | 553-13 | |
| of S-protein | VH: SEQ ID NO: 21 of PCT Publication | |
| RBD domain | No: WO 2021/248279 A1 | |
| SEQ ID | VL: SEQ ID NO: 49 of PCT Publication | |
| NO: 61 | No: WO 2021/248279 A1 | |
| aa 331-524 | 553-15 | |
| of S-protein | VH: SEQ ID NO: 22 of PCT Publication | |
| RBD domain | No: WO 2021/248279 A1 | |
| SEQ ID | VL: SEQ ID NO: 50 of PCT Publication | |
| NO: 61 | No: WO 2021/248279 A1 | |
| aa 331-524 | 553-17 | |
| of S-protein | VH: SEQ ID NO: 23 of PCT Publication | |
| RBD domain | No: WO 2021/248279 A1 | |
| SEQ ID | VL: SEQ ID NO: 51 of PCT Publication | |
| NO: 61 | No: WO 2021/248279 A1 | |
| aa 331-524 | 553-18 | |
| of S-protein | VH: SEQ ID NO: 24 of PCT Publication | |
| RBD domain | No: WO 2021/248279 A1 | |
| SEQ ID | VL: SEQ ID NO: 52 of PCT Publication | |
| NO: 61 | No: WO 2021/248279 A1 | |
| aa 331-524 | 553-20 | |
| of S-protein | VH: SEQ ID NO: 25 of PCT Publication | |
| RBD domain | No: WO 2021/248279 A1 | |
| SEQ ID | VL: SEQ ID NO: 53 of PCT Publication | |
| NO: 61 | No: WO 2021/248279 A1 | |
| aa 331-524 | 553-27 | |
| of S-protein | VH: SEQ ID NO: 26 of PCT Publication | |
| RBD domain | No: WO 2021/248279 A1 | |
| SEQ ID | VL: SEQ ID NO: 54 of PCT Publication | |
| NO: 61 | No: WO 2021/248279 A1 | |
| aa 331-524 | 553-60 | |
| of S-protein | VH: SEQ ID NO: 27 of PCT Publication | |
| RBD domain | No: WO 2021/248279 A1 | |
| SEQ ID | VL: SEQ ID NO: 55 of PCT Publication | |
| NO: 61 | No: WO 2021/248279 A1 | |
| aa 331-524 | 553-63 | |
| of S-protein | VH: SEQ ID NO: 28 of PCT Publication | |
| RBD domain | No: WO 2021/248279 A1 | |
| SEQ ID | VL: SEQ ID NO: 56 of PCT Publication | |
| NO: 61 | No: WO 2021/248279 A1 | |
| SARS-CoV-2 | N3-1 | |
| Spike protein | VH: SEQ ID NO: 1 of PCT Publication | |
| RBD | No: WO 2022/192661 A1 | |
| | VL: SEQ ID NO: 2 of PCT Publication | |
| | No: WO 2022/192661 A1 | |
| SARS-CoV-2 | N3-3 | |
| Spike protein | VH: SEQ ID NO: 1 of PCT Publication | |
| RBD | No: WO 2022/192661 A1 | |
| | VL: SEQ ID NO: 15 of PCT Publication | |
| | No: WO 2022/192661 A1 | |
| SARS-CoV-2 | 1D4 | |
| Spike protein | VH: SEQ ID NO: 18 of PCT Publication | |
| RBD | No: WO 2022/192661 A1 | |
| | VL: SEQ ID NO: 19 of PCT Publication | |
| | No: WO 2022/192661 A1 | |
| SARS-CoV-2 | N3-7 | |
| Spike protein | VH: SEQ ID NO: 1 of PCT Publication | |
| RBD | No: WO 2022/192661 A1 | |
| | VL: SEQ ID NO: 10 of PCT Publication | |
| | No: WO 2022/192661 A1 | |
| SARS-CoV-2 | N3-63A | |
| Spike protein | VH: SEQ ID NO: 1 of PCT Publication | |
| RBD | No: WO 2022/192661 A1 | |
| | VL: SEQ ID NO: 21 of PCT Publication | |
| | No: WO 2022/192661 A1 | |
| SARS-CoV-2 | N3-63B | |
| Spike protein | VH: SEQ ID NO: 1 of PCT Publication | |
| RBD | No: WO 2022/192661 A1 | |
| | VL: SEQ ID NO: 27 of PCT Publication | |
| | No: WO 2022/192661 A1 | |
| SARS-CoV-2 | N3-63C | |
| Spike protein | VH: SEQ ID NO: 1 of PCT Publication | |
| RBD | No: WO 2022/192661 A1 | |
| | VL: SEQ ID NO: 37 of PCT Publication | |
| | No: WO 2022/192661 A1 | |
| SARS-CoV-2 | 8B5 | |
| Spike protein | VH: SEQ ID NO: 22 of PCT Publication | |
| RBD | No: WO 2022/192661 A1 | |
| | VL: SEQ ID NO: 23 of PCT Publication | |
| | No: WO 2022/192661 A1 | |
| SARS-CoV-2 | 4A5 | |
| Spike protein | VH: SEQ ID NO: 24 of PCT Publication | |
| RBD | No: WO 2022/192661 A1 | |
| | VL: SEQ ID NO: 23 of PCT Publication | |
| | No: WO 2022/192661 A1 | |
| SARS-CoV-2 | 1D1 | |
| Spike protein | VH: SEQ ID NO: 18 of PCT Publication | |
| RBD | No: WO 2022/192661 A1 | |
| | VL: SEQ ID NO: 2 of PCT Publication | |
| | No: WO 2022/192661 A1 | |
| SARS-CoV-2 | 1D9 | |
| Spike protein | VH: SEQ ID NO: 18 of PCT Publication | |
| RBD | No: WO 2022/192661 A1 | |
| | VL: SEQ ID NO: 26 of PCT Publication | |
| | No: WO 2022/192661 A1 | |
| SARS-CoV-2 | 4A7 | |
| Spike protein | VH: SEQ ID NO: 24 of PCT Publication | |
| RBD | No: WO 2022/192661 A1 | |
| | VL: SEQ ID NO: 10 of PCT Publication | |
| | No: WO 2022/192661 A1 | |
| SARS-CoV-2 | 1D5 | |
| Spike protein | VH: SEQ ID NO: 18 of PCT Publication | |
| RBD | No: WO 2022/192661 A1 | |
| | VL: SEQ ID NO: 23 of PCT Publication | |
| | No: WO 2022/192661 A1 | |

In some embodiments, the Fabs comprise an amino acid sequence or are encoded by a nucleotide sequence set forth in Table 4 below. In particular aspects, the Fab comprises both heavy and light chain CDRs of an antibody set forth in Table 4 below. In other embodiments, the Fab comprises at least the heavy chain CDR sequences and the light chain CDR sequences of a universal light chain. In further aspects, a Fab comprises a VH having the amino acid sequence of the VH of an antibody set forth in Table 4 and a VL having the amino acid sequence of the VL of the same antibody as set forth in Table 4. In other aspects, a Fab comprises a VH having the amino acid sequence of the VH of an antibody set forth in Table 4 and a universal light chain VL sequence.

TABLE 4

| Antibody | Component | |
|---|---|---|
| Designation | Part | Sequence |
| mAb10933 | Amino Acids | |
| | VH | QVQLVESGGGLVKPGGSLRLSCAASGFTFSDYYMSWIRQAP |
| | | GKGLEWVSYITYSGSTIYYADSVKGRFTISRDNAKSSLYLQ |
| | | MNSLRAEDTAVYYCARDRGTTMVPFDYWGQGTLVTVSS |
| | | (SEQ ID NO: 12) |
| | CDR-H1 | GFTFSDYY (SEQ ID NO: 13) |
| | CDR-H2 | ITYSGSTI (SEQ ID NO: 14) |
| | CDR-H3 | ARDRGTTMVPFDY (SEQ ID NO: 15) |
| | VL | DIQMTQSPSSLSASVGDRVTITCQASQDITNYLNWYQQKPG |
| | | KAPKLLIYAASNLETGVPSRFSGSGSGTDFTFTISGLQPED |
| | | IATYYCQQYDNLPLTFGGGTKVEIK (SEQ ID NO: 16) |
| | CDR-L1 | QDITNY (SEQ ID NO: 17) |
| | CDR-L2 | AAS |
| | CDR-L3 | QQYDNLPLT (SEQ ID NO: 18) |
| | HC | QVQLVESGGGLVKPGGSLRLSCAASGFTFSDYYMSWIRQAP |
| | | GKGLEWVSYITYSGSTIYYADSVKGRFTISRDNAKSSLYLQ |
| | | MNSLRAEDTAVYYCARDRGTTMVPFDYWGQGTLVTVSSAST |
| | | KGPSVFPLAPSSKSTSGGTAALGCLVKDYFPEPVTVSWNSG |
| | | ALTSGVHTFPAVLQSSGLYSLSSVVTVPSSSLGTQTYICNV |
| | | NHKPSNTKVDKKVEPKSCDKTHTCPPCPAPELLGGPSVFLF |
| | | PPKPKDTLMISRTPEVTCVVVDVSHEDPEVKFNWYVDGVEV |
| | | HNAKTKPREEQYNSTYRVVSVLTVLHQDWLNGKEYKCKVSN |
| | | KALPAPIEKTISKAKGQPREPQVYTLPPSRDELTKNQVSLT |
| | | CLVKGFYPSDIAVEWESNGQPENNYKTTPPVLDSDGSFFLY |
| | | SKLTVDKSRWQQGNVFSCSVMHEALHNHYTQKSLSLSPGK |
| | | (SEQ ID NO: 19) |
| | LC | DIQMTQSPSSLSASVGDRVTITCQASQDITNYLNWYQQKPG |
| | | KAPKLLIYAASNLETGVPSRFSGSGSGTDFTFTISGLQPED |
| | | IATYYCQQYDNLPLTFGGGTKVEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC (SEQ ID NO: 20) |
| | VH | CAGGTGCAGCTGGTGGAGTCTGGGGGAGGCTTGGTCAAGCC |
| | | TGGAGGGTCCCTGAGACTCTCCTGTGCAGCCTCTGGATTCA |
| | | CCTTCAGTGACTACTACATGAGCTGGATCCGCCAGGCTCCA |
| | | GGGAAGGGGCTGGAGTGGGTTTCATACATTACTTATAGTGG |
| | | TAGTACCATATACTACGCAGACTCTGTGAAGGGCCGATTCA |
| | | CCATCTCCAGGGACAACGCCAAGAGCTCACTGTATCTGCAA |
| | | ATGAACAGCCTGAGAGCCGAGGACACGGCCGTGTATTACTG |
| | | TGCGAGAGATCGCGGTACAACTATGGTCCCCTTTGACTACT |
| | | GGGGCCAGGGAACCCTGGTCACCGTCTCCTCA (SEQ ID |
| | | NO: 21) |
| | CDR-H1 | GGATTCACCTTCAGTGACTACTAC (SEQ ID NO: 22) |
| | CDR-H2 | ATTACTTATAGTGGTAGTACCATA (SEQ ID NO: 23) |
| | CDR-H3 | GCGAGAGATCGCGGTACAACTATGGTCCCCTTTGACTAC |
| | | (SEQ ID NO: 24) |
| | VL | GACATCCAGATGACCCAGTCTCCATCCTCCCTGTCTGCATC |
| | | TGTAGGAGACAGAGTCACCATCACTTGCCAGGCGAGTCAGG |
| | | ACATTACCAACTATTTAAATTGGTATCAGCAGAAACCAGGG |
| | | AAAGCCCCTAAGCTCCTGATCTACGCTGCATCCAATTTGGA |
| | | AACAGGGGTCCCATCAAGGTTCAGTGGAAGTGGATCTGGGA |
| | | CAGATTTTACTTTCACCATCAGCGGCCTGCAGCCTGAAGAT |
| | | ATTGCAACATATTACTGTCAACAGTATGATAATCTCCCTCT |
| | | CACTTTCGGCGGAGGGACCAAGGTGGAGATCAAA (SEQ |
| | | ID NO: 25) |
| | CDR-L1 | CAGGACATTACCAACTAT (SEQ ID NO: 26) |
| | CDR-L2 | GCTGCATCC |
| | CDR-L3 | CAACAGTATGATAATCTCCCTCTCACT (SEQ ID NO: |
| | | 27) |
| | HC | CAGGTGCAGCTGGTGGAGTCTGGGGGAGGCTTGGTCAAGCC |
| | | TGGAGGGTCCCTGAGACTCTCCTGTGCAGCCTCTGGATTCA |
| | | CCTTCAGTGACTACTACATGAGCTGGATCCGCCAGGCTCCA |
| | | GGGAAGGGGCTGGAGTGGGTTTCATACATTACTTATAGTGG |
| | | TAGTACCATATACTACGCAGACTCTGTGAAGGGCCGATTCA |
| | | CCATCTCCAGGGACAACGCCAAGAGCTCACTGTATCTGCAA |
| | | ATGAACAGCCTGAGAGCCGAGGACACGGCCGTGTATTACTG |
| | | TGCGAGAGATCGCGGTACAACTATGGTCCCCTTTGACTACT |
| | | GGGGCCAGGGAACCCTGGTCACCGTCTCCTCAGCCTCCACC |
| | | AAGGGCCCATCGGTCTTCCCCCTGGCACCCTCCTCCAAGAG |
| | | CACCTCTGGGGGCACAGCGGCCCTGGGCTGCCTGGTCAAGG |
| | | ACTACTTCCCCGAACCGGTGACGGTGTCGTGGAACTCAGGC |
| | | GCCCTGACCAGCGGCGTGCACACCTTCCCGGCTGTCCTACA |
| | | GTCCTCAGGACTCTACTCCCTCAGCAGCGTGGTGACCGTGC |
| | | CCTCCAGCAGCTTGGGCACCCAGACCTACATCTGCAACGTG |
| | | AATCACAAGCCCAGCAACACCAAGGTGGACAAGAAAGTTGA |
| | | GCCCAAATCTTGTGACAAAACTCACACATGCCCACCGTGCC |
| | | CAGCACCTGAACTCCTGGGGGGACCGTCAGTCTTCCTCTTC |
| | | CCCCCAAAACCCAAGGACACCCTCATGATCTCCCGGACCCC |
| | | TGAGGTCACATGCGTGGTGGTGGACGTGAGCCACGAAGACC |
| | | CTGAGGTCAAGTTCAACTGGTACGTGGACGGCGTGGAGGTG |
| | | CATAATGCCAAGACAAAGCCGCGGGAGGAGCAGTACAACAG |
| | | CACGTACCGTGTGGTCAGCGTCCTCACCGTCCTGCACCAGG |
| | | ACTGGCTGAATGGCAAGGAGTACAAGTGCAAGGTCTCCAAC |
| | | AAAGCCCTCCCAGCCCCCATCGAGAAAACCATCTCCAAAGC |
| | | CAAAGGGCAGCCCCGAGAACCACAGGTGTACACCCTGCCCC |
| | | CATCCCGGGATGAGCTGACCAAGAACCAGGTCAGCCTGACC |
| | | TGCCTGGTCAAAGGCTTCTATCCCAGCGACATCGCCGTGGA |
| | | GTGGGAGAGCAATGGGCAGCCGGAGAACAACTACAAGACCA |
| | | CGCCTCCCGTGCTGGACTCCGACGGCTCCTTCTTCCTCTAC |
| | | AGCAAGCTCACCGTGGACAAGAGCAGGTGGCAGCAGGGGAA |
| | | CGTCTTCTCATGCTCCGTGATGCATGAGGCTCTGCACAACC |
| | LC | ACTACACGCAGAAGTCCCTCTCCCTGTCTCCGGGTAAATGA |
| | | (SEQ ID NO: 28) |
| | | GACATCCAGATGACCCAGTCTCCATCCTCCCTGTCTGCATC |
| | | TGTAGGAGACAGAGTCACCATCACTTGCCAGGCGAGTCAGG |
| | | ACATTACCAACTATTTAAATTGGTATCAGCAGAAACCAGGG |
| | | AAAGCCCCTAAGCTCCTGATCTACGCTGCATCCAATTTGGA |
| | | AACAGGGGTCCCATCAAGGTTCAGTGGAAGTGGATCTGGGA |
| | | CAGATTTTACTTTCACCATCAGCGGCCTGCAGCCTGAAGAT |
| | | ATTGCAACATATTACTGTCAACAGTATGATAATCTCCCTCT |
| | | CACTTTCGGCGGAGGGACCAAGGTGGAGATCAAACGAACTG |
| | | TGGCTGCACCATCTGTCTTCATCTTCCCGCCATCTGATGAG |
| | | CAGTTGAAATCTGGAACTGCCTCTGTTGTGTGCCTGCTGAA |
| | | TAACTTCTATCCCAGAGAGGCCAAAGTACAGTGGAAGGTGG |
| | | ATAACGCCCTCCAATCGGGTAACTCCCAGGAGAGTGTCACA |
| | | GAGCAGGACAGCAAGGACAGCACCTACAGCCTCAGCAGCAC |
| | | CCTGACGCTGAGCAAAGCAGACTACGAGAAACACAAAGTCT |
| | | ACGCCTGCGAAGTCACCCATCAGGGCCTGAGCTCGCCCGTC |
| | | ACAAAGAGCTTCAACAGGGGAGAGTGTTAG (SEQ ID |
| | | NO: 29) |
| mAb10934 | VH | EVQLVESGGGLVKPGGSLRLSCAASGITFSNAWMSWVRQAP |
| | | GKGLEWVGRIKSKTDGGTTDYAAPVKGRFTISRDDSKNTLY |
| | | LQMNSLKTEDTAVYYCTTARWDWYFDLWGRGTLVTVSS |
| | | (SEQ ID NO: 30) |
| | CDR-H1 | GITFSNAW (SEQ ID NO: 31) |
| | CDR-H2 | IKSKTDGGTT (SEQ ID NO: 32) |
| | CDR-H3 | TTARWDWYFDL (SEQ ID NO: 33) |
| | VL | DIQMTQSPSSLSASVGDRVTITCQASQDIWNYINWYQQKPG |
| | | KAPKLLIYDASNLKTGVPSRFSGSGSGTDFTFTISSLQPED |
| | | IATYYCQQHDDLPPTFGQGTKVEIK (SEQ ID NO: 34) |
| | CDR-L1 | QDIWNY (SEQ ID NO: 35) |
| | CDR-L2 | DAS |
| | CDR-L3 | QQHDDLPPT (SEQ ID NO: 36) |
| | HC | EVQLVESGGGLVKPGGSLRLSCAASGITESNAWMSWVRQAP |
| | | GKGLEWVGRIKSKTDGGTTDYAAPVKGRFTISRDDSKNTLY |
| | | LQMNSLKTEDTAVYYCTTARWDWYFDLWGRGTLVTVSSAST |
| | | KGPSVFPLAPSSKSTSGGTAALGCLVKDYFPEPVTVSWNSG |
| | | ALTSGVHTFPAVLQSSGLYSLSSVVTVPSSSLGTQTYICNV |
| | | NHKPSNTKVDKKVEPKSCDKTHTCPPCPAPELLGGPSVFLF |
| | | PPKPKDTLMISRTPEVTCVVVDVSHEDPEVKFNWYVDGVEV |
| | | HNAKTKPREEQYNSTYRVVSVLTVLHQDWLNGKEYKCKVSN |
| | | KALPAPIEKTISKAKGQPREPQVYTLPPSRDELTKNQVSLT |
| | | CLVKGFYPSDIAVEWESNGQPENNYKTTPPVLDSDGSFFLY |
| | | SKLTVDKSRWQQGNVFSCSVMHEALHNHYTQKSLSLSPGK |
| | | (SEQ ID NO: 37) |
| | LC | DIQMTQSPSSLSASVGDRVTITCQASQDIWNYINWYQQKPG |
| | | KAPKLLIYDASNLKTGVPSRFSGSGSGTDFTFTISSLQPED |
| | | IATYYCQQHDDLPPTFGQGTKVEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC (SEQ ID NO: 38) |
| | VH | GAGGTGCAGCTGGTGGAGTCTGGGGGAGGCTTGGTAAAGCC |
| | | TGGGGGGTCCCTTAGACTCTCCTGTGCAGCCTCTGGAATCA |
| | | CTTTCAGTAACGCCTGGATGAGTTGGGTCCGCCAGGCTCCA |
| | | GGGAAGGGGCTGGAGTGGGTTGGCCGTATTAAAAGCAAAAC |
| | | TGATGGTGGGACAACAGACTACGCCGCACCCGTGAAAGGCA |
| | | GATTCACCATCTCAAGAGATGATTCAAAAAACACGCTGTAT |
| | | CTACAAATGAACAGCCTGAAAACCGAGGACACAGCCGTGTA |
| | | TTACTGTACCACAGCGAGGTGGGACTGGTACTTCGATCTCT |
| | | GGGGCCGTGGCACCCTGGTCACTGTCTCCTCA (SEQ ID |
| | | NO: 39) |
| | CDR-H1 | GGAATCACTTTCAGTAACGCCTGG (SEQ ID NO: 40) |
| | CDR-H2 | ATTAAAAGCAAAACTGATGGTGGGACAACA (SEQ ID |
| | | NO: 41) |
| | CDR-H3 | ACCACAGCGAGGTGGGACTGGTACTTCGATCTC (SEQ ID |
| | | NO: 42) |
| | VL | GACATCCAGATGACCCAGTCTCCATCCTCCCTGTCTGCATC |
| | | TGTAGGAGACAGAGTCACCATCACTTGCCAGGCGAGTCAGG |
| | | ACATTTGGAATTATATAAATTGGTATCAGCAGAAACCAGGG |
| | | AAGGCCCCTAAGCTCCTGATCTACGATGCATCCAATTTGAA |
| | | AACAGGGGTCCCATCAAGGTTCAGTGGAAGTGGATCTGGGA |
| | | CAGATTTTACTTTCACCATCAGCAGCCTGCAGCCTGAAGAT |
| | | ATTGCAACATATTACTGTCAACAGCATGATGATCTCCCTCC |
| | | GACCTTCGGCCAAGGGACCAAGGTGGAAATCAAA (SEQ |
| | | ID NO: 43) |
| | CDR-L1 | CAGGACATTTGGAATTAT (SEQ ID NO: 44) |
| | CDR-L2 | GATGCATCC |
| | CDR-L3 | CAACAGCATGATGATCTCCCTCCGACC (SEQ ID NO: |
| | | 45) |
| | HC | GAGGTGCAGCTGGTGGAGTCTGGGGGAGGCTTGGTAAAGCC |
| | | TGGGGGGTCCCTTAGACTCTCCTGTGCAGCCTCTGGAATCA |
| | | CTTTCAGTAACGCCTGGATGAGTTGGGTCCGCCAGGCTCCA |
| | | GGGAAGGGGCTGGAGTGGGTTGGCCGTATTAAAAGCAAAAC |
| | | TGATGGTGGGACAACAGACTACGCCGCACCCGTGAAAGGCA |
| | | GATTCACCATCTCAAGAGATGATTCAAAAAACACGCTGTAT |
| | | CTACAAATGAACAGCCTGAAAACCGAGGACACAGCCGTGTA |
| | | TTACTGTACCACAGCGAGGTGGGACTGGTACTTCGATCTCT |
| | | GGGGCCGTGGCACCCTGGTCACTGTCTCCTCAGCCTCCACC |
| | | AAGGGCCCATCGGTCTTCCCCCTGGCACCCTCCTCCAAGAG |
| | LC | CACCTCTGGGGGCACAGCGGCCCTGGGCTGCCTGGTCAAGG |
| | | ACTACTTCCCCGAACCGGTGACGGTGTCGTGGAACTCAGGC |
| | | GCCCTGACCAGCGGCGTGCACACCTTCCCGGCTGTCCTACA |
| | | GTCCTCAGGACTCTACTCCCTCAGCAGCGTGGTGACCGTGC |
| | | CCTCCAGCAGCTTGGGCACCCAGACCTACATCTGCAACGTG |
| | | AATCACAAGCCCAGCAACACCAAGGTGGACAAGAAAGTTGA |
| | | GCCCAAATCTTGTGACAAAACTCACACATGCCCACCGTGCC |
| | | CAGCACCTGAACTCCTGGGGGGACCGTCAGTCTTCCTCTTC |
| | | CCCCCAAAACCCAAGGACACCCTCATGATCTCCCGGACCCC |
| | | TGAGGTCACATGCGTGGTGGTGGACGTGAGCCACGAAGACC |
| | | CTGAGGTCAAGTTCAACTGGTACGTGGACGGCGTGGAGGTG |
| | | CATAATGCCAAGACAAAGCCGCGGGAGGAGCAGTACAACAG |
| | | CACGTACCGTGTGGTCAGCGTCCTCACCGTCCTGCACCAGG |
| | | ACTGGCTGAATGGCAAGGAGTACAAGTGCAAGGTCTCCAAC |
| | | AAAGCCCTCCCAGCCCCCATCGAGAAAACCATCTCCAAAGC |
| | | CAAAGGGCAGCCCCGAGAACCACAGGTGTACACCCTGCCCC |
| | | CATCCCGGGATGAGCTGACCAAGAACCAGGTCAGCCTGACC |
| | | TGCCTGGTCAAAGGCTTCTATCCCAGCGACATCGCCGTGGA |
| | | GTGGGAGAGCAATGGGCAGCCGGAGAACAACTACAAGACCA |
| | | CGCCTCCCGTGCTGGACTCCGACGGCTCCTTCTTCCTCTAC |
| | | AGCAAGCTCACCGTGGACAAGAGCAGGTGGCAGCAGGGGAA |
| | | CGTCTTCTCATGCTCCGTGATGCATGAGGCTCTGCACAACC |
| | | ACTACACGCAGAAGTCCCTCTCCCTGTCTCCGGGTAAATGA |
| | | (SEQ ID NO: 46) |
| | | GACATCCAGATGACCCAGTCTCCATCCTCCCTGTCTGCATC |
| | | TGTAGGAGACAGAGTCACCATCACTTGCCAGGCGAGTCAGG |
| | | ACATTTGGAATTATATAAATTGGTATCAGCAGAAACCAGGG |
| | | AAGGCCCCTAAGCTCCTGATCTACGATGCATCCAATTTGAA |
| | | AACAGGGGTCCCATCAAGGTTCAGTGGAAGTGGATCTGGGA |
| | | CAGATTTTACTTTCACCATCAGCAGCCTGCAGCCTGAAGAT |
| | | ATTGCAACATATTACTGTCAACAGCATGATGATCTCCCTCC |
| | | GACCTTCGGCCAAGGGACCAAGGTGGAAATCAAACGAACTG |
| | | TGGCTGCACCATCTGTCTTCATCTTCCCGCCATCTGATGAG |
| | | CAGTTGAAATCTGGAACTGCCTCTGTTGTGTGCCTGCTGAA |
| | | TAACTTCTATCCCAGAGAGGCCAAAGTACAGTGGAAGGTGG |
| | | ATAACGCCCTCCAATCGGGTAACTCCCAGGAGAGTGTCACA |
| | | GAGCAGGACAGCAAGGACAGCACCTACAGCCTCAGCAGCAC |
| | | CCTGACGCTGAGCAAAGCAGACTACGAGAAACACAAAGTCT |
| | | ACGCCTGCGAAGTCACCCATCAGGGCCTGAGCTCGCCCGTC |
| | | ACAAAGAGCTTCAACAGGGGAGAGTGTTAG (SEQ ID |
| | | NO: 47) |
| mAb10987 | VH | QVQLVESGGGVVQPGRSLRLSCAASGFTFSNYAMYWVRQAP |
| | | GKGLEWVAVISYDGSNKYYADSVKGRFTISRDNSKNTLYLQ |
| | | MNSLRTEDTAVYYCASGSDYGDYLLVYWGQGTLVTVSS |
| | | (SEQ ID NO: 48) |
| | CDR-H1 | GFTFSNYA (SEQ ID NO: 49) |
| | CDR-H2 | ISYDGSNK (SEQ ID NO: 50) |
| | CDR-H3 | ASGSDYGDYLLVY (SEQ ID NO: 51) |
| | VL | QSALTQPASVSGSPGQSITISCTGTSSDVGGYNYVSWYQQH |
| | | PGKAPKLMIYDVSKRPSGVSNRFSGSKSGNTASLTISGLQS |
| | | EDEADYYCNSLTSISTWVFGGGTKLTVL (SEQ ID NO: |
| | | 52) |
| | CDR-L1 | SSDVGGYNY (SEQ ID NO: 53) |
| | CDR-L2 | DVS |
| | CDR-L3 | NSLTSISTWV (SEQ ID NO: 54) |
| | HC | QVQLVESGGGVVQPGRSLRLSCAASGFTFSNYAMYWVRQAP |
| | | GKGLEWVAVISYDGSNKYYADSVKGRFTISRDNSKNTLYLQ |
| | | MNSLRTEDTAVYYCASGSDYGDYLLVYWGQGTLVTVSSAST |
| | | KGPSVFPLAPSSKSTSGGTAALGCLVKDYFPEPVTVSWNSG |
| | | ALTSGVHTFPAVLQSSGLYSLSSVVTVPSSSLGTQTYICNV |
| | | NHKPSNTKVDKKVEPKSCDKTHTCPPCPAPELLGGPSVFLF |
| | | PPKPKDTLMISRTPEVTCVVVDVSHEDPEVKFNWYVDGVEV |
| | | HNAKTKPREEQYNSTYRVVSVLTVLHQDWLNGKEYKCKVSN |
| | | KALPAPIEKTISKAKGQPREPQVYTLPPSRDELTKNQVSLT |
| | | CLVKGFYPSDIAVEWESNGQPENNYKTTPPVLDSDGSFFLY |
| | | SKLTVDKSRWQQGNVFSCSVMHEALHNHYTQKSLSLSPGK |
| | | (SEQ ID NO: 55) |
| | LC | QSALTQPASVSGSPGQSITISCTGTSSDVGGYNYVSWYQQH |
| | | PGKAPKLMIYDVSKRPSGVSNRFSGSKSGNTASLTISGLQS |
| | | EDEADYYCNSLTSISTWVFGGGTKLTVLGQPKAAPSVTLEP |
| | | PSSEELQANKATLVCLISDFYPGAVTVAWKADSSPVKAGVE |
| | | TTTPSKQSNNKYAASSYLSLTPEQWKSHRSYSCQVTHEGST |
| | | VEKTVAPTECS (SEQ ID NO: 56) |
| | VH | CAGGTGCAGCTGGTGGAGTCTGGGGGAGGCGTGGTCCAGCC |
| | | TGGGAGGTCCCTGAGACTCTCCTGTGCAGCCTCTGGATTCA |
| | | CCTTCAGTAACTATGCTATGTACTGGGTCCGCCAGGCTCCA |
| | | GGCAAGGGGCTGGAGTGGGTGGCAGTTATATCATATGATGG |
| | | AAGTAATAAATACTATGCAGACTCCGTGAAGGGCCGATTCA |
| | | CCATCTCCAGAGACAATTCCAAGAACACGCTGTATCTGCAA |
| | | ATGAACAGCCTGAGAACTGAGGACACGGCTGTGTATTACTG |
| | | TGCGAGTGGCTCCGACTACGGTGACTACTTATTGGTTTACT |
| | | GGGGCCAGGGAACCCTGGTCACCGTCTCCTCA (SEQ ID |
| | | NO: 57) |
| | CDR-H1 | GGATTCACCTTCAGTAACTATGCT (SEQ ID NO: 58) |
| | CDR-H2 | ATATCATATGATGGAAGTAATAAA (SEQ ID NO: 59) |
| | CDR-H3 | GCGAGTGGCTCCGACTACGGTGACTACTTATTGGTTTAC |
| | | (SEQ ID NO: 60) |
| | VL | CAGTCTGCCCTGACTCAGCCTGCCTCCGTGTCTGGGTCTCC |
| | | TGGACAGTCGATCACCATCTCCTGCACTGGAACCAGCAGTG |
| | | ACGTTGGTGGTTATAACTATGTCTCCTGGTACCAACAACAC |
| | | CCAGGCAAAGCCCCCAAACTCATGATTTATGATGTCAGTAA |
| | | GCGGCCCTCAGGGGTTTCTAATCGCTTCTCTGGCTCCAAGT |
| | | CTGGCAACACGGCCTCCCTGACCATCTCTGGGCTCCAGTCT |
| | | GAGGACGAGGCTGATTATTACTGCAACTCTTTGACAAGCAT |
| | | CAGCACTTGGGTGTTCGGCGGAGGGACCAAGCTGACCGTCC |
| | | TA (SEQ ID NO: 61) |
| | CDR-L1 | AGCAGTGACGTTGGTGGTTATAACTAT (SEQ ID NO: |
| | | 62) |
| | CDR-L2 | GATGTCAGT |
| | CDR-L3 | AACTCTTTGACAAGCATCAGCACTTGGGTG (SEQ ID |
| | | NO: 63) |
| | HC | CAGGTGCAGCTGGTGGAGTCTGGGGGAGGCGTGGTCCAGCC |
| | | TGGGAGGTCCCTGAGACTCTCCTGTGCAGCCTCTGGATTCA |
| | | CCTTCAGTAACTATGCTATGTACTGGGTCCGCCAGGCTCCA |
| | | GGCAAGGGGCTGGAGTGGGTGGCAGTTATATCATATGATGG |
| | | AAGTAATAAATACTATGCAGACTCCGTGAAGGGCCGATTCA |
| | | CCATCTCCAGAGACAATTCCAAGAACACGCTGTATCTGCAA |
| | | ATGAACAGCCTGAGAACTGAGGACACGGCTGTGTATTACTG |
| | | TGCGAGTGGCTCCGACTACGGTGACTACTTATTGGTTTACT |
| | | GGGGCCAGGGAACCCTGGTCACCGTCTCCTCAGCCTCCACC |
| | | AAGGGCCCATCGGTCTTCCCCCTGGCACCCTCCTCCAAGAG |
| | | CACCTCTGGGGGCACAGCGGCCCTGGGCTGCCTGGTCAAGG |
| | | ACTACTTCCCCGAACCGGTGACGGTGTCGTGGAACTCAGGC |
| | | GCCCTGACCAGCGGCGTGCACACCTTCCCGGCTGTCCTACA |
| | | GTCCTCAGGACTCTACTCCCTCAGCAGCGTGGTGACCGTGC |
| | | CCTCCAGCAGCTTGGGCACCCAGACCTACATCTGCAACGTG |
| | | AATCACAAGCCCAGCAACACCAAGGTGGACAAGAAAGTTGA |
| | | GCCCAAATCTTGTGACAAAACTCACACATGCCCACCGTGCC |
| | | CAGCACCTGAACTCCTGGGGGGACCGTCAGTCTTCCTCTTC |
| | | CCCCCAAAACCCAAGGACACCCTCATGATCTCCCGGACCCC |
| | | TGAGGTCACATGCGTGGTGGTGGACGTGAGCCACGAAGACC |
| | | CTGAGGTCAAGTTCAACTGGTACGTGGACGGCGTGGAGGTG |
| | | CATAATGCCAAGACAAAGCCGCGGGAGGAGCAGTACAACAG |
| | | CACGTACCGTGTGGTCAGCGTCCTCACCGTCCTGCACCAGG |
| | | ACTGGCTGAATGGCAAGGAGTACAAGTGCAAGGTCTCCAAC |
| | | AAAGCCCTCCCAGCCCCCATCGAGAAAACCATCTCCAAAGC |
| | | CAAAGGGCAGCCCCGAGAACCACAGGTGTACACCCTGCCCC |
| | | CATCCCGGGATGAGCTGACCAAGAACCAGGTCAGCCTGACC |
| | | TGCCTGGTCAAAGGCTTCTATCCCAGCGACATCGCCGTGGA |
| | | GTGGGAGAGCAATGGGCAGCCGGAGAACAACTACAAGACCA |
| | | CGCCTCCCGTGCTGGACTCCGACGGCTCCTTCTTCCTCTAC |
| | | AGCAAGCTCACCGTGGACAAGAGCAGGTGGCAGCAGGGGAA |
| | LC | CGTCTTCTCATGCTCCGTGATGCATGAGGCTCTGCACAACC |
| | | ACTACACGCAGAAGTCCCTCTCCCTGTCTCCGGGTAAATGA |
| | | (SEQ ID NO: 64) |
| | | CAGTCTGCCCTGACTCAGCCTGCCTCCGTGTCTGGGTCTCC |
| | | TGGACAGTCGATCACCATCTCCTGCACTGGAACCAGCAGTG |
| | | ACGTTGGTGGTTATAACTATGTCTCCTGGTACCAACAACAC |
| | | CCAGGCAAAGCCCCCAAACTCATGATTTATGATGTCAGTAA |
| | | GCGGCCCTCAGGGGTTTCTAATCGCTTCTCTGGCTCCAAGT |
| | | CTGGCAACACGGCCTCCCTGACCATCTCTGGGCTCCAGTCT |
| | | GAGGACGAGGCTGATTATTACTGCAACTCTTTGACAAGCAT |
| | | CAGCACTTGGGTGTTCGGCGGAGGGACCAAGCTGACCGTCC |
| | | TAGGCCAGCCCAAGGCCGCCCCCTCCGTGACCCTGTTCCCC |
| | | CCCTCCTCCGAGGAGCTGCAGGCCAACAAGGCCACCCTGGT |
| | | GTGCCTGATCTCCGACTTCTACCCCGGCGCCGTGACCGTGG |
| | | CCTGGAAGGCCGACTCCTCCCCCGTGAAGGCCGGCGTGGAG |
| | | ACCACCACCCCCTCCAAGCAGTCCAACAACAAGTACGCCGC |
| | | CTCCTCCTACCTGTCCCTGACCCCCGAGCAGTGGAAGTCCC |
| | | ACCGGTCCTACTCCTGCCAGGTGACCCACGAGGGCTCCACC |
| | | GTGGAGAAGACCGTGGCCCCCACCGAGTGCTCCTGA (SEQ |
| | | ID NO: 65) |
| mAb10989 | VH | QVQLVQSGAEVKKPGASVKVSCKASGYIFTGYYMHWVRQAP |
| | | GQGLEWMGWINPNSGGANYAQKFQGRVTLTRDTSITTVYME |
| | | LSRLREDDTAVYYCARGSRYDWNQNNWEDPWGQGTLVTVSS |
| | | (SEQ ID NO: 66) |
| | CDR-H1 | GYIFTGYY (SEQ ID NO: 67) |
| | CDR-H2 | INPNSGGA (SEQ ID NO: 68) |
| | CDR-H3 | ARGSRYDWNQNNWEDP (SEQ ID NO: 69) |
| | VL | QSALTQPASVSGSPGQSITISCTGTSSDVGTYNYVSWYQQH |
| | | PGKAPKLMIFDVSNRPSGVSDRFSGSKSGNTASLTISGLQA |
| | | EDEADYYCSSFTTSSTVVFGGGTKLTVL (SEQ ID NO: |
| | | 70) |
| | CDR-L1 | SSDVGTYNY (SEQ ID NO: 71) |
| | CDR-L2 | DVS |
| | CDR-L3 | SSFTTSSTVV (SEQ ID NO: 72) |
| | HC | QVQLVQSGAEVKKPGASVKVSCKASGYIFTGYYMHWVRQAP |
| | | GQGLEWMGWINPNSGGANYAQKFQGRVTLTRDTSITTVYME |
| | | LSRLREDDTAVYYCARGSRYDWNQNNWEDPWGQGTLVTVSS |
| | | ASTKGPSVFPLAPSSKSTSGGTAALGCLVKDYFPEPVTVSW |
| | | NSGALTSGVHTFPAVLQSSGLYSLSSVVTVPSSSLGTQTYI |
| | | CNVNHKPSNTKVDKKVEPKSCDKTHTCPPCPAPELLGGPSV |
| | | FLFPPKPKDTLMISRTPEVTCVVVDVSHEDPEVKFNWYVDG |
| | | VEVHNAKTKPREEQYNSTYRVVSVLTVLHQDWLNGKEYKCK |
| | | VSNKALPAPIEKTISKAKGQPREPQVYTLPPSRDELTKNQV |
| | | SLTCLVKGFYPSDIAVEWESNGQPENNYKTTPPVLDSDGSE |
| | | FLYSKLTVDKSRWQQGNVFSCSVMHEALHNHYTQKSLSLSP |
| | | GK (SEQ ID NO: 73) |
| | LC | QSALTQPASVSGSPGQSITISCTGTSSDVGTYNYVSWYQQH |
| | | PGKAPKLMIFDVSNRPSGVSDRFSGSKSGNTASLTISGLQA |
| | | EDEADYYCSSFTTSSTVVFGGGTKLTVLGQPKAAPSVTLFP |
| | | PSSEELQANKATLVCLISDFYPGAVTVAWKADSSPVKAGVE |
| | | TTTPSKQSNNKYAASSYLSLTPEQWKSHRSYSCQVTHEGST |
| | | VEKTVAPTECS (SEQ ID NO: 74) |
| | VH | CAGGTGCAGCTGGTGCAGTCTGGGGCTGAGGTGAAGAAGCC |
| | | TGGGGCCTCAGTGAAGGTCTCCTGCAAGGCTTCTGGATACA |
| | | TCTTCACCGGCTACTATATGCACTGGGTGCGACAGGCCCCT |
| | | GGACAGGGGCTTGAGTGGATGGGATGGATCAACCCTAACAG |
| | | TGGTGGCGCAAACTATGCACAGAAGTTTCAGGGCAGGGTCA |
| | | CCCTGACCAGGGACACGTCCATCACCACAGTCTACATGGAA |
| | | CTGAGCAGGCTGAGATTTGACGACACGGCCGTGTATTACTG |
| | | TGCGAGAGGATCCCGGTATGACTGGAACCAGAACAACTGGT |
| | | TCGACCCCTGGGGCCAGGGAACCCTGGTCACCGTCTCCTCA |
| | | (SEQ ID NO: 75) |
| | CDR-H1 | GGATACATCTTCACCGGCTACTAT (SEQ ID NO: 76) |
| | CDR-H2 | ATCAACCCTAACAGTGGTGGCGCA (SEQ ID NO: 77) |
| | CDR-H3 | GCGAGAGGATCCCGGTATGACTGGAACCAGAACAACTGGTT |
| | | CGACCCC (SEQ ID NO: 78) |
| | VL | CAGTCTGCCCTGACTCAGCCTGCCTCCGTGTCTGGGTCTCC |
| | | TGGACAGTCGATCACCATCTCCTGCACTGGAACCAGCAGTG |
| | | ACGTTGGTACTTATAACTATGTCTCCTGGTACCAACAACAC |
| | | CCAGGCAAAGCCCCCAAACTCATGATTTTTGATGTCAGTAA |
| | | TCGGCCCTCAGGGGTTTCTGATCGCTTCTCTGGCTCCAAGT |
| | | CTGGCAACACGGCCTCCCTGACCATCTCTGGGCTCCAGGCT |
| | | GAGGACGAGGCTGATTATTACTGCAGCTCATTTACAACCAG |
| | | CAGCACTGTGGTTTTCGGCGGAGGGACCAAGCTGACCGTCC |
| | | TA (SEQ ID NO: 79) |
| | CDR-L1 | AGCAGTGACGTTGGTACTTATAACTAT (SEQ ID NO: |
| | | 80) |
| | CDR-L2 | GATGTCAGT |
| | CDR-L3 | AGCTCATTTACAACCAGCAGCACTGTGGTT (SEQ ID |
| | | NO: 81) |
| | HC | CAGGTGCAGCTGGTGCAGTCTGGGGCTGAGGTGAAGAAGCC |
| | | TGGGGCCTCAGTGAAGGTCTCCTGCAAGGCTTCTGGATACA |
| | | TCTTCACCGGCTACTATATGCACTGGGTGCGACAGGCCCCT |
| | | GGACAGGGGCTTGAGTGGATGGGATGGATCAACCCTAACAG |
| | | TGGTGGCGCAAACTATGCACAGAAGTTTCAGGGCAGGGTCA |
| | | CCCTGACCAGGGACACGTCCATCACCACAGTCTACATGGAA |
| | | CTGAGCAGGCTGAGATTTGACGACACGGCCGTGTATTACTG |
| | | TGCGAGAGGATCCCGGTATGACTGGAACCAGAACAACTGGT |
| | LC | TCGACCCCTGGGGCCAGGGAACCCTGGTCACCGTCTCCTCA |
| | | GCCTCCACCAAGGGCCCATCGGTCTTCCCCCTGGCACCCTC |
| | | CTCCAAGAGCACCTCTGGGGGCACAGCGGCCCTGGGCTGCC |
| | | TGGTCAAGGACTACTTCCCCGAACCGGTGACGGTGTCGTGG |
| | | AACTCAGGCGCCCTGACCAGCGGCGTGCACACCTTCCCGGC |
| | | TGTCCTACAGTCCTCAGGACTCTACTCCCTCAGCAGCGTGG |
| | | TGACCGTGCCCTCCAGCAGCTTGGGCACCCAGACCTACATC |
| | | TGCAACGTGAATCACAAGCCCAGCAACACCAAGGTGGACAA |
| | | GAAAGTTGAGCCCAAATCTTGTGACAAAACTCACACATGCC |
| | | CACCGTGCCCAGCACCTGAACTCCTGGGGGGACCGTCAGTC |
| | | TTCCTCTTCCCCCCAAAACCCAAGGACACCCTCATGATCTC |
| | | CCGGACCCCTGAGGTCACATGCGTGGTGGTGGACGTGAGCC |
| | | ACGAAGACCCTGAGGTCAAGTTCAACTGGTACGTGGACGGC |
| | | GTGGAGGTGCATAATGCCAAGACAAAGCCGCGGGAGGAGCA |
| | | GTACAACAGCACGTACCGTGTGGTCAGCGTCCTCACCGTCC |
| | | TGCACCAGGACTGGCTGAATGGCAAGGAGTACAAGTGCAAG |
| | | GTCTCCAACAAAGCCCTCCCAGCCCCCATCGAGAAAACCAT |
| | | CTCCAAAGCCAAAGGGCAGCCCCGAGAACCACAGGTGTACA |
| | | CCCTGCCCCCATCCCGGGATGAGCTGACCAAGAACCAGGTC |
| | | AGCCTGACCTGCCTGGTCAAAGGCTTCTATCCCAGCGACAT |
| | | CGCCGTGGAGTGGGAGAGCAATGGGCAGCCGGAGAACAACT |
| | | ACAAGACCACGCCTCCCGTGCTGGACTCCGACGGCTCCTTC |
| | | TTCCTCTACAGCAAGCTCACCGTGGACAAGAGCAGGTGGCA |
| | | GCAGGGGAACGTCTTCTCATGCTCCGTGATGCATGAGGCTC |
| | | TGCACAACCACTACACGCAGAAGTCCCTCTCCCTGTCTCCG |
| | | GGTAAATGA (SEQ ID NO: 82) |
| | | CAGTCTGCCCTGACTCAGCCTGCCTCCGTGTCTGGGTCTCC |
| | | TGGACAGTCGATCACCATCTCCTGCACTGGAACCAGCAGTG |
| | | ACGTTGGTACTTATAACTATGTCTCCTGGTACCAACAACAC |
| | | CCAGGCAAAGCCCCCAAACTCATGATTTTTGATGTCAGTAA |
| | | TCGGCCCTCAGGGGTTTCTGATCGCTTCTCTGGCTCCAAGT |
| | | CTGGCAACACGGCCTCCCTGACCATCTCTGGGCTCCAGGCT |
| | | GAGGACGAGGCTGATTATTACTGCAGCTCATTTACAACCAG |
| | | CAGCACTGTGGTTTTCGGCGGAGGGACCAAGCTGACCGTCC |
| | | TAGGCCAGCCCAAGGCCGCCCCCTCCGTGACCCTGTTCCCC |
| | | CCCTCCTCCGAGGAGCTGCAGGCCAACAAGGCCACCCTGGT |
| | | GTGCCTGATCTCCGACTTCTACCCCGGCGCCGTGACCGTGG |
| | | CCTGGAAGGCCGACTCCTCCCCCGTGAAGGCCGGCGTGGAG |
| | | ACCACCACCCCCTCCAAGCAGTCCAACAACAAGTACGCCGC |
| | | CTCCTCCTACCTGTCCCTGACCCCCGAGCAGTGGAAGTCCC |
| | | ACCGGTCCTACTCCTGCCAGGTGACCCACGAGGGCTCCACC |
| | | GTGGAGAAGACCGTGGCCCCCACCGAGTGCTCCTGA (SEQ |
| | | ID NO: 83) |

In further embodiments, the Fabs comprise an amino acid sequence set forth in Table 5 below. In particular aspects, the Fab comprises both heavy and light chain CDRs of an antibody set forth in Table 5 below. In other embodiments, the Fab comprises at least the heavy chain CDR sequences and the light chain CDR sequences of a universal light chain. In further aspects, a Fab comprises a VH having the amino acid sequence of the VH of an antibody set forth in Table 5 and a VL having the amino acid sequence of the VL of the same antibody as set forth in Table 5. In other aspects, a Fab comprises a VH having the amino acid sequence of the VH of an antibody set forth in Table 5 and a universal light chain VL sequence. All sequence identifiers for Table 5 are in relation to the sequence listing in WO 2021/045836 A1, which sequence identifiers are incorporated by reference herein.

TABLE 5
SEQ ID NO in WO 2021/045836 A1 (Sequence or SEQ ID NO of the disclosure)

| Antibody Designation | VH | CDR-H1 | CDR-H2 | CDR-H3 | VL | CDR-L1 | CDR-L2 | CDR-L3 |
|---|---|---|---|---|---|---|---|---|
| Antibody Designation | VH | CDR-H1 | CDR-H2 | CDR-H3 | VL | CDR-L1 | CDR-L2 | CDR-L3 |
| mAb10913 | 2 | 4 | 6 | 8 | 10 | 12 | 14 | 16 |
| | (281) | (282) | (283) | (284) | (285) | (286) | (DNN) | (287) |
| mAb10915 | 22 | 24 | 26 | 28 | 30 | 32 | 34 | 36 |
| | (288) | (289) | (290) | (291) | (292) | (293) | (KAS) | (294) |
| mAb10916 | 2 | 4 | 6 | 8 | 10 | 12 | 14 | 16 |
| | (281) | (282) | (283) | (284) | (285) | (286) | (DNN) | (287) |
| mAb10917 | 44 | 46 | 26 | 49 | 51 | 53 | 55 | 57 |
| | (295) | (296) | (290) | (297) | (298) | (299) | (AAS) | (300) |
| mAb10918 | 22 | 24 | 26 | 28 | 30 | 32 | 34 | 36 |
| | (288) | (289) | (290) | (291) | (292) | (293) | (KAS) | (294) |
| mAb10920 | 65 | 67 | 69 | 71 | 73 | 75 | 55 | 77 |
| | (301) | (302) | (303) | (304) | (305) | (306) | (AAS) | (307) |
| mAb10921 | 83 | 85 | 26 | 87 | 89 | 91 | 55 | 93 |
| | (308) | (309) | (290) | (310) | (311) | (312) | (AAS) | (313) |
| mAb10922 | 99 | 101 | 103 | 105 | 107 | 109 | 111 | 113 |
| | (314) | (315) | (316) | (317) | (318) | (319) | (WAS) | (320) |
| mAb10923 | 119 | 121 | 123 | 125 | 127 | 129 | 55 | 131 |
| | (321) | (322) | (323) | (324) | (325) | (326) | (AAS) | (327) |
| mAb10924 | 137 | 139 | 141 | 143 | 145 | 147 | 149 | 151 |
| | (328) | (329) | (330) | (331) | (332) | (333) | (GVS) | (334) |
| mAb10925 | 65 | 67 | 69 | 71 | 73 | 75 | 55 | 77 |
| | (301) | (302) | (303) | (304) | (305) | (306) | (AAS) | (307) |
| mAb10926 | 83 | 85 | 26 | 87 | 89 | 91 | 55 | 93 |
| | (308) | (309) | (290) | (310) | (311) | (312) | (AAS) | (313) |
| mAb10927 | 99 | 101 | 103 | 105 | 107 | 109 | 111 | 113 |
| | (314) | (315) | (316) | (317) | (318) | (319) | (WAS) | (320) |
| mAb10928 | 119 | 121 | 123 | 125 | 127 | 129 | 55 | 131 |
| | (321) | (322) | (323) | (324) | (325) | (326) | (AAS) | (327) |
| mAb10929 | 137 | 139 | 141 | 143 | 145 | 147 | 149 | 151 |
| | (328) | (329) | (330) | (331) | (332) | (333) | (GVS) | (334) |
| mAb10930 | 167 | 169 | 171 | 173 | 175 | 129 | 55 | 177 |
| | (335) | (336) | (337) | (338) | (339) | (326) | (AAS) | (340) |
| mAb10931 | 167 | 169 | 171 | 173 | 175 | 129 | 55 | 177 |
| | (335) | (336) | (337) | (338) | (339) | (326) | (AAS) | (340) |
| mAb10932 | 185 | 187 | 26 | 189 | 191 | 75 | 194 | 196 |
| | (341) | (342) | (290) | (343) | (344) | (306) | (DAS) | (345) |
| mAb10933 | 202 | 204 | 206 | 208 | 210 | 212 | 55 | 214 |
| | (12) | (13) | (14) | (15) | (16) | (17) | (AAS) | (18) |
| mAb10934 | 220 | 222 | 224 | 226 | 228 | 230 | 194 | 232 |
| | (30) | (31) | (32) | (33) | (34) | (35) | (DAS) | (36) |
| mAb10935 | 238 | 24 | 26 | 240 | 242 | 244 | 194 | 246 |
| | (346) | (289) | (290) | (347) | (348) | (349) | (DAS) | (350) |
| mAb10936 | 252 | 254 | 256 | 258 | 260 | 129 | 55 | 262 |
| | (351) | (352) | (353) | (354) | (355) | (326) | (AAS) | (356) |
| mAb10937 | 268 | 270 | 272 | 274 | 276 | 129 | 55 | 278 |
| | (357) | (358) | (359) | (360) | (361) | (326) | (AAS) | (362) |
| mAb10940 | 284 | 169 | 286 | 288 | 290 | 292 | 294 | 296 |
| | (363) | (336) | (364) | (365) | (366) | (367) | (GAS) | (368) |
| mAb10938 | 302 | 24 | 26 | 304 | 306 | 308 | 194 | 310 |
| | (369) | (289) | (290) | (370) | (371) | (372) | (DAS) | (373) |
| mAb10939 | 316 | 187 | 319 | 321 | 323 | 325 | 55 | 327 |
| | (374) | (342) | (375) | (376) | (377) | (378) | (AAS) | (379) |
| mAb10941 | 333 | 85 | 26 | 336 | 338 | 340 | 294 | 296 |
| | (380) | (309) | (290) | (381) | (382) | (383) | (GAS) | (368) |
| mAb10942 | 185 | 187 | 26 | 189 | 191 | 75 | 194 | 196 |
| | (341) | (342) | (290) | (343) | (344) | (306) | (DAS) | (345) |
| mAb10943 | 202 | 204 | 206 | 208 | 210 | 212 | 55 | 214 |
| | (12) | (13) | (14) | (15) | (16) | (17 | (AAS) | (18) |
| mAb10944 | 220 | 222 | 224 | 226 | 228 | 230 | 194 | 232 |
| | (30) | (31) | (32) | (33) | (34) | (35) | (DAS) | (36) |
| mAb10945 | 238 | 24 | 26 | 240 | 242 | 244 | 194 | 246 |
| | (346) | (289) | (290) | (347) | (348) | (349) | (DAS) | (350) |
| mAb10946 | 252 | 254 | 256 | 258 | 260 | 129 | 55 | 262 |
| | (351) | (352) | (353) | (354) | (355) | (326) | (AAS) | (356) |
| mAb10947 | 268 | 270 | 272 | 274 | 276 | 129 | 55 | 278 |
| | (357) | (358) | (359) | (360) | (361) | (326) | (AAS) | (362) |
| mAb10948 | 302 | 24 | 26 | 304 | 306 | 308 | 194 | 310 |
| | (369) | (289) | (290) | (370) | (371) | (372) | (DAS) | (373) |
| mAb10949 | 316 | 187 | 319 | 321 | 323 | 325 | 55 | 327 |
| | (374) | (342) | (375) | (376) | (377) | (378) | (AAS) | (379) |
| mAb10951 | 333 | 85 | 26 | 336 | 338 | 340 | 294 | 296 |
| | (380) | (309) | (290) | (381) | (382) | (383) | (GAS) | (368) |
| mAb10950 | 284 | 169 | 286 | 288 | 290 | 292 | 294 | 296 |
| | (363) | (336) | (364) | (365) | (366) | (367) | (GAS) | (368) |
| mAb10954 | 366 | 85 | 26 | 370 | 372 | 244 | 194 | 375 |
| | (384) | (309) | (290) | (385) | (386) | (349) | (DAS) | (387) |
| mAb10955 | 381 | 383 | 26 | 385 | 387 | 389 | 194 | 310 |
| | (388) | (389) | (290) | (390) | (391) | (392) | (DAS) | (373) |
| mAb10956 | 396 | 187 | 26 | 399 | 401 | 389 | 194 | 403 |
| | (393) | (342) | (290) | (394) | (395) | (392) | (DAS) | (396) |
| mAb10957 | 409 | 411 | 26 | 414 | 416 | 53 | 55 | 418 |
| | (397) | (398) | (290) | (399) | (400) | (299) | (AAS) | (401) |
| mAb10958 | 366 | 85 | 26 | 370 | 372 | 244 | 194 | 375 |
| | (384) | (309) | (290) | (385) | (386) | (349) | (DAS) | (387) |
| mAb10959 | 381 | 383 | 26 | 385 | 387 | 389 | 194 | 310 |
| | (388) | (389) | (290) | (390) | (391) | (392) | (DAS) | (373) |
| mAb10960 | 396 | 18 | 26 | 399 | 401 | 389 | 194 | 403 |
| | (393) | (342) | (290) | (394) | (395) | (392) | (DAS) | (396) |
| mAb10961 | 409 | 411 | 26 | 414 | 416 | 53 | 55 | 418 |
| | (397) | (398) | (290) | (399) | (400) | (299) | (AAS) | (401) |
| mAb10964 | 432 | 434 | 436 | 438 | 440 | 442 | 55 | 445 |
| | (402) | (403) | (404) | (405) | (406) | (407) | (AAS) | (408) |
| mAb10965 | 451 | 453 | 26 | 455 | 457 | 459 | 34 | 462 |
| | (409) | (410) | (290) | (411) | (412) | (413) | (KAS) | (414) |
| mAb10966 | 468 | 187 | 26 | 470 | 472 | 389 | 194 | 474 |
| | (415) | (342) | (290) | (416) | (417) | (392) | (DAS) | (418) |
| mAb10967 | 480 | 24 | 483 | 485 | 487 | 389 | 194 | 489 |
| | (419) | (289) | (420) | (421) | (422) | (392) | (DAS) | (423) |
| mAb10969 | 495 | 497 | 499 | 501 | 503 | 389 | 194 | 214 |
| | (424) | (425) | (50) | (426) | (427) | (392) | (DAS) | (18) |
| mAb10970 | 510 | 24 | 26 | 512 | 514 | 516 | 194 | 518 |
| | (428) | (289) | (290) | (429) | (430) | (431) | (DAS) | (432) |
| mAb10971 | 524 | 411 | 26 | 528 | 530 | 532 | 55 | 534 |
| | (433) | (398) | (290) | (434) | (435) | (436) | (AAS) | (437) |
| mAb10973 | 432 | 434 | 436 | 438 | 440 | 442 | 55 | 445 |
| | (402) | (403) | (404) | (405) | (406) | (407) | (AAS) | (408) |
| mAb10974 | 451 | 453 | 26 | 455 | 457 | 459 | 34 | 462 |
| | (409) | (410) | (290) | (411) | (412) | (413) | (KAS) | (414) |
| mAb10975 | 468 | 187 | 26 | 470 | 472 | 389 | 194 | 474 |
| | (415) | (342) | (290) | (416) | (417) | (392) | (DAS) | (418) |
| mAb10976 | 480 | 24 | 483 | 485 | 487 | 389 | 194 | 489 |
| | (419) | (289) | (420) | (421) | (422) | (392) | (DAS) | (423) |
| mAb10977 | 548 | 550 | 552 | 554 | 556 | 558 | 294 | 560 |
| | (438) | (439) | (440) | (441) | (442) | (443) | (GAS) | (444) |
| mAb10978 | 495 | 497 | 499 | 501 | 503 | 389 | 194 | 214 |
| | (424) | (425) | (50) | (426) | (427) | (392) | (DAS) | (18) |
| mAb10979 | 510 | 24 | 26 | 512 | 514 | 516 | 194 | 518 |
| | (428) | (289) | (290) | (429) | (430) | (431) | (DAS) | (432) |
| mAb10980 | 524 | 411 | 26 | 528 | 530 | 532 | 55 | 534 |
| | (433) | (398) | (290) | (434) | (435) | (436) | (AAS) | (437) |
| mAb10981 | 548 | 550 | 552 | 554 | 556 | 558 | 294 | 560 |
| | (438) | (439) | (440) | (441) | (442) | (443) | (GAS) | (444) |
| mAb10982 | 574 | 187 | 576 | 578 | 580 | 582 | 584 | 586 |
| | (445) | (342) | (446) | (447) | (448) | (449) | (GNS) | (450) |
| mAb10983 | 574 | 187 | 576 | 578 | 580 | 582 | 584 | 586 |
| | (445) | (342) | (446) | (447) | (448) | (449) | (GNS) | (450) |
| mAb10984 | 594 | 596 | 26 | 598 | 600 | 12 | 14 | 602 |
| | (451) | (452) | (290) | (453) | (454) | (286) | (DNN) | (455) |
| mAb10985 | 608 | 169 | 610 | 612 | 614 | 616 | 584 | 618 |
| | (456) | (336) | (457) | (458) | (459) | (460) | (GNS) | (461) |
| mAb10986 | 624 | 626 | 26 | 628 | 630 | 582 | 632 | 634 |
| | (462) | (463) | (290) | (464) | (465) | (449) | (GNT) | (466) |
| mAb10987 | 640 | 642 | 499 | 644 | 646 | 648 | 650 | 652 |
| | (48) | (49) | (50) | (51) | (52) | (53) | (DVS) | (54) |
| mAb10988 | 658 | 660 | 662 | 664 | 666 | 668 | 670 | 672 |
| | (467) | (468) | (469) | (470) | (471) | (472) | (SNN) | (473) |
| mAb10989 | 678 | 680 | 682 | 684 | 686 | 688 | 650 | 690 |
| | (66) | (67) | (68) | (69) | (70) | (71) | (DVS) | (72) |
| mAb10990 | 594 | 596 | 26 | 598 | 600 | 12 | 14 | 602 |
| | (451) | (452) | (290) | (453) | (454) | (286) | (DNN) | (455) |
| mAb10991 | 608 | 169 | 610 | 612 | 614 | 616 | 584 | 618 |
| | (456) | (336) | (457) | (458) | (459) | (460) | (GNS) | (461) |
| mAb10992 | 624 | 626 | 26 | 628 | 630 | 582 | 632 | 634 |
| | (462) | (463) | (290) | (464) | (465) | (449) | (GNT) | (466) |
| mAb10993 | 640 | 642 | 499 | 644 | 646 | 648 | 650 | 652 |
| | (48) | (49) | (50) | (51) | (52) | (53) | (DVS) | (54) |
| mAb10994 | 658 | 660 | 662 | 664 | 666 | 668 | 670 | 672 |
| | (467) | (468) | (469) | (470) | (471) | (472) | (SNN) | (473) |
| mAb10995 | 678 | 680 | 682 | 684 | 686 | 688 | 650 | 690 |
| | (66) | (67) | (68) | (69) | (70) | (71) | (DVS) | (72) |
| mAb10996 | 708 | 24 | 26 | 711 | 713 | 129 | 55 | 715 |
| | (474) | (289) | (290) | (475) | (476) | (326) | (AAS) | (477) |
| mAb10997 | 708 | 24 | 26 | 711 | 713 | 129 | 55 | 715 |
| | (474) | (289) | (290) | (475) | (476) | (326) | (AAS) | (477) |
| mAb10998 | 723 | 187 | 26 | 725 | 727 | 129 | 55 | 729 |
| | (478) | (342) | (290) | (479) | (480) | (326) | (AAS) | (481) |
| mAb10999 | 723 | 187 | 26 | 725 | 727 | 129 | 55 | 729 |
| | (478) | (342) | (290) | (479) | (480) | (326) | (AAS) | (481) |
| mAb11000 | 737 | 24 | 26 | 739 | 741 | 743 | 55 | 745 |
| | (482) | (289) | (290) | (483) | (484) | (485) | (AAS) | (486) |
| mAb11001 | 737 | 24 | 26 | 739 | 741 | 743 | 55 | 745 |
| | (482) | (289) | (290) | (483) | (484) | (485) | (AAS) | (486) |
| mAb11002 | 753 | 24 | 26 | 755 | 713 | 129 | 55 | 715 |
| | (487) | (289) | (290) | (488) | (476) | (326) | (AAS) | (477) |
| mAb11003 | 753 | 24 | 26 | 755 | 713 | 129 | 55 | 715 |
| | (487) | (289) | (290) | (488) | (476) | (326) | (AAS) | (477) |
| mAb10914 | 44 | 46 | 26 | 49 | 51 | 53 | 55 | 57 |
| | (295) | (296) | (290) | (297) | (298) | (299) | (AAS) | (300) |
| mAb11004 | 764 | 766 | 499 | 768 | 770 | 91 | 55 | 772 |
| | (489) | (490) | (50) | (491) | (492) | (312) | (AAS) | (493) |
| mAb11005 | 764 | 766 | 499 | 768 | 770 | 91 | 55 | 772 |
| | (489) | (490) | (50) | (491) | (492) | (312) | (AAS) | (493) |
| mAb11006 | 780 | 782 | 26 | 784 | 786 | 53 | 55 | 788 |
| | (494) | (495) | (290) | (496) | (497) | (299) | (AAS) | (498) |
| mAb11007 | 780 | 782 | 26 | 784 | 786 | 53 | 55 | 788 |
| | (494) | (495) | (290) | (496) | (497) | (299) | (AAS) | (498) |
| mAb11008 | 796 | 24 | 26 | 798 | 800 | 53 | 55 | 802 |
| | (499) | (289) | (290) | (500) | (501) | (299) | (AAS) | (502) |
| mAb11009 | 796 | 24 | 26 | 798 | 800 | 53 | 55 | 802 |
| | (499) | (289) | (290) | (500) | (501) | (299) | (AAS) | (502) |
| mAb11010 | 810 | 812 | 814 | 816 | 818 | 129 | 820 | 822 |
| | (503) | (504) | (505) | (506) | (507) | (326) | (TAS) | (508) |

In some embodiments, the Fabs comprise an amino acid sequence set forth in Table 5-2 below. In some aspects, the Fab comprises both heavy and light chain CDRs of an antibody set forth in Table 5-2 below. In certain aspects, a Fab comprises a VH having the amino acid sequence of the VH of an antibody set forth in Table 5-2 and a VL having the amino acid sequence of the VL of the same antibody as set forth in Table 5-2. In other aspects, a Fab comprises a VH having the amino acid sequence of the VH of an antibody set forth in Table 5-2 and a universal light chain VL sequence. The initial sequence identifiers for Table 5-2 are in relation to the sequence listing in WO 2023/287875A1, which sequence identifiers are incorporated by reference herein, while sequence identifiers presented parenthetically are those of the disclosure.

TABLE 5-2
SEQ ID NO in WO2023/287875A1 (Sequence or SEQ ID NO of the disclosure)

| Antibody Designation | VH | CDR-H1 | CDR-H2 | CDR-H3 | VL | CDR-L1 | CDR-L2 | CDR-L3 |
|---|---|---|---|---|---|---|---|---|
| Antibody Designation | VH | CDR-H1 | CDR-H2 | CDR-H3 | VL | CDR-L1 | CDR-L2 | CDR-L3 |
| mAb15163 | 2 | 4 | 6 | 8 | 10 | 12 | 14 | 16 |
| | (509) | (510) | (511) | (512) | (513) | (514) | (EVS) | (515) |
| mAb15164 | 22 | 24 | 26 | 28 | 30 | 32 | 34 | 36 |
| | (516) | (517) | (518) | (519) | (520) | (521) | (EGN) | (522) |
| mAb15165 | 42 | 44 | 26 | 47 | 49 | 51 | 34 | 36 |
| | (523) | (289) | (518) | (524) | (525) | (526) | (EGN) | (522) |
| mAb15166 | 58 | 60 | 62 | 64 | 66 | 51 | 68 | 36 |
| | (527) | (528) | (529) | (530) | (531) | (526) | (EGT) | (522) |
| mAb15167 | 74 | 76 | 78 | 80 | 82 | 84 | 86 | 88 |
| | (532) | (533) | (330) | (534) | (535) | (536) | (EDS) | (537) |
| mAb15170 | 94 | 96 | 98 | 100 | 102 | 104 | 106 | 108 |
| | (538) | (309) | (290) | (539) | (540) | (306) | (AAS) | (541) |
| mAb14296 | 114 | 116 | 118 | 120 | 122 | 124 | 126 | 128 |
| | (542) | (543) | (544) | (545) | (546) | (547) | (GAS) | (548) |
| mAb14297 | 134 | 136 | 138 | 140 | 142 | 144 | 126 | 146 |
| | (549) | (550) | (323) | (551) | (552) | (553) | (GAS) | (368) |
| mAb14312 | 152 | 154 | 156 | 158 | 160 | 162 | 164 | 166 |
| | (554) | (555) | (556) | (557) | (558) | (559) | (GNS) | (560) |
| mAb14313 | 172 | 174 | 176 | 178 | 180 | 182 | 184 | 186 |
| | (561) | (562) | (563) | (564) | (565) | (472) | (SND) | (566) |
| mAb14314 | 192 | 194 | 196 | 198 | 200 | 202 | 204 | 206 |
| | (567) | (568) | (569) | (570) | (571) | (572) | (DND) | (573) |
| mAb14315 | 212 | 214 | 216 | 218 | 220 | 222 | 126 | 224 |
| | (574) | (575) | (576) | (577) | (578) | (443) | (GAS) | (579) |
| mAb14316 | 230 | 232 | 234 | 236 | 238 | 240 | 242 | 244 |
| | (580) | (581) | (582) | (583) | (584) | (392) | (DAS) | (585) |
| mAb15150 | 250 | 252 | 254 | 256 | 258 | 260 | 262 | 264 |
| | (586) | (587) | (588) | (589) | (590) | (591) | (GAT) | (592) |
| mAb15151 | 270 | 272 | 274 | 276 | 278 | 280 | 106 | 282 |
| | (593) | (594) | (595) | (596) | (597) | (598) | (AAS) | (599) |
| mAb15156 | 288 | 290 | 292 | 294 | 296 | 298 | 300 | 302 |
| | (600) | (601) | (602) | (603) | (604) | (605) | (SDN) | (606) |
| mAb15157 | 308 | 310 | 312 | 314 | 316 | 318 | 320 | 322 |
| | (607) | (608) | (609) | (610) | (611) | (612) | (VNN) | (455) |
| mAb15158 | 328 | 330 | 332 | 334 | 336 | 338 | 106 | 340 |
| | (613) | (614) | (615) | (616) | (617) | (618) | (AAS) | (619) |
| mAb15159 | 346 | 96 | 98 | 350 | 352 | 354 | 106 | 356 |
| | (620) | (309) | (290) | (621) | (622) | (299) | (AAS) | (623) |
| mAb15160 | 362 | 364 | 366 | 368 | 370 | 372 | 106 | 374 |
| | (624) | (625) | (626) | (627) | (628) | (485) | (AAS) | (629) |
| mAb15161 | 380 | 382 | 384 | 386 | 388 | 390 | 392 | 394 |
| | (630) | (631) | (632) | (633) | (634) | (413) | (KAS) | (635) |
| mAb15162 | 400 | 402 | 98 | 405 | 407 | 409 | 242 | 411 |
| | (636) | (637) | (290) | (638) | (639) | (349) | (DAS) | (432) |
| mAb14280 | 417 | 419 | 421 | 423 | 425 | 427 | 14 | 429 |
| | (640) | (641) | (642) | (643) | (644) | (645) | (EVS) | (646) |
| mAb14281 | 435 | 437 | 138 | 440 | 442 | 444 | 446 | 448 |
| | (647) | (67) | (323) | (648) | (649) | (650) | (DKN) | (651) |
| mAb14282 | 454 | 456 | 458 | 460 | 462 | 84 | 465 | 467 |
| | (652) | (653) | (654) | (655) | (656) | (536) | (ELT) | (657) |
| mAb14283 | 473 | 475 | 477 | 479 | 481 | 483 | 485 | 487 |
| | (658) | (659) | (660) | (661) | (662) | (53) | (DVT) | (663) |
| mAb14284 | 493 | 495 | 497 | 499 | 501 | 503 | 505 | 507 |
| | (664) | (665) | (666) | (667) | (668) | (669) | (EVT) | (670) |
| mAb14285 | 513 | 515 | 517 | 519 | 521 | 523 | 525 | 36 |
| | (671) | (672) | (673) | (674) | (675) | (676) | (EGS) | (522) |
| mAb14286 | 531 | 533 | 535 | 537 | 539 | 483 | 542 | 544 |
| | (677) | (678) | (679) | (680) | (681) | (53) | (DVS) | (682) |
| mAb14287 | 550 | 552 | 554 | 556 | 558 | 84 | 14 | 560 |
| | (683) | (684) | (685) | (686) | (687) | (536) | (EVS) | (688) |
| mAb14288 | 566 | 568 | 570 | 572 | 574 | 576 | 578 | 580 |
| | (689) | (690) | (691) | (692) | (693) | (286) | (ENN) | (694) |
| mAb14289 | 586 | 588 | 590 | 592 | 594 | 596 | 126 | 598 |
| | (695) | (696) | (697) | (698) | (699) | (700) | (GAS) | (701) |
| mAb14290 | 604 | 606 | 608 | 610 | 612 | 614 | 126 | 616 |
| | (702) | (703) | (704) | (705) | (706) | (707) | (GAS) | (708) |
| mAb14291 | 622 | 624 | 626 | 628 | 630 | 632 | 634 | 636 |
| | (709) | (710) | (711) | (712) | (713) | (714) | (LGS) | (715) |
| mAb14292 | 642 | 644 | 646 | 648 | 650 | 652 | 634 | 655 |
| | (716) | (717) | (718) | (719) | (720) | (721) | (LGS) | (722) |
| mAb14293 | 661 | 663 | 665 | 667 | 669 | 124 | 126 | 671 |
| | (723) | (724) | (725) | (726) | (727) | (547) | (GAS) | (728) |
| mAb14295 | 677 | 679 | 78 | 682 | 684 | 686 | 126 | 688 |
| | (729) | (730) | (330) | (731) | (732) | (733) | (GAS) | (734) |
| mAb13459 | 694 | 696 | 698 | 700 | 702 | 704 | 706 | 708 |
| | (735) | (336) | (337) | (736) | (737) | (738) | (SAS) | (739) |
| mAb14230 | 714 | 696 | 716 | 718 | 720 | 722 | 126 | 724 |
| | (740) | (336) | (741) | (742) | (743) | (744) | (GAS) | (745) |
| mAb14231 | 730 | 732 | 734 | 736 | 738 | 740 | 106 | 742 |
| | (746) | (747) | (748) | (749) | (750) | (326) | (AAS) | (751) |
| mAb14232 | 748 | 750 | 497 | 752 | 754 | 756 | 505 | 758 |
| | (752) | (753) | (666) | (754) | (755) | (756) | (EVT) | (757) |
| mAb14233 | 764 | 766 | 768 | 770 | 772 | 774 | 776 | 778 |
| | (758) | (759) | (303) | (760) | (761) | (762) | (KDS) | (763) |
| mAb14234 | 784 | 786 | 788 | 790 | 792 | 794 | 796 | 798 |
| | (764) | (504) | (765) | (766) | (767) | (768) | (GNT) | (769) |
| mAb14235 | 804 | 806 | 497 | 808 | 810 | 812 | 505 | 814 |
| | (770) | (771) | (666) | (772) | (773) | (71) | (EVT) | (774) |
| mAb14247 | 820 | 822 | 497 | 825 | 827 | 756 | 14 | 829 |
| | (775) | (776) | (666) | (777) | (778) | (756) | (EVS) | (779) |
| mAb14248 | 835 | 837 | 839 | 841 | 843 | 845 | 847 | 849 |
| | (780) | (781) | (782) | (783) | (784) | (785) | (KIS) | (786) |
| mAb14249 | 855 | 857 | 859 | 861 | 863 | 865 | 106 | 867 |
| | (787) | (788) | (789) | (790) | (791) | (407) | (AAS) | (792) |
| mAb14255 | 873 | 76 | 876 | 878 | 880 | 84 | 86 | 36 |
| | (793) | (533) | (794) | (795) | (796) | (536) | (EDS) | (522) |
| mAb14256 | 887 | 889 | 891 | 893 | 895 | 897 | 164 | 899 |
| | (797) | (798) | (799) | (800) | (801) | (802) | (GNS) | (803) |
| mAb14257 | 905 | 154 | 908 | 910 | 912 | 914 | 916 | 166 |
| | (804) | (555) | (805) | (806) | (807) | (808) | (GHT) | (560) |
| mAb14258 | 922 | 924 | 926 | 928 | 930 | 576 | 933 | 935 |
| | (809) | (810) | (811) | (812) | (813) | (286) | (RNN) | (814) |
| mAb14259 | 941 | 943 | 945 | 947 | 949 | 951 | 634 | 655 |
| | (815) | (816) | (817) | (818) | (819) | (820) | (LGS) | (722) |
| mAb14260 | 957 | 959 | 961 | 963 | 965 | 967 | 969 | 971 |
| | (821) | (315) | (822) | (823) | (318) | (319) | (WAS) | (320) |
| mAb13457 | 694 | 696 | 698 | 700 | 977 | 704 | 706 | 979 |
| | (735) | (336) | (337) | (736) | (824) | (738) | (SAS) | (825) |
| mAb13458 | 694 | 696 | 698 | 700 | 983 | 704 | 706 | 985 |
| | (735) | (336) | (337) | (736) | (826) | (738) | (SAS) | (827) |
| mAb14294 | 989 | 991 | 993 | 995 | 997 | 999 | 1001 | 1003 |
| | (828) | (829) | (830) | (831) | (832) | (833) | (GAS) | (834) |
| mAb17090 | 493 | 495 | 497 | 499 | 501 | 503 | 505 | 507 |
| | (664) | (665) | (666) | (667) | (668) | (669) | (EVT) | (670) |

In some embodiments, the Fab comprises the heavy and light chain CDRs of antibody “mAb14287” as set forth in Table 5-2. Accordingly, in some embodiments, the Fab comprises a VH comprising CDR-H1, CDR-H2, and CDR-H3 having the amino acid sequences of SEQ ID NOs: 684, 685, and 686, respectively, and a VL comprising CDR-L1, CDR-L2, and CDR-L3 having the amino acid sequences of SEQ ID NO:536, EVS, and SEQ ID NO: 688, respectively. In some embodiments, the Fab comprises a VH having at least 90%, at least 95%, at least 96%, at least 97%, at least 98%, at least 99%, or 100% sequence identity to the amino acid sequence of SEQ ID NO:683 and a VL having at least 90%, at least 95%, at least 96%, at least 97%, at least 98%, at least 99%, or 100% sequence identity to the amino acid sequence of SEQ ID NO:687. In some embodiments, the Fab comprises a VH comprising the amino acid sequence of SEQ ID NO:683 and a VL comprising the amino acid sequence of SEQ ID NO:687.

In some embodiments, the Fab comprises the heavy and light chain CDRs of antibody “mAb15160” as set forth in Table 5-2. Accordingly, in some embodiments, the Fab comprises a VH comprising CDR-H1, CDR-H2, and CDR-H3 having the amino acid sequences of SEQ ID NOs: 625, 626, 627, respectively, and a VL comprising CDR-L1, CDR-L2, and CDR-L3 having the amino acid sequences of SEQ ID NO:485, AAS, and SEQ ID NO: 629, respectively. In some embodiments, the Fab comprises a VH having at least 90%, at least 95%, at least 96%, at least 97%, at least 98%, at least 99%, or 100% sequence identity to the amino acid sequence of SEQ ID NO:624 and a VL having at least 90%, at least 95%, at least 96%, at least 97%, at least 98%, at least 99%, or 100% sequence identity to the amino acid sequence of SEQ ID NO:628. In some embodiments, the Fab comprises a VH comprising the amino acid sequence of SEQ ID NO:624 and a VL comprising the amino acid sequence of SEQ ID NO:628.

In some embodiments, the Fab comprises the heavy and light chain CDRs of antibody “mAb14315” as set forth in Table 5-2. Accordingly, in some embodiments, the Fab comprises a VH comprising CDR-H1, CDR-H2, and CDR-H3 having the amino acid sequences of SEQ ID NOs: 575, 576, 577, respectively, and a VL comprising CDR-L1, CDR-L2, and CDR-L3 having the amino acid sequences of SEQ ID NO:443, GAS, and SEQ ID NO: 579, respectively. In some embodiments, the Fab comprises a VH having at least 90%, at least 95%, at least 96%, at least 97%, at least 98%, at least 99%, or 100% sequence identity to the amino acid sequence of SEQ ID NO:574 and a VL having at least 90%, at least 95%, at least 96%, at least 97%, at least 98%, at least 99%, or 100% sequence identity to the amino acid sequence of SEQ ID NO:578. In some embodiments, the Fab comprises a VH comprising the amino acid sequence of SEQ ID NO:574 and a VL comprising the amino acid sequence of SEQ ID NO:578.

### 6.6. Linkers 1-3

The use of appropriate linkers separating the three antibody fragment domains, e.g., Fab domains and/or Fc regions, (referred to herein as L-1, L-2, and L-3) allows circularization of the precursor molecules by positioning the Fc and/or Fab domains at approximately 60° angles from one another, thereby allowing the split inteins at the termini of the molecules to come into contact with one another and undergo trans-splicing. Typically, linker L1 is divided in the precursor molecule, forming the termini of the extein sequences flanked by the split intein, but becomes contiguous upon circularization following trans-splicing and excision of the split intein sequences.

Suitable linkers for use as L-1, L-2, and L-3 typically consist of 3 to 20 amino acids. In some embodiments, linkers L-1, L-2, and/or L-3 consist of 3 to 10 amino acids. In some embodiments, linkers L-1, L-2, and/or L-3 consist of 3 to 7 amino acids. In some embodiments, linkers L-1, L-2, and/or L-3 consist of 5 to 7 amino acids.

In some embodiments, L-1, L-2 and/or L-3 do not contain a cysteine.

In further embodiments, L-1, L-2 and/or L-3 do not contain a tryptophan.

In yet further embodiments, L-1, L-2 and/or L-3 do not contain a methionine.

In further embodiments, L-1, L-2 and/or L-3 do not contain an N-linked glycosylation site (e.g., as defined by the motif NXT or NXS).

L-1 typically has a serine and/or a threonine that, as discussed in Section 6.11, forms the first position of the extein when L-1 is split into L-1A and L-1B in the precursor molecule. In some embodiments, L-2 and L-3 also comprise a serine and/or threonine. In some embodiments, the serine or threonine is within one amino acid from the center of the linker (e.g., at the third, fourth or fifth position in the case of a 7 amino acid linker) or at the center of the linker (e.g., at the fourth position in the case of a 7 amino acid linker).

In some embodiments, L-1, L-2 and/or L-3 are predicted to lack secondary structure. Structural analysis tools (e.g., MOE, Pymol, Coot, Chimera, and Maestro) are known to those skilled in the art, and can be used to predict secondary peptide structures.

Exemplary amino acid sequences useful for use as L-1, L-2 and/or L-3 are QLGTVEG (SEQ ID NO: 84), TLNSEES (SEQ ID NO: 85), VMSSGDQ (SEQ ID NO: 86), KLASYNP (SEQ ID NO: 87), FIDSGVG (SEQ ID NO: 88), THFSQQD (SEQ ID NO: 89), NHSSLHD (SEQ ID NO: 90) and 7-amino acid glycine-serine linkers with serine at the 4^{th} position, or a 3-amino acid or 5-amino acid fragment of any of the foregoing, e.g., a 3- or 5-amino acid fragment in which a serine or threonine is at the center. In some embodiments, the glycine-serine linker is GGGSGGG (SEQ ID NO: 91), or a 5-amino acid or 3-amino acid fragment thereof, e.g., GGSGG (SEQ ID NO: 92) or GSG, respectively.

L-1, L-2 and/or L-3 may be identical or they may be different from one another.

### 6.7. Linkers L4-L6

In certain aspects, the present disclosure provides circularized antibody molecules in which two or more components are connected to one another by a peptide linker. By way of example and not limitation, linkers can be used to connect a counterpart domain (e.g., an Fd domain or a light chain of a Fab or an Fc domain) to a fusion partner.

A linker can range from 2 amino acids to 60 or more amino acids, and in certain aspects a linker ranges from 3 amino acids to 50 amino acids, from 4 to 30 amino acids, from 5 to 25 amino acids, from 10 to 25 amino acids, 10 amino acids to 60 amino acids, from 12 amino acids to 20 amino acids, from 20 amino acids to 50 amino acids, or from 25 amino acids to 35 amino acids in length.

In particular aspects, a linker is at least 3 amino acids, at least 4 amino acids, at least 5 amino acids, at least 6 amino acids or at least 7 amino acids in length and optionally is up to 30 amino acids, up to 40 amino acids, up to 50 amino acids or up to 60 amino acids in length.

In some embodiments, the linker ranges from 5 amino acids to 50 amino acids in length, e.g., ranges from 5 to 50, from 5 to 45, from 5 to 40, from 5 to 35, from 5 to 30, from 5 to 25, or from 5 to 20 amino acids in length. In other embodiments of the foregoing, the linker ranges from 6 amino acids to 50 amino acids in length, e.g., ranges from 6 to 50, from 6 to 45, from 6 to 40, from 6 to 35, from 6 to 30, from 6 to 25, or from 6 to 20 amino acids in length. In yet other embodiments, the linker ranges from 7 amino acids to 50 amino acids in length, e.g., ranges from 7 to 50, from 7 to 45, from 7 to 40, from 7 to 35, from 7 to 30, from 7 to 25, or from 7 to 20 amino acids in length.

Charged (e.g., charged hydrophilic linkers) and/or flexible linkers are particularly preferred.

Examples of flexible linkers that can be used in the circularized antibody molecules of the disclosure include those disclosed by Chen et al., 2013, Adv Drug Deliv Rev. 65 (10): 1357-1369 and Klein et al., 2014, Protein Engineering, Design & Selection 27 (10): 325-330. Particularly useful flexible linkers are or comprise repeats of glycines and serines, e.g., a monomer or multimer of GnS (SEQ ID NO: 93) or SGn (SEQ ID NO: 94), where n is an integer from 1 to 10, e.g., 12, 3, 4, 5, 6, 7, 8, 9 or 10. In one embodiment, the linker is or comprises a monomer or multimer of repeat of G4S (SEQ ID NO: 95) e.g., (GGGGS), (SEQ ID NO: 95).

Polyglycine linkers can suitably be used in the circularized antibody molecules of the disclosure. In some embodiments, a peptide linker comprises two consecutive glycines (2Gly), three consecutive glycines (3Gly), four consecutive glycines (4Gly) (SEQ ID NO: 96), five consecutive glycines (5Gly) (SEQ ID NO: 97), six consecutive glycines (6Gly) (SEQ ID NO: 98), seven consecutive glycines (7Gly) (SEQ ID NO: 99), eight consecutive glycines (8Gly) (SEQ ID NO: 100) or nine consecutive glycines (9Gly) (SEQ ID NO: 101).

Exemplary linker sequences are set forth in Table 6 below.

TABLE 6
Linker Sequences
Linker Sequence
GGGGSLALGPGGGGGSLALGPGGGGGSLALGPGGS (SEQ ID NO: 102)
GGGGSGGGGSGGGGSGGGGSGGGGS (SEQ ID NO: 103)
(GGGGS)ₙ (SEQ ID NO: 104), optionally where n = 1-10, e.g., 1, 2, 3, 4,
5, 6, 7, 8, 9, or 10
(GGGS)ₙ (SEQ ID NO: 105), optionally where n = 1-10, e.g., 1, 2, 3, 4, 5,
6, 7, 8, 9, or 10
(GGS)ₙ (SEQ ID NO: 106), optionally where n = 1-10, e.g., 1, 2, 3, 4, 5,
6, 7, 8, 9, or 10
(GS)ₙ (SEQ ID NO: 107), optionally where n = 1-10, e.g., 1, 2, 3, 4, 5, 6,
7, 8, 9, or 10
(GSGGS)ₙ (SEQ ID NO: 108), optionally where n = 1-10, e.g., 1, 2, 3, 4,
5, 6, 7, 8, 9, or 10
ADAAP (SEQ ID NO: 109)
ADAAPTVSIFP (SEQ ID NO: 110)
ADAAPTVSIFPP (SEQ ID NO: 111)
AKTTAP (SEQ ID NO: 112)
AKTTAPSVYPLAP (SEQ ID NO: 113)
AKTTPKLEEGEFSEARV (SEQ ID NO: 114)
AKTTPKLGG (SEQ ID NO: 115)
AKTTPP (SEQ ID NO: 116)
AKTTPPSVTPLAP (SEQ ID NO: 117)
ASTKGP (SEQ ID NO: 118)
ASTKGPSVFPLAPASTKGPSVFPLAP (SEQ ID NO: 119)
EGKSSGSGSESKST (SEQ ID NO: 120)
GEGESGEGESGEGES (SEQ ID NO: 121)
GEGESGEGESGEGESGEGES (SEQ ID NO: 122)
GEGGSGEGGSGEGGS (SEQ ID NO: 123)
GENKVEYAPALMALS (SEQ ID NO: 124)
GGEGSGGEGSGGEGS (SEQ ID NO: 125)
GGGESGGEGSGEGGS (SEQ ID NO: 126)
GGGESGGGESGGGES (SEQ ID NO: 127)
GGGGSGGGGS (SEQ ID NO: 128)
GGGGSGGGGSGGGGS (SEQ ID NO: 129)
GGGGSGGGGSGGGGSGGGGS (SEQ ID NO: 130)
GGGKSGGGKSGGGKS (SEQ ID NO: 131)
GGGKSGGKGSGKGGS (SEQ ID NO: 132)
GGGS (SEQ ID NO: 133)
GGGSG (SEQ ID NO: 134)
GGKGSGGKGSGGKGS (SEQ ID NO: 135)
GGSG (SEQ ID NO: 136)
GGSGG (SEQ ID NO: 92)
GGSGGGGSG (SEQ ID NO: 137)
GGSGGGGSGGGGS (SEQ ID NO: 138)
GHEAAAVMQVQYPAS (SEQ ID NO: 139)
GKGGSGKGGSGKGGS (SEQ ID NO: 140)
GKGKSGKGKSGKGKS (SEQ ID NO: 141)
GKGKSGKGKSGKGKSGKGKS (SEQ ID NO: 142)
GKPGSGKPGSGKPGS (SEQ ID NO: 143)
GKPGSGKPGSGKPGSGKPSGS (SEQ ID NO: 144)
GPAKELTPLKEAKVS (SEQ ID NO: 145)
GSAGSAAGSGEF (SEQ ID NO: 146)
GSGGG (SEQ ID NO: 147)
GSGSG (SEQ ID NO: 148)
GSS
GSSG (SEQ ID NO: 149)
GSSGGSGGSG (SEQ ID NO: 150)
GSSGGSGGSGG (SEQ ID NO: 151)
GSSGGSGGSGGS (SEQ ID NO: 152)
GSSGGSGGSGGSG (SEQ ID NO: 153)
GSSGGSGGSGGSGGGS (SEQ ID NO: 154)
GSSGGSGGSGS (SEQ ID NO: 155)
GSSGT (SEQ ID NO: 156)
GSSSG (SEQ ID NO: 157)
GSTSGSGKPGSGEGSTKG (SEQ ID NO: 158)
GTAAAGAGAAGGAAAGAAG (SEQ ID NO: 159)
GTSGSSGSGSGGSGSGGGG (SEQ ID NO: 160)
IRPRAIGGSKPRVA (SEQ ID NO: 161)
KESGSVSSEQLAQFRSLD (SEQ ID NO: 162)
KTTPKLEEGEFSEAR (SEQ ID NO: 163)
PRGASKSGSASQTGSAPGS (SEQ ID NO: 164)
QPKAAP (SEQ ID NO: 165)
QPKAAPSVTLFPP (SEQ ID NO: 166)
RADAAAA(G4S)4 (SEQ ID NO: 167)
RADAAAAGGPGS (SEQ ID NO: 168)
RADAAP (SEQ ID NO: 169)
RADAAPTVS (SEQ ID NO: 170)
SAKTTP (SEQ ID NO: 171)
SAKTTPKLEEGEFSEARV (SEQ ID NO: 172)
SAKTTPKLGG (SEQ ID NO: 173)
STAGDTHLGGEDFD (SEQ ID NO: 174)
TVAAP (SEQ ID NO: 175)
TVAAPSVFIFPP (SEQ ID NO: 176)
TVAAPSVFIFPPTVAAPSVFIFPP (SEQ ID NO: 177)

### 6.8. Split Inteins

The present disclosure relates to circularized antibody molecules, the circularization of which is achieved by split intein-mediated protein splicing.

Inteins are protein elements that are capable of ligating sequences of other proteins with a peptide bond, a process known as protein splicing by analogy to the splicing of RNA introns from pre-mRNA (Perler et al., 1994, Nucl Acids Res. 22:1125-1127).

Several forms of inteins have been characterized, including full-length inteins, mini-inteins, and split inteins. Full-length and mini-inteins are cis-splicing inteins, wherein they join the two flanking sequences of a precursor protein and self-excise from the newly formed protein. Full-length inteins are typically 350-550 amino acids in size and also contain a homing endonuclease domain, whereas mini-inteins have only the ˜140-aa splicing domain and lack the endonuclease domain.

A split intein is a trans-splicing intein, in which two complementary domains of the intein are not directly linked via a peptide bond but are expressed either on two separate polypeptide sequences, or at the N- and C-termini of a single polypeptide chain.

When two distinct polypeptides comprise complementary domains of a split intein, trans-splicing generates a single protein by ligating the two polypeptides. For instance, pairing of the complementary domains of a split intein can splice two precursor polypeptides (e.g., exteins) into a fused polypeptide product, wherein one precursor protein consists of a polypeptide (N-extein) fused to the N-terminal intein fragment (N-intein), and another precursor protein consists of the C-terminal intein fragment (C-intein) fused to another polypeptide (C-extein). Upon trans-splicing of the two precursor proteins, the intein fragments self-excise, and the N-extein and the C-extein are joined with a peptide bond.

When a single polypeptide sequence comprises complementary domains of a split intein, e.g., C-intein and N-intein, at its N- and C-termini, respectively, the two ends of the polypeptide sequence are ligated, which results in a circular protein.

Most inteins that have been characterized are full-length or mini inteins. Although naturally occurring split inteins are relatively rare, they can be generated from both full-length and mini inteins. A non-limiting list of inteins that are either split inteins or can be used to generate split inteins is provided in Table 1 of PCT Publication No. WO 2021/099607 A1, which is incorporated by reference in its entirety.

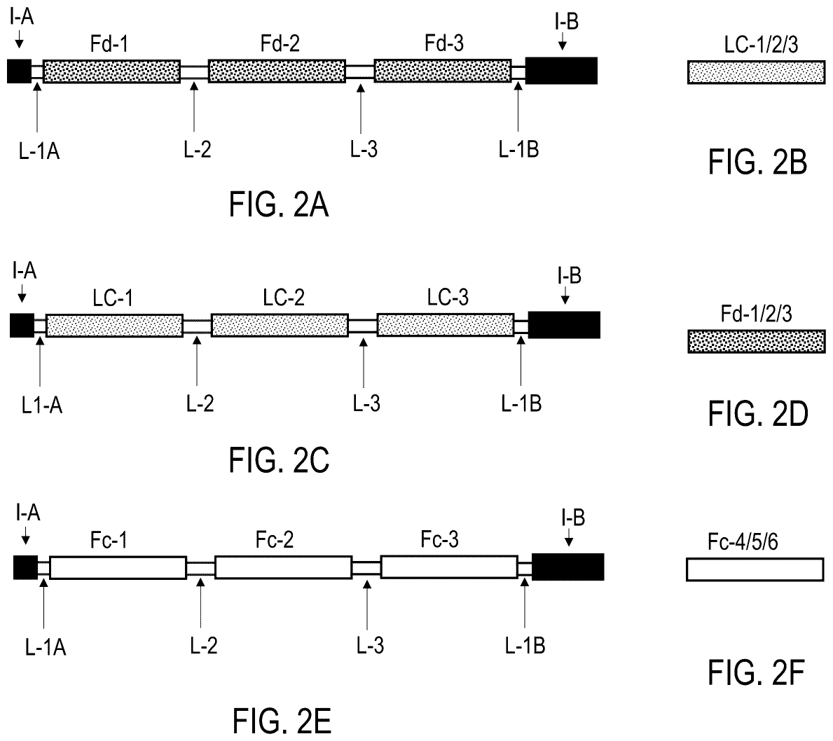

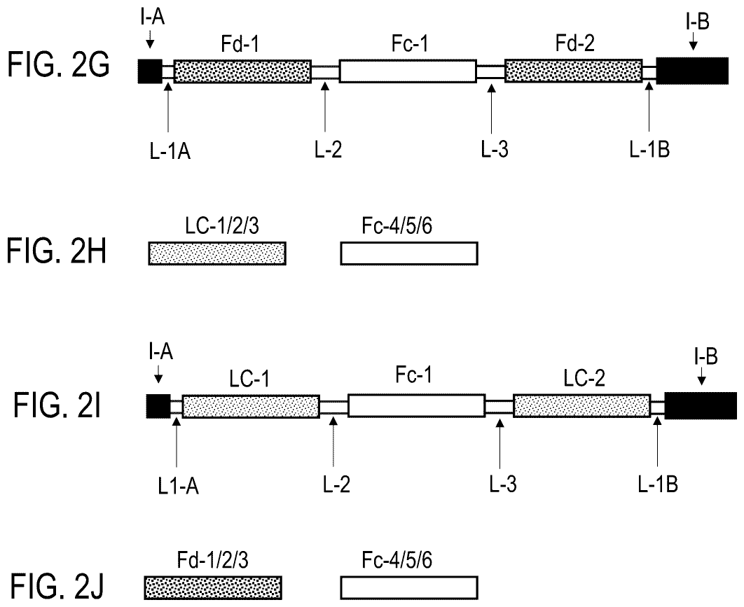

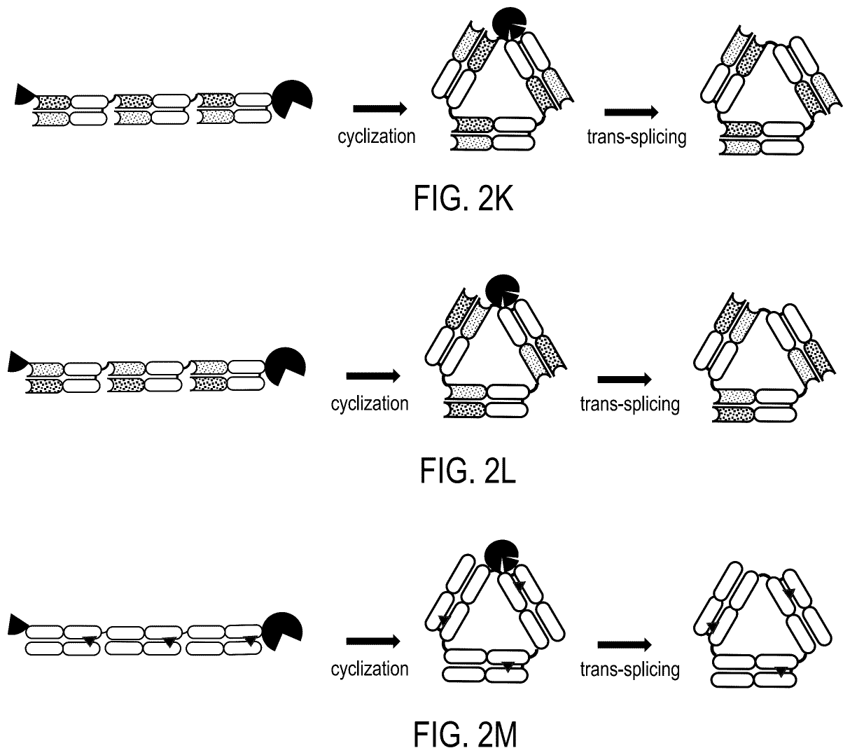

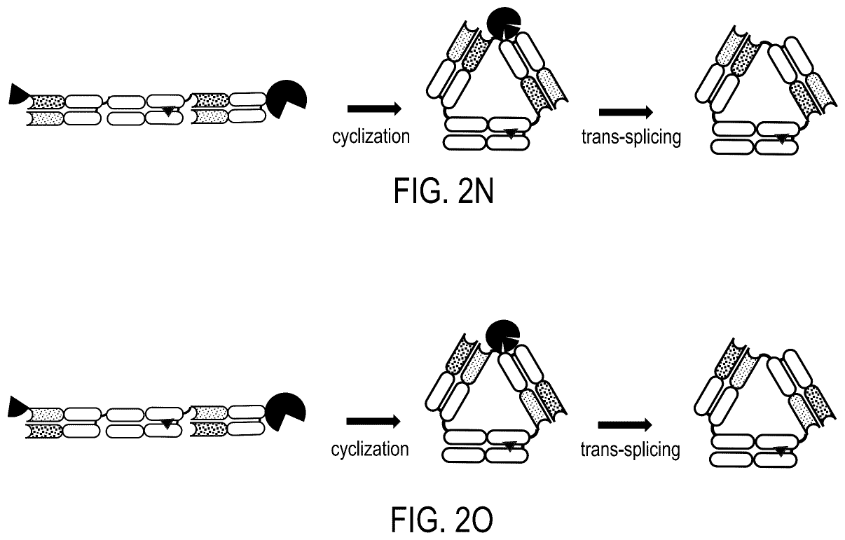

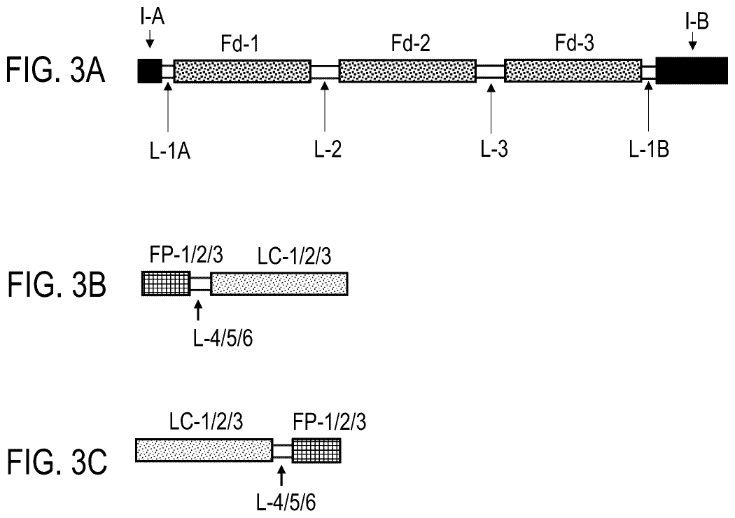

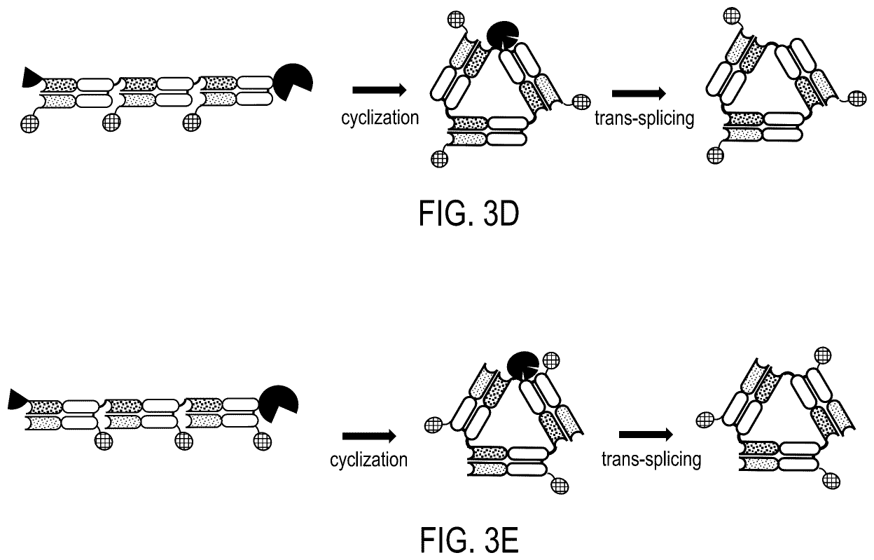

In some embodiments, the circularized antibody molecule is a circularized tri-Fab, comprising three Fab domains. In some embodiments, the circularized tri-Fab is obtainable from a precursor in which one of the polypeptide chains comprises three Fd domains flanked by two complementary split inteins having the configuration shown in FIGS. 2A and 3A.

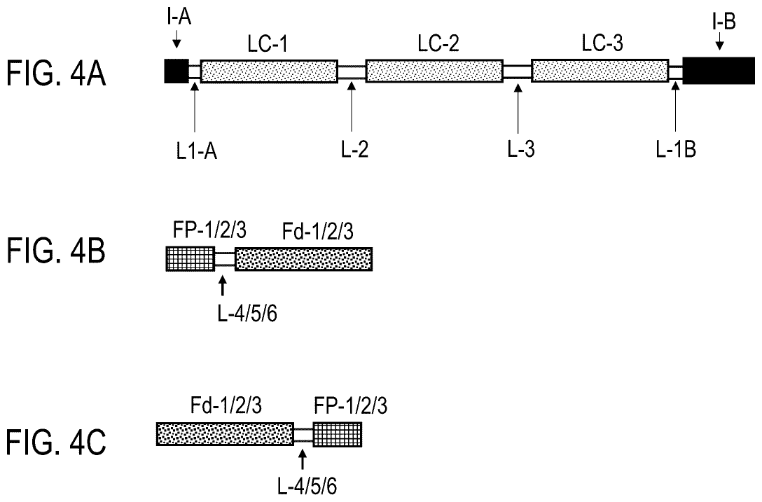

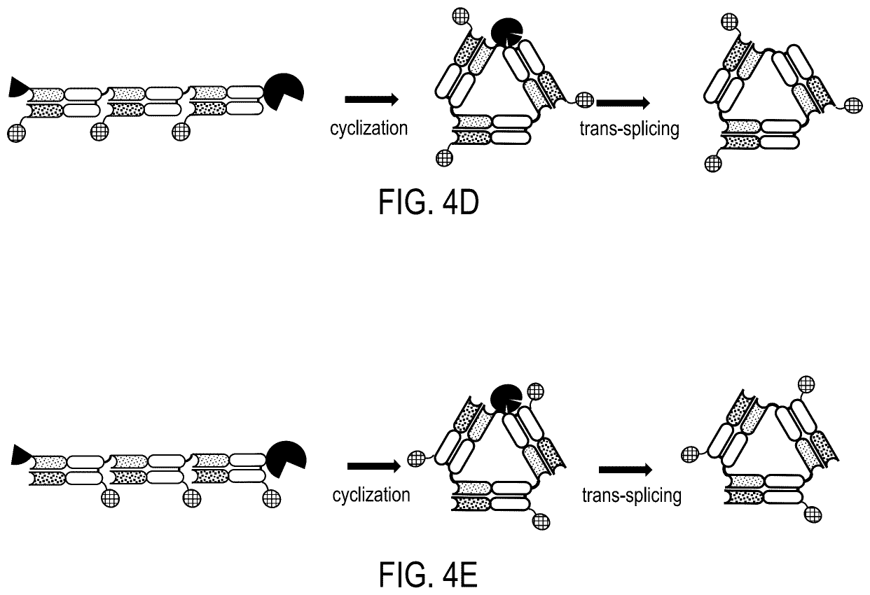

In other embodiments, the circularized tri-Fab is obtainable from precursor in which one of the polypeptide chains comprises three light chains flanked by two complementary split inteins having the configuration shown in FIGS. 2C and 4A.

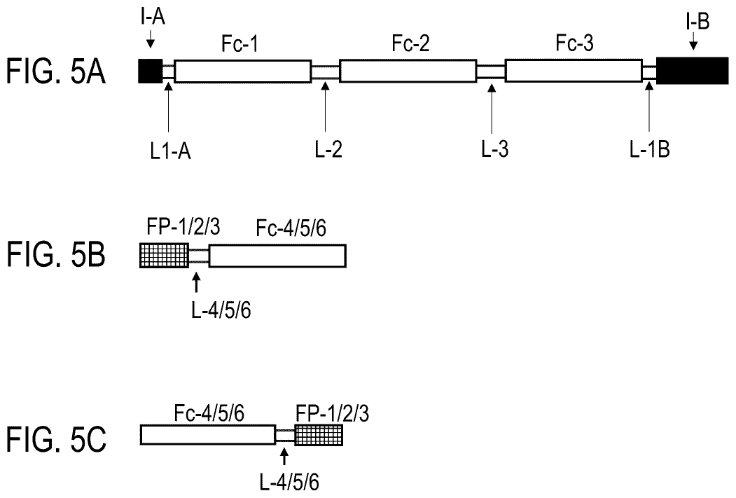

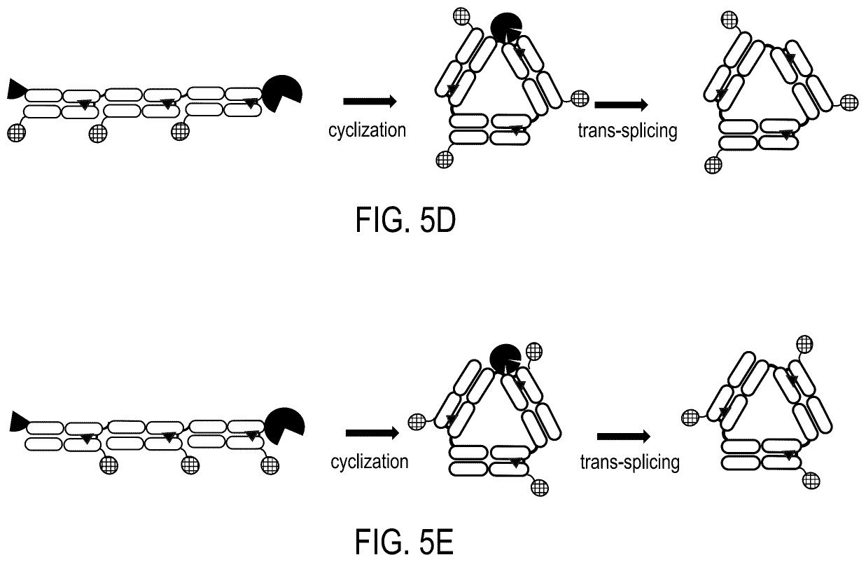

In some embodiments, the circularized antibody molecule is a circularized tri-Fc, comprising three Fc domains. In some embodiments, the circularized tri-Fc is obtainable from a precursor in which one of the polypeptide chains comprises three Fc domains (e.g., Fc (knob) or Fc (hole) domains) flanked by two complementary split inteins having the configuration shown in FIGS. 2E and 5A.

In some embodiments, the circularized antibody molecule comprises both Fab and Fc domains. In some embodiments, the circularized antibody molecule is obtainable from a precursor in which one of the polypeptide chains comprises two Fd domains and one Fc domain (e.g., an Fc (knob) or Fc (hole) domain) flanked by two complementary split inteins having the configuration shown in FIG. 2G. In some other embodiments, the circularized antibody molecule is obtainable from a precursor in which one of the polypeptide chains comprises one Fd domain and two Fc domains (e.g., Fc (knob) or Fc (hole) domains) flanked by two complementary split inteins.

In further embodiments, the circularized antibody molecule comprises both Fab and Fc domains. In some embodiments, the circularized antibody molecule is obtainable from a precursor in which one of the polypeptide chains comprises two light chains and one Fc domain (e.g., an Fc (knob) or Fc (hole) domain) flanked by two complementary split inteins having the configuration shown in FIG. 2I. In some other embodiments, the circularized antibody molecule is obtainable from a precursor in which one of the polypeptide chains comprises one light chain and two Fc domains (e.g., Fc (knob) or Fc (hole) domains) flanked by two complementary split inteins. In some embodiments, the split intein is a naturally occurring split intein.

In some embodiments, the split intein is a DnaE intein, e.g., a DnaE-Npu, DnaE-Ssp, DnaE-Aha, DnaE-Aov, DnaE-Asp, DnaE-Ava, DnaE-Cra (CS505), DnaE-Csp (CCY0110), DnaE-Csp (PCC8801), DnaE-Cwa, DnaE-Maer (NIES843), DnaE-Mcht (PCC7420), DnaE-Oli, DnaE-Sel (PC7942), DnaE-Ssp (PCC7002), DnaE-Tel, DnaE-Ter, or DnaE-Tvu intein.

In some other embodiments, the split intein is a NrdJ-1, NrdA-1, GP41.1, IMPDH-1, or GP41.8 intein.

In further embodiments, the split intein is a non-naturally occurring split intein, wherein one or both of split intein portions are derived from full-length or mini inteins, e.g., a split intein derived from RecA, DnaB, Psp Pol-I, or Pfu intein.

Amino acid sequences of exemplary split inteins are set forth in Table 7.

TABLE 7
Exemplary Split Inteins

| Intein Name | Intein Sequence |
|---|---|
| GP41.1 (N-terminal | CLDLKTQVQTPQGMKEISNIQVGDLVLSNTGYNEVLNVFPKSK |
| domain) | KKSYKITLEDGKEIICSEEHLFPTQTGEMNISGGLKEGMCLYVK |
| | E (SEQ ID NO: 178) |
| GP41.1 (C-terminal | MMLKKILKIEELDERELIDIEVSGNHLFYANDILTHN (SEQ ID |
| domain) | NO: 179) |
| GP41.8 (N-terminal | CLSLDTMVVTNGKAIEIRDVKVGDWLESECGPVQVTEVLPIIKQ |
| domain) | PVFEIVLKSGKKIRVSANHKFPTKDGLKTINSGLKVGDFLRSRA |
| | K (SEQ ID NO: 180) |
| GP41.8 (C-terminal) | MCEIFENEIDWDEIASIEYVGVEETIDINVTNDRLFFANGILTHN |
| | (SEQ ID NO: 181) |
| NrdJ-1 (N-terminal | CLVGSSEIITRNYGKTTIKEVVEIFDNDKNIQVLAFNTHTDNIEW |
| domain) | APIKAAQLTRPNAELVELEINTLHGVKTIRCTPDHPVYTKNRDY |
| | VRADELTDDDELVVAI (SEQ ID NO: 182) |
| NrdJ-1 (C-terminal | MEAKTYIGKLKSRKIVSNEDTYDIQTSTHNFFANDILVHN (SEQ |
| domain) | ID NO: 183) |
| NrdA-2 (N-terminal | CLTGDAKIDVLIDNIPISQISLEEVVNLFNEGKEIYVLSYNIDTKEV |
| domain) | EYKEISDAGLISESAEVLEIIDEETGQKIVCTPDHKVYTLNRGYV |
| | SAKDLKEDDELVFS (SEQ ID NO: 184) |
| NrdA-2 (C-terminal | MGLKIIKRESKEPVFDITVKDNSNFFANNILVHN (SEQ ID NO: |
| domain) | 185) |
| IMPDH-1 (N-terminal | CFVPGTLVNTENGLKKIEEIKVGDKVFSHTGKLQEVVDTLIFDR |
| domain) | DEEIISINGIDCTKNHEFYVIDKENANRVNEDNIHLFARWVHAEE |
| | LDMKKHLLIELE (SEQ ID NO: 186) |
| IMPDH-1 (C-terminal | MKFKLKEITSIETKHYKGKVHDLTVNQDHSYNVRGTVVHN |
| domain) | (SEQ ID NO: 187) |
| DnaE-Npu (N- | CLSYETEILTVEYGLLPIGKIVEKRIECTVYSVDNNGNIYTQPVA |
| fragment) | QWHDRGEQEVFEYCLEDGSLIRATKDHKFMTVDGQMLPIDEIF |
| | ERELDLMRVDNLPN (SEQ ID NO: 188) |
| DnaE-Npu (C- | MIKIATRKYLGKQNVYDIGVERDHNFALKNGFIASN (SEQ ID |
| fragment) | NO: 189) |
| DnaE-Ssp (N- | CLSFGTEILTVEYGPLPIGKIVSEEINCSVYSVDPEGRVYTQAIA |
| fragment) | QWHDRGEQEVLEYELEDGSVIRATSDHRFLTTDYQLLAIEEIFA |
| | RQLDLLTLENIKQTEEALDNHRLPFPLLDAGTIK (SEQ ID NO: |
| | 190) |
| DnaE-Ssp (C- | MVKVIGRRSLGVQRIFDIGLPQDHNFLLANGAIAAN (SEQ ID |
| fragment) | NO: 191) |
| DnaE-Aha (N- | CLSYDTEIWTVEYGAMPIGKIVEEKIECSVYTVDENGFVYTQPIA |
| fragment) | QWHPRGQQEIIEYTLEDGRKIRATKDHKMMTESGEMLPIEEIF |
| | QRELDLKVETFHEMSLLRRGAK (SEQ ID NO: 192) |
| DnaE-Aha (C- | MVKIIKRQSLGRQNVYDVCVETDHNFVLANGCVASN (SEQ ID |
| fragment) | NO: 193) |
| DnaE-Aov (N- | CLSADTEILTVEYGFLPIGEIVGKAIECRVYSVDGNGNIYTQSIA |
| fragment) | QWHNRGEQEVFEYTLEDGSIIRATKDHKFMTTDGEMLPIDEIFA |
| | RQLDLMQVQGLH (SEQ ID NO: 194) |
| DnaE-Aov (C- | MVKITARKFVGRENVYDIGVEHHHNFAIKNGLIASN (SEQ ID |
| fragment) | NO: 195) |
| DnaE-Asp (N- | CLSYDTEVLTVEYGFVPIGEIVEKGIECSVFSINNNGIVYTQPIAQ |
| fragment) | WHHRGKQEVFEYCLEDGSIIKATKDHKFMTQDGKMLPIDEIFE |
| | QELDLLQVKGLPE (SEQ ID NO: 196) |
| DnaE-Asp (C- | MIKIASRKFLGVENVYDIGVRRDHNFFIKNGLIASN (SEQ ID NO: |
| fragment) | 197) |
| DnaE-Ava (N- | CLSYDTEVLTVEYGFVPIGEIVDKGIECSVFSIDSNGIVYTQPIAQ |
| fragment) | WHHRGKQEVFEYCLEDGSIIKATKDHKFMTQDGKMLPIDEIFE |
| | QELDLLQVKGLPE (SEQ ID NO: 198) |
| DnaE-Ava (C- | MIKIASRKFLGVENVYDIGVGRDHNFFVKNGLIASN (SEQ ID |
| fragment) | NO: 199) |
| DnaE-Cra(CS505) | CLSYETEVLTLEYGFVPIGEIVNKQMVCTVFSLNDSGNVYTQPI |
| (N-fragment) | GQWHDRGVQDLYEYCLDDGSTIRATKDHKFMTTQGEMVPIDE |
| | IFHQGWELVQVSGISKLVQQRTLPFIIVDRKL (SEQ ID NO: 200) |
| DnaE-Cra(CS505) | MVKIVSRRYLGKADVYDIGVAKDHNFIIKNGLVASN (SEQ ID |
| (C-fragment) | NO: 201) |
| DnaE-Csp(CCY0110) | CLSYDTEILTVEYGPMPIGKIVEENINCSVYTVNKNGFVYTQSIA |
| (N-fragment) | QWHHRGEQEVFEYYLEDGETIRATKDHKFMTTEGKMLPIDEIF |
| | ENNLDLKKLTV (SEQ ID NO: 202) |
| DnaE-Csp(CCY0110) | MVKIIERRSLGKQNVYDIGVEKDHNFLLSNNLIASN (SEQ ID |
| (C-fragment) | NO: 203) |
| DnaE-Csp(PCC8801) | CLSYDTEILTVEYGAIPIGKVVEENIDCTVYTVDKNGFVYTQNIA |
| (N-fragment) | QWHLRGQQEVFEYYLDDGSILRATKDHQFMTLEGEMLPIHEIF |
| | ERGLELKKIKI (SEQ ID NO: 204) |
| DnaE-Csp(PCC8801) | MVKIVSYRSLGKQFVYDIGVAQDHNFLLANGSIASN (SEQ ID |
| (C-fragment) | NO: 205) |
| DnaE-Cwa (N- | CLSYDTEILTVEYGAMYIGKIVEENINCTVYTVDKNGFVYTQTIA |
| fragment) | QWHNRGEQEIFEYDLEDGSKIKATKDHKFMTIDGEMLPIDEIFE |
| | KNLDLKQVVSHPDDYLV (SEQ ID NO: 206) |
| DnaE-Cwa (C- | MVKIIGCRSLGTQKVYDIGVEKDHNFLLANGSIASN (SEQ ID |
| fragment) | NO: 207) |
| DnaE- | CLGGETLILTEEYGLLPIAKIVSEEINCTVYTVDQNGFVYSQPIS |
| Maer(NIES843) (N- | QWHERGLQEVFEYTLENGQTIQATKDHKFMTSDGEMLAIDTIF |
| fragment) | ERGLDLKSSDFS (SEQ ID NO: 208) |
| DnaE- | MVKIIGRQSLGRKPVYDIGVEKDHNFLLGNGLIASN (SEQ ID |
| Maer(NIES843) (C- | NO: 209) |
| fragment) | |
| DnaE- | CLSYDTQILTVEYGAVAIGEIVEKQIECTVYSVDENGYVYTQPIA |
| Mcht(PCC7420) (N- | QWHNRGEQEVFEYLLEDGATIRATKDHKFMTDEDQMLPIDQIF |
| fragment) | EQGLELKQVEVLQPVF (SEQ ID NO: 210) |
| DnaE- | MVKIVRRQSLGVQNVYDIGVEKDHNFCLASGEIASN (SEQ ID |
| Mcht(PCC7420) (C- | NO: 211) |
| fragment) | |
| DnaE-Oli (N- | CLSYNTEVLTVEYGPLPIGKIVDEQIHCRVYSVDENGFVYTQAI |
| fragment) | AQWHDRGYQEIFAYELADGSVIRATKDHQFMTEDGQMFPIDEI |
| | WEKGLDLKKLPTVQDLPAAVGYTVS (SEQ ID NO: 212) |
| DnaE-Oli (C- | MVKIVRRQSLGVQNVYDIGVEKDHNFCLASGEIASN (SEQ ID |
| fragment) | NO: 211) |
| DnaE-Sel(PC7942) | CLAADTEVLTVEYGPIAIGKLVEENIRCQVYCCNPDGYIYSQPIG |
| (N-fragment) | QWHQRGEQEVIEYELSDGRIIRATADHRFMTEEGEMLSLDEIF |
| | ERSLELKQIPTPLLAIAQPSPLATA (SEQ ID NO: 213) |
| DnaE-Sel(PC7942) | MVKIVRRRSLGVQPVYDLGVATVHNFVLANGLVASN (SEQ ID |
| (C-fragment) | NO: 214) |
| DnaE-Ssp(PCC7002) | CLAGGTPVVTVEYGVLPIQTIVEQELLCHVYSVDAQGLIYAQLIE |
| (N-fragment) | QWHQRGDRLLYEYELENGQMIRATPDHRFLTTTGELLPIDEIFT |
| | QNLDLAAWAVPDSLPRTA (SEQ ID NO: 215) |
| DnaE-Ssp(PCC7002) | MVKIIRRKFIGHAPTYDIGLSQDHNFLLGQGLIAAN (SEQ ID NO: |
| (C-fragment) | 216) |
| DnaE-Tel (N- | CLSGETAVMTVEYGAVPIRRLVQERLSCHVYSLDGQGHLYTQ |
| fragment) | PIAQWHFQGFRPVYEYQLEDGSTICATPDHRFMTTRGQMLPIE |
| | QIFQEGLELWQVAIAPRQALLQGLKPAVQMSG (SEQ ID NO: |
| | 217) |
| DnaE-Tel (C- | MKIVGRRLMGWQAVYDIGLAADHNFVLANGAIAAN (SEQ ID |
| fragment) | NO: 218) |
| DnaE-Ter (N- | CLTYETEIMTVEYGPLPIGKIVEYRIECTVYTVDKNGYIYTQPIAQ |
| fragment) | WHNRGMQEVYEYSLEDGTVIRATPEHKFMTEDGQMLPIDEIFE |
| | RNLDLKCLGTLE (SEQ ID NO: 219) |
| DnaE-Ter (C- | MVKIVSRKLAKTENVYDIGVTKDHNFVLANGLIASN (SEQ ID |
| fragment) | NO: 220) |
| DnaE-Tvu (N- | CLSGETAVMTVEYGAIPIRRLVQERLICQVYSLDPQGHLYTQPI |
| fragment) | AQWHFQGFRPVYAYQLEDGSTICATPDHRFMTTSGQMLPIEQI |
| | FREGLELWQVAIAPPGALAQGLKPAVQMSC (SEQ ID NO: 221) |
| DnaE-Tvu (C- | MKIVGRRLVGWQAVYDIGLAGDHNFLLANGAIAAN (SEQ ID |
| fragment) | NO: 222) |
| AceL-TerL-11 Int^{N} | CVYGDTMVETEDGKIKIEDLYKRLA (SEQ ID NO: 223) |
| AceL-TerL-11 Int^{C} | MFRTNTNNIKILSPNGFSNFNGIQKVERNLYQHIIFDDDTEIKTSI |
| | NHPFGKDKILARDVKVGDYLNSKKVLYNELVNENIFLYDPINVE |
| | KESLYITNGVVSHN (SEQ ID NO: 224) |
| GS033-TerA-6 Int^{N} | SISQESYINIEVNGKVETIKIGDLYKKLSFNERKFNE (SEQ ID |
| | NO: 225) |
| GS033-TerA-6 Int^{C} | MKLPESVVKNNINLKIETPYGFENFYGVNKIKKDKYIHLEFTNGE |
| | KLKCSLDHPLSTIDGIVKAKDLDKYTEVYTKFGGCFLKKSKVINE |
| | SIELYDIVNSGLKHLYYSNNIISHN (SEQ ID NO: 226) |
| TerA-1-CP21-BP Int^{N} | CCLENTRVQVRNKYTNKIETLTIKELYARLQELKKS (SEQ ID |
| | NO: 227) |
| TerA-1-CP21-BP Int^{C} | MSEIQDINPYEILTPQGFKPFVDIIKSIQTTGITITLEDSREISVTLD |
| | HKFKHLNDYKEAKYFKVGDKLQCSKIIKIENIEGEFYEPLEVQD |
| | HEYIANDFINHN (SEQ ID NO: 228) |
| TerA-1-CP81-BP Int^{N} | CVAGDTKITVRNKKTGVIEDITMEELYNRIG (SEQ ID NO: 229) |
| TerA-1-CP81-BP Int^{C} | MYEVLTPNGFSDFDDISREKKDVYKVITEDDFIKVTKGHKFETP |
| | NGFKQLKHLKINDLIKYKNKFSKIVLIDYVGVEYVYDLINVHKNN |
| | EYYTNNFVSHN (SEQ ID NO: 230) |
| AceL 1-ClpC-1 Int^{N} | CFSKKTSIKLRNKKTGDLEEIDISDLIYELHIS (SEQ ID NO: 231) |
| AceL 1-ClpC-1 Int^{C} | MIKLYNKKQNKKFTKSYDLGDYQILTDSGYIGLVSLHETIPYEV |
| | WKLKLSNGYELECADDHIIFDNEMNEIFVKNLELGDRVKVDDG |
| | YAVVIELVNTGLLESMYDFELVEDSNRRYYTNGILSHN (SEQ ID |
| | NO: 232) |
| AceL 1-ClpC-2 Int^{N} | CVSPNTKIKIRNSSTGEISEVTIAEFNKMI (SEQ ID NO: 233) |
| AceL 1-ClpC-2 Int^{C} | MKKIVKSVSVEGFEVLSDNGWVPIKNVHTTVPYELYNLRTANG |
| | LRLECADNHIVFTSKLKEVYVKDLNVDDKIMTEDGVSLVSSIEK |
| | TKAKVTMYDLEVDSEDHRYYTDGILSHN (SEQ ID NO: 234) |
| AceL 1-RadA1-1 Int^{N} | CVHPNTLVKIKIDSTGEERTITVKDLHELIKSVK (SEQ ID NO: |
| | 235) |
| AceL 1-RadA1-1 Int^{C} | MKRKFIESISADNISIMTDTGWEKVKGSHVTIEYKVFNLVTDRLS |
| | LQCADDHIVFKEDFSEVFVKDLEVGDLIQTVNGLESVTEVYETD |
| | DLVNMHDLEIDSKNHRYYTDGILSHN (SEQ ID NO: 236) |
| AceL 1-TerL-10 Int^{N} | CVSGDTKVTLKDNDTGKIINVNIEEMVSVSSLDV (SEQ ID NO: |
| | 237) |
| AceL 1-TerL-10 Int^{C} | MEVGKMSKSYKVLSPSGFVDFAGIQKITRSKYRHFIFDDGTEIK |
| | CSLNHRFGEEEIVASTLHHGTELQGKKILYAEDVEDDIDLYDLL |
| | NVANGNLYYTNGLVSHN (SEQ ID NO: 238) |
| AceL 1-TerL-2 Int^{N} | CFFFNTIISVETNNQQYETRIGILYYSMVSKERNLTILEKIKIKLYD |
| | LLFILEKH (SEQ ID NO: 239) |
| AceL 1-TerL-2 Int^{C} | MLRIFKRCLIYLIKKMIEFIELYEYKKISLDECDINKKILNSISLMDL |
| | KVETDTGYETSSNIHITQPFKHYNIETVDGYEIICADNHILFDEEF |
| | NEVFTKDLKIGDLIKTKNGNSVIKSIYIDTHKSSMFDLTIDHPNHR |
| | FYTNGILSHN (SEQ ID NO: 240) |
| AceL 1-TerL-3 Int^{N} | CVDGNTIVETEDGKIKIEDLYKKL (SEQ ID NO: 241) |
| AceL 1-TerL-3 Int^{C} | MFITNTDNIKILSPSGFSNFNGIQKVERNLYQHIIFDDESEIKTSIN |
| | HPFGKNKILARNVKVGDYLSSKKVLYNELVNEKIFLYDPINVEK |
| | ENLYITNGVVSHN (SEQ ID NO: 242) |
| AceL 1-TerL-4 Int^{N} | CVDGNTIVETEDGKIKIEDLYKKM (SEQ ID NO: 243) |
| AceL 1-TerL-4 Int^{C} | MFRTNTDNIKILSPSGFSIFNGIQKVERDLYQHIIFDDKSEIKTSIN |
| | HPFGKDKILARNIKVGDYLNSKKVLYNELVAEKITLYDPINVEKE |
| | NLYITNGVISHN (SEQ ID NO: 244) |
| AceL 1-TerL-5 Int^{N} | CVDGNTIVETEDGKIKIEDLYKKL (SEQ ID NO: 241) |
| AceL 1-TerL-5 Int^{C} | MFRTNTDNIKILSPSGFSNFNGIQKVERDLYQHIIFDDKSEIKTSI |
| | NHPFGKDKILARNIKVGDYLNSKKVLYNELVNEKITLYDPINVEK |
| | ENLYITNGVISHN (SEQ ID NO: 245) |
| AceL 1-UvsW-3-1 Int^{N} | CRTYDSTMDIDVGNSDFAEYLLNNSKK (SEQ ID NO: 246) |
| AceL 1-UvsW-3-1 Int^{C} | MKFNIPIGELAESIAKYKGVLLNDNCEINIKDLDCKVNTPSGTATI |
| | NIIIKKEKLEGIKLLLANGVEIKCANKHILRYNNADVFADSLAIGDS |
| | VETINGNVKVSSINNIDDTTFYDIGIDAPYLYYDADGVLHHN |
| | (SEQ ID NO: 247) |
| AceL 1-gp41-1 Int^{N} | CFFSDGEINTRNISNKEIKSIKIGKIFTNISKGHTNI (SEQ ID NO: |
| | 248) |
| AceL 1-gp41-1 Int^{C} | MLDNYEIIEADSLLEGKYDRPLYDKFIEAYEVDNLEVDTPNGWI |
| | KIEGIGKTIEFYEWEIQTSGGKHLICADKHLLYRCDNMNFYNKK |
| | CDITEIYCQDLNIGDFIMTKDGPEMLMDIYKNGNKSNMYDLQLS |
| | EGSNKQYYTNDILSHN (SEQ ID NO: 249) |
| AceL 1-gp46 Int^{N} | CVDESTLIDVQIIDFEPNLENLEFLDKTDEGKRIFLYIKKSNKSLY |
| | EKIEKFRKGQ (SEQ ID NO: 250) |
| AceL 1-gp46 Int^{C} | MLTLKIGDLYELSKNINILESDIRVSTPGGLKKVFAVDITAKNSDV |
| | FSIKVNKHELLCSPDHLIRSEDMWVKSKDLKINSVIDTKYGKLTV |
| | KEISILDIKSDLMDLHVDGSEYYTNDIISHN (SEQ ID NO: 251) |
| VidaL-T4Lh-1 Int^{N} | CVHPDTKVTIRRKLC (SEQ ID NO: 252) |
| VidaL-T4Lh-1 Int^{C} | MKELLDLYTEKEINKLLERYTIDQIIDYSQPHVVSVGSIKEEMDS |
| | GNFIFVDSPDGYVAVSDFVDKGNFEEYRFTYDKKIIRTNEGHLF |
| | QTHLGWETSKNLYKMYLAGHPIYILHKNGGYKKIDIEKTGNVIPI |
| | VDIVVEHKNHRYYTDGLSSHN (SEQ ID NO: 253) |
| VidaL-UvsX-2 Int^{N} | CLPKEAVVQIRLTKKG (SEQ ID NO: 254) |
| VidaL-UvsX-2 Int^{C} | MIEEKKVTVQELRELYLSGEYTIEIDTPDGYQTIGKWFDKGVLS |
| | MVRVATATYETVCAFNHMIQLADNTWVQACELDVGVDIQTAA |
| | GIQPVMLVEDTSDAECYDFEVMHPNHRYYGDGIVSHN (SEQ |
| | ID NO: 255) |
| VidaL-TerL-6 Int^{N} | SLAHETIVSINDNNTLTSMCIGDLYDYM (SEQ ID NO: 256) |
| VidaL-TerL-6 Int^{C} | MDYHSNQVSRIFGVGMSKVHLGFKKNTKNLKVLTPNGHEEFY |
| | GINKIRVDEYIRIKFKEHKEIRCSIDHPFIQENDLPIKAKHIDKSKH |
| | IKCIDGFTTLEYSHVVNKQIELYDIVNSGSEYIYFSNGILSHN |
| | (SEQ ID NO: 257) |
| VidaL-TerL-7-1 Int^{N} | CLWGASTVNVFDSLTGKNIDIKLEDLYQKL (SEQ ID NO: 258) |
| VidaL-TerL-7-1 Int^{C} | MESYTFRKNTRYKIMTPAGYQNFGGIRKLNKNVHYIVELSNKKI |
| | LKCSTTHPFIYNDREIFANKLKVGSLLDSTSKKKISVISIELDKSKI |
| | DLYDIVEVNNGNIFNVDGIVSHN (SEQ ID NO: 259) |
| VidaL-TerL-3 Int^{N} | CVSASTIITLQDTHGNIFDSQIGDLYNTIGK (SEQ ID NO: 260) |
| VidaL-TerL-3 Int^{C} | MSKIFKENTNGYKVLTPAGFQDFAGVSMMGIKPLLRLEFERGA |
| | YVECTYDHKFYIDLETCKPAQDIAVGNTVVTSEGDIKLLNKIELG |
| | YSEPVYDLIQVEGGHRYYTNKILSSN (SEQ ID NO: 261) |
| VidaL-TerL-1 Int^{N} | CVQADTKYTIRNKISGDVLNVTAEEFHKMQKK (SEQ ID NO: |
| | 262) |
| VidaL-TerL-1 Int^{C} | MKLSNFTNRKFIETIDASEWEVETCEGFKPIISSNKTIEYVVYKIE |
| | LENGLSIKCADTHILIDKNLQEIYAKDSFNKIIFTKFGNSKVISVET |
| | LNISENMYDLSVDSEDHTYYTDDILSHN (SEQ ID NO: 263) |
| VidaL-gp46-1 Int^{N} | CLCINTIVKVKNTKTGVIYETTIGELYNGAME (SEQ ID NO: 264) |
| VidaL-gp46-1 Int^{C} | MSTISQTVNRKFVNSFSLVDLEIETDSGWQPVTDIHKTIPYTVW |
| | HIETQSGLTLDCADTHILFDHNYNEIFVKDIIPNQTKIISKHGPEL |
| | VLTVIEQSQQENMFDLTVDHPDHRFYSNNILSHN (SEQ ID NO: |
| | 265) |
| VidaL-gp41-1 Int^{N} | CVIAETEVKIIELYNIDSFIKTGILNSQGVVSWEETSSHGRTR |
| | (SEQ ID NO: 266) |
| VidaL-gp41-1 Int^{C} | MNLLDKRVEWLSKWYPVDKLQQLSEDKLVLLYNNSQPKKVRM |
| | GSLEGVPTSSYRISSPDGYVTAHAWRNKGTKECVTLTTDSGN |
| | SITASTDHFFEMSDGKWKYAGCLFPGQCISTESGTETVTSVVA |
| | AGKHTVYDFYIDHENHRYYTNGISSHN (SEQ ID NO: 267) |
| GS013-ter-3 Int^{N} | CLGGDTEIEILDDNGIVQKTSMENLYERL (SEQ ID NO: 268) |
| GS013-ter-3 Int^{C} | MFKINKNIKVKTPDGFKDFSGIQKVYKPFYHWIIFDDGSEIKCSD |
| | NHSFGKEKIKASTIKVDDILQEKKVLYNEIVEEGIYLYDLLDVGE |
| | DNLYYSNNIVSHN (SEQ ID NO: 269) |
| GS013-ter-2 Int^{N} | CLGGDTEIEILDDNGIVQKTSMENLYERL (SEQ ID NO: 268) |
| GS013-ter-2 Int^{C} | MSVGKMFKINKNIKVKTPDGFKDFSGIQKVYKPFYHWIIFDDGS |
| | EIKCSDNHSFGKEKIKASTIKVDDILQEKKVLYNEIVEEGIYLYDL |
| | LDVGEDNLYYSNNIVSHN (SEQ ID NO: 270) |
| GS013-ter-1 Int^{N} | CFNTNTTVRLRNKLTGEIIEVTIGEFYEKIKKESNTDLP (SEQ ID |
| | NO: 271) |
| GS013-ter-1 Int^{C} | MSKFIEEXXTDEWEVETPSGWQSFSGVGKTIEYEEWEVVTET |
| | GKSLICADKHILLNDKWQEVYCEDCSIDDCIQTKNXAEKILQLKK |
| | TSRIXNMYDLLDVDNGNIFYSNEIVSHN (SEQ ID NO: 272) |
| GS020-ter-7 Int^{N} | CVDGSSIITIKNKETNLIEKITIEELYNKLL (SEQ ID NO: 273) |
| GS020-ter-7 IntC | MKTNTKYEILGPEGFVDFKGIQKLKKKTRQIFFECGLTLRASYN |
| | HKIYDYFGDEIIIKDVVIGSKIKSHNGYLIVNSIKDFDYESDVYDVI |
| | DSGDSHLYYTNNIVSHN (SEQ ID NO: 274) |
| WTAceL-TerL-3 Int^{N} | CVDGNTIVETEDGKIKIEDLYKKL (SEQ ID NO: 241) |
| WTAceL-TerL-3 Int^{C} | MFITNTDNIKILSPSGFSNFNGIQKVERNLYQHIIFDDESEIKTSIN |
| | HPFGKNKILARNVKVGDYLSSKKVLYNELVNEKIFLYDPINVEK |
| | ENLYITNGVVSHN (SEQ ID NO: 242) |
| WTAceL-TerL-4 Int^{N} | CVDGNTIVETEDGKIKIEDLYKKM (SEQ ID NO: 243) |
| WTAceL-TerL-4 Int^{C} | MFRTNTDNIKILSPSGFSIFNGIQKVERDLYQHIIFDDKSEIKTSIN |
| | HPFGKDKILARNIKVGDYLNSKKVLYNELVAEKITLYDPINVEKE |
| | NLYITNGVISHN (SEQ ID NO: 244) |
| WTAceL-TerL-5 Int^{N} | CVDGNTIVETEDGKIKIEDLYKKL (SEQ ID NO: 241) |
| WTAceL-TerL-5 Int^{C} | MFRTNTDNIKILSPSGFSNFNGIQKVERDLYQHIIFDDKSEIKTSI |
| | NHPFGKDKILARNIKVGDYLNSKKVLYNELVNEKITLYDPINVEK |
| | ENLYITNGVISHN (SEQ ID NO: 245) |
| Aes^{N} | SVVGDTIIDVSGKKMTIAEFYDSTPDVFMRRNDEARDWVKRVG |
| | GKTSLSVNTYSGEVERKNINYIMKHTVKKRMFKIKAGGKEVIVT |
| | ADHSVMVKRDGKIIDVKPTEMKQTDRVVKWMLT (SEQ ID NO: |
| | 275) |
| Aes^{C} | MIEFIEFEIEDLGVMEIDVYDIEVDGNHNFFGNDILVHN (SEQ ID |
| | NO: 276) |
| CL^{N} | SVVGDTIIDVSGKKMTIAEFYDSTPD (SEQ ID NO: 277) |
| CL^{C} | EARDWVKRVGGKTSLSVNTYSGEVERKNINYIMKHTVKKRMF |
| | KIKAGGKEVIVTADHSVMVKRDGKIIDVKPTEMKQTDRVVKWM |
| | LTGSHMIEFIEFEIEDLGVMEIDVYDIEVDGNHNFFGNDILVHN |
| | (SEQ ID NO: 278) |

### 6.9. Fusion Partners

The circularized antibody molecules of the disclosure can comprise a fusion partner. In some embodiments, the fusion partner is on a polypeptide chain comprising a counterpart domain. The fusion partner can be N-terminal to the counterpart domain and/or C-terminal to the counterpart domain. The fusion partner can be from the counterpart domain by a linker. Such linkers are sometimes referred to herein as L-4, L-5 and L-6, and exemplary linker suitable for separating the fusion partners from counterpart domains are described in Section 6.7.

Suitable fusion partners include, but are not limited to, single chain antibodies and antibody fragments (e.g., as described in Section 6.9.1) and receptors, e.g., soluble receptor sequences (e.g., as described in Section 6.9.2).

### 6.9.1. Single Chain Antibodies and Antibody Fragments

In some embodiments, the fusion partner is a single chain antibody fragment, for example single chain Fvs as described in Section 6.9.1.1 and single domain antibodies as described in Section 6.9.1.2. In some embodiments, the single chain antibody fragments bind to a coronavirus spike protein, for example, comprising binding sequences as set forth in Section 6.5.1.

### 6.9.1.1. Single Chain Fvs

Single chain Fv or “scFv” antibody fragments comprise the VH and VL domains of an antibody in a single polypeptide chain, are capable of being expressed as a single chain polypeptide and retain the specificity of the intact antibodies from which they are derived. Generally, the scFv polypeptide further comprises a polypeptide linker between the VH and VL domain that enables the scFv to form the desired structure for target binding. Examples of linkers suitable for connecting the VH and VL chains of an scFv are the linkers identified in Section 6.7.

Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The scFv can comprise VH and VL sequences from any suitable species, such as murine, human or humanized VH and VL sequences.

To create an scFv-encoding nucleic acid, the VH and VL-encoding DNA fragments are operably linked to another fragment encoding a linker, e.g., encoding any of the linkers described in Section 6.7 (typically a repeat of a sequence containing the amino acids glycine and serine, such as the amino acid sequence (Gly4˜Ser) 3 (SEQ ID NO: 129), such that the VH and VL sequences can be expressed as a contiguous single-chain protein, with the VL and VH regions joined by the flexible linker (see, e.g., Bird et al., 1988, Science 242:423-426; Huston et al., 1988, Proc. Natl. Acad. Sci. USA 85:5879-5883; McCafferty et al., 1990, Nature 348:552-554).

### 6.9.1.2. Single Domain Antibodies

In some embodiments of the disclosure, a circularized antibody molecule of the disclosure comprises single domain antibodies, e.g., as fusion partners. The term “single domain antibody” (“sdAb”) is used interchangeably herein with the term “nanobody” to refer to monomeric variable heavy-chain-only antibodies (HcAb), which can bind to an antibody without a counterpart light chain, and antigen-binding fragments (e.g., VHH) and derivatives (e.g. (VHH) 2) thereof. Examples include, but are not limited to, antibodies naturally devoid of light chains, single domain antibodies derived from conventional four-chain antibodies, engineered antibodies and single domain scaffolds other than those derived from antibodies.

Single domain antibodies may be derived from any species including, but not limited to mouse, human, camel, llama, goat, rabbit, bovine, alpaca, and cartilaginous fishes (from immunoglobulin-like structures known as Ig-NAR).

In some embodiments, the single domain antibody is or comprises a “VHH” domain, which is an antibody fragment consisting of the VH domain of camelid heavy-chain antibody or a camelized form of a VH fragment of a classical antibody.

Single domain antibodies can be humanized using well-known methods.

### 6.9.2. Receptors

In some embodiments, the circularized antibody molecules comprise fusion partners that are or comprise receptors or fragments thereof, e.g., useful for targeting the Fab domains of the circularized antibody molecules to a particular cell or block the binding of a ligand to its receptor.

In some embodiments, the receptor is a TNF superfamily receptor, e.g., 4-1BB OX40, CD40 or TRAIL receptor.

In some embodiments, the receptor is ACE2, which mediates coronavirus entry into human cells. Examples of ACE2 receptors are set forth in Section 6.9.2.1.

### 6.9.2.1. ACE2 Receptors

In some embodiments, the fusion partner for a circularized antibody molecule of the disclosure is ACE2. Such fusion partners are sometimes referred to herein as “ACE2 moieties”.

SARS-CoV-2 docks on a host cell's extracellular surface by binding to ACE2, an enzymatic receptor expressed on a variety of cells, including respiratory epithelia.

The amino acid sequence for human ACE2 is assigned the NCBI reference sequence NP_001358344.1 and the UniProtKB accession number Q9BYF1, reproduced below with the signal peptide underlined.

(SEQ ID NO: 1)
MSSSSWLLLS LVAVTAAQST IEEQAKTFLD KFNHEAEDLF
YQSSLASWNY NTNITEENVQ NMNNAGDKWS AFLKEQSTLA
QMYPLQEIQN LTVKLQLQAL QQNGSSVLSE DKSKRLNTIL
NTMSTIYSTG KVCNPDNPQE CLLLEPGLNE IMANSLDYNE
RLWAWESWRS EVGKQLRPLY EEYVVLKNEM ARANHYEDYG
DYWRGDYEVN GVDGYDYSRG QLIEDVEHTF EEIKPLYEHL
HAYVRAKLMN AYPSYISPIG CLPAHLLGDM WGRFWTNLYS
LTVPFGQKPN IDVTDAMVDQ AWDAQRIFKE AEKFFVSVGL
PNMTQGFWEN SMLTDPGNVQ KAVCHPTAWD LGKGDFRILM
CTKVTMDDFL TAHHEMGHIQ YDMAYAAQPF LLRNGANEGF
HEAVGEIMSL SAATPKHLKS IGLLSPDFQE DNETEINFLL
KQALTIVGTL PFTYMLEKWR WMVFKGEIPK DQWMKKWWEM
KREIVGVVEP VPHDETYCDP ASLFHVSNDY SFIRYYTRTL
YQFQFQEALC QAAKHEGPLH KCDISNSTEA GQKLFNMLRL
GKSEPWTLAL ENVVGAKNMN VRPLLNYFEP LFTWLKDQNK
NSFVGWSTDW SPYADQSIKV RISLKSALGD KAYEWNDNEM
YLFRSSVAYA MRQYFLKVKN QMILFGEEDV RVANLKPRIS
FNFFVTAPKN VSDIIPRTEV EKAIRMSRSR INDAFRLNDN
SLEFLGIQPT LGPPNQPPVS IWLIVFGVVM GVIVVGIVIL
IFTGIRDRKK KNKARSGENP YASIDISKGE NNPGFQNTDD
VQTSF

Under normal circumstances, ACE2 contributes to the regulation of vascular tone and blood pressure by cleaving angiotensin precursors, which it achieves via its peptidase domain (PD). PD is the largest domain of ACE2, corresponding to amino acids 18 to 615, the sequence of which is reproduced below.

(SEQ ID NO: 2)
QSTIEEQAKTFLDKFNHEAEDLFYQSSLASWNYNTNITEENVQNMNNAG
DKWSAFLKEQSTLAQMYPLQEIQNLTVKLQLQALQQNGSSVLSEDKSKR
LNTILNTMSTIYSTGKVCNPDNPQECLLLEPGLNEIMANSLDYNERLWA
WESWRSEVGKQLRPLYEEYVVLKNEMARANHYEDYGDYWRGDYEVNGVD
GYDYSRGQLIEDVEHTFEEIKPLYEHLHAYVRAKLMNAYPSYISPIGCL
PAHLLGDMWGRFWTNLYSLTVPFGQKNIDVTDAMVDQAWDAQRIFKEAE
KFFVSVGLPNMTQGFWENSMLTDPGNVQKAVCHPTAWDLGKGDFRLMCT
KVTMDDFLTAHHEMGHIQYDMAYAAQPFLLRNGANEGFHEAVGEIMSLS
AATPKHLKSIGLLSPDFQEDNETEINFLLKQALTIVGTLPFTYMLEKWR
WMVFKGEIPKDQWMKKWWEMKREIVGVVEPVPHDETYCDPASLFHVSND
YSFIRYYTRTLYQFQFQEALCQAAKHEGPLHKCDISNSTEAGQKLFNML
RLGKSEPWTLALENVVGAKNMN
VRPLLNYFEPLFTWLKDQNKNSFVGWSTDWSPYAD

The other domain of ACE2 is its collectrin-like domain (CLD), which contains an extracellular neck domain (ND) that facilitates dimerization, and a single transmembrane domain (TM). The extracellular portion of ACE2 consists of the PD and ND and corresponds to amino acids 18 to 740. The amino acid sequence of the extracellular portion of ACE is reproduced below.

(SEQ ID NO: 3)
QSTIEEQAKTFLDKFNHEAEDLFYQSSLASWNYNTNITEENVQNMNNAG
DKWSAFLKEQSTLAQMYPLQEIQNLTVKLQLQALQQNGSSVLSEDKSKR
LNTILNTMSTIYSTGKVCNPDNPQECLLLEPGLNEIMANSLDYNERLWA
WESWRSEVGKQLRPLYEEYVVLKNEMARANHYEDYGDYWRGDYEVNGVD
GYDYSRGQLIEDVEHTFEEIKPLYEHLHAYVRAKLMNAYPSYISPIGCL
PAHLLGDMWGRFWTNLYSLTVPFGQKPNIDVTDAMVDQAWDAQRIFKEA
EKFFVSVGLPNMTQGFWENSMLTDPGNVQKAVCHPTAWDLGKGDFRILM
CTKVTMDDFLTAHHEMGHIQYDMAYAAQPFLLRNGANEGFHEAVGEIMS
LSAATPKHLKSIGLLSPDFQEDNETEINFLLKQALTIVGTLPFTYMLEK
WRWMVFKGEIPKDQWMKKWWEMKREIVGVVEPVPHDETYCDPASLFHVS
NDYSFIRYYTRTLYQFQFQEALCQAAKHEGPLHKCDISNSTEAGQKLFN
MLRLGKSEPWTLALENVVGAKNMNVRPLLNYFEPLFTWLKDQNKNSFVG
WSTDWSPYADQSIKVRISLKSALGDKAYEWNDNEMYLFRSSVAYAMRQY
FLKVKNQMILFGEEDVRVANLKPRISFNFFVTAPKNVSDIIPRTEVEKA
IRMSRSRINDAFRLNDNSLEFLGIQPTLGPPNQPPVS

SARS-CoV or SARS-CoV-2 interaction with ACE2 involves large viral protrusions called spike(S) proteins. The S protein of SARS-CoV or SARS-CoV-2 consists of two subunits: S1 and S2. The receptor binding domain (RBD) of S1 is responsible for binding the ACE2-PD via polar interactions. More specifically, an extended loop of RBD spans over the α1 helix of ACE2-PD like a bridge, yet it also interacts with the α2 helix and the loop that connects the β3 and β4 strands of ACE2-PD (Yan et al., 2020, Science. 367:1444-1448).

In some embodiments, the circularized antibody molecules of the disclosure comprise an ACE2 moiety as a fusion partner, where the ACE2 moiety has an amino acid sequence with at least 70% sequence identity to an extracellular portion of human ACE2 that is capable of binding the RBD of SARS-CoV or SARS-CoV-2 RBD, for example an amino acid sequence having at least 70% sequence identity to the PD of human ACE2 (SEQ ID NO:2).

In certain aspects, the ACE2 moiety has an amino acid sequence that is at least 75%, at least 80%, at least 85%, or at least 90% identical to the PD of human ACE2, corresponding to amino acids 18 to 615 (SEQ ID NO:2), and in various embodiments has an amino acid sequence that is at least about 91%, at least about 92%, at least about 93%, at least about 94%, at least about 95%, at least about 96%, at least about 97%, at least about 98%, at least about 99%, or 100% identical to the PD of human ACE2.

In further aspects, the ACE2 moiety has an amino acid sequence that is at least 75%, at least 80%, at least 85%, or at least 90% identical to the extracellular region of ACE2 (i.e., PD+ND), corresponding to amino acids 18 to 740 (SEQ ID NO:3), and in various embodiments has an amino acid sequence that is at about 91%, at least about 92%, at least about 93%, at least about 94%, at least about 95%, at least about 96%, at least about 97%, at least about 98%, at least about 99%, or 100% identical to the PD+ND of human ACE2.

Several affinity-enhancing mutations of ACE2 have been reported. For instance, hydrophobic substitutions of T27 of ACE2 increases hydrophobic packing with aromatic residues of the S protein, whereas D30E mutation allows interaction with K417 of the S protein (Yan et al., 2020, Science. 367:1444-1448). Moreover, a slew of single amino acid substitutions that enhance ACE2 binding affinity to RBD have been characterized (Chan et al., 2020, Science. 369:1261-1265; Laurini et al., 2021. ACS Nano 15 (4): 6929-6948), which may be utilized to generate ACE2 moieties with enhanced binding affinity to an RBD, e.g., a SARS-CoV-2 RBD.

In some embodiments, the PD portion of an ACE2 moiety can include one or more amino acid substitutions that increase binding affinity to an RBD, e.g., a SARS-CoV2 RBD. These substitutions may involve the amino acids 19, 23, 24, 25, 26, 27, 29, 30, 31, 33, 34, 35, 39, 40, 41, 42, 65, 69, 72, 75, 76, 79, 82, 89, 90, 91, 92, 324, 325, 330, 357, 386, 393, or 519 of human ACE2, for example, one or more of the amino acid substitutions listed in

### Table 8:

TABLE 8
ACE2-PD Amino Acid Substitutions Enhancing S Protein Binding Affinity

| | | | | | | | |
|---|---|---|---|---|---|---|---|
| S19P | E23F | Q24T | A25V | K26I | T27M | L29F | D30I |
| | | | | K26A | T27L | | D30E |
| | | | | K26D | T27A | | |
| | | | | | T27D | | |
| | | | | | T27K | | |
| | | | | | T27H | | |
| | | | | | T27W | | |
| | | | | | T27Y | | |
| | | | | | T27F | | |
| | | | | | T27C | | |
| K31W | N33D | H34V | E35V | L39K | F40D | Y41R | Q42M |
| K31Y | | H34S | E35C | L39R | F40R | | Q42L |
| | | H34P | E35D | | | | Q42I |
| | | H34K | | | | | Q42V |
| | | | | | | | Q42K |
| | | | | | | | Q42C |
| A65W | W69I | F72Y | E75A | Q76M | L79I | M82C | Q89I |
| | W69V | | E75S | Q76I | L79V | | Q89D |
| | W69T | | E75T | Q76V | L79T | | Q89P |
| | W69K | | E75K | Q76T | L79W | | |
| | | | E75R | Q76R | L79Y | | |
| | | | E75W | Q76Y | L79F | | |
| | | | E75G | | L79P | | |
| N90M | L91P | T92M | T324E | Q325P | N330L | L351F | R357K |
| N90L | | T92L | T324P | | N330H | | |
| N90I | | T92I | | | N330W | | |
| N90V | | T92V | | | N330Y | | |
| N90A | | T92A | | | N330F | | |
| N90S | | T92N | | | | | |
| N90T | | T92Q | | | | | |
| N90Q | | T92D | | | | | |
| N90D | | T92E | | | | | |
| N90E | | T92K | | | | | |
| N90K | | T92R | | | | | |
| N90R | | T92H | | | | | |
| N90H | | T92W | | | | | |
| N90W | | T92Y | | | | | |
| N90Y | | T92F | | | | | |
| N90F | | T92P | | | | | |
| N90P | | T92G | | | | | |
| N90G | | T92C | | | | | |
| N90C | | | | | | | |
| A386L | R393K | R518G | | | | | |
| A386D | | | | | | | |

In some embodiments, the PD portion of the ACE2 moiety includes a combination of two or more amino acid substitutions that enhance its affinity to an RBD, e.g., a SARS-CoV-2 RBD as compared to the corresponding wildtype sequence. In certain specific embodiments, The PD portion of the ACE2 moiety comprises two, three, four, five, six or more of the substitutions listed in Table 8.

In some embodiments, the ACE2 moiety comprises one or more amino acid substitutions associated with high levels of enhanced binding to an RBD, e.g., a SARS-CoV-2 RBD. For instance, these amino acid substitution combinations can include one or more substitutions at the amino acids 25, 27, 31, 34, 42, 79, 90, 92, 324, 325, 330, and 386 of human ACE2, for example one or more of the substitutions sets forth in Table 8, which are associated with the highest binding affinity increases to the SARS-CoV-2 RBD.

In certain aspects, the ACE moiety can include combinations of amino acid substitutions that have been shown to be associated with increased RBD affinity. For instance, one such example is ACE2v2.4, which combines the amino acid substitutions T27Y, L79T, and N330Y (Chan et al., 2020, Science. 369:1261-1265). Accordingly, in certain embodiments, the combinations of amino acid substitutions of the ACE2 moiety can include the amino acid substitutions T27Y, L79T, and N330Y, optionally with one or more additional substitutions. In some embodiments, the ACE2 moiety comprises the PD of ACE2 (e.g., an amino acid sequence having the sequence of SEQ ID NO:2) with the amino acid substitutions T27Y, L79T, and N330Y. In further embodiments, the ACE2 moiety comprises the PD+ND of ACE2 (e.g., an amino acid sequence having the sequence of SEQ ID NO: 3) with the amino acid substitutions T27Y, L79T, and N330Y.

### 6.10. Nucleic Acids and Host Cells

In another aspect, the disclosure provides nucleic acids encoding the precursors of the circularized antibody molecules of the disclosure. In some embodiments, the precursors are encoded by a single nucleic acid. In other embodiments, the circularized antibody molecules can be encoded by a plurality (e.g., two, three, four or more) nucleic acids.

A single nucleic acid can encode a precursor of a circularized antibody molecule that comprises two or more polypeptide chains. For separate control of expression, the open reading frames encoding two or more polypeptide chains can be under the control of separate transcriptional regulatory elements (e.g., promoters and/or enhancers). The open reading frames encoding two or more polypeptides can also be controlled by the same transcriptional regulatory elements, and separated by internal ribosome entry site (IRES) sequences allowing for translation into separate polypeptides.

In some embodiments, a precursor of a circularized antibody molecule comprising two or more polypeptide chains is encoded by two or more nucleic acids. The number of nucleic acids encoding a circularized antibody molecule can be equal to or less than the number of polypeptide chains in the circularized antibody molecule (for example, when more than one polypeptide chains are encoded by a single nucleic acid).

The precursor polypeptides are in general secreted polypeptides and therefore contain an N-terminal extension (also known as the signal sequence or signal peptide) which is necessary for the transport/secretion of the polypeptide into the cell culture medium. In general, the signal sequence can be derived from any gene encoding a secreted polypeptide. If a heterologous signal sequence is used, it preferably is one that is recognized and processed (e.g., cleaved by a signal peptidase) by the host cell. In mammalian cell expression, the native immunoglobulin signal sequence may be used, although other mammalian signal sequences may be suitable, such as signal sequences from secreted polypeptides, e.g., for immunoglobulins from human or murine origin, as well as viral secretory signal sequences, for example, the herpes simplex glycoprotein D signal sequence. The term “precursor” as used herein refers to polypeptide with a signal sequence or without a signal sequence, e.g., a polypeptide from which the signal sequence has been processed unless dictated otherwise (e.g., in the context of circularization of a precursor with a pair of split inteins at its N- and C-termini).

The nucleic acids of the disclosure can be DNA or RNA (e.g., mRNA).

In another aspect, the disclosure provides host cells and vectors containing the nucleic acids of the disclosure. The nucleic acids may be present in a single vector or separate vectors present in the same host cell or separate host cell, as described in more detail herein below.

### 6.10.1. Vectors

The disclosure provides vectors comprising nucleotide sequences encoding a precursor of a circularized antibody molecule or a component thereof described herein, for example one of two or more polypeptide chains of a precursor of a circularized antibody molecule. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes can be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by co-transformation. Additional elements may also be needed for optimal synthesis of mRNA. These elements may include splice signals, as well as transcriptional promoters, enhancers, and termination signals.

Once the expression vector or DNA sequence containing the precursor or polypeptide chain has been prepared for expression, the expression vectors can be transfected or introduced into an appropriate host cell. Various techniques may be employed to achieve this, such as, for example, protoplast fusion, calcium phosphate precipitation, electroporation, retroviral transduction, viral transfection, gene gun, lipid-based transfection or other conventional techniques. Methods and conditions for culturing the resulting transfected cells and for recovering the expressed polypeptides are known to those skilled in the art, and may be varied or optimized depending upon the specific expression vector and mammalian host cell employed, based upon the present description.

### 6.10.2. Cells

The disclosure also provides host cells comprising a nucleic acid of the disclosure.

In one embodiment, the host cells are genetically engineered to comprise one or more nucleic acids described herein.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The disclosure also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### 6.11. Methods of Producing Circularized Antibody Molecules

The circularized antibody molecules of the disclosure can be produced by trans-splicing of linear precursor polypeptides by split inteins through a process known as “split intein circular ligation of peptides and proteins” (SICLOPPS).

Split inteins are complementary intein portions that form an active intein complex upon binding to one another. The active intein then self-excises from the precursor and catalyzes ligation of the flanking sequences of the polypeptide with a peptide bond. Intein self-excision is a posttranslational process that does not require auxiliary enzymes or cofactors. The intein is preferably selected to be capable of circularizing at physiological temperature and pH.

To produce a circularized protein, a linear precursor polypeptide can be designed, wherein a target sequence to be circularized, which is often referred to as an extein, is interposed between two complementary portions of a split intein. When expressed in an appropriate host system, the circularization of the protein is achieved in five steps.

First, two complementary portions of the split intein physically come together to form an active intein in a conformation that also forces the extein into a loop configuration (i), wherein the N-terminal extein residue immediately adjacent to first split intein sequence typically comprises a cysteine, serine, or threonine, and serves as the nucleophile for the transesterification reaction that generates the branched and lariat intermediates in protein splicing and circular ligation, respectively (Scott et al., 2001, Chemistry & Biology 8 (8): 801-815).

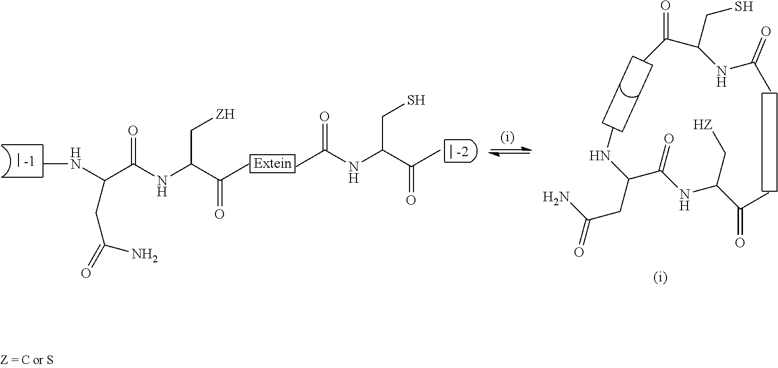

In the context of the linear precursor polypeptides of the disclosure, the N-terminal extein residue represents the N-terminus of the first portion of the first linker (L-1A). Preferably, that N-terminal residue is a serine or a threonine.

After the formation of the loop, the ester isomer of the amino acid at the junction between one of the split intein portions and the extein is stabilized such that the other portion of the split intein can then react with the ester to produce a thioester intermediate (ii).

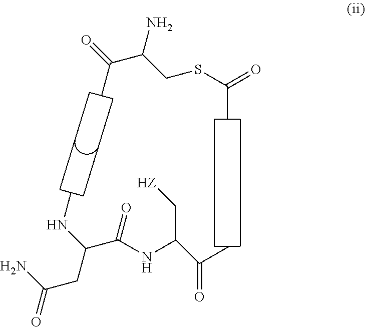

The active intein then catalyzes the formation of a lariat intermediate via transesterification (iii).

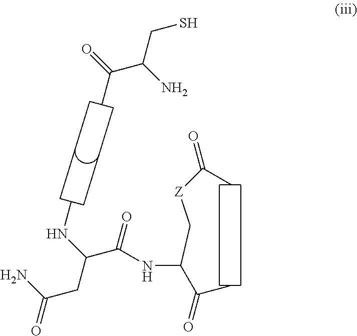

Following Asp side chain cyclization, the intein complex is excised from the cyclized extein (i.e., extein in a lactone form) (iv). Finally, thermodynamic rearrangement results in a stable cyclic product (i.e., lactam form) (v).

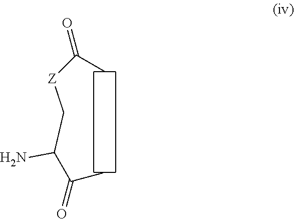

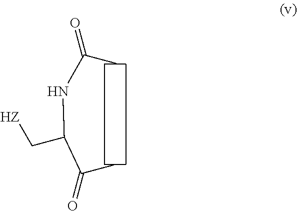

Split inteins can be used to produce the circularized antibody molecules of the disclosure, e.g., circularized tri-Fabs, without the use of any auxiliary enzymes or cofactors.

To produce a circularized antibody molecule, e.g., a circularized tri-Fab, through SICLOPPS process, two types of polypeptides are co-expressed in a suitable host cell, wherein the first type of polypeptide is a precursor polypeptide, which comprises two complementary split inteins flanking a plurality of cyclizable domains connected via linkers that were described in Section 6.6, and the second type of polypeptide comprises a counterpart domain that can pair with the corresponding cyclizable domain. Next, cyclizable and counterpart domains of the co-expressed polypeptides associate to form a linear polypeptide comprising a plurality of antibody fragment domains, which then circularizes via split intein-mediated trans-splicing.

In some embodiments, the intein is capable of being spliced without the addition of exogenous reducing agents, although reducing agents can be added to improve yield by driving the reaction to completion.

In some embodiments, the precursor further comprises a purification tag (e.g., a strep tag, a his tag or a C tag) incorporated in the intein that can be used to remove molecules that have not circularized.

### 6.12. Pharmaceutical Compositions

The circularized antibody molecules of the disclosure may be in the form of compositions comprising the circularized antibody molecule and one or more carriers, excipients and/or diluents. The compositions may be formulated for specific uses, such as for veterinary uses or pharmaceutical uses in humans. The form of the composition (e.g., dry powder, liquid formulation, etc.) and the excipients, diluents and/or carriers used will depend upon the intended uses of the circularized antibody molecule and, for therapeutic uses, the mode of administration.

For therapeutic uses, the compositions may be supplied as part of a sterile, pharmaceutical composition that includes a pharmaceutically acceptable carrier. This composition can be in any suitable form (depending upon the desired method of administering it to a patient). The pharmaceutical composition can be administered to a patient by a variety of routes such as orally, transdermally, subcutaneously, intranasally, intravenously, intramuscularly, intratumorally, intrathecally, topically or locally. The most suitable route for administration in any given case will depend on the particular circularized antibody molecule, the subject, and the nature and severity of the disease and the physical condition of the subject. Typically, the pharmaceutical composition will be administered intravenously or subcutaneously.

Pharmaceutical compositions can be conveniently presented in unit dosage forms containing a predetermined amount of a circularized antibody molecule of the disclosure per dose. The quantity of circularized antibody molecule included in a unit dose will depend on the disease being treated, as well as other factors as are well known in the art. Such unit dosages may be in the form of a lyophilized dry powder containing an amount of circularized antibody molecule suitable for a single administration, or in the form of a liquid. Dry powder unit dosage forms may be packaged in a kit with a syringe, a suitable quantity of diluent and/or other components useful for administration. Unit dosages in liquid form may be conveniently supplied in the form of a syringe pre-filled with a quantity of circularized antibody molecule suitable for a single administration.

The pharmaceutical compositions may also be supplied in bulk from containing quantities of circularized antibody molecule suitable for multiple administrations.

Pharmaceutical compositions may be prepared for storage as lyophilized formulations or aqueous solutions by mixing a circularized antibody molecule having the desired degree of purity with optional pharmaceutically-acceptable carriers, excipients or stabilizers typically employed in the art (all of which are referred to herein as “carriers”), i.e., buffering agents, stabilizing agents, preservatives, isotonifiers, non-ionic detergents, antioxidants, and other miscellaneous additives. See, Remington's Pharmaceutical Sciences, 16th edition (Osol, ed. 1980). Such additives should be nontoxic to the recipients at the dosages and concentrations employed.

Buffering agents help to maintain the pH in the range which approximates physiological conditions. They may be present at a wide variety of concentrations, but will typically be present in concentrations ranging from about 2 mM to about 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives may be added to retard microbial growth, and can be added in amounts ranging from about 0.2%-1% (w/v). Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” can be added to ensure isotonicity of liquid compositions of the present disclosure and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trehalose; and trisaccacharides such as raffinose; and polysaccharides such as dextran. Stabilizers may be present in amounts ranging from 0.5 to 10 wt % per wt of circularized antibody molecule.

Non-ionic surfactants or detergents (also known as “wetting agents”) may be added to help solubilize the glycoprotein as well as to protect the glycoprotein against agitation-induced aggregation, which also permits the formulation to be exposed to shear surface stressed without causing denaturation of the protein. Suitable non-ionic surfactants include polysorbates (20, 80, etc.), polyoxamers (184, 188, etc.), and pluronic polyols. Non-ionic surfactants may be present in a range of about 0.05 mg/mL to about 1.0 mg/mL, for example about 0.07 mg/mL to about 0.2 mg/mL.

Additional miscellaneous excipients include bulking agents (e.g., starch), chelating agents (e.g., EDTA), antioxidants (e.g., ascorbic acid, methionine, vitamin E), and cosolvents.

The circularized antibody molecules of the disclosure can be formulated as pharmaceutical compositions comprising the circularized antibody molecules, for example containing one or more pharmaceutically acceptable excipients or carriers. To prepare pharmaceutical or sterile compositions comprising the circularized antibody molecules of the present disclosure, a circularized antibody molecule preparation can be combined with one or more pharmaceutically acceptable excipient or carrier.

For example, formulations of circularized antibody molecules can be prepared by mixing circularized antibody molecules with physiologically acceptable carriers, excipients, or stabilizers in the form of, e.g., lyophilized powders, slurries, aqueous solutions, lotions, or suspensions (see, e.g., Hardman et al., 2001, Goodman and Gilman's The Pharmacological Basis of Therapeutics, McGraw-Hill, New York, N.Y.; Gennaro, 2000, Remington: The Science and Practice of Pharmacy, Lippincott, Williams, and Wilkins, New York, N.Y.; Avis, et al. (eds.), 1993, Pharmaceutical Dosage Forms: General Medications, Marcel Dekker, NY; Lieberman, et al. (eds.), 1990, Pharmaceutical Dosage Forms: Tablets, Marcel Dekker, NY; Lieberman, et al. (eds.), 1990, Pharmaceutical Dosage Forms: Disperse Systems, Marcel Dekker, NY; Weiner and Kotkoskie, 2000, Excipient Toxicity and Safety, Marcel Dekker, Inc., New York, N.Y.).

An effective amount for a particular subject may vary depending on factors such as the condition being treated, the overall health of the subject, the method route and dose of administration and the severity of side effects (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

A composition of the present disclosure may also be administered via one or more routes of administration using one or more of a variety of methods known in the art. As will be appreciated by the skilled artisan, the route and/or mode of administration will vary depending upon the desired results. Selected routes of administration for circularized antibody molecules include intravenous, intramuscular, intradermal, intraperitoneal, subcutaneous, spinal, or other general routes of administration, for example by injection or infusion. General administration may represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a composition of the disclosure can be administered via a non-general route, such as a topical, epidermal, or mucosal route of administration, for example, intranasally, orally, vaginally, rectally, sublingually or topically. In one embodiment, the circularized antibody molecules are administered by infusion. In another embodiment, the circularized antibody molecule of the disclosure is administered subcutaneously.

### 6.13. Methods of Use

The present disclosure provides methods for using and applications for the circularized antibody molecules of the disclosure.

Due to their valency, the circularized antibody molecules of the disclosure are useful for clustering cell surface molecules. In some embodiments, methods of clustering cell surface molecules comprise contacting a cell that expresses the cell surface molecules with a circularized antibody molecule as described herein whose Fab domains bind to the cell surface molecule. In some embodiments, the cell surface molecules are trimeric receptors, e.g., TNFα receptors.

The circularized antibody molecules of the disclosure are useful for cross-linking different cells, e.g., a first cell and a second cell. In some embodiments, methods of cross-linking a first cell and a second cell, comprise contacting the first cell and the second cell with a circularized antibody molecule as described herein, e.g., where the Fab domains of the circularized antibody molecule bind to the first cell and the circularized antibody molecule comprises scFvs that binds to the second cell, optionally wherein the scFvs are configured as fusion partners.

The circularized antibody molecules of the disclosure are also useful for inhibiting an infectious agent, e.g., to treat or prevent an infection. In some embodiments, method of inhibiting an infectious agent comprise contacting the infectious agent with a circularized antibody molecule as described herein, e.g., where the Fab domains of the circularized antibody molecule binds to the infectious agent (e.g., SARS-CoV2, such as via its spike protein, and in some embodiments to its receptor binding domain). In some embodiments, the circularized antibody molecule further comprises at least a binding portion of the cellular receptor for the infectious agent (e.g., the ACE2 receptor), optionally wherein the at least a binding portion of the cellular receptor is configured as a fusion partner.

The methods described herein can be utilized in vitro, ex vivo or in vivo, e.g., for therapeutic applications.

### 6.13.1. Circularized Antibody Molecules That Bind to Spike Proteins

With respect to circularized antibody molecule that bind to a coronavirus spike protein, the disclosure provides a method of preventing or treating a disease or condition in which an interaction between a RBD of a coronavirus and cellular ACE2 is implicated. The methods comprise administering a circularized antibody molecule whose Fab domains bind to a coronavirus protein, e.g., a coronavirus spike protein (e.g., Fab domains having sequences as defined in Section 6.5.1). Optionally, the circularized antibody molecule further comprises at least a binding portion of the ACE2 receptor as a fusion partner (e.g., comprises an ACE2 moiety as described in Section 6.9.2.1).

In some embodiments, the disease or condition is prevented or treated by neutralization of the spike protein. In various embodiments, neutralization of the spike proteins comprises (a) inhibiting the ability of spike protein to bind to a receptor such as ACE2, (b) inhibiting cleavage of the spike protein by a protease such as TMPRSS2, (c) inhibiting the spike protein from mediating (i) viral entry into a host cell or (ii) viral reproduction in a host cell, or (d) any combination of two, three, or all four of (a), (b), (c) (i), and (c) (ii).

Accordingly, in some embodiments, the circularized antibody molecules and pharmaceutical compositions of the disclosure can be used to inhibit an interaction between a RBD of a coronavirus and cellular ACE2. In some embodiments, the disclosure provides methods of inhibiting the interaction between the RBD of SARS-CoV. In other embodiments, the disclosure provides methods of inhibiting the interaction between the RBD of SARS-CoV-2. Accordingly, in some embodiments, the disclosure provides methods of inhibiting an interaction between a RBD of a coronavirus and cellular ACE2, comprising administering to a subject in need thereof a circularized antibody molecule pharmaceutical composition as described herein.

In some embodiments, the disclosure provides methods of administrating a circularized antibody molecule pharmaceutical composition as described herein to a subject who has been exposed to a coronavirus but is not diagnosed with an infection. In other embodiments, the subject has been tested positive for a coronavirus but is asymptomatic. In yet other embodiments, the subject has been tested positive for a coronavirus and is presymptomatic. In further embodiments, the subject has been tested positive for a coronavirus and is symptomatic. In other embodiments, the subject has developed COVID-19 or another coronavirus-mediated disease or condition.

In some embodiments, the disclosure provides a method of reducing the severity of coronavirus infection, comprising administering to a subject in need thereof the circularized antibody molecule pharmaceutical composition as described herein.

In some other embodiments, the disclosure provides a method of reducing the viral load of a coronavirus, comprising administering to a subject in need thereof the circularized antibody molecule pharmaceutical composition as described herein.

In further embodiments, the disclosure provides a method of preventing disease progression in a subject with a coronavirus infection, comprising administering to a subject in need thereof the circularized antibody molecule pharmaceutical composition as described herein.

In some embodiments, the disclosure provides a method of reducing the duration of a coronavirus infection, comprising administering to a subject in need thereof the circularized antibody molecule pharmaceutical composition as described herein.

In other embodiments, the disclosure provides a method of reducing the risk of severe disease or death in a subject with a coronavirus infection, comprising administering to a subject in need thereof the circularized antibody molecule pharmaceutical composition as described herein.

### 7. Numbered Embodiments

While various specific embodiments have been illustrated and described, it will be appreciated that various changes can be made without departing from the spirit and scope of the disclosure(s). The present disclosure is exemplified by the numbered embodiments set forth below.
1. A polypeptide comprising a first split intein, three antibody fragments (a first antibody fragment, a second antibody fragment, and a third antibody fragment), and a second split intein, wherein the splicing of the first split intein and the second split intein results in a circularized antibody molecule comprising the three antibody fragments, optionally wherein the three antibody fragments are Fab domains, Fc domains, or a combination thereof (e.g., as described in Section 6.2 or any one of FIGS. 1-5).
2. The polypeptide of embodiment 1, wherein the angle between each pair of antibody fragments ranges between 55° and 65°.
3. The polypeptide of embodiment 1, wherein the angle between each pair of antibody fragments ranges between 58° and 62°.
4. The polypeptide of any one of embodiments 1 to 3, wherein the angle between each pair of antibody fragments is 62° or less.
5. The polypeptide of any one of embodiments 1 to 3, wherein the angle between each pair of antibody fragments domains is 61° or less.
6. The polypeptide of any one of embodiments 1 to 5, wherein the angle between each pair of antibody fragments is 60°+1°.
7. The polypeptide of any one of embodiments 1 to 5, wherein the angle between each pair of antibody fragments is 60°+0.5°.
8. The polypeptide of any one of embodiments 1 to 7, which comprises four polypeptide chains.
9. The polypeptide of any one of embodiments 1 to 8, which comprises a first polypeptide chain comprising the split inteins and a first component of each of the three antibody fragments (a “cyclizable domain” such as an Fc, an Fd domain or a light chain (LC)), wherein each cyclizable domain is associated with a counterpart domain (e.g., another Fc domain, an LC, or an Fd, respectively) on a separate polypeptide chain.
10. The polypeptide of embodiment 9, wherein one, two or all three of the polypeptide chains comprising the counterpart domains further comprise a fusion partner.
11. The polypeptide of any one of embodiments 1 to 10, which has the polypeptide chain depicted in FIG. 1A and three of the polypeptide chains depicted in any one of FIGS. 1B-1D.
12. The polypeptide of any one of embodiments 1 to 10, which has the polypeptide chain depicted in FIG. 2A and three of the polypeptide chains depicted in FIG. 2B.
13. The polypeptide of any one of embodiments 1 to 10, which has the polypeptide chain depicted in FIG. 2C and three of the polypeptide chains depicted in FIG. 2D.
14. The polypeptide of any one of embodiments 1 to 10, which has the polypeptide chain depicted in FIG. 2E and three of the polypeptide chains depicted in FIG. 2F.
15. The polypeptide of any one of embodiments 1 to 10, which has the polypeptide chain depicted in FIG. 2G, and wherein each antibody fragment comprises a cyclizable domain associated with a counterpart domain depicted in FIG. 2H.
16. The polypeptide of any one of embodiments 1 to 10, which has the polypeptide chain depicted in FIG. 2I, and wherein each antibody fragment comprises a cyclizable domain associated with a counterpart domain depicted in FIG. 2J.
17. The polypeptide of any one of embodiments 1 to 10, which has the polypeptide chain depicted in FIG. 3A and three of the polypeptide chains depicted in any one of FIGS. 3B and 3C
18. The polypeptide of any one of embodiments 1 to 10, which has the polypeptide chain depicted in FIG. 4A and three of the polypeptide chains depicted in any one of FIGS. 4B and 4C.
19. The polypeptide of any one of embodiments 1 to 10, which has the polypeptide chain depicted in FIG. 5A and three of the polypeptide chains depicted in any one of FIGS. 5B and 5C.
20. The polypeptide of any one of embodiments 1 to 19, wherein:
   (a) each pair of antibody fragments in the polypeptide is separated by a linker; and/or
   (b) each pair of antibody fragments in the circularized antibody molecule is separated by a linker.
21. The polypeptide of embodiment 20, wherein each linker is 3 to 20 amino acids.
22. The polypeptide of embodiment 20, wherein each linker is 3 to 10 amino acids.
23. The polypeptide of embodiment 20, wherein each linker is 3 to 7 amino acids.
24. The polypeptide of embodiment 20, wherein each linker is 5 to 7 amino acids.
25. The polypeptide of any one of any one of embodiments 20 to 24, wherein each linker does not contain a cysteine.
26. The polypeptide of any one of embodiments 20 to 25, wherein each of the linkers does not contain a tryptophan.
27. The polypeptide of any one of embodiments 20 to 26, wherein each of the linkers does not contain a methionine.
28. The polypeptide of any one of embodiments 20 to 27, wherein each of the linkers does not contain an N-linked glycosylation site (e.g., as defined by the motif NXT or NXS).
29. The polypeptide of any one of embodiments 20 to 28, wherein each of the linkers comprises a serine and/or a threonine.
30. The polypeptide of embodiment 29, wherein each of the linkers comprises a serine or threonine within one amino acid from the center of the linker (e.g., at the third, fourth or fifth position in the case of a 7 amino acid linker).
31. The polypeptide of embodiment 29, wherein each of the linkers comprises a serine or threonine at the center of the linker (e.g., at the fourth position in the case of a 7 amino acid linker).
32. The polypeptide of any one of embodiments 20 to 31, wherein each of the linkers is predicted to lack secondary structure.
33. The polypeptide of any one of embodiments 20 to 32, wherein each of the linkers has (e.g., comprises or consists of) an amino acid sequence selected from QLGTVEG (SEQ ID NO: 84), TLNSEES (SEQ ID NO: 85), VMSSGDQ (SEQ ID NO: 86), KLASYNP (SEQ ID NO: 87), FIDSGVG (SEQ ID NO: 88), THFSQQD (SEQ ID NO: 89), NHSSLHD (SEQ ID NO: 90), a glycine-serine linker such as GGGSGGG (SEQ ID NO: 91), or a 3-amino acid or 5-amino acid fragment of any of the foregoing.
34. The polypeptide of any one of embodiments 20 to 33, wherein all the linkers are identical.
35. The polypeptide of any one of embodiments 1 to 34, which comprises a first polypeptide chain comprising, in N- to C-terminal orientation:
   (a) the first split intein (I-A)
   (b) a first portion of a first linker (L-1A),
   (c) a first Fd domain (Fd-1),
   (d) a second linker (L-2),
   (e) a second Fd domain (Fd-2),
   (f) a third linker (L-3),
   (g) a third Fd domain (Fd-3),
   (h) a second portion of the first linker (L-1B), and
   (i) the second split intein (I-B),
   wherein upon splicing of the first split intein (I-A) and the second split intein (I-B), a circularized polypeptide chain is produced in which the first linker becomes contiguous.
36. The polypeptide of embodiment 35, wherein the (L-1), the second linker (L-2) and the third linker (L-3) are each 3, 5 or 7 amino acids.
37. The polypeptide of embodiment 35, wherein the first linker (L-1), the second linker (L-2) and the third linker (L-3) are 3 to 20 amino acids.
38. The polypeptide of embodiment 35, wherein the first linker (L-1), the second linker (L-2) and the third linker (L-3) are 3 to 10 amino acids.
39. The polypeptide of embodiment 35, wherein the first linker (L-1), the second linker (L-2) and the third linker (L-3) are 3 to 7 amino acids.
40. The polypeptide of embodiment 35, wherein the first linker (L-1), the second linker (L-2) and the third linker (L-3) are 5 to 7 amino acids.
41. The polypeptide of any one of embodiments 35 to 40, wherein the first linker (L-1), the second linker (L-2) and the third linker (L-3) do not contain a cysteine.
42. The polypeptide of any one of embodiments 35 to 41, wherein the first linker (L-1), the second linker (L-2) and the third linker (L-3) do not contain a tryptophan.
43. The polypeptide of any one of embodiments 35 to 42, wherein the first linker (L-1), the second linker (L-2) and the third linker (L-3) do not contain a methionine.
44. The polypeptide of any one of embodiments 35 to 43, wherein the first linker (L-1), the second linker (L-2) and the third linker (L-3) do not contain an N-linked glycosylation site (e.g., as defined by the motif NXT or NXS).
45. The polypeptide of any one of embodiments 35 to 44, wherein the first linker (L-1), the second linker (L-2) and the third linker (L-3) comprise a serine and/or a threonine.
46. The polypeptide of embodiment 45, wherein the first linker (L-1), the second linker (L-2) and the third linker (L-3) comprise a serine or threonine within one amino acid from the center of the linker (e.g., at the third, fourth or fifth position in the case of a 7 amino acid linker).
47. The polypeptide of embodiment 45, wherein the first linker (L-1), the second linker (L-2) and the third linker (L-3) comprise a serine or threonine at the center of the linker (e.g., at the fourth position in the case of a 7 amino acid linker).
48. The polypeptide of any one of embodiments 35 to 47, wherein each of the first linker (L-1), the second linker (L-2) and the third linker (L-3) is predicted to lack secondary structure.
49. The polypeptide of any one of embodiments 35 to 48, wherein one, two or all three of the first linker (L-1), the second linker (L-2) and the third linker (L-3) has (e.g., comprises or consists of) an amino acid sequence selected from QLGTVEG (SEQ ID NO: 84), TLNSEES (SEQ ID NO: 85), VMSSGDQ (SEQ ID NO: 86), KLASYNP (SEQ ID NO: 87), FIDSGVG (SEQ ID NO: 88), THFSQQD (SEQ ID NO: 89), NHSSLHD (SEQ ID NO: 90), a glycine-serine linker such as GGGSGGG (SEQ ID NO: 91), or a 3-amino acid or 5-amino acid fragment of any of the foregoing.
50. The polypeptide of any one of embodiments 35 to 49, wherein the first linker (L-1), the second linker (L-2) and the third linker (L-3) are identical.
51. The polypeptide of any one of embodiments 35 to 50, which comprises a second polypeptide chain comprising a first light chain (LC-1), a third polypeptide chain comprising a second light chain (LC-2) and a fourth polypeptide chain comprising a third light chain (LC-3).
52. The polypeptide of embodiment 51, wherein the first light chain (LC-1), second light chain (LC-2) and third light chain (LC-3) are universal light chains.
53. The polypeptide of embodiment 51 or embodiment 52, wherein the second polypeptide chain comprises a first fusion partner (FP-1), the third polypeptide chain comprises a second fusion partner (FP-2), and the fourth polypeptide chain comprises a third fusion partner (FP-3).
54. The polypeptide of embodiment 53, wherein the first fusion partner (FP-1) is N-terminal to the first light chain (LC-1), the second fusion partner (FP-2) is N-terminal to the second light chain (LC-2), and the third fusion partner (FP-3) is N-terminal to the third light chain (LC-3).
55. The polypeptide of embodiment 53 or embodiment 54, wherein the first fusion partner (FP-1) and the first light chain (LC-1) are separated by a fourth linker (L-4), the second fusion partner (FP-2) and the second light chain (LC-2) are separated by a fifth linker (L-5), and the third fusion partner (FP-3) and the third light chain (LC-3) are separated by a sixth linker (L-6).
56. The polypeptide of embodiment 55, wherein the fourth linker (L-4), the fifth linker (L-5) and the sixth linker (L-6) are the same and/or selected from Section 6.7 or Table 6.
57. The polypeptide of any one of embodiments 53 to 56, wherein the first fusion partner (FP-1), the second fusion partner (FP-2) and the third fusion partner (FP-3) are the same.
58. The polypeptide any one of embodiments 53 to 57, wherein the first fusion partner (FP-1), the second fusion partner (FP-2) and the third fusion partner (FP-3) are single chain antibody or antibody fragments, optionally scFvs or sdAbs.
59. The polypeptide any one of embodiments 53 to 57, wherein the first fusion partner (FP-1), the second fusion partner (FP-2) and the third fusion partner (FP-3) are receptors or receptor fragments, optionally soluble receptor fragments.
60. The polypeptide of any one of embodiments 51 to 59, wherein the second polypeptide, the third polypeptide and the fourth polypeptide are the same.
61. The polypeptide of any one of embodiments 1 to 34, which comprises a first polypeptide chain comprising, in N- to C-terminal orientation:
   (a) the first split intein
   (b) a first portion of a first linker (L-1A),
   (c) a first light chain (LC-1),
   (d) a second linker (L-2),
   (e) a second light chain (LC-2),
   (f) a third linker (L-3),
   (g) a third light chain (LC-3),
   (h) a second portion of the first linker (L-1B), and
   (i) the second split intein,
   wherein upon splicing of the first split intein and the second split intein, a circularized polypeptide chain is produced in which the first linker (L-1) becomes contiguous.
62. The polypeptide of embodiment 61, wherein the first light chain (LC-1), second light chain (LC-2) and third light chain (LC-3) are universal light chains.
63. The polypeptide of embodiment 61 or embodiment 62, wherein the first linker (L-1), the second linker (L-2) and the third linker (L-3) are each 3, 5 or 7 amino acids.
64. The polypeptide of embodiment 63, wherein the first linker (L-1), the second linker (L-2) and the third linker (L-3) are 3 to 20 amino acids.
65. The polypeptide of embodiment 63, wherein the first linker (L-1), the second linker (L-2) and the third linker (L-3) are 3 to 10 amino acids.
66. The polypeptide of embodiment 63, wherein the first linker (L-1), the second linker (L-2) and the third linker (L-3) are 3 to 7 amino acids.
67. The polypeptide of embodiment 63, wherein the first linker (L-1), the second linker (L-2) and the third linker (L-3) are 5 to 7 amino acids.
68. The polypeptide of any one of embodiments 61 to 67, wherein the first linker (L-1), the second linker (L-2) and the third linker (L-3) do not contain a cysteine.
69. The polypeptide of any one of embodiments 61 to 68, wherein the first linker (L-1), the second linker (L-2) and the third linker (L-3) do not contain a tryptophan.
70. The polypeptide of any one of embodiments 61 to 69, wherein the first linker (L-1), the second linker (L-2) and the third linker (L-3) do not contain a methionine.
71. The polypeptide of any one of embodiments 61 to 70, wherein the first linker (L-1), the second linker (L-2) and the third linker (L-3) do not contain an N-linked glycosylation site (e.g., as defined by the motif NXT or NXS).
72. The polypeptide of any one of embodiments 61 to 71, wherein the first linker (L-1), the second linker (L-2) and the third linker (L-3) comprise a serine and/or a threonine.
73. The polypeptide of embodiment 72, wherein the first linker (L-1), the second linker (L-2) and the third linker (L-3) comprise a serine or threonine within one amino acid from the center of the linker (e.g., at the third, fourth or fifth position in the case of a 7 amino acid linker).
74. The polypeptide of embodiment 72, wherein the first linker (L-1), the second linker (L-2) and the third linker (L-3) comprise a serine or threonine at the center of the linker (e.g., at the fourth position in the case of a 7 amino acid linker).
75. The polypeptide of any one of embodiments 61 to 74, wherein each of the first linker (L-1), the second linker (L-2) and the third linker (L-3) is predicted to lack secondary structure.
76. The polypeptide of any one of embodiments 61 to 75, wherein one, two or all three of the first linker (L-1), the second linker (L-2) and the third linker (L-3) has (e.g., comprises or consists of) an amino acid sequence selected from QLGTVEG (SEQ ID NO: 84), TLNSEES (SEQ ID NO: 85), VMSSGDQ (SEQ ID NO: 86), KLASYNP (SEQ ID NO: 87), FIDSGVG (SEQ ID NO: 88), THFSQQD (SEQ ID NO: 89), NHSSLHD (SEQ ID NO: 90), a glycine-serine linker such as GGGSGGG (SEQ ID NO: 91), or a 3-amino acid or 5-amino acid fragment of any of the foregoing.
77. The polypeptide of any one of embodiments 61 to 76, wherein the first linker (L-1), the second linker (L-2) and the third linker (L-3) are identical.
78. The polypeptide of any one of embodiments 61 to 77, which comprises a second polypeptide chain comprising a Fd domain, a third polypeptide chain comprising a second Fd domain (Fd-2) and a fourth polypeptide chain comprising a third Fd domain (Fd-3).
79. The polypeptide of embodiment 78, wherein the second polypeptide chain comprises a first fusion partner (FP-1), the third polypeptide chain comprises a second fusion partner (FP-2), and the fourth polypeptide chain comprises a third fusion partner (FP-3).
80. The polypeptide of embodiment 79, wherein the first fusion partner (FP-1) is N-terminal to the first Fd domain (Fd-1), the second fusion partner (FP-2) is N-terminal to the second Fd domain (Fd-2), and the third fusion partner (FP-3) is N-terminal to the third Fd domain (Fd-3).
81. The polypeptide of embodiment 79 or embodiment 80, wherein the first fusion partner (FP-1) and the first Fd domain (Fd-1) are separated by a fourth linker (L-4), the second fusion partner (FP-2) and the second Fd domain (Fd-2) are separated by a fifth linker (L-5), and the third fusion partner (FP-3) and the third Fd domain (Fd-3) are separated by a sixth linker (L-6).
82. The polypeptide of embodiment 81, wherein the fourth linker (L-4), the fifth linker (L-5) and the sixth linker (L-6) are the same and/or selected from Section 6.7 or Table 6.
83. The polypeptide of any one of embodiments 79 to 82, wherein the first fusion partner (FP-1), the second fusion partner (FP-2) and the third fusion partner (FP-3) are the same.
84. The polypeptide any one of embodiments 79 to 83, wherein the first fusion partner (FP-1), the second fusion partner (FP-2) and the third fusion partner (FP-3) are single chain antibody or antibody fragments, optionally scFvs or sdAbs.
85. The polypeptide any one of embodiments 79 to 83, wherein the first fusion partner (FP-1), the second fusion partner (FP-2) and the third fusion partner (FP-3) are receptors or receptor fragments, optionally soluble receptor fragments.
86. The polypeptide of any one of embodiments 78 to 85, wherein the second polypeptide, the third polypeptide and the fourth polypeptide are the same.
87. The polypeptide of any one of embodiments 1 to 86, wherein the three Fab domains are identical.
88. The polypeptide of any one of embodiments 1 to 86, wherein at least two Fab domains differ from one another.
89. The polypeptide of any one of embodiments 1 to 88, which is trivalent.
90. The polypeptide of any one of embodiments 1 to 88, which is hexavalent.
91. The polypeptide of any one of embodiments 1 to 90, which lacks a CH2 domain.
92. The polypeptide of any one of embodiments 1 to 91, which lacks a CH3 domain.
93. The polypeptide of any one of embodiments 1 to 34, which comprises a first polypeptide chain comprising, in N- to C-terminal orientation:
   (a) the first split intein
   (b) a first portion of a first linker (L-1A),
   (c) a first Fc domain (Fc-1),
   (d) a second linker (L-2),
   (e) a second Fc domain (Fc-2),
   (f) a third linker (L-3),
   (g) a third Fc domain (Fc-3),
   (h) a second portion of the first linker (L-1B), and
   (i) the second split intein,
   wherein upon splicing of the first split intein and the second split intein, a circularized polypeptide chain is produced in which the first linker (L-1) becomes contiguous.
94. The polypeptide of embodiment 93, wherein the first Fc domain (Fc-1), second Fc domain (Fc-2) and third Fc-domain (Fc-3) are Fc (knob) domains.
95. The polypeptide of embodiment 93, wherein the first Fc domain (Fc-1), second Fc domain (Fc-2) and third Fc-domain (Fc-3) are Fc (hole) domains.
96. The polypeptide of any one of embodiments 93 to 95, wherein the first linker (L-1), the second linker (L-2) and the third linker (L-3) are each 3, 5 or 7 amino acids.
97. The polypeptide of embodiment 93, wherein the first linker (L-1), the second linker (L-2) and the third linker (L-3) are 3 to 20 amino acids.
98. The polypeptide of embodiment 93, wherein the first linker (L-1), the second linker (L-2) and the third linker (L-3) are 3 to 10 amino acids.
99. The polypeptide of embodiment 93, wherein the first linker (L-1), the second linker (L-2) and the third linker (L-3) are 3 to 7 amino acids.
100. The polypeptide of embodiment 93, wherein the first linker (L-1), the second linker (L-2) and the third linker (L-3) are 5 to 7 amino acids.
101. The polypeptide of any one of embodiments 93 to 100, wherein the first linker (L-1), the second linker (L-2) and the third linker (L-3) do not contain a cysteine.
102. The polypeptide of any one of embodiments 93 to 101, wherein the first linker (L-1), the second linker (L-2) and the third linker (L-3) do not contain a tryptophan.
103. The polypeptide of any one of embodiments 93 to 102, wherein the first linker (L-1), the second linker (L-2) and the third linker (L-3) do not contain a methionine.
104. The polypeptide of any one of embodiments 93 to 103, wherein the first linker (L-1), the second linker (L-2) and the third linker (L-3) do not contain an N-linked glycosylation site (e.g., as defined by the motif NXT or NXS).
105. The polypeptide of any one of embodiments 93 to 104, wherein the first linker (L-1), the second linker (L-2) and the third linker (L-3) comprise a serine and/or a threonine.
106. The polypeptide of embodiment 105, wherein the first linker (L-1), the second linker (L-2) and the third linker (L-3) comprise a serine or threonine within one amino acid from the center of the linker (e.g., at the third, fourth or fifth position in the case of a 7 amino acid linker).
107. The polypeptide of embodiment 105, wherein the first linker (L-1), the second linker (L-2) and the third linker (L-3) comprise a serine or threonine at the center of the linker (e.g., at the fourth position in the case of a 7 amino acid linker).
108. The polypeptide of any one of embodiments 93 to 107, wherein each of the first linker (L-1), the second linker (L-2) and the third linker (L-3) is predicted to lack secondary structure.
109. The polypeptide of any one of embodiments 93 to 108, wherein one, two or all three of the first linker (L-1), the second linker (L-2) and the third linker (L-3) has (e.g., comprises or consists of) an amino acid sequence selected from QLGTVEG (SEQ ID NO: 84), TLNSEES (SEQ ID NO: 85), VMSSGDQ (SEQ ID NO: 86), KLASYNP (SEQ ID NO: 87), FIDSGVG (SEQ ID NO: 88), THFSQQD (SEQ ID NO: 89), NHSSLHD (SEQ ID NO: 90), a glycine-serine linker such as GGGSGGG (SEQ ID NO: 91), or a 3-amino acid or 5-amino acid fragment of any of the foregoing.
110. The polypeptide of any one of embodiments 93 to 109, wherein the first linker (L-1), the second linker (L-2) and the third linker (L-3) are identical.
111. The polypeptide of any one of embodiments 93 to 110, which comprises a second polypeptide chain comprising a fourth Fc domain (Fc-4), a third polypeptide chain comprising a fifth Fc domain (Fc-5) and a fourth polypeptide chain comprising a sixth Fc domain (Fc-6).
112. The polypeptide of embodiment 111, wherein the second polypeptide chain comprises a first fusion partner (FP-1), the third polypeptide chain comprises a second fusion partner (FP-2), and the fourth polypeptide chain comprises a third fusion partner (FP-3).
113. The polypeptide of embodiment 112, wherein the first fusion partner (FP-1) is N-terminal to the fourth Fc domain (Fc-4), the second fusion partner (FP-2) is N-terminal to the fifth Fc domain (Fc-5), and the third fusion partner (FP-3) is N-terminal to the sixth Fc domain (Fc-6).
114. The polypeptide of embodiment 111 or embodiment 112, wherein the first fusion partner (FP-1) and the fourth Fc domain (Fc-4) are separated by a fourth linker (L-4), the second fusion partner (FP-2) and the fifth Fc domain (Fc-5) are separated by a fifth linker (L-5), and the third fusion partner (FP-3) and the sixth F domain (Fc-6) are separated by a sixth linker (L-6).
115. The polypeptide of embodiment 114, wherein the fourth linker (L-4), the fifth linker (L-5) and the sixth linker (L-6) are the same and/or selected from Section 6.7 or Table 6.
116. The polypeptide of any one of embodiments 112 to 115, wherein the first fusion partner (FP-1), the second fusion partner (FP-2) and the third fusion partner (FP-3) are the same.
117. The polypeptide any one of embodiments 112 to 116, wherein the first fusion partner (FP-1), the second fusion partner (FP-2) and the third fusion partner (FP-3) are single chain antibody or antibody fragments, optionally scFvs or sdAbs.
118. The polypeptide any one of embodiments 112 to 116, wherein the first fusion partner (FP-1), the second fusion partner (FP-2) and the third fusion partner (FP-3) are receptors or receptor fragments, optionally soluble receptor fragments.
119. The polypeptide of any one of embodiments 111 to 118, wherein the second polypeptide, the third polypeptide and the fourth polypeptide are the same.
120. The polypeptide of any one of embodiments 93 to 119, which is trivalent.
121. The polypeptide of any one of embodiments 1 to 120, which lacks a hinge domain.
122. The polypeptide of any one of embodiments 1 to 121, wherein the first split intein is an intein-C and the second split intein is an N-intein.
123. The polypeptide of any one of embodiments 1 to 122, wherein the first split intein and the second split intein comprise:
   (a) an amino acid sequence as set forth in Section 6.8, e.g., any of the sequences set forth in Table 7; or
   (b) an amino acid sequence as defined in U.S. Pat. No. 11,161,899B2, US Patent Publication No. US20160319287A1, PCT Publication No. WO 2021/099607A1, PCT Publication No. WO 2015/086825A1, PCT Publication No. WO 2017/132580A2, US Patent No. U.S. Pat. No. 8,394,604B2 or PCT Publication No. WO 2013/045632A1, optionally wherein the split intein comprises a purification tag (e.g., his tag, strep tag, or C tag).
124. A circularized antibody molecule comprising three antibody fragments (a first antibody fragment, a second antibody fragment, and a third antibody fragment), optionally wherein the three antibody fragments are Fab domains, Fc domains, or a combination thereof (e.g., as described in Section 6.2 or any one of FIGS. 1-5).
125. The circularized antibody molecule of embodiment 124, wherein the angle between each pair of antibody fragments ranges between 55° and 65°.
126. The circularized antibody molecule of embodiment 124, wherein the angle between each pair of antibody fragments ranges between 58° and 62°.
127. The circularized antibody molecule of any one of embodiments 124 to 126, wherein the angle between each pair of antibody fragments is 62° or less.
128. The circularized antibody molecule of any one of embodiments 124 to 126, wherein the angle between each pair of antibody fragments domains is 61° or less.
129. The circularized antibody molecule of any one of embodiments 124 to 128, wherein the angle between each pair of antibody fragments is 60°+1°.
130. The circularized antibody molecule of any one of embodiments 124 to 128, wherein the angle between each pair of antibody fragments is 60°+0.5°.
131. The circularized antibody molecule of any one of embodiments 124 to 130, which comprises four polypeptide chains.
132. The circularized antibody molecule of any one of embodiments 124 to 131, which comprises a first polypeptide chain comprising the split inteins and a first component of each of the three antibody fragments (a “cyclizable domain” such as an Fc, an Fd domain or a light chain (LC)), wherein each cyclizable domain is associated with a counterpart domain (e.g., another Fc domain, an LC, or an Fd, respectively) on a separate polypeptide chain.
133. The circularized antibody molecule of embodiment 132, wherein one, two or all three of the polypeptide chains comprising the counterpart domains further comprise a fusion partner.
134. The circularized antibody molecule of any one of embodiments 124 to 133, wherein:
   (a) each pair of antibody fragments in the polypeptide is separated by a linker; and/or
   (b) each pair of antibody fragments in the circularized antibody molecule is separated by a linker.
135. The circularized antibody molecule of embodiment 134, wherein each linker is 3 to 20 amino acids.
136. The circularized antibody molecule of embodiment 134, wherein each linker is 3 to 10 amino acids.
137. The circularized antibody molecule of embodiment 134, wherein each linker is 3 to 7 amino acids.
138. The circularized antibody molecule of embodiment 134, wherein each linker is 5 to 7 amino acids.
139. The circularized antibody molecule of any one of any one of embodiments 134 to 138, wherein each linker does not contain a cysteine.
140. The circularized antibody molecule of any one of embodiments 134 to 139, wherein each of the linkers does not contain a tryptophan.
141. The circularized antibody molecule of any one of embodiments 134 to 140, wherein each of the linkers does not contain a methionine.
142. The circularized antibody molecule of any one of embodiments 134 to 141, wherein each of the linkers does not contain an N-linked glycosylation site (e.g., as defined by the motif NXT or NXS).
143. The circularized antibody molecule of any one of embodiments 134 to 142, wherein each of the linkers comprises a serine and/or a threonine.
144. The circularized antibody molecule of embodiment 143, wherein each of the linkers comprises a serine or threonine within one amino acid from the center of the linker (e.g., at the third, fourth or fifth position in the case of a 7 amino acid linker).
145. The circularized antibody molecule of embodiment 143, wherein each of the linkers comprises a serine or threonine at the center of the linker (e.g., at the fourth position in the case of a 7 amino acid linker).
146. The circularized antibody molecule of any one of embodiments 134 to 145, wherein each of the linkers is predicted to lack secondary structure.
147. The circularized antibody molecule of any one of embodiments 134 to 146, wherein each of the linkers has (e.g., comprises or consists of) an amino acid sequence selected from QLGTVEG (SEQ ID NO: 84), TLNSEES (SEQ ID NO: 85), VMSSGDQ (SEQ ID NO: 86), KLASYNP (SEQ ID NO: 87), FIDSGVG (SEQ ID NO: 88), THFSQQD (SEQ ID NO: 89), NHSSLHD (SEQ ID NO: 90), a glycine-serine linker such as GGGSGGG (SEQ ID NO: 91), or a 3-amino acid or 5-amino acid fragment of any of the foregoing.
148. The circularized antibody molecule of any one of embodiments 134 to 147, wherein all the linkers are identical.
149. The circularized antibody molecule of any one of embodiments 124 to 148, wherein the angle between each pair of Fab domains ranges between 55° and 65°.
150. The circularized antibody molecule of any one of embodiments 124 to 148, wherein the angle between each pair of Fab domains ranges between 58° and 62°.
151. The circularized antibody molecule of any one of embodiments 124 to 150, wherein the angle between each pair of Fab domains 62° or less.
152. The circularized antibody molecule of any one of embodiments 124 to 150, wherein the angle between each pair of Fab domains is 61° or less.
153. The circularized antibody molecule of any one of embodiments 124 to 152, wherein the angle between each pair of Fab domains is 60°+1°.
154. The circularized antibody molecule of any one of embodiments 124 to 152, wherein the angle between each pair of Fab domains is 60°+0.5°.
155. The circularized antibody molecule of any one of embodiments 124 to 154, which comprises a first polypeptide chain comprising a first linker (L-1), a first Fd domain (Fd-1), a second linker (L-2), a second Fd domain (Fd-2), a third linker (L-3) and a third Fd domain (Fd-3).
156. The circularized antibody molecule of embodiment 155, wherein the first linker (L-1), the second linker (L-2) and the third linker (L-3) are each 3, 5 or 7 amino acids.
157. The circularized antibody molecule of embodiment 124, wherein the first linker (L-1), the second linker (L-2) and the third linker (L-3) are 3 to 20 amino acids.
158. The circularized antibody molecule of embodiment 124, wherein the first linker (L-1), the second linker (L-2) and the third linker (L-3) are 3 to 10 amino acids.
159. The circularized antibody molecule of embodiment 124, wherein the first linker (L-1), the second linker (L-2) and the third linker (L-3) are 3 to 7 amino acids.
160. The circularized antibody molecule of embodiment 124, wherein the first linker (L-1), the second linker (L-2) and the third linker (L-3) are 5 to 7 amino acids.
161. The circularized antibody molecule of any one of embodiments 155 to 160, wherein the first linker (L-1), the second linker (L-2) and the third linker (L-3) do not contain a cysteine.
162. The circularized antibody molecule of any one of embodiments 155 to 161, wherein the first linker (L-1), the second linker (L-2) and the third linker (L-3) do not contain a tryptophan.
163. The circularized antibody molecule of any one of embodiments 155 to 162, wherein the first linker (L-1), the second linker (L-2) and the third linker (L-3) do not contain a methionine.
164. The circularized antibody molecule of any one of embodiments 155 to 163, wherein the first linker (L-1), the second linker (L-2) and the third linker (L-3) do not contain an N-linked glycosylation site (e.g., as defined by the motif NXT or NXS).
165. The circularized antibody molecule of any one of embodiments 155 to 164, wherein the first linker (L-1), the second linker (L-2) and the third linker (L-3) comprise a serine and/or a threonine.
166. The circularized antibody molecule of embodiment 165, wherein the first linker (L-1), the second linker (L-2) and the third linker (L-3) comprise a serine or threonine within one amino acid from the center of the linker (e.g., at the third, fourth or fifth position in the case of a 7 amino acid linker).
167. The circularized antibody molecule of embodiment 165, wherein the first linker (L-1), the second linker (L-2) and the third linker (L-3) comprise a serine or threonine at the center of the linker (e.g., at the fourth position in the case of a 7 amino acid linker).
168. The circularized antibody molecule of any one of embodiments 155 to 167, wherein each of the first linker (L-1), the second linker (L-2) and the third linker (L-3) is predicted to lack secondary structure.
169. The circularized antibody molecule of any one of embodiments 155 to 168, wherein one, two or all three of the first linker (L-1), the second linker (L-2) and the third linker (L-3) has (e.g., comprises or consists of) an amino acid sequence selected from QLGTVEG (SEQ ID NO: 84), TLNSEES (SEQ ID NO: 85), VMSSGDQ (SEQ ID NO: 86), KLASYNP (SEQ ID NO: 87), FIDSGVG (SEQ ID NO: 88), THFSQQD (SEQ ID NO: 89), NHSSLHD (SEQ ID NO: 90), a glycine-serine linker such as GGGSGGG (SEQ ID NO: 91), or a 3-amino acid or 5-amino acid fragment of any of the foregoing.
170. The circularized antibody molecule of any one of embodiments 155 to 169, wherein the first linker (L-1), the second linker (L-2) and the third linker (L-3) are identical.
171. The circularized antibody molecule of any one of embodiments 155 to 170, which comprises a second polypeptide chain comprising a first light chain (LC-1), a third polypeptide chain comprising a second light chain (LC-2) and a fourth polypeptide chain comprising a third light chain (LC-3).
172. The circularized antibody molecule of embodiment 171, wherein the first light chain (LC-1), second light chain (LC-2) and the third light chain (LC-3) are universal light chains.
173. The circularized antibody molecule of embodiment 171 or embodiment 172, wherein the second polypeptide chain comprises a first fusion partner (FP-1), third polypeptide chain comprises a second fusion partner (FP-2), and the fourth polypeptide chain comprises a third fusion partner (FP-3).
174. The circularized antibody molecule of embodiment 173, wherein the first fusion partner (FP-1) is N-terminal to the first light chain (LC-1), the second fusion partner (FP-2) is N-terminal to the second light chain (LC-2), and the third fusion partner (FP-3) is N-terminal to the third light chain (LC-3).
175. The circularized antibody molecule of embodiment 173 or embodiment 174, wherein the first fusion partner (FP-1) and the first light chain (LC-1) are separated by a fourth linker (L-4), the second fusion partner (FP-2) and the second light chain (LC-2) are separated by a fifth linker (L-5), and the third fusion partner (FP-3) and the third light chain (LC-3) are separated by a sixth linker (L-6).
176. The circularized antibody molecule of embodiment 175, wherein the fourth linker (L-4), the fifth linker (L-5) and the sixth linker (L-6) are the same and/or selected from Section 6.7 or Table 6.
177. The circularized antibody molecule of any one of embodiments 173 to 176, wherein the first fusion partner (FP-1), the second fusion partner (FP-2) and the third fusion partner (FP-3) are the same.
178. The circularized antibody molecule of any one of embodiments 173 to 177, wherein the first fusion partner (FP-1), the second fusion partner (FP-2) and the third fusion partner (FP-3) are single chain antibody or antibody fragments, optionally scFvs or sdAbs.
179. The circularized antibody molecule of any one of embodiments 173 to 177, wherein the first fusion partner (FP-1), the second fusion partner (FP-2) and the third fusion partner (FP-3) are receptors or receptor fragments, optionally soluble receptor fragments.
180. The circularized antibody molecule of any one of embodiments 171 to 178, wherein the second polypeptide, the third polypeptide and the fourth polypeptide are the same.
181. The circularized antibody molecule of any one of embodiments 124 to 154, which comprises a first polypeptide chain comprising (L-1), a first light chain (LC-1), a second linker (L-2), a second light chain (LC-2), a third linker (L-3), and a third light chain (LC-3).
182. The circularized antibody molecule of embodiment 181, wherein the first light chain (LC-1), second light chain (LC-2) and third light chain (LC-3) are universal light chains.
183. The circularized antibody molecule of embodiment 181 or embodiment 182, wherein the first linker (L-1), the second linker (L-2) and the third linker (L-3) are each 3, 5 or 7 amino acids.
184. The circularized antibody molecule of embodiment 181, wherein the first linker (L-1), the second linker (L-2) and the third linker (L-3) are 3 to 20 amino acids.
185. The circularized antibody molecule of embodiment 181, wherein the first linker (L-1), the second linker (L-2) and the third linker (L-3) are 3 to 10 amino acids.
186. The circularized antibody molecule of embodiment 181, wherein the first linker (L-1), the second linker (L-2) and the third linker (L-3) are 3 to 7 amino acids.
187. The circularized antibody molecule of embodiment 181, wherein the first linker (L-1), the second linker (L-2) and the third linker (L-3) are 5 to 7 amino acids.
188. The circularized antibody molecule of any one of embodiments 181 to 187, wherein the first linker (L-1), the second linker (L-2) and the third linker (L-3) do not contain a cysteine.
189. The circularized antibody molecule of any one of embodiments 181 to 188, wherein the first linker (L-1), the second linker (L-2) and the third linker (L-3) do not contain a tryptophan.
190. The circularized antibody molecule of any one of embodiments 181 to 189, wherein the first linker (L-1), the second linker (L-2) and the third linker (L-3) do not contain a methionine.
191. The circularized antibody molecule of any one of embodiments 181 to 190, wherein the first linker (L-1), the second linker (L-2) and the third linker (L-3) do not contain an N-linked glycosylation site (e.g., as defined by the motif NXT or NXS).
192. The circularized antibody molecule of any one of embodiments 181 to 191, wherein the first linker (L-1), the second linker (L-2) and the third linker (L-3) comprise a serine and/or a threonine.
193. The circularized antibody molecule of embodiment 192, wherein the first linker (L-1), the second linker (L-2) and the third linker (L-3) comprise a serine or threonine within one amino acid from the center of the linker (e.g., at the third, fourth or fifth position in the case of a 7 amino acid linker).
194. The circularized antibody molecule of embodiment 192, wherein the first linker (L-1), the second linker (L-2) and the third linker (L-3) comprise a serine or threonine at the center of the linker (e.g., at the fourth position in the case of a 7 amino acid linker).
195. The circularized antibody molecule of any one of embodiments 181 to 194, wherein each of the first linker (L-1), the second linker (L-2) and the third linker (L-3) is predicted to lack secondary structure.
196. The circularized antibody molecule of any one of embodiments 181 to 195, wherein one, two or all three of the first linker (L-1), the second linker (L-2) and the third linker (L-3) has (e.g., comprises or consists of) an amino acid sequence selected from QLGTVEG (SEQ ID NO: 84), TLNSEES (SEQ ID NO: 85), VMSSGDQ (SEQ ID NO: 86), KLASYNP (SEQ ID NO: 87), FIDSGVG (SEQ ID NO: 88), THFSQQD (SEQ ID NO: 89), NHSSLHD (SEQ ID NO: 90), a glycine-serine linker such as GGGSGGG (SEQ ID NO: 91), or a 3-amino acid or 5-amino acid fragment of any of the foregoing.
197. The circularized antibody molecule of any one of embodiments 181 to 196, wherein the first linker (L-1), the second linker (L-2) and the third linker (L-3) are identical.
198. The circularized antibody molecule of any one of embodiments 181 to 197, which comprises a second polypeptide chain comprising a Fd domain, a third polypeptide chain comprising a second Fd domain (Fd-2) and a fourth polypeptide chain comprising a third Fd domain (Fd-3).
199. The circularized antibody molecule of embodiment 198, wherein the second polypeptide chain comprises a first fusion partner (FP-1), the third polypeptide chain comprises a second fusion partner (FP-2), and the fourth polypeptide chain comprises a third fusion partner (FP-3).
200. The circularized antibody molecule of embodiment 199, wherein the first fusion partner (FP-1) is N-terminal to the first Fd domain (Fd-1), the second fusion partner (FP-2) is N-terminal to the second Fd domain (Fd-2), and the third fusion partner (FP-3) is N-terminal to the third Fd domain (Fd-3).
201. The circularized antibody molecule of embodiment 199 or embodiment 200, wherein the first fusion partner (FP-1) and the first Fd domain (Fd-1) are separated by a fourth linker (L-4), the second fusion partner (FP-2) and the second Fd domain (Fd-2) are separated by a fifth linker (L-5), and the third fusion partner (FP-3) and the third Fd domain (Fd-3) are separated by a sixth linker (L-6).
202. The circularized antibody molecule of embodiment 201, wherein the fourth linker (L-4), the fifth linker (L-5) and the sixth linker (L-6) are the same and/or selected from Section 6.7 or Table 6.
203. The circularized antibody molecule of any one of embodiments 199 to 202, wherein the first fusion partner (FP-1), the second fusion partner (FP-2) and the third fusion partner (FP-3) are the same.
204. The polypeptide any one of embodiments 199 to 203, wherein the first fusion partner (FP-1), the second fusion partner (FP-2) and the third fusion partner (FP-3) are single chain antibody or antibody fragments, optionally scFvs or sdAbs.
205. The polypeptide any one of embodiments 199 to 203, wherein the first fusion partner (FP-1), the second fusion partner (FP-2) and the third fusion partner (FP-3) are receptors or receptor fragments, optionally soluble receptor fragments.
206. The circularized antibody molecule of any one of embodiments 198 to 205, wherein the second polypeptide, the third polypeptide and the fourth polypeptide are the same.
207. The circularized antibody molecule of any one of embodiments 124 to 206, wherein the three antibody fragments are identical.
208. The circularized antibody molecule of any one of embodiments 124 to 206, wherein:
   (a) at least one antibody fragment is a Fab domain, or
   (b) at least two antibody fragments are Fab domains, optionally wherein the two Fab domains differ from one another; or
   (c) all three antibody fragments are Fab domains, optionally wherein two of the Fab domains or all three Fab domains differ from one another.
209. The circularized antibody molecule of any one of embodiments 124 to 208, which is bivalent.
210. The circularized antibody molecule of any one of embodiments 124 to 208, which is trivalent.
211. The circularized antibody molecule of embodiment 210, which is monospecific.
212. The circularized antibody molecule of embodiment 210, which is multispecific.
213. The circularized antibody molecule of embodiment 212, which is bispecific.
214. The circularized antibody molecule of embodiment 212, which is trispecific.
215. The circularized antibody molecule of any one of embodiments 124 to 208, which is tetravalent.
216. The circularized antibody molecule of embodiment 215, which is monospecific.
217. The circularized antibody molecule of embodiment 215, which is multispecific.
218. The circularized antibody molecule of embodiment 217, which is bispecific.
219. The circularized antibody molecule of embodiment 217, which is trispecific.
220. The circularized antibody molecule of any one of embodiments 124 to 208, which is pentavalent.
221. The circularized antibody molecule of embodiment 220, which is monospecific.
222. The circularized antibody molecule of embodiment 220, which is multispecific.
223. The circularized antibody molecule of embodiment 222, which is bispecific.
224. The circularized antibody molecule of embodiment 222, which is trispecific.
225. The circularized antibody molecule of any one of embodiments 124 to 208, which is hexavalent.
226. The circularized antibody molecule of embodiment 225, which is monospecific.
227. The circularized antibody molecule of embodiment 225, which is multispecific.
228. The circularized antibody molecule of embodiment 227, which is bispecific.
229. The circularized antibody molecule of embodiment 227, which is trispecific.
230. The circularized antibody molecule of any one of embodiments 124 to 229, which lacks a CH2 domain.
231. The circularized antibody molecule of any one of embodiments 124 to 230, which lacks a CH3 domain.
232. The circularized antibody molecule of any one of embodiments 124 to 231, which lacks a hinge domain.
233. A circularized antibody molecule comprising three Fc regions.
234. The circularized antibody molecule of embodiment 233, wherein the angle between each pair of Fc regions ranges between 55° and 65°.
235. The circularized antibody molecule of embodiment 233, wherein the angle between each pair of Fc regions ranges between 58° and 62°.
236. The circularized antibody molecule of any one of embodiments 233 to 235, wherein the angle between each pair of Fc regions 62° or less.
237. The circularized antibody molecule of any one of embodiments 233 to 235, wherein the angle between each pair of Fc regions is 61° or less.
238. The circularized antibody molecule of any one of embodiments 233 to 237, wherein the angle between each pair of Fc regions is 60°+1°.
239. The circularized antibody molecule of any one of embodiments 233 to 237, wherein the angle between each pair of Fc regions is 60°+0.5°.
240. The circularized antibody molecule of any one of embodiments 233 to 239, which comprises a first polypeptide chain comprising a first linker (L-1), a first Fc domain (Fc-1), a second linker (L-2), a second Fc domain (Fc-2), a third linker (L-3) and a third Fc domain (Fc-3).
241. The circularized antibody molecule of embodiment 240, wherein the first linker (L-1), the second linker (L-2) and the third linker (L-3) are each 3, 5 or 7 amino acids.
242. The circularized antibody molecule of embodiment 233, wherein the first linker (L-1), the second linker (L-2) and the third linker (L-3) are 3 to 20 amino acids.
243. The circularized antibody molecule of embodiment 233, wherein the first linker (L-1), the second linker (L-2) and the third linker (L-3) are 3 to 10 amino acids.
244. The circularized antibody molecule of embodiment 233, wherein the first linker (L-1), the second linker (L-2) and the third linker (L-3) are 3 to 7 amino acids.
245. The circularized antibody molecule of embodiment 233, wherein the first linker (L-1), the second linker (L-2) and the third linker (L-3) are 5 to 7 amino acids.
246. The circularized antibody molecule of any one of embodiments 240 to 245, wherein the first linker (L-1), the second linker (L-2) and the third linker (L-3) do not contain a cysteine.
247. The circularized antibody molecule of any one of embodiments 240 to 246, wherein the first linker (L-1), the second linker (L-2) and the third linker (L-3) do not contain a tryptophan.
248. The circularized antibody molecule of any one of embodiments 240 to 247, wherein the first linker (L-1), the second linker (L-2) and the third linker (L-3) do not contain a methionine.
249. The circularized antibody molecule of any one of embodiments 240 to 248, wherein the first linker (L-1), the second linker (L-2) and the third linker (L-3) do not contain an N-linked glycosylation site (e.g., as defined by the motif NXT or NXS).
250. The circularized antibody molecule of any one of embodiments 240 to 249, wherein the first linker (L-1), the second linker (L-2) and the third linker (L-3) comprise a serine and/or a threonine.
251. The circularized antibody molecule of embodiment 250, wherein the first linker (L-1), the second linker (L-2) and the third linker (L-3) comprise a serine or threonine within one amino acid from the center of the linker (e.g., at the third, fourth or fifth position in the case of a 7 amino acid linker).
252. The circularized antibody molecule of embodiment 250, wherein the first linker (L-1), the second linker (L-2) and the third linker (L-3) comprise a serine or threonine at the center of the linker (e.g., at the fourth position in the case of a 7 amino acid linker).
253. The circularized antibody molecule of any one of embodiments 240 to 252, wherein each of the first linker (L-1), the second linker (L-2) and the third linker (L-3) is predicted to lack secondary structure.
254. The circularized antibody molecule of any one of embodiments 240 to 253, wherein one, two or all three of the first linker (L-1), the second linker (L-2) and the third linker (L-3) has (e.g., comprises or consists of) an amino acid sequence selected from QLGTVEG (SEQ ID NO: 84), TLNSEES (SEQ ID NO: 85), VMSSGDQ (SEQ ID NO: 86), KLASYNP (SEQ ID NO: 87), FIDSGVG (SEQ ID NO: 88), THFSQQD (SEQ ID NO: 89), NHSSLHD (SEQ ID NO: 90), a glycine-serine linker such as GGGSGGG (SEQ ID NO: 91), or a 3-amino acid or 5-amino acid fragment of any of the foregoing.
255. The circularized antibody molecule of any one of embodiments 240 to 254, wherein the first linker (L-1), the second linker (L-2) and the third linker (L-3) are identical.
256. The circularized antibody molecule of any one of embodiments 240 to 255, which comprises a second polypeptide chain comprising a fourth Fc domain (Fc-4), a third polypeptide chain comprising a fifth Fc domain (Fc-5) and a fourth polypeptide chain comprising a sixth Fc domain (Fc-6).
257. The circularized antibody molecule of embodiment 255 or embodiment 256, wherein the second polypeptide chain comprises a first fusion partner (FP-1), third polypeptide chain comprises a second fusion partner (FP-2), and the fourth polypeptide chain comprises a third fusion partner (FP-3).
258. The circularized antibody molecule of embodiment 257, wherein the first fusion partner (FP-1) is N-terminal to the fourth Fc domain (Fc-4), the second fusion partner (FP-2) is N-terminal to the fifth Fc domain (Fc-5), and the third fusion partner (FP-3) is N-terminal to the sixth Fc domain (Fc-6).
259. The circularized antibody molecule of embodiment 257 or embodiment 258, wherein the first fusion partner (FP-1) and the fourth Fc domain (Fc-4) are separated by a fourth linker (L-4), the second fusion partner (FP-2) and the fifth Fc domain (Fc-5) are separated by a fifth linker (L-5), and the sixth Fc domain (Fc-6) and the third light chain (LC-3) are separated by a sixth linker (L-6).
260. The circularized antibody molecule of embodiment 259, wherein the fourth linker (L-4), the fifth linker (L-5) and the sixth linker (L-6) are the same and/or selected from Section 6.7 or Table 6.
261. The circularized antibody molecule of any one of embodiments 257 to 260, wherein the first fusion partner (FP-1), the second fusion partner (FP-2) and the third fusion partner (FP-3) are the same.
262. The circularized antibody molecule of any one of embodiments 257 to 261, wherein the first fusion partner (FP-1), the second fusion partner (FP-2) and the third fusion partner (FP-3) are single chain antibody or antibody fragments, optionally scFvs or sdAbs.
263. The circularized antibody molecule of any one of embodiments 257 to 261, wherein the first fusion partner (FP-1), the second fusion partner (FP-2) and the third fusion partner (FP-3) are receptors or receptor fragments, optionally soluble receptor fragments.
264. The circularized antibody molecule of any one of embodiments 233 to 263, which is bivalent.
265. The circularized antibody molecule of any one of embodiments 233 to 263, which is trivalent.
266. The circularized antibody molecule of embodiment 265, which is monospecific.
267. The circularized antibody molecule of embodiment 265, which is multispecific.
268. The circularized antibody molecule of embodiment 267, which is bispecific.
269. The circularized antibody molecule of embodiment 267, which is trispecific.
270. A method of producing the circularized antibody molecule of any one of embodiments 124 to 269, comprising exposing the polypeptide of any one of embodiments 1 to 123 to conditions which result in splicing of the first split intein and the second split intein.
271. The method of embodiment 270, which further comprises purifying or enriching for the circularized antibody molecule.
272. A population of circularized antibody molecules according to any one of embodiments 124 to 269, or produced by the method of embodiment 270 or embodiment 271.
273. The population of embodiment 272, which is at least 50% homogeneous.
274. The population of embodiment 272, which is at least 60% homogeneous.
275. The population of embodiment 272, which is at least 70% homogeneous.
276. A nucleic acid or plurality of nucleic acids encoding the polypeptide of any one of embodiments 1 to 123 or a precursor of the circularized antibody molecule of any one of embodiments 124 to 269.
277. A host cell engineered to express the polypeptide of any one of embodiments 1 to 123, a precursor of the circularized antibody molecule of any one of embodiments 124 to 269, or the nucleic acid(s) of embodiment 276.
278. A method of producing the polypeptide of any one of embodiments 1 to 123 or the circularized antibody molecule of any one of embodiments 124 to 269 or its precursor, comprising culturing the host cell of embodiment 277 and recovering the polypeptide expressed thereby, optionally further comprising purifying the polypeptide, the circularized antibody molecule, and/or the precursor.
279. A pharmaceutical composition comprising the circularized antibody molecule of any one of embodiments 124 to 269, or the population of any one of embodiments 272 to 275 and an excipient.
280. A method for clustering cell surface molecules, comprising contacting a cell that expresses the cell surface molecules with the circularized antibody molecule of any one of embodiments 124 to 269, which comprises Fab domains and/or fusion partners which bind to the cell surface molecule, optionally wherein the cell surface molecule is in the form of a population according to one of embodiments 272 to 275 or a pharmaceutical composition according to embodiment 279.
281. The method of embodiment 280, wherein the cell surface molecules are trimeric receptors.
282. The method of embodiment 281, wherein the trimeric receptors are TNFα receptors. 283. A method for cross-linking a first cell and a second cell, comprising contacting the first cell and the second cell with the circularized antibody molecule of any one of embodiments 124 to 269, optionally wherein the cell surface molecule is in the form of a population according to one of embodiments 272 to 275 or a pharmaceutical composition according to embodiment 279, wherein:
   (a) the Fab domains of the circularized antibody molecule binds to the first cell; and
   (b) the circularized antibody molecule comprises fusion partners, e.g., single chain antibodies or antibody fragments and/or receptors as defined in any one of embodiments 173 to 180, 199 to 206, and 257 to 263.
284. A method for inhibiting an infectious agent, e.g., to treat or prevent an infection, comprising contacting the infectious agent with the circularized antibody molecule of any one of embodiments 124 to 269, optionally wherein the cell surface molecule is in the form of a population according to one of embodiments 272 to 275 or a pharmaceutical composition according to embodiment 279, wherein:
   (a) the circularized antibody molecule comprises Fab domains that binds to the infectious agent (e.g., SARS-CoV2, such as via its spike protein, and in some embodiments to its receptor binding domain); and/or
   (b) the circularized antibody molecule comprises at least a binding portion of the cellular receptor for the infectious agent (e.g., the ACE2 receptor), optionally wherein the at least a binding portion of the cellular receptor is configured as a fusion partner as defined in any one of embodiments 173 to 180, 199 to 206, and 257 to 263.
285. The method of any one of embodiments 280 to 284, which is performed in vitro or ex vivo.
286. The method of any one of embodiments 280 to 284, which is performed in vivo and comprises administering the circularized antibody molecule to a subject in need thereof.
287. A circularized antibody molecule of any one of embodiments 124 to 269, a population according to one of embodiments 272 to 275 or a pharmaceutical composition according to embodiment 279, which is suitable (e.g., comprises Fab domains and/or fusion partners) suitable for performing the method of any one of embodiments 280 to 286.

### 8. EXAMPLES

### 8.1. Materials and Methods

### 8.1.1. Construct Design, Production, and Purification

Exemplary tri-Fab constructs were designed to utilize split-intein mediated circularization to produce cyclic constructs, wherein a polypeptide comprising either three Fd or three light chain (LC) sequences flanked by two split inteins was polymerized with LC or Fd peptides, respectively, and circularized through the formation of an intein complex by joining the two split inteins. Linear tri-Fab constructs were designed similarly, but comprised either inactive or no split inteins.

DNA fragments encoding each polypeptide were codon optimized and cloned into a suitable mammalian expression plasmid. Pairs of plasmids that encode the components of a single tri-Fab construct (e.g., the exemplary pairs depicted in FIGS. 2A and 2B; 2C and 2D; 3A and 3B; 3A and 3C; 4A and 4B; and 4A and 4C) were combined in 1:1 ratio and expressed in Expi293 cells by transient transfection (Thermo Fisher Scientific). Proteins in 50 mL of Expi297 supernatant were purified with Protein L chromatography following the manufacturer's protocol. Purified constructs were analyzed via reducing and non-reducing SDS-PAGE and Intact Mass Analysis.

### 8.1.2. Intact Mass Analysis

Mass spectrometry (MS)-based assessments of the linear control and tri-Fab samples were conducted to determine the molecular weights of linear control and tri-Fab samples under non-reduced and reduced conditions, and to assess the conformance of the experimentally determined values to the predicted molecular weights based on the amino acid sequence. Non-reduced samples were injected onto a Waters ACQUITY reversed-phase UPLC BEH300 C4 column (1.7 μm, 2.1 mm×50 mm), equilibrated with 99% mobile phase A (0.1% formic acid in Milli-Q water) and 1% mobile phase B (0.1% formic acid in acetonitrile) prior to sample injection, using a gradient of increasing acetonitrile concentration to elute samples for MS analysis. MS data were acquired using a Waters Xevo G2S QTof MS with an electrospray ionization (ESI) source operating in positive ion mode. The same samples were reduced with 10 mM tris(2-carboxyethyl) phosphine hydrochloride (TCEP HCl) at 50° C. for 30 minutes, then were injected onto the same LC/MS system described above for intact mass analysis.

### 8.1.3. Metadynamics Simulation

Metadynamics simulation was used to study the effect of linker length on free energy of cyclic tri-Fab constructs, to help optimize the angle between two adjacent Fab domains. Metadynamics simulation is a widely used technique that allows exploration of the free energy landscape. The result of metadynamics would provide free energy as a function of collective variables (CVs). In our simulations, a CV is defined as the distance between the center of mass of amino acids at the ends of the linker. There are total of three CVs defined based on three linkers for a single tri-Fab construct. For a single tri-Fab construct with a specific linker, three 50 ns metadynamics simulations were run with two out of three CVs at a time (total 150 ns metadynamics simulations time). For the metadynamics simulations, the initial Gaussian hill height, Gaussian width and the bias deposition interval were set to 0.01 kcal/mol, 0.05 Å, and 0.001 ps respectively.

Cyclic tri-Fab constructs were modeled from their amino acid sequences using MOE and Discovery Studio v20.1. The initial structures were energy minimized in Discovery Studio. All simulation systems were built using Maestro and all Molecular Dynamics (MD) and metadynamics simulations were run using Desmond v7.0 (Schrödinger, Inc.). The initial structures from Discovery Studio were then prepared using Protein Preparation script, and then the simulation system was built using System Builder script within Maestro. The simulation box size was calculated by maintaining 10 Å distance between the cyclic tri-Fab construct and simulation box boundary. SPC water model was used, and neutralizing ions were added to the system. An Optimized Potentials for Liquid Simulations (OPLS4) force field was used for the simulations. After minimization and relaxation of the system, MD equilibration run was performed for 1.2 ns and then metadynamics was performed as explained earlier in three steps. All simulations were run with temperature maintained at 300 K and pressure maintained at 1.013 bar using MTK thermostat-barostat method (NPT ensemble). The metadynamics simulation data was analyzed using metadynamics analysis tool in Desmond (Schrödinger, Inc.).

### 8.1.4. SEC-MALS Analysis

Size exclusion chromatography coupled with multi-angle laser light scattering (SEC-MALS) was utilized to assess the presence of potential production byproducts such as oligomeric cyclic tri-Fabs (e.g., hexa-Fabs, nona-Fabs, dodeca-Fabs, etc.) and to determine the radius of gyration (R_{g}) for each construct evaluated.

SEC analysis was conducted on a Waters Acquity UPLC H-Class system. 10 μg of each protein sample was injected into a two-column tandem setup consisting of Acquity BEH SEC columns (200 Å, 1.7 μm, 4.6×150 mm). Flow rate was set at 0.3 ml/min. Mobile phase buffer contained 10 mM sodium phosphate, 500 mM NaCl, PH 7.0. UV absorbance at 280 nm, light scattering and refractive index changes were monitored using Wyatt microOptilab refractive index and Wyatt-microDawn light scattering detector. Bovine serum albumin was injected using the same parameter settings as a system suitability control. Data was collected and analyzed using ASTRA software (Version 7.3.1 Wyatt Technology).

### 8.1.5. DLS Analysis

Dynamic light scattering (DLS) was used to assess the size distribution of particles in construct samples and determine the hydrodynamic radii of tri-Fab constructs. All samples were run in six replicates.

Samples were loaded neat in replicates of six (30 μL per replicate) onto a 384-well plate, and data was collected on a DynaPro PlateReader II (Wyatt Technology). Thirty 4-second data acquisitions were obtained for each well at 25° C. Data analysis of the resulting autocorrelation functions was performed with Dynamics software (version 7, Wyatt Technology) using the manufacturer's implementation of a non-negative least squares minimization with regularization.

### 8.1.6. Negative Stain EM

Negative stain electron microscopy (EM) was conducted to qualitatively assess the conformational distribution of tri-Fab constructs.

Purified samples at a protein concentration of approximately 0.02 mg/mL were applied to 400 mesh carbon film Cu grids (Electron Microscopy Sciences) and negative-stained with NanoW (Nanoprobes) or Vitroease Methylamine Tungstate (Thermo Fisher). Negative stain EM grids were inserted into a Glacios TEM (Thermo Fisher) and imaged with a Ceta camera (Thermo Fisher). Automated data collection was performed at a nominal magnification of 73,000× and 2 Å pixel size using EPU. A total of 550 micrographs were collected and EM data were processed using RELION 4.0, wherein 450,000 particles were picked using the Laplacian of Gaussian (LoG) algorithm to generate 2D templates that were subsequently used for template-based particle picking. Particle images were subjected to multiple rounds of 2D classification, selecting particles belonging to class averages with clear features after each round.

### 8.2. Example 1. Optimization of Linkers Between Fab Domains

Given that each Fab domain of cyclic tri-Fab is connected to the two adjacent Fab domains via linkers, selection of optimal linkers is vital for two reasons. First, the linker must enable formation of triangular molecules with approximately 60° angles, and minimize the likelihood of concatenation. Second, it needs to enable efficient trans-splicing of intein complex.

Using metadynamics simulation, as described in Section 8.1.3, three Fab structures were arranged in cyclic conformations and two adjacent heavy chain termini were connected via suitable linkers. A suitable linker was defined as a linker that: (i) lacks cysteine, tryptophan, methionine; (ii) lacks N-linked glycosylation sites, such as those that comprise NXT or NXS motifs; (iii) comprises a serine and/or a threonine; (iv) has minimal immunogenicity; (v) lacks secondary structures; and allows 14-17 Å distance between the Fab termini.

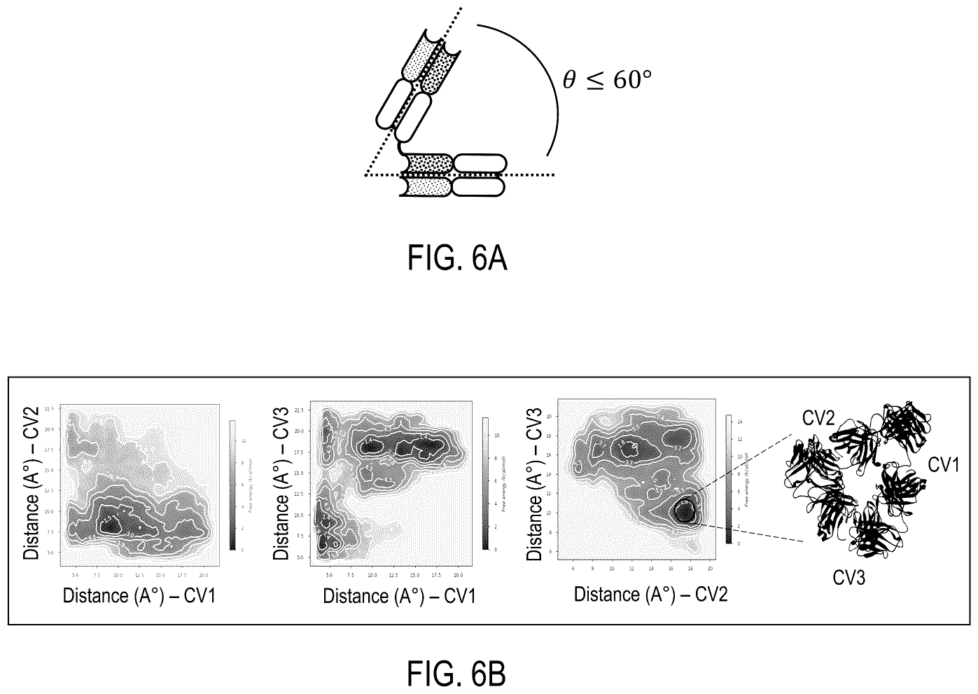

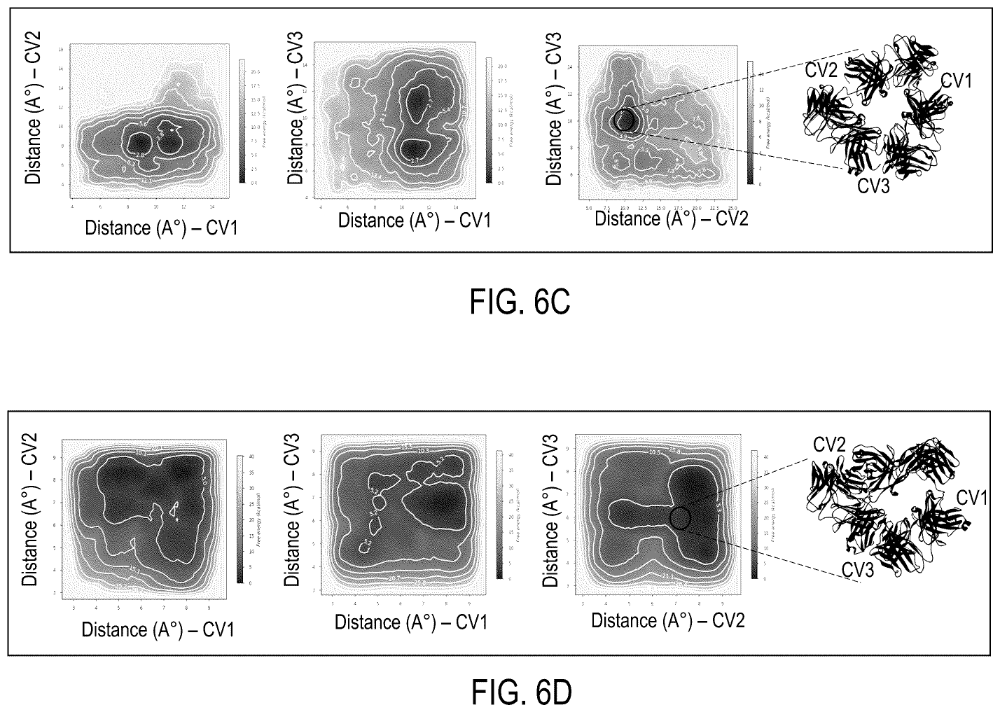

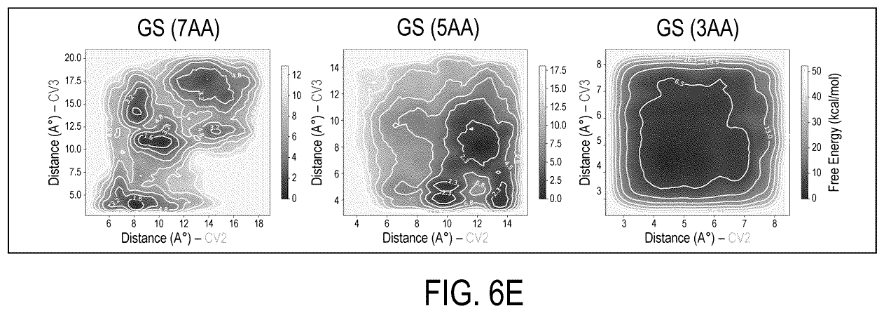

A 7aa linker that fits the above criteria, FIGSGVG (SEQ ID NO: 279), and two shorter linkers derived from this linker (IDSGV (SEQ ID NO: 280) and DSG), were evaluated to determine the optimal linker length. Simulations suggested that the lowest energy conformations were achieved with mean linker lengths between 4 Å-20 Å for the tri-Fab constructs comprising the 7aa linker (FIG. 6B), whereas the constructs with 5aa or 3aa linkers were associated with mean linker lengths between 8 Å-12 Å or 3 Å-9 Å, respectively (FIGS. 6C and 6D). By visual inspection of the simulation conformations, constructs with 3aa linkers displayed deviations from optimal triangular formation in terms of angles formed by Fab domains of cyclic tri-Fab (FIG. 6D), whereas constructs with 7aa linkers might have some out of plane distortions. Similar results were obtained with another 7aa linker, GGGSGGG (SEQ ID NO: 91), and 5aa and 3aa linkers derived from this linker (GGSGG (SEQ ID NO: 92) and GSG) (FIG. 6E). Taken together, these simulation results suggest that the 5aa linker was the more optimal linker followed by 7aa and 3aa variants respectively.

### 8.3. Example 2. Generation of Cyclic Tri-Fabs

A polypeptide pair encoding a cyclic tri-Fab construct as depicted in FIGS. 2A and 2B and a control polypeptide pair that lacks split inteins and encodes a linear tri-Fab construct were designed as described in Section 8.1.1. Each pair was expressed in Expi297 cells and protein products in Expi297 supernatants were purified as described in Section 8.1.1.

The protein yields for the linear and circularized antibody molecules were 39 μg/mL (1.95 mg/50 mL) and 41.5 μg/mL (2.075 mg/50 mL), respectively, suggesting the production efficiencies for linear and cyclic constructs were comparable.

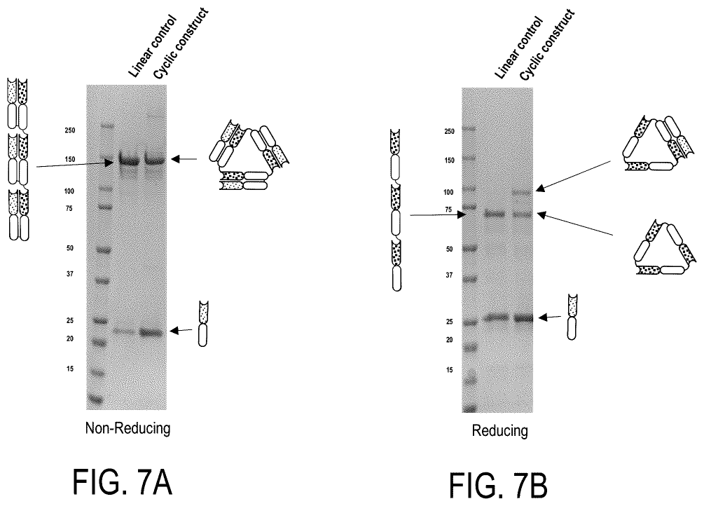

Non-reducing SDS-PAGE analysis of the two constructs revealed that the co-expression of their respective polypeptide pairs resulted in the formation of tri-Fab constructs, which were approximately 150 kDa in size (FIG. 7A). Reducing SDS-PAGE analysis of the same constructs showed fully or partially dissociated tri-Fab constructs (FIG. 7B). No intein-comprising constructs were observed with either SDS-PAGE analysis. These results suggest that in situ intein-circularization of cyclic tri-Fabs was associated with complete removal of intein complexes from the circularized product.
8.4. Example 3. Intact Mass Analysis Confirming the Formation of Cyclic Tri-Fab

To confirm the formation of the cyclic tri-Fab structure, linear tri-Fab and intein-reacted cyclic tri-Fab were subject to intact mass analysis under mass spectrometry as described in Section 8.1.1. Under non-reducing conditions, cyclic tri-Fab exhibited with an observed mass of 141,699 Daltons (Da), which was 16 Da smaller than the linear tri-Fab of 141,714 Dalton (Table 9). Under reducing conditions, a similar decrease in molecular weight (−16 Da) was observed also with cyclic tri-Fd (VH-CH1 domains in Fab) in comparison to linear tri-Fd (Table 9). The decreases in mass corresponded to the cyclization of the tri-Fab as a result of a new peptide bond formation and the leaving of the H₂O group. In addition, under reducing conditions, a unique mass species of 95, 167 Da was observed only with the cyclic tri-Fab, corresponding to cyclic tri-Fd associated with one light chain (LC), which confirmed the identity of the additional high molecular weight band observed with reducing SDS-PAGE (Table 9, FIG. 7B).

TABLE 9
Intact Mass Analysis of Linear and Intein-Cyclized tri-Fabs

| | Calculated | Observed | Error |
|---|---|---|---|
| Sample | Mass (Da) | Mass (Da) | (Da) |
| Linear | Intact tri-Fab* | 141717.3 | 141714.0 |
| tri-Fabs | LC | 23278.6 | 23279.0 |
| | tri-Fd | 71911.7 | 71909.0 |
| | tri-Fd + LC | 95188.3 | ND |
| Cyclic | Intact tri-Fab* | 141699.3 | 141699.0 |
| tri-Fabs | LC | 23278.6 | 23279.0 |
| | tri-Fd | 71893.7 | 71893.0 |
| | tri-Fd + LC | 95170.3 | 95167.0 |

*Non-reducing condition

### 8.5. Example 4. Structural Analysis of Tri-Fab Constructs

To evaluate the structural characteristics of linear and circular tri-Fab constructs, the shape factor (ρ) of each construct was calculated using gyration and hydrodynamic radii (R_{g} and Rₕ) as described in Sections 8.1.4 and 8.1.5.

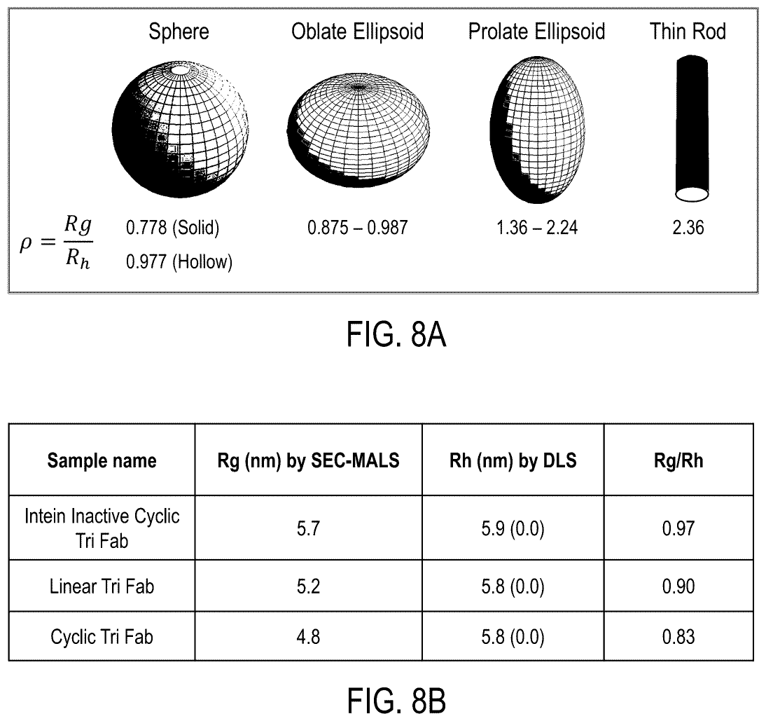

R_{g} represents mass distribution about the center of mass from angular dependence of static light scattering, whereas Rₕ represents the radius of an equivalent hard-sphere diffusing light at the same rate as the molecule under observation, determined from dynamic light scattering. The ratio of R_{g} and Rn result in the shape factor, p (FIG. 8A).

Three constructs were evaluated: a cyclic tri-Fab, a linear tri-Fab that lacks split intein sequences, and a linear tri-Fab that comprises inactive split Intein sequences. The cyclic tri-Fab construct was associated with the most compact spherical shape (FIG. 8B). The linear tri-Fab constructs were associated with more elongated structures (FIG. 8B).
8.6. Example 5. Direct Visualization of Tri-Fab Construct Structure Via Negative Stain Electron Microscopy

To further evaluate the structures of cyclic tri-Fab constructs and assess the conformational distribution of tri-Fabs in each sample, negative stain EM was used as described in Section 8.1.6.

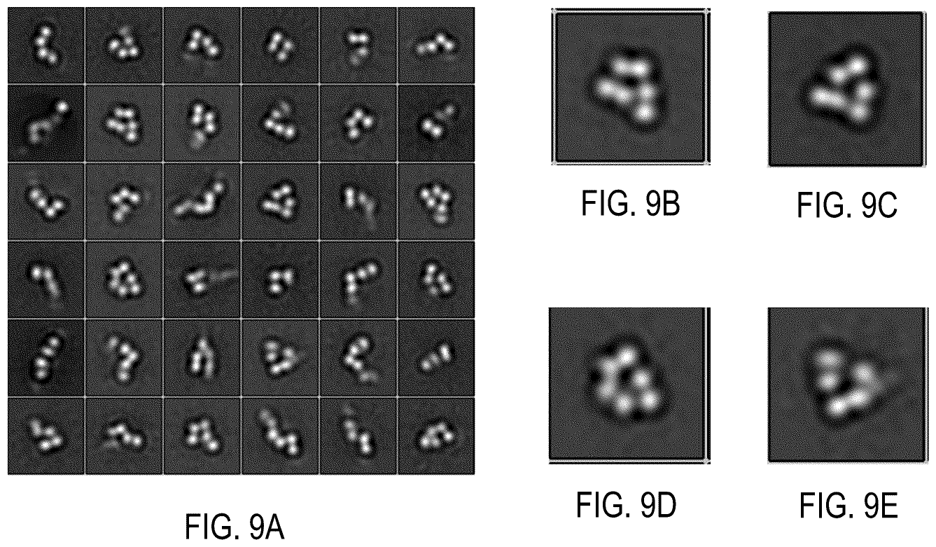

138,000 particles in the cyclic tri-Fab sample e were arranged into the top populated classes after the third 2D classification run. Cyclic tri-Fab samples displayed tri-Fabs linked together in different orientations and conformations (FIG. 9A). Yet, a higher number of top classes were putative cyclic molecules and higher percentages of particles were categorized as cyclic in the evaluated sample (FIGS. 9B-9E).

### 9. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

1. A polypeptide comprising a first split intein, three antibody fragments, and a second split intein, wherein the splicing of the first split intein and the second split intein results in a circularized antibody molecule comprising the three antibody fragments, wherein the three antibody fragments are Fab domains, Fc domains, or a combination thereof.

2. The polypeptide of claim 1, wherein the angle between each pair of antibody fragments is 60°±1°.

3. The polypeptide of claim 1, which comprises four polypeptide chains.

4. The polypeptide of claim 1, wherein:
(a) each pair of antibody fragments in the polypeptide is separated by a linker of 3 to 7 amino acids; and/or
(b) each pair of antibody fragments in the circularized antibody molecule is separated by a linker of 3 to 7 amino acids.

5. (canceled)

6. The polypeptide of claim 4, wherein each linker does not contain a cysteine, a tryptophan, or a methionine.

7. The polypeptide of claim 4, wherein each of the linkers does not contain an N-linked glycosylation site (as defined by the motif NXT or NXS).

8. The polypeptide of claim 4, wherein each of the linkers comprises a serine or threonine within one amino acid from the center of the linker.

9. The polypeptide of claim 4, wherein each of the linkers comprises or consists of an amino acid sequence selected from QLGTVEG (SEQ ID NO: 84), TLNSEES (SEQ ID NO: 85), VMSSGDQ (SEQ ID NO: 86), KLASYNP (SEQ ID NO: 87), FIDSGVG (SEQ ID NO: 88), THFSQQD (SEQ ID NO: 89), NHSSLHD (SEQ ID NO: 90), a glycine-serine linker, or a 3-amino acid or 5-amino acid fragment of any of the foregoing.

10. The polypeptide of claim 4, wherein all the linkers are identical.

11. The polypeptide of claim 1, which comprises a first polypeptide chain comprising, in N- to C-terminal orientation:
(a) the first split intein (I-A)
(b) a first portion of a first linker (L-1A),
(c) a first Fd domain (Fd-1),
(d) a second linker (L-2),
(e) a second Fd domain (Fd-2),
(f) a third linker (L-3),
(g) a third Fd domain (Fd-3),
(h) a second portion of the first linker (L-1B), and
(i) the second split intein (I-B),
wherein upon splicing of the first split intein (I-A) and the second split intein (I-B), a circularized polypeptide chain is produced in which the first linker becomes contiguous.

12. The polypeptide of claim 11, which comprises a second polypeptide chain comprising a first light chain (LC-1), a third polypeptide chain comprising a second light chain (LC-2) and a fourth polypeptide chain comprising a third light chain (LC-3).

13. The polypeptide of claim 12, wherein the second polypeptide chain comprises a first fusion partner (FP-1), the third polypeptide chain comprises a second fusion partner (FP-2), and the fourth polypeptide chain comprises a third fusion partner (FP-3).

14. The polypeptide claim 13, wherein the first fusion partner (FP-1), the second fusion partner (FP-2) and the third fusion partner (FP-3) are (1) single chain antibody or antibody fragments, or (2) receptors or receptor fragments.

15. The polypeptide of claim 12, wherein the second polypeptide, the third polypeptide and the fourth polypeptide are the same.

16. The polypeptide of claim 1, which comprises a first polypeptide chain comprising, in N- to C-terminal orientation:
(a) the first split intein
(b) a first portion of a first linker (L-1A),
(c) a first light chain (LC-1),
(d) a second linker (L-2),
(e) a second light chain (LC-2),
(f) a third linker (L-3),
(g) a third light chain (LC-3),
(h) a second portion of the first linker (L-1B), and
(i) the second split intein,
wherein upon splicing of the first split intein and the second split intein, a circularized polypeptide chain is produced in which the first linker (L-1) becomes contiguous.

17. The polypeptide of claim 16, wherein the first linker (L-1), the second linker (L-2) and the third linker (L-3) are identical.

18. The polypeptide of claim 16, which comprises a second polypeptide chain comprising a Fd domain, a third polypeptide chain comprising a second Fd domain (Fd-2) and a fourth polypeptide chain comprising a third Fd domain (Fd-3).

19. The polypeptide of claim 18, wherein the second polypeptide chain comprises a first fusion partner (FP-1), the third polypeptide chain comprises a second fusion partner (FP-2), and the fourth polypeptide chain comprises a third fusion partner (FP-3).

20. The polypeptide claim 19, wherein the first fusion partner (FP-1), the second fusion partner (FP-2) and the third fusion partner (FP-3) are (1) single chain antibody or antibody fragments or (2) receptors or receptor fragments.

21. The polypeptide of claim 18, wherein the second polypeptide, the third polypeptide and the fourth polypeptide are the same.

22. The polypeptide of claim 1, wherein the antibody fragments are Fab domains and the three Fab domains are identical.

23. The polypeptide of claim 1, wherein the antibody fragments are Fab domains and at least two Fab domains differ from one another.

24. The polypeptide of claim 1, which is trivalent.

25. The polypeptide of claim 1, which is hexavalent.

26. The polypeptide of claim 1, which lacks a CH2 domain and/or a CH3 domain.

27. The polypeptide of claim 1, which lacks a hinge domain.

28. The polypeptide of claim 1, wherein the first split intein is an intein-C and the second split intein is an N-intein.

29. The polypeptide of claim 1, wherein the first split intein and the second split intein comprise an amino acid sequence set forth in Table 7.

30. A circularized antibody molecule comprising three antibody fragments, wherein the three antibody fragments are Fab domains, Fc domains, or a combination thereof.

31.-47. (canceled)

48. The circularized antibody molecule of claim 30, which is bivalent or trivalent.

49.-51. (canceled)

52. The circularized antibody molecule of claim 30, which is, tetravalent, pentavalent, or hexavalent.

53.-56. (canceled)

57. A circularized antibody molecule comprising three Fc regions.

58. (canceled)

59. A method of producing a circularized antibody molecule, comprising exposing the polypeptide of claim 1 to conditions which result in splicing of the first split intein and the second split intein.

60. A population of circularized antibody molecules according to claim 30.

61. (canceled)

62. A nucleic acid or plurality of nucleic acids encoding the polypeptide claim 1.

63. A host cell engineered to express the polypeptide of claim 1.

64. A method of producing a polypeptide, comprising culturing the host cell of claim 63 and recovering the polypeptide expressed thereby.

65. A pharmaceutical composition comprising the circularized antibody molecule of claim 30.

66. A method for clustering cell surface molecules, comprising contacting a cell that expresses the cell surface molecules with the circularized antibody molecule of claim 30, which comprises Fab domains and/or fusion partners which bind to the cell surface molecule.

67. The method of claim 66, wherein the cell surface molecules are trimeric receptors.

68. A method for cross-linking a first cell and a second cell, comprising contacting the first cell and the second cell with the circularized antibody molecule of claim 30, wherein:
(a) the Fab domains of the circularized antibody molecule binds to the first cell; and
(b) the circularized antibody molecule comprises fusion partners.

69. A method for inhibiting an infectious agent, comprising contacting the infectious agent with the circularized antibody molecule of claim 30, wherein:
(a) the circularized antibody molecule comprises Fab domains that binds to the infectious agent; and/or
(b) the circularized antibody molecule comprises at least a binding portion of the cellular receptor for the infectious agent.

70. (canceled)

71. (canceled)


SEQUENCE LISTING (834 sequences)

SEQ ID NO: 1 - protein, 805 aa, Homo sapiens [sequence omitted]

SEQ ID NO: 2 - protein, 596 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKNIDVTDAMVDQAWDAQRIFKEAEKFFVSVGLPNMTQGFWENSMLTDPGNVQKAVCHPTAWDLGKGDFRLMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 3 - protein, 723 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 4 - protein, 104 aa, Homo sapiens
GPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 5 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 8 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 9 - protein, 107 aa, synthetic construct
GQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 107 aa, synthetic construct
GQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSPGK

SEQ ID NO: 11 - protein, 104 aa, Homo sapiens
GPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 12 - protein, 120 aa, synthetic construct
QVQLVESGGGLVKPGGSLRLSCAASGFTFSDYYMSWIRQAPGKGLEWVSYITYSGSTIYYADSVKGRFTISRDNAKSSLYLQMNSLRAEDTAVYYCARDRGTTMVPFDYWGQGTLVTVSS

SEQ ID NO: 13 - protein, 8 aa, synthetic construct
GFTFSDYY

SEQ ID NO: 14 - protein, 8 aa, synthetic construct
ITYSGSTI

SEQ ID NO: 15 - protein, 13 aa, synthetic construct
ARDRGTTMVPFDY

SEQ ID NO: 16 - protein, 107 aa, synthetic construct
DIQMTQSPSSLSASVGDRVTITCQASQDITNYLNWYQQKPGKAPKLLIYAASNLETGVPSRFSGSGSGTDFTFTISGLQPEDIATYYCQQYDNLPLTFGGGTKVEIK

SEQ ID NO: 17 - protein, 6 aa, synthetic construct
QDITNY

SEQ ID NO: 18 - protein, 9 aa, synthetic construct
QQYDNLPLT

SEQ ID NO: 19 - protein, 450 aa, synthetic construct
QVQLVESGGGLVKPGGSLRLSCAASGFTFSDYYMSWIRQAPGKGLEWVSYITYSGSTIYYADSVKGRFTISRDNAKSSLYLQMNSLRAEDTAVYYCARDRGTTMVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 214 aa, synthetic construct
DIQMTQSPSSLSASVGDRVTITCQASQDITNYLNWYQQKPGKAPKLLIYAASNLETGVPSRFSGSGSGTDFTFTISGLQPEDIATYYCQQYDNLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - DNA, 360 bp, synthetic construct
  source 1..360: mol_type = other DNA
caggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattacttatagtggtagtaccatatactacgcagactctgtgaagggccgattcaccatctccagggacaacgccaagagctcactgtatctgcaaatgaacagcctgagagccgaggacacggccgtgtattactgtgcgagagatcgcggtacaactatggtcccctttgactactggggccagggaaccctggtcaccgtctcctca

SEQ ID NO: 22 - DNA, 24 bp, synthetic construct
  source 1..24: mol_type = other DNA
ggattcaccttcagtgactactac

SEQ ID NO: 23 - DNA, 24 bp, synthetic construct
  source 1..24: mol_type = other DNA
attacttatagtggtagtaccata

SEQ ID NO: 24 - DNA, 39 bp, synthetic construct
  source 1..39: mol_type = other DNA
gcgagagatcgcggtacaactatggtcccctttgactac

SEQ ID NO: 25 - DNA, 321 bp, synthetic construct
  source 1..321: mol_type = other DNA
gacatccagatgacccagtctccatcctccctgtctgcatctgtaggagacagagtcaccatcacttgccaggcgagtcaggacattaccaactatttaaattggtatcagcagaaaccagggaaagcccctaagctcctgatctacgctgcatccaatttggaaacaggggtcccatcaaggttcagtggaagtggatctgggacagattttactttcaccatcagcggcctgcagcctgaagatattgcaacatattactgtcaacagtatgataatctccctctcactttcggcggagggaccaaggtggagatcaaa

SEQ ID NO: 26 - DNA, 18 bp, synthetic construct
  source 1..18: mol_type = other DNA
caggacattaccaactat

SEQ ID NO: 27 - DNA, 27 bp, synthetic construct
  source 1..27: mol_type = other DNA
caacagtatgataatctccctctcact

SEQ ID NO: 28 - DNA, 1353 bp, synthetic construct
  source 1..1353: mol_type = other DNA
caggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattacttatagtggtagtaccatatactacgcagactctgtgaagggccgattcaccatctccagggacaacgccaagagctcactgtatctgcaaatgaacagcctgagagccgaggacacggccgtgtattactgtgcgagagatcgcggtacaactatggtcccctttgactactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 29 - DNA, 645 bp, synthetic construct
  source 1..645: mol_type = other DNA
gacatccagatgacccagtctccatcctccctgtctgcatctgtaggagacagagtcaccatcacttgccaggcgagtcaggacattaccaactatttaaattggtatcagcagaaaccagggaaagcccctaagctcctgatctacgctgcatccaatttggaaacaggggtcccatcaaggttcagtggaagtggatctgggacagattttactttcaccatcagcggcctgcagcctgaagatattgcaacatattactgtcaacagtatgataatctccctctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 30 - protein, 120 aa, synthetic construct
EVQLVESGGGLVKPGGSLRLSCAASGITFSNAWMSWVRQAPGKGLEWVGRIKSKTDGGTTDYAAPVKGRFTISRDDSKNTLYLQMNSLKTEDTAVYYCTTARWDWYFDLWGRGTLVTVSS

SEQ ID NO: 31 - protein, 8 aa, synthetic construct
GITFSNAW

SEQ ID NO: 32 - protein, 10 aa, synthetic construct
IKSKTDGGTT

SEQ ID NO: 33 - protein, 11 aa, synthetic construct
TTARWDWYFDL

SEQ ID NO: 34 - protein, 107 aa, synthetic construct
DIQMTQSPSSLSASVGDRVTITCQASQDIWNYINWYQQKPGKAPKLLIYDASNLKTGVPSRFSGSGSGTDFTFTISSLQPEDIATYYCQQHDDLPPTFGQGTKVEIK

SEQ ID NO: 35 - protein, 6 aa, synthetic construct
QDIWNY

SEQ ID NO: 36 - protein, 9 aa, synthetic construct
QQHDDLPPT

SEQ ID NO: 37 - protein, 450 aa, synthetic construct
EVQLVESGGGLVKPGGSLRLSCAASGITFSNAWMSWVRQAPGKGLEWVGRIKSKTDGGTTDYAAPVKGRFTISRDDSKNTLYLQMNSLKTEDTAVYYCTTARWDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 214 aa, synthetic construct
DIQMTQSPSSLSASVGDRVTITCQASQDIWNYINWYQQKPGKAPKLLIYDASNLKTGVPSRFSGSGSGTDFTFTISSLQPEDIATYYCQQHDDLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - DNA, 360 bp, synthetic construct
  source 1..360: mol_type = other DNA
gaggtgcagctggtggagtctgggggaggcttggtaaagcctggggggtcccttagactctcctgtgcagcctctggaatcactttcagtaacgcctggatgagttgggtccgccaggctccagggaaggggctggagtgggttggccgtattaaaagcaaaactgatggtgggacaacagactacgccgcacccgtgaaaggcagattcaccatctcaagagatgattcaaaaaacacgctgtatctacaaatgaacagcctgaaaaccgaggacacagccgtgtattactgtaccacagcgaggtgggactggtacttcgatctctggggccgtggcaccctggtcactgtctcctca

SEQ ID NO: 40 - DNA, 24 bp, synthetic construct
  source 1..24: mol_type = other DNA
ggaatcactttcagtaacgcctgg

SEQ ID NO: 41 - DNA, 30 bp, synthetic construct
  source 1..30: mol_type = other DNA
attaaaagcaaaactgatggtgggacaaca

SEQ ID NO: 42 - DNA, 33 bp, synthetic construct
  source 1..33: mol_type = other DNA
accacagcgaggtgggactggtacttcgatctc

SEQ ID NO: 43 - DNA, 321 bp, synthetic construct
  source 1..321: mol_type = other DNA
gacatccagatgacccagtctccatcctccctgtctgcatctgtaggagacagagtcaccatcacttgccaggcgagtcaggacatttggaattatataaattggtatcagcagaaaccagggaaggcccctaagctcctgatctacgatgcatccaatttgaaaacaggggtcccatcaaggttcagtggaagtggatctgggacagattttactttcaccatcagcagcctgcagcctgaagatattgcaacatattactgtcaacagcatgatgatctccctccgaccttcggccaagggaccaaggtggaaatcaaa

SEQ ID NO: 44 - DNA, 18 bp, synthetic construct
  source 1..18: mol_type = other DNA
caggacatttggaattat

SEQ ID NO: 45 - DNA, 27 bp, synthetic construct
  source 1..27: mol_type = other DNA
caacagcatgatgatctccctccgacc

SEQ ID NO: 46 - DNA, 1353 bp, synthetic construct
  source 1..1353: mol_type = other DNA
gaggtgcagctggtggagtctgggggaggcttggtaaagcctggggggtcccttagactctcctgtgcagcctctggaatcactttcagtaacgcctggatgagttgggtccgccaggctccagggaaggggctggagtgggttggccgtattaaaagcaaaactgatggtgggacaacagactacgccgcacccgtgaaaggcagattcaccatctcaagagatgattcaaaaaacacgctgtatctacaaatgaacagcctgaaaaccgaggacacagccgtgtattactgtaccacagcgaggtgggactggtacttcgatctctggggccgtggcaccctggtcactgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 47 - DNA, 645 bp, synthetic construct
  source 1..645: mol_type = other DNA
gacatccagatgacccagtctccatcctccctgtctgcatctgtaggagacagagtcaccatcacttgccaggcgagtcaggacatttggaattatataaattggtatcagcagaaaccagggaaggcccctaagctcctgatctacgatgcatccaatttgaaaacaggggtcccatcaaggttcagtggaagtggatctgggacagattttactttcaccatcagcagcctgcagcctgaagatattgcaacatattactgtcaacagcatgatgatctccctccgaccttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 48 - protein, 120 aa, synthetic construct
QVQLVESGGGVVQPGRSLRLSCAASGFTFSNYAMYWVRQAPGKGLEWVAVISYDGSNKYYADSVKGRFTISRDNSKNTLYLQMNSLRTEDTAVYYCASGSDYGDYLLVYWGQGTLVTVSS

SEQ ID NO: 49 - protein, 8 aa, synthetic construct
GFTFSNYA

SEQ ID NO: 50 - protein, 8 aa, synthetic construct
ISYDGSNK

SEQ ID NO: 51 - protein, 13 aa, synthetic construct
ASGSDYGDYLLVY

SEQ ID NO: 52 - protein, 110 aa, synthetic construct
QSALTQPASVSGSPGQSITISCTGTSSDVGGYNYVSWYQQHPGKAPKLMIYDVSKRPSGVSNRFSGSKSGNTASLTISGLQSEDEADYYCNSLTSISTWVFGGGTKLTVL

SEQ ID NO: 53 - protein, 9 aa, synthetic construct
SSDVGGYNY

SEQ ID NO: 54 - protein, 10 aa, synthetic construct
NSLTSISTWV

SEQ ID NO: 55 - protein, 450 aa, synthetic construct
QVQLVESGGGVVQPGRSLRLSCAASGFTFSNYAMYWVRQAPGKGLEWVAVISYDGSNKYYADSVKGRFTISRDNSKNTLYLQMNSLRTEDTAVYYCASGSDYGDYLLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 216 aa, synthetic construct
QSALTQPASVSGSPGQSITISCTGTSSDVGGYNYVSWYQQHPGKAPKLMIYDVSKRPSGVSNRFSGSKSGNTASLTISGLQSEDEADYYCNSLTSISTWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 57 - DNA, 360 bp, synthetic construct
  source 1..360: mol_type = other DNA
caggtgcagctggtggagtctgggggaggcgtggtccagcctgggaggtccctgagactctcctgtgcagcctctggattcaccttcagtaactatgctatgtactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagaactgaggacacggctgtgtattactgtgcgagtggctccgactacggtgactacttattggtttactggggccagggaaccctggtcaccgtctcctca

SEQ ID NO: 58 - DNA, 24 bp, synthetic construct
  source 1..24: mol_type = other DNA
ggattcaccttcagtaactatgct

SEQ ID NO: 59 - DNA, 24 bp, synthetic construct
  source 1..24: mol_type = other DNA
atatcatatgatggaagtaataaa

SEQ ID NO: 60 - DNA, 39 bp, synthetic construct
  source 1..39: mol_type = other DNA
gcgagtggctccgactacggtgactacttattggtttac

SEQ ID NO: 61 - DNA, 330 bp, synthetic construct
  source 1..330: mol_type = other DNA
cagtctgccctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaagcggccctcaggggtttctaatcgcttctctggctccaagtctggcaacacggcctccctgaccatctctgggctccagtctgaggacgaggctgattattactgcaactctttgacaagcatcagcacttgggtgttcggcggagggaccaagctgaccgtccta

SEQ ID NO: 62 - DNA, 27 bp, synthetic construct
  source 1..27: mol_type = other DNA
agcagtgacgttggtggttataactat

SEQ ID NO: 63 - DNA, 30 bp, synthetic construct
  source 1..30: mol_type = other DNA
aactctttgacaagcatcagcacttgggtg

SEQ ID NO: 64 - DNA, 1353 bp, synthetic construct
  source 1..1353: mol_type = other DNA
caggtgcagctggtggagtctgggggaggcgtggtccagcctgggaggtccctgagactctcctgtgcagcctctggattcaccttcagtaactatgctatgtactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagaactgaggacacggctgtgtattactgtgcgagtggctccgactacggtgactacttattggtttactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 65 - DNA, 651 bp, synthetic construct
  source 1..651: mol_type = other DNA
cagtctgccctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaagcggccctcaggggtttctaatcgcttctctggctccaagtctggcaacacggcctccctgaccatctctgggctccagtctgaggacgaggctgattattactgcaactctttgacaagcatcagcacttgggtgttcggcggagggaccaagctgaccgtcctaggccagcccaaggccgccccctccgtgaccctgttccccccctcctccgaggagctgcaggccaacaaggccaccctggtgtgcctgatctccgacttctaccccggcgccgtgaccgtggcctggaaggccgactcctcccccgtgaaggccggcgtggagaccaccaccccctccaagcagtccaacaacaagtacgccgcctcctcctacctgtccctgacccccgagcagtggaagtcccaccggtcctactcctgccaggtgacccacgagggctccaccgtggagaagaccgtggcccccaccgagtgctcctga

SEQ ID NO: 66 - protein, 123 aa, synthetic construct
QVQLVQSGAEVKKPGASVKVSCKASGYIFTGYYMHWVRQAPGQGLEWMGWINPNSGGANYAQKFQGRVTLTRDTSITTVYMELSRLRFDDTAVYYCARGSRYDWNQNNWFDPWGQGTLVTVSS

SEQ ID NO: 67 - protein, 8 aa, synthetic construct
GYIFTGYY

SEQ ID NO: 68 - protein, 8 aa, synthetic construct
INPNSGGA

SEQ ID NO: 69 - protein, 16 aa, synthetic construct
ARGSRYDWNQNNWFDP

SEQ ID NO: 70 - protein, 110 aa, synthetic construct
QSALTQPASVSGSPGQSITISCTGTSSDVGTYNYVSWYQQHPGKAPKLMIFDVSNRPSGVSDRFSGSKSGNTASLTISGLQAEDEADYYCSSFTTSSTVVFGGGTKLTVL

SEQ ID NO: 71 - protein, 9 aa, synthetic construct
SSDVGTYNY

SEQ ID NO: 72 - protein, 10 aa, synthetic construct
SSFTTSSTVV

SEQ ID NO: 73 - protein, 453 aa, synthetic construct
QVQLVQSGAEVKKPGASVKVSCKASGYIFTGYYMHWVRQAPGQGLEWMGWINPNSGGANYAQKFQGRVTLTRDTSITTVYMELSRLRFDDTAVYYCARGSRYDWNQNNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 216 aa, synthetic construct
QSALTQPASVSGSPGQSITISCTGTSSDVGTYNYVSWYQQHPGKAPKLMIFDVSNRPSGVSDRFSGSKSGNTASLTISGLQAEDEADYYCSSFTTSSTVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 75 - DNA, 369 bp, synthetic construct
  source 1..369: mol_type = other DNA
caggtgcagctggtgcagtctggggctgaggtgaagaagcctggggcctcagtgaaggtctcctgcaaggcttctggatacatcttcaccggctactatatgcactgggtgcgacaggcccctggacaggggcttgagtggatgggatggatcaaccctaacagtggtggcgcaaactatgcacagaagtttcagggcagggtcaccctgaccagggacacgtccatcaccacagtctacatggaactgagcaggctgagatttgacgacacggccgtgtattactgtgcgagaggatcccggtatgactggaaccagaacaactggttcgacccctggggccagggaaccctggtcaccgtctcctca

SEQ ID NO: 76 - DNA, 24 bp, synthetic construct
  source 1..24: mol_type = other DNA
ggatacatcttcaccggctactat

SEQ ID NO: 77 - DNA, 24 bp, synthetic construct
  source 1..24: mol_type = other DNA
atcaaccctaacagtggtggcgca

SEQ ID NO: 78 - DNA, 48 bp, synthetic construct
  source 1..48: mol_type = other DNA
gcgagaggatcccggtatgactggaaccagaacaactggttcgacccc

SEQ ID NO: 79 - DNA, 330 bp, synthetic construct
  source 1..330: mol_type = other DNA
cagtctgccctgactcagcctgcctccgtgtctgggtctcctggacagtcgatcaccatctcctgcactggaaccagcagtgacgttggtacttataactatgtctcctggtaccaacaacacccaggcaaagcccccaaactcatgatttttgatgtcagtaatcggccctcaggggtttctgatcgcttctctggctccaagtctggcaacacggcctccctgaccatctctgggctccaggctgaggacgaggctgattattactgcagctcatttacaaccagcagcactgtggttttcggcggagggaccaagctgaccgtccta

SEQ ID NO: 80 - DNA, 27 bp, synthetic construct
  source 1..27: mol_type = other DNA
agcagtgacgttggtacttataactat

SEQ ID NO: 81 - DNA, 30 bp, synthetic construct
  source 1..30: mol_type = other DNA
agctcatttacaaccagcagcactgtggtt

SEQ ID NO: 82 - DNA, 1362 bp, synthetic construct
  source 1..1362: mol_type = other DNA
caggtgcagctggtgcagtctggggctgaggtgaagaagcctggggcctcagtgaaggtctcctgcaaggcttctggatacatcttcaccggctactatatgcactgggtgcgacaggcccctggacaggggcttgagtggatgggatggatcaaccctaacagtggtggcgcaaactatgcacagaagtttcagggcagggtcaccctgaccagggacacgtccatcaccacagtctacatggaactgagcaggctgagatttgacgacacggccgtgtattactgtgcgagaggatcccggtatgactggaaccagaacaactggttcgacccctggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 83 - DNA, 651 bp, synthetic construct
  source 1..651: mol_type = other DNA
cagtctgccctgactcagcctgcctccgtgtctgggtctcctggacagtcgatcaccatctcctgcactggaaccagcagtgacgttggtacttataactatgtctcctggtaccaacaacacccaggcaaagcccccaaactcatgatttttgatgtcagtaatcggccctcaggggtttctgatcgcttctctggctccaagtctggcaacacggcctccctgaccatctctgggctccaggctgaggacgaggctgattattactgcagctcatttacaaccagcagcactgtggttttcggcggagggaccaagctgaccgtcctaggccagcccaaggccgccccctccgtgaccctgttccccccctcctccgaggagctgcaggccaacaaggccaccctggtgtgcctgatctccgacttctaccccggcgccgtgaccgtggcctggaaggccgactcctcccccgtgaaggccggcgtggagaccaccaccccctccaagcagtccaacaacaagtacgccgcctcctcctacctgtccctgacccccgagcagtggaagtcccaccggtcctactcctgccaggtgacccacgagggctccaccgtggagaagaccgtggcccccaccgagtgctcctga

SEQ ID NO: 84 - protein, 7 aa, synthetic construct
QLGTVEG

SEQ ID NO: 85 - protein, 7 aa, synthetic construct
TLNSEES

SEQ ID NO: 86 - protein, 7 aa, synthetic construct
VMSSGDQ

SEQ ID NO: 87 - protein, 7 aa, synthetic construct
KLASYNP

SEQ ID NO: 88 - protein, 7 aa, synthetic construct
FIDSGVG

SEQ ID NO: 89 - protein, 7 aa, synthetic construct
THFSQQD

SEQ ID NO: 90 - protein, 7 aa, synthetic construct
NHSSLHD

SEQ ID NO: 91 - protein, 7 aa, synthetic construct
GGGSGGG

SEQ ID NO: 92 - protein, 5 aa, synthetic construct
GGSGG

SEQ ID NO: 93 - protein, 11 aa, synthetic construct
  VARIANT 1..10: note = This region may encompass 1-10 residues
GGGGGGGGGGS

SEQ ID NO: 94 - protein, 11 aa, synthetic construct
  VARIANT 2..11: note = This region may encompass 1-10 residues
SGGGGGGGGGG

SEQ ID NO: 95 - protein, 5 aa, synthetic construct
GGGGS

SEQ ID NO: 96 - protein, 4 aa, synthetic construct
GGGG

SEQ ID NO: 97 - protein, 5 aa, synthetic construct
GGGGG

SEQ ID NO: 98 - protein, 6 aa, synthetic construct
GGGGGG

SEQ ID NO: 99 - protein, 7 aa, synthetic construct
GGGGGGG

SEQ ID NO: 100 - protein, 8 aa, synthetic construct
GGGGGGGG

SEQ ID NO: 101 - protein, 9 aa, synthetic construct
GGGGGGGGG

SEQ ID NO: 102 - protein, 35 aa, synthetic construct
GGGGSLALGPGGGGGSLALGPGGGGGSLALGPGGS

SEQ ID NO: 103 - protein, 25 aa, synthetic construct
GGGGSGGGGSGGGGSGGGGSGGGGS

SEQ ID NO: 104 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 105 - protein, 40 aa, synthetic construct
  VARIANT 1..40: note = This sequence may encompass 1-10 GGGS repeating units
GGGSGGGSGGGSGGGSGGGSGGGSGGGSGGGSGGGSGGGS

SEQ ID NO: 106 - protein, 30 aa, synthetic construct
  VARIANT 1..30: note = This sequence may encompass 1-10 GGS repeating units
GGSGGSGGSGGSGGSGGSGGSGGSGGSGGS

SEQ ID NO: 107 - protein, 20 aa, synthetic construct
  VARIANT 1..20: note = This sequence may encompass 1-10 GS repeating units
GSGSGSGSGSGSGSGSGSGS

SEQ ID NO: 108 - protein, 50 aa, synthetic construct
  VARIANT 1..50: note = This sequence may encompass 1-10 GSGGS repeating units
GSGGSGSGGSGSGGSGSGGSGSGGSGSGGSGSGGSGSGGSGSGGSGSGGS

SEQ ID NO: 109 - protein, 5 aa, synthetic construct
ADAAP

SEQ ID NO: 110 - protein, 11 aa, synthetic construct
ADAAPTVSIFP

SEQ ID NO: 111 - protein, 12 aa, synthetic construct
ADAAPTVSIFPP

SEQ ID NO: 112 - protein, 6 aa, synthetic construct
AKTTAP

SEQ ID NO: 113 - protein, 13 aa, synthetic construct
AKTTAPSVYPLAP

SEQ ID NO: 114 - protein, 17 aa, synthetic construct
AKTTPKLEEGEFSEARV

SEQ ID NO: 115 - protein, 9 aa, synthetic construct
AKTTPKLGG

SEQ ID NO: 116 - protein, 6 aa, synthetic construct
AKTTPP

SEQ ID NO: 117 - protein, 13 aa, synthetic construct
AKTTPPSVTPLAP

SEQ ID NO: 118 - protein, 6 aa, synthetic construct
ASTKGP

SEQ ID NO: 119 - protein, 26 aa, synthetic construct
ASTKGPSVFPLAPASTKGPSVFPLAP

SEQ ID NO: 120 - protein, 14 aa, synthetic construct
EGKSSGSGSESKST

SEQ ID NO: 121 - protein, 15 aa, synthetic construct
GEGESGEGESGEGES

SEQ ID NO: 122 - protein, 20 aa, synthetic construct
GEGESGEGESGEGESGEGES

SEQ ID NO: 123 - protein, 15 aa, synthetic construct
GEGGSGEGGSGEGGS

SEQ ID NO: 124 - protein, 15 aa, synthetic construct
GENKVEYAPALMALS

SEQ ID NO: 125 - protein, 15 aa, synthetic construct
GGEGSGGEGSGGEGS

SEQ ID NO: 126 - protein, 15 aa, synthetic construct
GGGESGGEGSGEGGS

SEQ ID NO: 127 - protein, 15 aa, synthetic construct
GGGESGGGESGGGES

SEQ ID NO: 128 - protein, 10 aa, synthetic construct
GGGGSGGGGS

SEQ ID NO: 129 - protein, 15 aa, synthetic construct
GGGGSGGGGSGGGGS

SEQ ID NO: 130 - protein, 20 aa, synthetic construct
GGGGSGGGGSGGGGSGGGGS

SEQ ID NO: 131 - protein, 15 aa, synthetic construct
GGGKSGGGKSGGGKS

SEQ ID NO: 132 - protein, 15 aa, synthetic construct
GGGKSGGKGSGKGGS

SEQ ID NO: 133 - protein, 4 aa, synthetic construct
GGGS

SEQ ID NO: 134 - protein, 5 aa, synthetic construct
GGGSG

SEQ ID NO: 135 - protein, 15 aa, synthetic construct
GGKGSGGKGSGGKGS

SEQ ID NO: 136 - protein, 4 aa, synthetic construct
GGSG

SEQ ID NO: 137 - protein, 9 aa, synthetic construct
GGSGGGGSG

SEQ ID NO: 138 - protein, 13 aa, synthetic construct
GGSGGGGSGGGGS

SEQ ID NO: 139 - protein, 15 aa, synthetic construct
GHEAAAVMQVQYPAS

SEQ ID NO: 140 - protein, 15 aa, synthetic construct
GKGGSGKGGSGKGGS

SEQ ID NO: 141 - protein, 15 aa, synthetic construct
GKGKSGKGKSGKGKS

SEQ ID NO: 142 - protein, 20 aa, synthetic construct
GKGKSGKGKSGKGKSGKGKS

SEQ ID NO: 143 - protein, 15 aa, synthetic construct
GKPGSGKPGSGKPGS

SEQ ID NO: 144 - protein, 21 aa, synthetic construct
GKPGSGKPGSGKPGSGKPSGS

SEQ ID NO: 145 - protein, 15 aa, synthetic construct
GPAKELTPLKEAKVS

SEQ ID NO: 146 - protein, 12 aa, synthetic construct
GSAGSAAGSGEF

SEQ ID NO: 147 - protein, 5 aa, synthetic construct
GSGGG

SEQ ID NO: 148 - protein, 5 aa, synthetic construct
GSGSG

SEQ ID NO: 149 - protein, 4 aa, synthetic construct
GSSG

SEQ ID NO: 150 - protein, 10 aa, synthetic construct
GSSGGSGGSG

SEQ ID NO: 151 - protein, 11 aa, synthetic construct
GSSGGSGGSGG

SEQ ID NO: 152 - protein, 12 aa, synthetic construct
GSSGGSGGSGGS

SEQ ID NO: 153 - protein, 13 aa, synthetic construct
GSSGGSGGSGGSG

SEQ ID NO: 154 - protein, 16 aa, synthetic construct
GSSGGSGGSGGSGGGS

SEQ ID NO: 155 - protein, 11 aa, synthetic construct
GSSGGSGGSGS

SEQ ID NO: 156 - protein, 5 aa, synthetic construct
GSSGT

SEQ ID NO: 157 - protein, 5 aa, synthetic construct
GSSSG

SEQ ID NO: 158 - protein, 18 aa, synthetic construct
GSTSGSGKPGSGEGSTKG

SEQ ID NO: 159 - protein, 19 aa, synthetic construct
GTAAAGAGAAGGAAAGAAG

SEQ ID NO: 160 - protein, 19 aa, synthetic construct
GTSGSSGSGSGGSGSGGGG

SEQ ID NO: 161 - protein, 14 aa, synthetic construct
IRPRAIGGSKPRVA

SEQ ID NO: 162 - protein, 18 aa, synthetic construct
KESGSVSSEQLAQFRSLD

SEQ ID NO: 163 - protein, 15 aa, synthetic construct
KTTPKLEEGEFSEAR

SEQ ID NO: 164 - protein, 19 aa, synthetic construct
PRGASKSGSASQTGSAPGS

SEQ ID NO: 165 - protein, 6 aa, synthetic construct
QPKAAP

SEQ ID NO: 166 - protein, 13 aa, synthetic construct
QPKAAPSVTLFPP

SEQ ID NO: 167 - protein, 27 aa, synthetic construct
RADAAAAGGGGSGGGGSGGGGSGGGGS

SEQ ID NO: 168 - protein, 12 aa, synthetic construct
RADAAAAGGPGS

SEQ ID NO: 169 - protein, 6 aa, synthetic construct
RADAAP

SEQ ID NO: 170 - protein, 9 aa, synthetic construct
RADAAPTVS

SEQ ID NO: 171 - protein, 6 aa, synthetic construct
SAKTTP

SEQ ID NO: 172 - protein, 18 aa, synthetic construct
SAKTTPKLEEGEFSEARV

SEQ ID NO: 173 - protein, 10 aa, synthetic construct
SAKTTPKLGG

SEQ ID NO: 174 - protein, 14 aa, synthetic construct
STAGDTHLGGEDFD

SEQ ID NO: 175 - protein, 5 aa, synthetic construct
TVAAP

SEQ ID NO: 176 - protein, 12 aa, synthetic construct
TVAAPSVFIFPP

SEQ ID NO: 177 - protein, 24 aa, synthetic construct
TVAAPSVFIFPPTVAAPSVFIFPP

SEQ ID NO: 178 - protein, 88 aa, synthetic construct
CLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 179 - protein, 37 aa, synthetic construct
MMLKKILKIEELDERELIDIEVSGNHLFYANDILTHN

SEQ ID NO: 180 - protein, 89 aa, synthetic construct
CLSLDTMVVTNGKAIEIRDVKVGDWLESECGPVQVTEVLPIIKQPVFEIVLKSGKKIRVSANHKFPTKDGLKTINSGLKVGDFLRSRAK

SEQ ID NO: 181 - protein, 45 aa, synthetic construct
MCEIFENEIDWDEIASIEYVGVEETIDINVTNDRLFFANGILTHN

SEQ ID NO: 182 - protein, 105 aa, synthetic construct
CLVGSSEIITRNYGKTTIKEVVEIFDNDKNIQVLAFNTHTDNIEWAPIKAAQLTRPNAELVELEINTLHGVKTIRCTPDHPVYTKNRDYVRADELTDDDELVVAI

SEQ ID NO: 183 - protein, 40 aa, synthetic construct
MEAKTYIGKLKSRKIVSNEDTYDIQTSTHNFFANDILVHN

SEQ ID NO: 184 - protein, 106 aa, synthetic construct
CLTGDAKIDVLIDNIPISQISLEEVVNLFNEGKEIYVLSYNIDTKEVEYKEISDAGLISESAEVLEIIDEETGQKIVCTPDHKVYTLNRGYVSAKDLKEDDELVFS

SEQ ID NO: 185 - protein, 34 aa, synthetic construct
MGLKIIKRESKEPVFDITVKDNSNFFANNILVHN

SEQ ID NO: 186 - protein, 101 aa, synthetic construct
CFVPGTLVNTENGLKKIEEIKVGDKVFSHTGKLQEVVDTLIFDRDEEIISINGIDCTKNHEFYVIDKENANRVNEDNIHLFARWVHAEELDMKKHLLIELE

SEQ ID NO: 187 - protein, 40 aa, synthetic construct
MKFKLKEITSIETKHYKGKVHDLTVNQDHSYNVRGTVVHN

SEQ ID NO: 188 - protein, 102 aa, synthetic construct
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 189 - protein, 36 aa, synthetic construct
MIKIATRKYLGKQNVYDIGVERDHNFALKNGFIASN

SEQ ID NO: 190 - protein, 123 aa, synthetic construct
CLSFGTEILTVEYGPLPIGKIVSEEINCSVYSVDPEGRVYTQAIAQWHDRGEQEVLEYELEDGSVIRATSDHRFLTTDYQLLAIEEIFARQLDLLTLENIKQTEEALDNHRLPFPLLDAGTIK

SEQ ID NO: 191 - protein, 36 aa, synthetic construct
MVKVIGRRSLGVQRIFDIGLPQDHNFLLANGAIAAN

SEQ ID NO: 192 - protein, 110 aa, synthetic construct
CLSYDTEIWTVEYGAMPIGKIVEEKIECSVYTVDENGFVYTQPIAQWHPRGQQEIIEYTLEDGRKIRATKDHKMMTESGEMLPIEEIFQRELDLKVETFHEMSLLRRGAK

SEQ ID NO: 193 - protein, 36 aa, synthetic construct
MVKIIKRQSLGRQNVYDVCVETDHNFVLANGCVASN

SEQ ID NO: 194 - protein, 101 aa, synthetic construct
CLSADTEILTVEYGFLPIGEIVGKAIECRVYSVDGNGNIYTQSIAQWHNRGEQEVFEYTLEDGSIIRATKDHKFMTTDGEMLPIDEIFARQLDLMQVQGLH

SEQ ID NO: 195 - protein, 36 aa, synthetic construct
MVKITARKFVGRENVYDIGVEHHHNFAIKNGLIASN

SEQ ID NO: 196 - protein, 102 aa, synthetic construct
CLSYDTEVLTVEYGFVPIGEIVEKGIECSVFSINNNGIVYTQPIAQWHHRGKQEVFEYCLEDGSIIKATKDHKFMTQDGKMLPIDEIFEQELDLLQVKGLPE

SEQ ID NO: 197 - protein, 36 aa, synthetic construct
MIKIASRKFLGVENVYDIGVRRDHNFFIKNGLIASN

SEQ ID NO: 198 - protein, 102 aa, synthetic construct
CLSYDTEVLTVEYGFVPIGEIVDKGIECSVFSIDSNGIVYTQPIAQWHHRGKQEVFEYCLEDGSIIKATKDHKFMTQDGKMLPIDEIFEQELDLLQVKGLPE

SEQ ID NO: 199 - protein, 36 aa, synthetic construct
MIKIASRKFLGVENVYDIGVGRDHNFFVKNGLIASN

SEQ ID NO: 200 - protein, 118 aa, synthetic construct
CLSYETEVLTLEYGFVPIGEIVNKQMVCTVFSLNDSGNVYTQPIGQWHDRGVQDLYEYCLDDGSTIRATKDHKFMTTQGEMVPIDEIFHQGWELVQVSGISKLVQQRTLPFIIVDRKL

SEQ ID NO: 201 - protein, 36 aa, synthetic construct
MVKIVSRRYLGKADVYDIGVAKDHNFIIKNGLVASN

SEQ ID NO: 202 - protein, 99 aa, synthetic construct
CLSYDTEILTVEYGPMPIGKIVEENINCSVYTVNKNGFVYTQSIAQWHHRGEQEVFEYYLEDGETIRATKDHKFMTTEGKMLPIDEIFENNLDLKKLTV

SEQ ID NO: 203 - protein, 36 aa, synthetic construct
MVKIIERRSLGKQNVYDIGVEKDHNFLLSNNLIASN

SEQ ID NO: 204 - protein, 99 aa, synthetic construct
CLSYDTEILTVEYGAIPIGKVVEENIDCTVYTVDKNGFVYTQNIAQWHLRGQQEVFEYYLDDGSILRATKDHQFMTLEGEMLPIHEIFERGLELKKIKI

SEQ ID NO: 205 - protein, 36 aa, synthetic construct
MVKIVSYRSLGKQFVYDIGVAQDHNFLLANGSIASN

SEQ ID NO: 206 - protein, 106 aa, synthetic construct
CLSYDTEILTVEYGAMYIGKIVEENINCTVYTVDKNGFVYTQTIAQWHNRGEQEIFEYDLEDGSKIKATKDHKFMTIDGEMLPIDEIFEKNLDLKQVVSHPDDYLV

SEQ ID NO: 207 - protein, 36 aa, synthetic construct
MVKIIGCRSLGTQKVYDIGVEKDHNFLLANGSIASN

SEQ ID NO: 208 - protein, 100 aa, synthetic construct
CLGGETLILTEEYGLLPIAKIVSEEINCTVYTVDQNGFVYSQPISQWHERGLQEVFEYTLENGQTIQATKDHKFMTSDGEMLAIDTIFERGLDLKSSDFS

SEQ ID NO: 209 - protein, 36 aa, synthetic construct
MVKIIGRQSLGRKPVYDIGVEKDHNFLLGNGLIASN

SEQ ID NO: 210 - protein, 104 aa, synthetic construct
CLSYDTQILTVEYGAVAIGEIVEKQIECTVYSVDENGYVYTQPIAQWHNRGEQEVFEYLLEDGATIRATKDHKFMTDEDQMLPIDQIFEQGLELKQVEVLQPVF

SEQ ID NO: 211 - protein, 36 aa, synthetic construct
MVKIVRRQSLGVQNVYDIGVEKDHNFCLASGEIASN

SEQ ID NO: 212 - protein, 112 aa, synthetic construct
CLSYNTEVLTVEYGPLPIGKIVDEQIHCRVYSVDENGFVYTQAIAQWHDRGYQEIFAYELADGSVIRATKDHQFMTEDGQMFPIDEIWEKGLDLKKLPTVQDLPAAVGYTVS

SEQ ID NO: 213 - protein, 113 aa, synthetic construct
CLAADTEVLTVEYGPIAIGKLVEENIRCQVYCCNPDGYIYSQPIGQWHQRGEQEVIEYELSDGRIIRATADHRFMTEEGEMLSLDEIFERSLELKQIPTPLLAIAQPSPLATA

SEQ ID NO: 214 - protein, 36 aa, synthetic construct
MVKIVRRRSLGVQPVYDLGVATVHNFVLANGLVASN

SEQ ID NO: 215 - protein, 107 aa, synthetic construct
CLAGGTPVVTVEYGVLPIQTIVEQELLCHVYSVDAQGLIYAQLIEQWHQRGDRLLYEYELENGQMIRATPDHRFLTTTGELLPIDEIFTQNLDLAAWAVPDSLPRTA

SEQ ID NO: 216 - protein, 36 aa, synthetic construct
MVKIIRRKFIGHAPTYDIGLSQDHNFLLGQGLIAAN

SEQ ID NO: 217 - protein, 117 aa, synthetic construct
CLSGETAVMTVEYGAVPIRRLVQERLSCHVYSLDGQGHLYTQPIAQWHFQGFRPVYEYQLEDGSTICATPDHRFMTTRGQMLPIEQIFQEGLELWQVAIAPRQALLQGLKPAVQMSG

SEQ ID NO: 218 - protein, 35 aa, synthetic construct
MKIVGRRLMGWQAVYDIGLAADHNFVLANGAIAAN

SEQ ID NO: 219 - protein, 101 aa, synthetic construct
CLTYETEIMTVEYGPLPIGKIVEYRIECTVYTVDKNGYIYTQPIAQWHNRGMQEVYEYSLEDGTVIRATPEHKFMTEDGQMLPIDEIFERNLDLKCLGTLE

SEQ ID NO: 220 - protein, 36 aa, synthetic construct
MVKIVSRKLAKTENVYDIGVTKDHNFVLANGLIASN

SEQ ID NO: 221 - protein, 117 aa, synthetic construct
CLSGETAVMTVEYGAIPIRRLVQERLICQVYSLDPQGHLYTQPIAQWHFQGFRPVYAYQLEDGSTICATPDHRFMTTSGQMLPIEQIFREGLELWQVAIAPPGALAQGLKPAVQMSC

SEQ ID NO: 222 - protein, 35 aa, synthetic construct
MKIVGRRLVGWQAVYDIGLAGDHNFLLANGAIAAN

SEQ ID NO: 223 - protein, 25 aa, synthetic construct
CVYGDTMVETEDGKIKIEDLYKRLA

SEQ ID NO: 224 - protein, 104 aa, synthetic construct
MFRTNTNNIKILSPNGFSNFNGIQKVERNLYQHIIFDDDTEIKTSINHPFGKDKILARDVKVGDYLNSKKVLYNELVNENIFLYDPINVEKESLYITNGVVSHN

SEQ ID NO: 225 - protein, 37 aa, synthetic construct
SISQESYINIEVNGKVETIKIGDLYKKLSFNERKFNE

SEQ ID NO: 226 - protein, 115 aa, synthetic construct
MKLPESVVKNNINLKIETPYGFENFYGVNKIKKDKYIHLEFTNGEKLKCSLDHPLSTIDGIVKAKDLDKYTEVYTKFGGCFLKKSKVINESIELYDIVNSGLKHLYYSNNIISHN

SEQ ID NO: 227 - protein, 36 aa, synthetic construct
CCLENTRVQVRNKYTNKIETLTIKELYARLQELKKS

SEQ ID NO: 228 - protein, 104 aa, synthetic construct
MSEIQDINPYEILTPQGFKPFVDIIKSIQTTGITITLEDSREISVTLDHKFKHLNDYKEAKYFKVGDKLQCSKIIKIENIEGEFYEPLEVQDHEYIANDFINHN

SEQ ID NO: 229 - protein, 31 aa, synthetic construct
CVAGDTKITVRNKKTGVIEDITMEELYNRIG

SEQ ID NO: 230 - protein, 100 aa, synthetic construct
MYEVLTPNGFSDFDDISREKKDVYKVITEDDFIKVTKGHKFETPNGFKQLKHLKINDLIKYKNKFSKIVLIDYVGVEYVYDLINVHKNNEYYTNNFVSHN

SEQ ID NO: 231 - protein, 33 aa, synthetic construct
CFSKKTSIKLRNKKTGDLEEIDISDLIYELHIS

SEQ ID NO: 232 - protein, 125 aa, synthetic construct
MIKLYNKKQNKKFTKSYDLGDYQILTDSGYIGLVSLHETIPYEVWKLKLSNGYELECADDHIIFDNEMNEIFVKNLELGDRVKVDDGYAVVIELVNTGLLESMYDFELVEDSNRRYYTNGILSHN

SEQ ID NO: 233 - protein, 30 aa, synthetic construct
CVSPNTKIKIRNSSTGEISEVTIAEFNKMI

SEQ ID NO: 234 - protein, 115 aa, synthetic construct
MKKIVKSVSVEGFEVLSDNGWVPIKNVHTTVPYELYNLRTANGLRLECADNHIVFTSKLKEVYVKDLNVDDKIMTEDGVSLVSSIEKTKAKVTMYDLEVDSEDHRYYTDGILSHN

SEQ ID NO: 235 - protein, 34 aa, synthetic construct
CVHPNTLVKIKIDSTGEERTITVKDLHELIKSVK

SEQ ID NO: 236 - protein, 115 aa, synthetic construct
MKRKFIESISADNISIMTDTGWEKVKGSHVTIEYKVFNLVTDRLSLQCADDHIVFKEDFSEVFVKDLEVGDLIQTVNGLESVTEVYETDDLVNMHDLEIDSKNHRYYTDGILSHN

SEQ ID NO: 237 - protein, 34 aa, synthetic construct
CVSGDTKVTLKDNDTGKIINVNIEEMVSVSSLDV

SEQ ID NO: 238 - protein, 105 aa, synthetic construct
MEVGKMSKSYKVLSPSGFVDFAGIQKITRSKYRHFIFDDGTEIKCSLNHRFGEEEIVASTLHHGTELQGKKILYAEDVEDDIDLYDLLNVANGNLYYTNGLVSHN

SEQ ID NO: 239 - protein, 55 aa, synthetic construct
CFFFNTIISVETNNQQYETRIGILYYSMVSKERNLTILEKIKIKLYDLLFILEKH

SEQ ID NO: 240 - protein, 150 aa, synthetic construct
MLRIFKRCLIYLIKKMIEFIELYEYKKISLDECDINKKILNSISLMDLKVETDTGYETSSNIHITQPFKHYNIETVDGYEIICADNHILFDEEFNEVFTKDLKIGDLIKTKNGNSVIKSIYIDTHKSSMFDLTIDHPNHRFYTNGILSHN

SEQ ID NO: 241 - protein, 24 aa, synthetic construct
CVDGNTIVETEDGKIKIEDLYKKL

SEQ ID NO: 242 - protein, 104 aa, synthetic construct
MFITNTDNIKILSPSGFSNFNGIQKVERNLYQHIIFDDESEIKTSINHPFGKNKILARNVKVGDYLSSKKVLYNELVNEKIFLYDPINVEKENLYITNGVVSHN

SEQ ID NO: 243 - protein, 24 aa, synthetic construct
CVDGNTIVETEDGKIKIEDLYKKM

SEQ ID NO: 244 - protein, 104 aa, synthetic construct
MFRTNTDNIKILSPSGFSIFNGIQKVERDLYQHIIFDDKSEIKTSINHPFGKDKILARNIKVGDYLNSKKVLYNELVAEKITLYDPINVEKENLYITNGVISHN

SEQ ID NO: 245 - protein, 104 aa, synthetic construct
MFRTNTDNIKILSPSGFSNFNGIQKVERDLYQHIIFDDKSEIKTSINHPFGKDKILARNIKVGDYLNSKKVLYNELVNEKITLYDPINVEKENLYITNGVISHN

SEQ ID NO: 246 - protein, 27 aa, synthetic construct
CRTYDSTMDIDVGNSDFAEYLLNNSKK

SEQ ID NO: 247 - protein, 135 aa, synthetic construct
MKFNIPIGELAESIAKYKGVLLNDNCEINIKDLDCKVNTPSGTATINIIIKKEKLEGIKLLLANGVEIKCANKHILRYNNADVFADSLAIGDSVETINGNVKVSSINNIDDTTFYDIGIDAPYLYYDADGVLHHN

SEQ ID NO: 248 - protein, 37 aa, synthetic construct
CFFSDGEINTRNISNKEIKSIKIGKIFTNISKGHTNI

SEQ ID NO: 249 - protein, 148 aa, synthetic construct
MLDNYEIIEADSLLEGKYDRPLYDKFIEAYEVDNLEVDTPNGWIKIEGIGKTIEFYEWEIQTSGGKHLICADKHLLYRCDNMNFYNKKCDITEIYCQDLNIGDFIMTKDGPEMLMDIYKNGNKSNMYDLQLSEGSNKQYYTNDILSHN

SEQ ID NO: 250 - protein, 56 aa, synthetic construct
CVDESTLIDVQIIDFEPNLENLEFLDKTDEGKRIFLYIKKSNKSLYEKIEKFRKGQ

SEQ ID NO: 251 - protein, 122 aa, synthetic construct
MLTLKIGDLYELSKNINILESDIRVSTPGGLKKVFAVDITAKNSDVFSIKVNKHELLCSPDHLIRSEDMWVKSKDLKINSVIDTKYGKLTVKEISILDIKSDLMDLHVDGSEYYTNDIISHN

SEQ ID NO: 252 - protein, 15 aa, synthetic construct
CVHPDTKVTIRRKLC

SEQ ID NO: 253 - protein, 155 aa, synthetic construct
MKELLDLYTEKEINKLLERYTIDQIIDYSQPHVVSVGSIKEEMDSGNFIFVDSPDGYVAVSDFVDKGNFEEYRFTYDKKIIRTNEGHLFQTHLGWETSKNLYKMYLAGHPIYILHKNGGYKKIDIEKTGNVIPIVDIVVEHKNHRYYTDGLSSHN

SEQ ID NO: 254 - protein, 16 aa, synthetic construct
CLPKEAVVQIRLTKKG

SEQ ID NO: 255 - protein, 123 aa, synthetic construct
MIEEKKVTVQELRELYLSGEYTIEIDTPDGYQTIGKWFDKGVLSMVRVATATYETVCAFNHMIQLADNTWVQACELDVGVDIQTAAGIQPVMLVEDTSDAECYDFEVMHPNHRYYGDGIVSHN

SEQ ID NO: 256 - protein, 28 aa, synthetic construct
SLAHETIVSINDNNTLTSMCIGDLYDYM

SEQ ID NO: 257 - protein, 132 aa, synthetic construct
MDYHSNQVSRIFGVGMSKVHLGFKKNTKNLKVLTPNGHEEFYGINKIRVDEYIRIKFKEHKEIRCSIDHPFIQENDLPIKAKHIDKSKHIKCIDGFTTLEYSHVVNKQIELYDIVNSGSEYIYFSNGILSHN

SEQ ID NO: 258 - protein, 30 aa, synthetic construct
CLWGASTVNVFDSLTGKNIDIKLEDLYQKL

SEQ ID NO: 259 - protein, 114 aa, synthetic construct
MESYTFRKNTRYKIMTPAGYQNFGGIRKLNKNVHYIVELSNKKILKCSTTHPFIYNDREIFANKLKVGSLLDSTSKKKISVISIELDKSKIDLYDIVEVNNGNIFNVDGIVSHN

SEQ ID NO: 260 - protein, 31 aa, synthetic construct
CVSASTIITLQDTHGNIFDSQIGDLYNTIGK

SEQ ID NO: 261 - protein, 114 aa, synthetic construct
MSKIFKENTNGYKVLTPAGFQDFAGVSMMGIKPLLRLEFERGAYVECTYDHKFYIDLETCKPAQDIAVGNTVVTSEGDIKLLNKIELGYSEPVYDLIQVEGGHRYYTNKILSSN

SEQ ID NO: 262 - protein, 32 aa, synthetic construct
CVQADTKYTIRNKISGDVLNVTAEEFHKMQKK

SEQ ID NO: 263 - protein, 121 aa, synthetic construct
MKLSNFTNRKFIETIDASEWEVETCEGFKPIISSNKTIEYVVYKIELENGLSIKCADTHILIDKNLQEIYAKDSFNKIIFTKFGNSKVISVETLNISENMYDLSVDSEDHTYYTDDILSHN

SEQ ID NO: 264 - protein, 32 aa, synthetic construct
CLCINTIVKVKNTKTGVIYETTIGELYNGAME

SEQ ID NO: 265 - protein, 124 aa, synthetic construct
MSTISQTVNRKFVNSFSLVDLEIETDSGWQPVTDIHKTIPYTVWHIETQSGLTLDCADTHILFDHNYNEIFVKDIIPNQTKIISKHGPELVLTVIEQSQQENMFDLTVDHPDHRFYSNNILSHN

SEQ ID NO: 266 - protein, 43 aa, synthetic construct
CVIAETEVKIIELYNIDSFIKTGILNSQGVVSWEETSSHGRTR

SEQ ID NO: 267 - protein, 154 aa, synthetic construct
MNLLDKRVEWLSKWYPVDKLQQLSEDKLVLLYNNSQPKKVRMGSLEGVPTSSYRISSPDGYVTAHAWRNKGTKECVTLTTDSGNSITASTDHFFEMSDGKWKYAGCLFPGQCISTESGTETVTSVVAAGKHTVYDFYIDHENHRYYTNGISSHN

SEQ ID NO: 268 - protein, 29 aa, synthetic construct
CLGGDTEIEILDDNGIVQKTSMENLYERL

SEQ ID NO: 269 - protein, 103 aa, synthetic construct
MFKINKNIKVKTPDGFKDFSGIQKVYKPFYHWIIFDDGSEIKCSDNHSFGKEKIKASTIKVDDILQEKKVLYNEIVEEGIYLYDLLDVGEDNLYYSNNIVSHN

SEQ ID NO: 270 - protein, 108 aa, synthetic construct
MSVGKMFKINKNIKVKTPDGFKDFSGIQKVYKPFYHWIIFDDGSEIKCSDNHSFGKEKIKASTIKVDDILQEKKVLYNEIVEEGIYLYDLLDVGEDNLYYSNNIVSHN

SEQ ID NO: 271 - protein, 39 aa, synthetic construct
CFNTNTTVRLRNKLTGEIIEVTIGEFYEKIKKESNTDLP

SEQ ID NO: 272 - protein, 115 aa, synthetic construct
  VARIANT 8: note = Any amino acid
  VARIANT 9: note = Any amino acid
  VARIANT 78: note = Any amino acid
  VARIANT 92: note = Any amino acid
MSKFIEEXXTDEWEVETPSGWQSFSGVGKTIEYEEWEVVTETGKSLICADKHILLNDKWQEVYCEDCSIDDCIQTKNXAEKILQLKKTSRIXNMYDLLDVDNGNIFYSNEIVSHN

SEQ ID NO: 273 - protein, 31 aa, synthetic construct
CVDGSSIITIKNKETNLIEKITIEELYNKLL

SEQ ID NO: 274 - protein, 108 aa, synthetic construct
MKTNTKYEILGPEGFVDFKGIQKLKKKTRQIFFECGLTLRASYNHKIYDYFGDEIIIKDVVIGSKIKSHNGYLIVNSIKDFDYESDVYDVIDSGDSHLYYTNNIVSHN

SEQ ID NO: 275 - protein, 120 aa, synthetic construct
SVVGDTIIDVSGKKMTIAEFYDSTPDVFMRRNDEARDWVKRVGGKTSLSVNTYSGEVERKNINYIMKHTVKKRMFKIKAGGKEVIVTADHSVMVKRDGKIIDVKPTEMKQTDRVVKWMLT

SEQ ID NO: 276 - protein, 39 aa, synthetic construct
MIEFIEFEIEDLGVMEIDVYDIEVDGNHNFFGNDILVHN

SEQ ID NO: 277 - protein, 26 aa, synthetic construct
SVVGDTIIDVSGKKMTIAEFYDSTPD

SEQ ID NO: 278 - protein, 129 aa, synthetic construct
EARDWVKRVGGKTSLSVNTYSGEVERKNINYIMKHTVKKRMFKIKAGGKEVIVTADHSVMVKRDGKIIDVKPTEMKQTDRVVKWMLTGSHMIEFIEFEIEDLGVMEIDVYDIEVDGNHNFFGNDILVHN

SEQ ID NO: 279 - protein, 7 aa, synthetic construct
FIGSGVG

SEQ ID NO: 280 - protein, 5 aa, synthetic construct
IDSGV

SEQ ID NO: 281 - protein, 120 aa, synthetic construct
EVQLVESGGGLVQPGGSLRLSCAASGFTFRNYEMNWVRQAPGKGLEWVSYITSSGSTMYYADSVKGRFTISRDNAKNSLYLQMNSLRAEDTAVYYCARDGFYYYYAMDVWGQGTTVTVSS

SEQ ID NO: 282 - protein, 8 aa, synthetic construct
GFTFRNYE

SEQ ID NO: 283 - protein, 8 aa, synthetic construct
ITSSGSTM

SEQ ID NO: 284 - protein, 13 aa, synthetic construct
ARDGFYYYYAMDV

SEQ ID NO: 285 - protein, 111 aa, synthetic construct
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSVGRVFGTGTKVTVL

SEQ ID NO: 286 - protein, 8 aa, synthetic construct
SSNIGNNY

SEQ ID NO: 287 - protein, 12 aa, synthetic construct
GTWDSSLSVGRV

SEQ ID NO: 288 - protein, 116 aa, synthetic construct
EVQLVESGGGLVQPGGSLRLSCAASGFTVSSNYMNWVRQAPGKGLEWVSVIYSGGSTFYADSVKGRFTISRHNSKNTLYLQMNSLRPEDTAVYYCARDLGGYFDYWGQGTLVTVSS

SEQ ID NO: 289 - protein, 8 aa, synthetic construct
GFTVSSNY

SEQ ID NO: 290 - protein, 7 aa, synthetic construct
IYSGGST

SEQ ID NO: 291 - protein, 10 aa, synthetic construct
ARDLGGYFDY

SEQ ID NO: 292 - protein, 106 aa, synthetic construct
DIQMTQSPSTLSASVGDRVTITCRASQSFSSWLAWYQQKPGKAPKLLIYKASSLESGVPSRFSGSGSGTEFTLTISSLQPDDFATYYCQQYNSYYTFGQGTKLEIK

SEQ ID NO: 293 - protein, 6 aa, synthetic construct
QSFSSW

SEQ ID NO: 294 - protein, 8 aa, synthetic construct
QQYNSYYT

SEQ ID NO: 295 - protein, 117 aa, synthetic construct
EVQLVESGGGLVQPGGSLRLSCAASGLIVSRNYMIWVRQAPGKGLEWVSVIYSGGSTFYADSVKGRFTISRHNSKNTLYLQMNSLRAEDTAVYYCARDLGTGGMDVWGQGTTVTVSS

SEQ ID NO: 296 - protein, 8 aa, synthetic construct
GLIVSRNY

SEQ ID NO: 297 - protein, 11 aa, synthetic construct
ARDLGTGGMDV

SEQ ID NO: 298 - protein, 107 aa, synthetic construct
DIQLTQSPSFLSASVGDRVTITCWASQGISSYLAWYQQKPGKAPKLLIYAASTLQSGVPSRFSGSGSGTEFTLTISSLQPEDFATYYCQQLNSYPLTFGGGTKVEIK

SEQ ID NO: 299 - protein, 6 aa, synthetic construct
QGISSY

SEQ ID NO: 300 - protein, 9 aa, synthetic construct
QQLNSYPLT

SEQ ID NO: 301 - protein, 120 aa, synthetic construct
QVQLVESGGGVVQPGRSLRLSCAASGFTFSYYGMHWVRQAPGKGLEWVAVIWYDGSNKFYADSVKGRFTISRDNSKNTLYLQMNSLRAEDTAVYYCAAARPGYYYGMDVWGQGTTVTVSS

SEQ ID NO: 302 - protein, 8 aa, synthetic construct
GFTFSYYG

SEQ ID NO: 303 - protein, 8 aa, synthetic construct
IWYDGSNK

SEQ ID NO: 304 - protein, 13 aa, synthetic construct
AAARPGYYYGMDV

SEQ ID NO: 305 - protein, 107 aa, synthetic construct
DIQMTQSPSSLSASVGDRVTITCRASQGISNYLAWFQQKPGKAPNSLIYAASSLQRGVPSKFSGSGSGTDFTLTISSLQPEDFATYYCQQYNSYPRTFGQGTKVEIK

SEQ ID NO: 306 - protein, 6 aa, synthetic construct
QGISNY

SEQ ID NO: 307 - protein, 9 aa, synthetic construct
QQYNSYPRT

SEQ ID NO: 308 - protein, 117 aa, synthetic construct
EVQLVESGGGLVQPGGSLRLSCAASGITVSSNYMSWVRQAPGKGLEWVSVIYSGGSTFYADSVKGRFTISRHNSKNTLYLQMNSLRAEDTAVYYCARERNFDAFDIWGQGTMVTVSS

SEQ ID NO: 309 - protein, 8 aa, synthetic construct
GITVSSNY

SEQ ID NO: 310 - protein, 11 aa, synthetic construct
ARERNFDAFDI

SEQ ID NO: 311 - protein, 108 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQANSFPLFTFGPGTKVDIK

SEQ ID NO: 312 - protein, 6 aa, synthetic construct
QGISSW

SEQ ID NO: 313 - protein, 10 aa, synthetic construct
QQANSFPLFT

SEQ ID NO: 314 - protein, 119 aa, synthetic construct
EVQLVESGGGLVQPGGSLRLSCAASGFTFSIYEMNWVRQAPGKGLEWVSYITSSGTTIYYADSVKGRFTISRDNAKNSLYLQMNSLRAEDTAVYYCASSSSSGYYFDYWGQGTLVTVSS

SEQ ID NO: 315 - protein, 8 aa, synthetic construct
GFTFSIYE

SEQ ID NO: 316 - protein, 8 aa, synthetic construct
ITSSGTTI

SEQ ID NO: 317 - protein, 12 aa, synthetic construct
ASSSSSGYYFDY

SEQ ID NO: 318 - protein, 113 aa, synthetic construct
DIVMTQSPDSLAVSLGERATINCKSSQSVLYSSNNKNYLAWYQQKPGQPPNLLIYWASTRESGVPDRFSGSGSGTDFTLTISSLQAEDVAVYYCQQYYSTPPTFGQGTKLEIK

SEQ ID NO: 319 - protein, 12 aa, synthetic construct
QSVLYSSNNKNY

SEQ ID NO: 320 - protein, 9 aa, synthetic construct
QQYYSTPPT

SEQ ID NO: 321 - protein, 121 aa, synthetic construct
QVQLVQSGAEVKKPGASVKVSCKASGYTFINYYIHWVRQAPGQGLEWMGWINPNSGGTNYAQKFQGRVTMTRDTSISTAYMELSRLRSDDTAVYYCAIITIFGVVTWFDPWGQGTLVTVSS

SEQ ID NO: 322 - protein, 8 aa, synthetic construct
GYTFINYY

SEQ ID NO: 323 - protein, 8 aa, synthetic construct
INPNSGGT

SEQ ID NO: 324 - protein, 14 aa, synthetic construct
AIITIFGVVTWFDP

SEQ ID NO: 325 - protein, 107 aa, synthetic construct [sequence omitted]

SEQ ID NO: 326 - protein, 6 aa, synthetic construct
QSISSY

SEQ ID NO: 327 - protein, 9 aa, synthetic construct
QQSYSTPLT

SEQ ID NO: 328 - protein, 115 aa, synthetic construct
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCARDPLLIDYWGQGTLVTVSS

SEQ ID NO: 329 - protein, 8 aa, synthetic construct
GGSISSYY

SEQ ID NO: 330 - protein, 7 aa, synthetic construct
IYYSGST

SEQ ID NO: 331 - protein, 9 aa, synthetic construct
ARDPLLIDY

SEQ ID NO: 332 - protein, 108 aa, synthetic construct
EIVLTQSPGTLSLSPGERATLSCRASQSVSRSYLAWYQQKPGQAPRLLIYGVSSRATGIPDRFSGSGSGTDFTLTISRLEPEDFAVYYCQQYGSSPQTFGQGTKLEIK

SEQ ID NO: 333 - protein, 7 aa, synthetic construct
QSVSRSY

SEQ ID NO: 334 - protein, 9 aa, synthetic construct
QQYGSSPQT

SEQ ID NO: 335 - protein, 125 aa, synthetic construct
EVQLVESGGGLVQPGRSLRLSCAASGFTFDDYAMYWVRQAPGKGLEWVSGISWNSGSIGYADSVKGRFTISRDNAKNSLYLQMNSLRAADTALYYCAKDKDWNSRGYYYYGMDVWGQGTTVTVSS

SEQ ID NO: 336 - protein, 8 aa, synthetic construct
GFTFDDYA

SEQ ID NO: 337 - protein, 8 aa, synthetic construct
ISWNSGSI

SEQ ID NO: 338 - protein, 18 aa, synthetic construct
AKDKDWNSRGYYYYGMDV

SEQ ID NO: 339 - protein, 107 aa, synthetic construct
DVVMTQSPSSLSASVGDRVTITCRASQSISSYLNWYQQKPGKAPKLLIYAASSLQSGVPSRFSGSGSGTDFTLTISSLQPEDFATYYCQQSYITPYTFGQGTKVEIK

SEQ ID NO: 340 - protein, 9 aa, synthetic construct
QQSYITPYT

SEQ ID NO: 341 - protein, 116 aa, synthetic construct
EVQLVESGGGLVQSGGSLRLSCAASGLTVSSNYMSWVRQAPGKGLEWVSVIYSGGSTFYSDSVKGRFTISRHNSKNTLYLQMNSLRAEDTAVYYCARARGDAFDIWGQGTMVTVSS

SEQ ID NO: 342 - protein, 8 aa, synthetic construct
GLTVSSNY

SEQ ID NO: 343 - protein, 10 aa, synthetic construct
ARARGDAFDI

SEQ ID NO: 344 - protein, 108 aa, synthetic construct
DIQMTQSPSSLSASVGDRVTITCQASQGISNYLNWYQQKPGKAPKLLISDASNLETGVPSRFSGSGSGTDFTFTISSLQPEDIATYYCQQYDNLRFFTFGPGTKVDIK

SEQ ID NO: 345 - protein, 10 aa, synthetic construct
QQYDNLRFFT

SEQ ID NO: 346 - protein, 120 aa, synthetic construct
EVQLVESGGGLVQPGGSLRLSCAASGFTVSSNYMSWVRQAPGKGLEWVSLIYSGGSTYYADSVKGRFTISRHNSKNTLYLQMNSLRSEDTAVYYCARVPLGDYYYGMDVWGQGTTVTVSS

SEQ ID NO: 347 - protein, 14 aa, synthetic construct
ARVPLGDYYYGMDV

SEQ ID NO: 348 - protein, 107 aa, synthetic construct
DIQMTQSPSSLSASVGDRVTITCQASQDINKYLNWYQQKPGKAPKLLIYDASNLETGVPSRFSGSGSGTDFTFTISSLQPEDIATYYCQQSDNLPLTFGGGTKVEIK

SEQ ID NO: 349 - protein, 6 aa, synthetic construct
QDINKY

SEQ ID NO: 350 - protein, 9 aa, synthetic construct
QQSDNLPLT

SEQ ID NO: 351 - protein, 124 aa, synthetic construct
QVQLVESGGGVVQPGRSLRLSCAASGFTFRNYGMHWVRQAPGKGLEWVSVIWYDGSNRYYADSVKGRFTISRDTSKNTLYLQMNSLRAEDTAVYYCARDPPITGTTGGDAFDIWGQGTMVTVSS

SEQ ID NO: 352 - protein, 8 aa, synthetic construct
GFTFRNYG

SEQ ID NO: 353 - protein, 8 aa, synthetic construct
IWYDGSNR

SEQ ID NO: 354 - protein, 17 aa, synthetic construct
ARDPPITGTTGGDAFDI

SEQ ID NO: 355 - protein, 107 aa, synthetic construct
DIQMTQSPSSLSASVGDRVTITCRASQSISSYLNWYQQKPGKAPHLLIYAASSLQSGVPSRFSGSGSGTDFTLTISSLQPEDFATYYCQQSYSTPYTFGQGTKLEIK

SEQ ID NO: 356 - protein, 9 aa, synthetic construct
QQSYSTPYT

SEQ ID NO: 357 - protein, 119 aa, synthetic construct
QITLKESGPTLVKPTQTLTLTCTFSGFSLSTNVVGVGWIRQPPGKALEWLALIYWNDDKRYSPSLKSRLTITKDTSKNQVVLTMTNMDPVDTATYYCAHGPTGDYFDYWGQGTLVTVSS

SEQ ID NO: 358 - protein, 10 aa, synthetic construct
GFSLSTNVVG

SEQ ID NO: 359 - protein, 7 aa, synthetic construct
IYWNDDK

SEQ ID NO: 360 - protein, 11 aa, synthetic construct
AHGPTGDYFDY

SEQ ID NO: 361 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQTYSTLMYTFGQGTKLEIK

SEQ ID NO: 362 - protein, 10 aa, synthetic construct
QQTYSTLMYT

SEQ ID NO: 363 - protein, 122 aa, synthetic construct
EVQLVESGGGLVQPGRSLRLSCAASGFTFDDYAMHWVRQAPGKGLEWVSGVSWNSGTIDYADSVKGRFTIIRDNAKNSLYLQMNSLRAEDTALYYCSKDMGRLDYYSGLDVWGQGTTVTVSS

SEQ ID NO: 364 - protein, 8 aa, synthetic construct
VSWNSGTI

SEQ ID NO: 365 - protein, 15 aa, synthetic construct
SKDMGRLDYYSGLDV

SEQ ID NO: 366 - protein, 108 aa, synthetic construct
EIVLTQSPGTLSLSPGERATLSCRASQSVSSSDFAWYQQKPGQAPRLLIYGASSRATGIPDRFSGSGSGTDFTLTISRLEPEDFAVYYCQQYGSSPWTFGQGTKVEIK

SEQ ID NO: 367 - protein, 7 aa, synthetic construct
QSVSSSD

SEQ ID NO: 368 - protein, 9 aa, synthetic construct
QQYGSSPWT

SEQ ID NO: 369 - protein, 118 aa, synthetic construct
EVQLVESGGGLVQPGGSLRLSCAVSGFTVSSNYMSWVRQAPGKGLEWVSLIYSGGSTFYADSVKGRFTISRHNSKNTLYLQMNSLRAEDTAVYYCAREGGYSYDYNYWGQGTLVTVSS

SEQ ID NO: 370 - protein, 12 aa, synthetic construct
AREGGYSYDYNY

SEQ ID NO: 371 - protein, 107 aa, synthetic construct
DIQMTQSPSSLSASVGDRVTITCQASQDIRNYLNWYQQKPGKAPKLLIYDASNLETGVPSRFSGIGSGTDFTFTISSLQPEDIATYYCQQYDNLPITFGQGTRLEIK

SEQ ID NO: 372 - protein, 6 aa, synthetic construct
QDIRNY

SEQ ID NO: 373 - protein, 9 aa, synthetic construct
QQYDNLPIT

SEQ ID NO: 374 - protein, 117 aa, synthetic construct
EVQLVESGGGLVQPGGSLRLSCAASGLTVSSNYMNWVRQVPGKGLEWVSVIYSGGSKFYVDSVKGRFTISRHNSKNTLYLQMNSLRPEDTAVYYCTRDAQYYGMDVWGQGTTVTVSS

SEQ ID NO: 375 - protein, 7 aa, synthetic construct
IYSGGSK

SEQ ID NO: 376 - protein, 11 aa, synthetic construct
TRDAQYYGMDV

SEQ ID NO: 377 - protein, 108 aa, synthetic construct
DIQMTQSPSSLSASVGDRVTITCRASQSISTYLHWYQQKPGEAPKLLIYAASSLQSGVPSRFSGSGSGTDFTLTISSLQPEDFATYYCQQSYSTPPEIFGQGTKLEIK

SEQ ID NO: 378 - protein, 6 aa, synthetic construct
QSISTY

SEQ ID NO: 379 - protein, 10 aa, synthetic construct
QQSYSTPPEI

SEQ ID NO: 380 - protein, 118 aa, synthetic construct
EVQLVESGGGLVQPGGSLRLSCAASGITVSSNYMTWVRQAPGKGLEWVSVIYSGGSTFYADSVKGRFTISRHNSKNTLYLQMNNLRAEDTAVYYCVRDLEVRGGMDVWGQGTTVTVSS

SEQ ID NO: 381 - protein, 12 aa, synthetic construct
VRDLEVRGGMDV

SEQ ID NO: 382 - protein, 108 aa, synthetic construct
EIVLTQSPGTLSLSPGERATLSCRASQSFSSSYLAWYQQKPGQAPRLLIYGASSRATGIPDRFSGSGSGTDFTLTISRLEPEDFAVYYCQQYGSSPWTFGQGTKVEIK

SEQ ID NO: 383 - protein, 7 aa, synthetic construct
QSFSSSY

SEQ ID NO: 384 - protein, 119 aa, synthetic construct
EVQLVESGGGLVQPGGSLRLSCAASGITVSSNYMSWVRQAPGKGLEWVSLIYSGGSTYYADSVKGRFTISRDNSKNTLYLQMNSLRVEDTAVYYCARDHGMAAAGYNYWGQGTLVTVSS

SEQ ID NO: 385 - protein, 13 aa, synthetic construct
ARDHGMAAAGYNY

SEQ ID NO: 386 - protein, 107 aa, synthetic construct
DIQMTQSPSSLSASVGDRVTITCQASQDINKYLNWYQQKPGKAPKLLIYDASNLETGVPSRFSGSGSGTDFTFTINSLQPEDFATYFCQQYDNLPPAFGQGTKVEIK

SEQ ID NO: 387 - protein, 9 aa, synthetic construct
QQYDNLPPA

SEQ ID NO: 388 - protein, 115 aa, synthetic construct
EVQLVESGGGLVQPGGSLRVSCAASGFTVSRNYMSWVRQAPGKGLEWVSVIYSGGSTFYADSVKGRFTISRDNSKNTLYLQMNSLRAEDTAIYYCARYIPRFDPWGQGTLVTVSS

SEQ ID NO: 389 - protein, 8 aa, synthetic construct
GFTVSRNY

SEQ ID NO: 390 - protein, 9 aa, synthetic construct
ARYIPRFDP

SEQ ID NO: 391 - protein, 107 aa, synthetic construct
DIQMTQSPSSLSASVGDRVTITCQASQDISNYLNWFQQKPGRAPKLLIYDASNLETGVPSRFSGSGSGTDFTFTISSLQPEDIATYYCQQYDNLPITFGQGTRLEIK

SEQ ID NO: 392 - protein, 6 aa, synthetic construct
QDISNY

SEQ ID NO: 393 - protein, 117 aa, synthetic construct
EVQLVESGGGLVQPGGSLRLSCAASGLTVSSNYMSWVRQAPGKGLEWVSVIYSGGSTFYADSVKGRFTISRDNSKNTLYLQMNSLRAEDTAVYYCARDLIKYGMDVWGQGTTVTVSS

SEQ ID NO: 394 - protein, 11 aa, synthetic construct
ARDLIKYGMDV

SEQ ID NO: 395 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDNLPPHFGPGTKVDIK

SEQ ID NO: 396 - protein, 9 aa, synthetic construct
QQYDNLPPH

SEQ ID NO: 397 - protein, 117 aa, synthetic construct
EVQLVESGGGLVQPGGSLRLSCAASGVTVSSNYMSWVRQAPGKGLEWVSVIYSGGSTFYADSVKGRFTISRDNSKNTLFLQMNSLRIEDTAVYYCARDLGPYGMDVWGQGTTVTVSS

SEQ ID NO: 398 - protein, 8 aa, synthetic construct
GVTVSSNY

SEQ ID NO: 399 - protein, 11 aa, synthetic construct
ARDLGPYGMDV

SEQ ID NO: 400 - protein, 108 aa, synthetic construct
DIQLTQSPSFLSASVGDRVTITCRASQGISSYLAWYQQKPGKAPKLLIYAASTLQSGVPSRFSGSGSGTQFILTISSLQPEDFATYYCQQLNSYPGFTFGPGTKVDIK

SEQ ID NO: 401 - protein, 10 aa, synthetic construct
QQLNSYPGFT

SEQ ID NO: 402 - protein, 118 aa, synthetic construct
QVQLQESGPGLVKPSETLSLTCTVSGGSINNYYWIWIRQPPGKGLEWIGYIYHSGSTNYNPSLKSRVTISVDTSKNQFSLKVSSVTAADTAVYYCANMVRGVYEDDYWGQGTLVTVSS

SEQ ID NO: 403 - protein, 8 aa, synthetic construct
GGSINNYY

SEQ ID NO: 404 - protein, 7 aa, synthetic construct
IYHSGST

SEQ ID NO: 405 - protein, 12 aa, synthetic construct
ANMVRGVYEDDY

SEQ ID NO: 406 - protein, 107 aa, synthetic construct
DIQMTQSPSSLSASVGDRVTITCRASQSISNYLNWYQQKPGKAPKLLIFAASSLRSGVPSRFSGSGSGTDFTLTISSLHPEDFATYYCQQNYNTPLTFGPGTKVDIK

SEQ ID NO: 407 - protein, 6 aa, synthetic construct
QSISNY

SEQ ID NO: 408 - protein, 9 aa, synthetic construct
QQNYNTPLT

SEQ ID NO: 409 - protein, 122 aa, synthetic construct
EVQLVESGGGLIQPGGSLRLSCAASVFTVSYNYMSWVRQAPGKGLEWVSVIYSGGSTYYADSVKGRFTISRDNSKNTLYLQMNSLRAEDTAVYYCARALPGWGGSFEYFDYWGQGTLVTVSS

SEQ ID NO: 410 - protein, 8 aa, synthetic construct
VFTVSYNY

SEQ ID NO: 411 - protein, 16 aa, synthetic construct
ARALPGWGGSFEYFDY

SEQ ID NO: 412 - protein, 107 aa, synthetic construct
DIQMTQSPSTLSASVGDRVTITCRASQSISSWLAWYQQKPGKAPKLLIYKASSLESGVSSRFSGSGSGTEFTLTISSLQPDDFATYYCQQYNSYSVTFGQGTKVEIK

SEQ ID NO: 413 - protein, 6 aa, synthetic construct
QSISSW

SEQ ID NO: 414 - protein, 9 aa, synthetic construct
QQYNSYSVT

SEQ ID NO: 415 - protein, 121 aa, synthetic construct
EVQLVESGGGLVQPGGSLRLSCAASGLTVSSNYMSWVRQAPGKGLEWVSVIYSGGSTFYADSVKGRFTISRDNSKNTLYLQMNSLRPEDTAVYYCAREGRIAATGYGMDVWGQGTTVTVSS

SEQ ID NO: 416 - protein, 15 aa, synthetic construct
AREGRIAATGYGMDV

SEQ ID NO: 417 - protein, 107 aa, synthetic construct
DIQMTQSPSSLSASVGDRVTITCQASQDISNYLNWYQQKPGKAPKLLIYDASNLGTGVPSRFSGSRSGTDFTFTISSLQPEDIATYYCHQYDNLPQTFGPGTKVDIK

SEQ ID NO: 418 - protein, 9 aa, synthetic construct
HQYDNLPQT

SEQ ID NO: 419 - protein, 114 aa, synthetic construct
EVQLVESGGGLVQPGGSLRLSCAASGFTVSSNYMSWVRQAPGKGLEWVSVIYPGGSTFYADSVKGRFTISRDNSKNTLYLQMNSLRAEDTAVYYCARDQRDFAWGQGTLVTVSS

SEQ ID NO: 420 - protein, 7 aa, synthetic construct
IYPGGST

SEQ ID NO: 421 - protein, 8 aa, synthetic construct
ARDQRDFA

SEQ ID NO: 422 - protein, 107 aa, synthetic construct
DIQMTQSPSSLSASVGDRVTITCQASQDISNYLNWYQQKPGKAPKLLIYDASNLETGVPSRFSGSGSGTDFTFTISGLQPEDIATYYCQQYDNLPPTFGQGTRLEIK

SEQ ID NO: 423 - protein, 9 aa, synthetic construct
QQYDNLPPT

SEQ ID NO: 424 - protein, 127 aa, synthetic construct
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARDGRTITMVRGVIDDAFDIWGQGTMVTVSS

SEQ ID NO: 425 - protein, 8 aa, synthetic construct
GFTFSSYA

SEQ ID NO: 426 - protein, 20 aa, synthetic construct
ARDGRTITMVRGVIDDAFDI

SEQ ID NO: 427 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDNLPLTFGQGTRLEIK

SEQ ID NO: 428 - protein, 120 aa, synthetic construct
EVQLVESGGGLVQPGGSLRLSCAASGFTVSSNYMSWVRQAPGKGLEWVSVIYSGGSTYYADSVKGRFTISRDNSKNTLYLQMNRLRADDTAVYYCVRDPVGRYYYGMDVWGQGTTVTVSS

SEQ ID NO: 429 - protein, 14 aa, synthetic construct
VRDPVGRYYYGMDV

SEQ ID NO: 430 - protein, 107 aa, synthetic construct
DIQMTQSPSSLSASVGDRVTITCQASQDINNYLNWYQQKPGKAPKLLIYDASNLETGVPSRFSGSGSGTDFTFTISSLQPEDIATYYCQQYDNLPRTFGGGTKVEIK

SEQ ID NO: 431 - protein, 6 aa, synthetic construct
QDINNY

SEQ ID NO: 432 - protein, 9 aa, synthetic construct
QQYDNLPRT

SEQ ID NO: 433 - protein, 117 aa, synthetic construct
EVQLVESGGGLIQPGGSLRLSCAASGVTVSSNYMSWVRQAPGKGLEWVSVIYSGGSTFYSDSVKGRFSISRDNSKNTLYLQMNSLRAEDTAVYYCARDLYYYGMDVWGQGTTVTVSS

SEQ ID NO: 434 - protein, 11 aa, synthetic construct
ARDLYYYGMDV

SEQ ID NO: 435 - protein, 108 aa, synthetic construct
DIQLTQSPSFLSASVGDRVTITCRASQGISTYLAWYQQKPGKAPKLLIYAASTLQSGVPSRFSGSGSGTEFTLTISSLQPEDFATYYCQQLDSYPGLTFGGGTKVEIK

SEQ ID NO: 436 - protein, 6 aa, synthetic construct
QGISTY

SEQ ID NO: 437 - protein, 10 aa, synthetic construct
QQLDSYPGLT

SEQ ID NO: 438 - protein, 123 aa, synthetic construct
QVQLVQSGAEVKKPGSSVKVSCKASGGTFSSYAITWVRQAPGQGLEWMGRIIPMFGIANYAQKFQGRVTITADKSTSTAYMEVSSLRSEDTAVYYCARTPFYYDSSGYYLDYWGQGTLVTVSS

SEQ ID NO: 439 - protein, 8 aa, synthetic construct
GGTFSSYA

SEQ ID NO: 440 - protein, 8 aa, synthetic construct
IIPMFGIA

SEQ ID NO: 441 - protein, 16 aa, synthetic construct
ARTPFYYDSSGYYLDY

SEQ ID NO: 442 - protein, 108 aa, synthetic construct
EIVLTQSPGTLSLSPGERATLSCRASQSVSSNYLAWYQQSPGQAPRLLIYGASSRATGIPDRFSGSGSGTDFTLTISRLEPEDFAVYYCQQYVRSPRTFGQGTKVEIK

SEQ ID NO: 443 - protein, 7 aa, synthetic construct
QSVSSNY

SEQ ID NO: 444 - protein, 9 aa, synthetic construct
QQYVRSPRT

SEQ ID NO: 445 - protein, 119 aa, synthetic construct
EVQLVESGGGLVQPGGSLRLSCAASGLTVSSNYMSWVRQAPGKGLEWVSVIYSGGTTYYADSVKGRFTISRHNSKNTLSLQMNSLRAEDTAVYYCARGEGANYYGMDVWGQGTTVTVSS

SEQ ID NO: 446 - protein, 7 aa, synthetic construct
IYSGGTT

SEQ ID NO: 447 - protein, 13 aa, synthetic construct
ARGEGANYYGMDV

SEQ ID NO: 448 - protein, 112 aa, synthetic construct
QSVLTQPPSVSGAPGQRVTISCTGSSSNIGTGYDVHWYQQLPGTAPKLLIYGNSNRPSGVPDRFSGSKSGTSASLAITGLQAEDEADYYCQSYDSSLSGYVVFGGGTKLTVL

SEQ ID NO: 449 - protein, 9 aa, synthetic construct
SSNIGTGYD

SEQ ID NO: 450 - protein, 12 aa, synthetic construct
QSYDSSLSGYVV

SEQ ID NO: 451 - protein, 119 aa, synthetic construct
EVQLVESGGGLVQPGGSLRLSCAASGLTVNRNYMSWVRQAPGKGLEWVSLIYSGGSTYYADSVKGRFTISRDNSKNTLYLQMNSLRLEDTAVYYCARGELGIPYGMDVWGQGTTVTVSS

SEQ ID NO: 452 - protein, 8 aa, synthetic construct
GLTVNRNY

SEQ ID NO: 453 - protein, 13 aa, synthetic construct
ARGELGIPYGMDV

SEQ ID NO: 454 - protein, 110 aa, synthetic construct
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAGVFGGGTKLTVL

SEQ ID NO: 455 - protein, 11 aa, synthetic construct
GTWDSSLSAGV

SEQ ID NO: 456 - protein, 128 aa, synthetic construct
EVQLVESGGGLVQPGRSLRLSCAASGFTFDDYAMHWVRQAPGKGLEWVSGISWNRGSIGYADSVKGRFTISRDNAKNSLYLQMSSLRAEDTALYYCAKDGERWDSVVVPSARNGMDVWGQGTTVTVSS

SEQ ID NO: 457 - protein, 8 aa, synthetic construct
ISWNRGSI

SEQ ID NO: 458 - protein, 21 aa, synthetic construct
AKDGERWDSVVVPSARNGMDV

SEQ ID NO: 459 - protein, 112 aa, synthetic construct
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSYVFGTGTKVTVL

SEQ ID NO: 460 - protein, 9 aa, synthetic construct
SSNIGAGYD

SEQ ID NO: 461 - protein, 12 aa, synthetic construct
QSYDSSLSGSYV

SEQ ID NO: 462 - protein, 119 aa, synthetic construct
EVQLVESGGGLVQPGGSLRLSCAASEFTVSSNYMSWVRQAPGKGLEWVSVIYSGGSTFYADSVKGRFTISRDNSKNTLYLQMNSLRAEDTAVYYCARALPYGDLHFDYWGQGTLVTVSS

SEQ ID NO: 463 - protein, 8 aa, synthetic construct
EFTVSSNY

SEQ ID NO: 464 - protein, 13 aa, synthetic construct
ARALPYGDLHFDY

SEQ ID NO: 465 - protein, 112 aa, synthetic construct
QSVLTQPPSVSGAPGQRVTISCTGSSSNIGTGYDVHWYQQLPGTAPKLLIYGNTNRPSGVPDRFSGSKSGTSASLAITGLQAEDEADYYCQSYDSSLSDSYVFGTGTKVTVL

SEQ ID NO: 466 - protein, 12 aa, synthetic construct
QSYDSSLSDSYV

SEQ ID NO: 467 - protein, 127 aa, synthetic construct
EVQLVESGGGLVQPGGSLRLSCTASGFTFSNYEMNWVRQAPGKGLEWVSYISSRSGSTLHYADSVKGRFTISRDNAKNSLYLQMNSLRAEDTAVYYCARRGDGTSSLIHHYYYMDVWGKGTTVTVSS

SEQ ID NO: 468 - protein, 8 aa, synthetic construct
GFTFSNYE

SEQ ID NO: 469 - protein, 9 aa, synthetic construct
ISSRSGSTL

SEQ ID NO: 470 - protein, 19 aa, synthetic construct
ARRGDGTSSLIHHYYYMDV

SEQ ID NO: 471 - protein, 110 aa, synthetic construct
QSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGPVFGGGTKLTVL

SEQ ID NO: 472 - protein, 8 aa, synthetic construct
SSNIGSNT

SEQ ID NO: 473 - protein, 11 aa, synthetic construct
AAWDDSLNGPV

SEQ ID NO: 474 - protein, 117 aa, synthetic construct
EVQMVESGGGLVQPGGSLRLSCAASGFTVSSNYMTWVRQAPGKGLEWVSVIYSGGSTYYADSVKGRFTISRHNSKNTLYLQMNSLRAEDTAVYYCARDGVSYGMDVWGQGTTVTVSS

SEQ ID NO: 475 - protein, 11 aa, synthetic construct
ARDGVSYGMDV

SEQ ID NO: 476 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 477 - protein, 10 aa, synthetic construct
QQSYSTPPIT

SEQ ID NO: 478 - protein, 119 aa, synthetic construct
EVQLVESGGGLVHPGGSLRLSCAASGLTVSSNYMSWVRQAPGKGLEWVSVIYSGGSTYYADSVKGRFTISRHNSKNTLFLQMNSLRAEDTAVYYCARDRPITGTTLDVWGQGTTVTVSS

SEQ ID NO: 479 - protein, 13 aa, synthetic construct
ARDRPITGTTLDV

SEQ ID NO: 480 - protein, 108 aa, synthetic construct
DIQMTQSPSSLFASVGDRVTITCRASQSISSYLNWYQQKPGKAPKLLIYAASSLQSGVPSRFSGSGSGTDFTLTISSLQPEDFAFYYCQQTYSTPPITFGQGTRLEIK

SEQ ID NO: 481 - protein, 10 aa, synthetic construct
QQTYSTPPIT

SEQ ID NO: 482 - protein, 117 aa, synthetic construct
EVQLVESGGGLVQPGGSLRLSCAASGFTVSSNYMNWVRQAPGKGLEWVSVIYSGGSTFYADSVRGRFTISRHNSKNTLYLQMNSLRAEDTAVYYCARDGSAYGMDVWGQGTTVTVSS

SEQ ID NO: 483 - protein, 11 aa, synthetic construct
ARDGSAYGMDV

SEQ ID NO: 484 - protein, 108 aa, synthetic construct
DIQMTQSPSSLSASVGDRVTITCRASQSISSFLNWYQQKPGKAPKLLIYAASSLQSGVPSRFSGSGSGTDFTLTISSLQPEDFATYYCQQSYSSPPITFGQGTRLEIK

SEQ ID NO: 485 - protein, 6 aa, synthetic construct
QSISSF

SEQ ID NO: 486 - protein, 10 aa, synthetic construct
QQSYSSPPIT

SEQ ID NO: 487 - protein, 115 aa, synthetic construct
EVQLVESGGGLVQPGGSLRLSCAASGFTVSSNYMSWVRQAPGKGLEWVSVIYSGGSTFYADSVKGRFTISRHNSKNTLYLQMNSLRAEDTAVYYCAREGYGMDVWGQGTTVTVSS

SEQ ID NO: 488 - protein, 9 aa, synthetic construct
AREGYGMDV

SEQ ID NO: 489 - protein, 120 aa, synthetic construct
QVQLVESGGGVVQPGRSLRLSCVASGFTFSIYGMDWVRQAPGKGLEWVAVISYDGSNKYYADSVKGRFTISRDNSKNTLYLQMNSLRPEDTAVYYCAKDREAAADAFDIWGQGTMVTVSS

SEQ ID NO: 490 - protein, 8 aa, synthetic construct
GFTFSIYG

SEQ ID NO: 491 - protein, 13 aa, synthetic construct
AKDREAAADAFDI

SEQ ID NO: 492 - protein, 107 aa, synthetic construct
DIQMTQSPSSVSASVGDRVTITCRASQGISSWLAWYQQKPGKAPKLLIYAASSLHSGVPSRFSGSGSGTDFTLTISSLQPEDFATYYCQQANSFRLTFGGGTKVEIK

SEQ ID NO: 493 - protein, 9 aa, synthetic construct
QQANSFRLT

SEQ ID NO: 494 - protein, 116 aa, synthetic construct
EVQLVESGGGLVQPGGSLRLSCAASGFIVSRNYMSWVRQAPGKGLEWVSVIYSGGSTFYADSVKGRFTISRHNSKNTLYLQMNSLKAEDTAVYYCARELRGYFDYWGQGTLVTVSS

SEQ ID NO: 495 - protein, 8 aa, synthetic construct
GFIVSRNY

SEQ ID NO: 496 - protein, 10 aa, synthetic construct
ARELRGYFDY

SEQ ID NO: 497 - protein, 107 aa, synthetic construct
DIQLTQSPSFLSASVGDRVTITCWASQGISSYLAWYQQKPGKAPKLLIYAASTLQSGVPSRFSGSGSGTEFTLTISSLQPEDFATYYCQLLNSYPYTFGQGTKLEIK

SEQ ID NO: 498 - protein, 9 aa, synthetic construct
QLLNSYPYT

SEQ ID NO: 499 - protein, 117 aa, synthetic construct
EVQLVESGGGLVQPGGSLRLSCAASGFTVSSNYMTWVRQAPGKGLEWVSVIYSGGSTFYADSVKGRFTISRHNSENTLYLQMNSLRAEDTAVYYCARDLAAAGTDYWGQGALVTVSS

SEQ ID NO: 500 - protein, 11 aa, synthetic construct
ARDLAAAGTDY

SEQ ID NO: 501 - protein, 107 aa, synthetic construct
DIQLTQSPSFLSASVGDRVTITCWASQGISSYLAWYQQKPGKAPKLLIYAASTLQSGVPSRFSGSGSGTEFTLTISSLQPDDFATYYCQHLNSYLYTFGQGTKLEIK

SEQ ID NO: 502 - protein, 9 aa, synthetic construct
QHLNSYLYT

SEQ ID NO: 503 - protein, 120 aa, synthetic construct
QVQLVESGGGVVQPGRSLRLSCAASGFTFSSYGMHWVRQAPGKGLEWVAVISYDGSYKYYADSVKGRFTISRDNSKNTLYLQMNSLRAEDTAVYYCAIDGGTVTTIFDYWGQGTLVTVSS

SEQ ID NO: 504 - protein, 8 aa, synthetic construct
GFTFSSYG

SEQ ID NO: 505 - protein, 8 aa, synthetic construct
ISYDGSYK

SEQ ID NO: 506 - protein, 13 aa, synthetic construct
AIDGGTVTTIFDY

SEQ ID NO: 507 - protein, 108 aa, synthetic construct
DIQMTQSPSSLSASVGDRVTITCRASQSISSYLNWYQQKPGKAPKLLIYTASSLQSGVPSRFSGSGSGTDFTLTISSLQPEDFATYYCQQSYSTPPWTFGQGTKVEIK

SEQ ID NO: 508 - protein, 10 aa, synthetic construct
QQSYSTPPWT

SEQ ID NO: 509 - protein, 125 aa, synthetic construct
EVQLVETGGGLIQPGESLRLSCAASGFIVSTNYMNWVRQAPGKGLEWVSVIYRGGSTYYADSVKGRFTISRDNSKNTLFLQMNSLRAEDTAVYYCARGHEDFDYDTPASGAFDIWGQGTMVTVSS

SEQ ID NO: 510 - protein, 8 aa, synthetic construct
GFIVSTNY

SEQ ID NO: 511 - protein, 7 aa, synthetic construct
IYRGGST

SEQ ID NO: 512 - protein, 19 aa, synthetic construct
ARGHEDFDYDTPASGAFDI

SEQ ID NO: 513 - protein, 110 aa, synthetic construct
QSALTQPPSASGSPGQSVTISCTGTSSDVGGHNYVSWYQQHPGKAPKVMIYEVSKRPSGVPDRFSGSKSGNTASLTVSGLQAEDEADYYCSSYAGSNKFVFGTGTKVTVL

SEQ ID NO: 514 - protein, 9 aa, synthetic construct
SSDVGGHNY

SEQ ID NO: 515 - protein, 10 aa, synthetic construct
SSYAGSNKFV

SEQ ID NO: 516 - protein, 123 aa, synthetic construct
EVQLVESGGGLVQPGGSLRLSCAASGFAVSSNYMTWVRQAPGKGLEWVATIYSGDSTYYADSVKGRFTISRHNSKNTLSLQMNSLRADDTAIYYCARLSGVLSGTYNGAFDIWGQGTMVTVSS

SEQ ID NO: 517 - protein, 8 aa, synthetic construct
GFAVSSNY

SEQ ID NO: 518 - protein, 7 aa, synthetic construct
IYSGDST

SEQ ID NO: 519 - protein, 17 aa, synthetic construct
ARLSGVLSGTYNGAFDI

SEQ ID NO: 520 - protein, 110 aa, synthetic construct
QSALTQPASVSGSPGQSITISCTGTSSDFGNYNIVSWYQQHPGQAPKVMIYEGNKRPSGVSNRFSGSKSGHTASLTISGLQAEDEADYYCSSYAGSSTWVFGGGTKLTVL

SEQ ID NO: 521 - protein, 9 aa, synthetic construct
SSDFGNYNI

SEQ ID NO: 522 - protein, 10 aa, synthetic construct
SSYAGSSTWV

SEQ ID NO: 523 - protein, 122 aa, synthetic construct
EVQLVESGGGLVQPGGSLRLSCAASGFTVSSNYMSWVRQAPGKGLEWVSAIYSGDSTYYADSVKGRFTISKDNSKNTLYLQMNSLRGEDTAVYYCARLGGALQAYDGAFDYWGQGTLATVSS

SEQ ID NO: 524 - protein, 16 aa, synthetic construct
ARLGGALQAYDGAFDY

SEQ ID NO: 525 - protein, 110 aa, synthetic construct
QSALTQPASVSGSPGQSITISCTGTSSDFGSYNLVSWYQQHPGKAPKLMIYEGNKRPSGVSIRFSGSKSGNTASLTISGLQAEDEADYYCSSYAGSSTWVFGGGTKLTVL

SEQ ID NO: 526 - protein, 9 aa, synthetic construct
SSDFGSYNL

SEQ ID NO: 527 - protein, 121 aa, synthetic construct
EVQLVESGGGLVQPGGSLRLSCAASGFTVTSNYMSWVRQAPGKGLEWVSAIYSGESTYYADSVKGRFTISRDSSKNTLYLQMNSLRVEDTAVYYCASIHDYSNYVGGFDPWGQGTLVTVSS

SEQ ID NO: 528 - protein, 8 aa, synthetic construct
GFTVTSNY

SEQ ID NO: 529 - protein, 7 aa, synthetic construct
IYSGEST

SEQ ID NO: 530 - protein, 15 aa, synthetic construct
ASIHDYSNYVGGFDP

SEQ ID NO: 531 - protein, 110 aa, synthetic construct
QSALTQPASVSGSPGQSITISCTGPSSDFGSYNLVSWYQQHPGKAPKLMIYEGTKRPSGVSNRFSGSKSGNTASLTISGLQAEDEADYYCSSYAGSSTWVFGGGTKLTVL

SEQ ID NO: 532 - protein, 122 aa, synthetic construct
QVQLQESGPGLVKPSETLSLTCTVSGGSISGYYWSWIRQPPGKGLEWIGYIYYSGSTNYNPSLKSRVTMSVDTSKNQFSLKLSSVTAADTAVYYCARYPLYSESSGYHFDYWGHGTLVTVSS

SEQ ID NO: 533 - protein, 8 aa, synthetic construct
GGSISGYY

SEQ ID NO: 534 - protein, 16 aa, synthetic construct
ARYPLYSESSGYHFDY

SEQ ID NO: 535 - protein, 110 aa, synthetic construct
QSALTQPASVSGSPGQSITISCTGTSSDVGSYNLVSWYQQDPGKAPKLMLYEDSKRPSGVSNRFSGSKSGNTASLTISGLQAEDEADYYCSSYAGRSTWVFGGGTKLTVL

SEQ ID NO: 536 - protein, 9 aa, synthetic construct
SSDVGSYNL

SEQ ID NO: 537 - protein, 10 aa, synthetic construct
SSYAGRSTWV

SEQ ID NO: 538 - protein, 117 aa, synthetic construct
EVQLVESGGGLVQPGGSLRLSCAATGITVSSNYMSWVRQAPGKGLEWVSLIYSGGSTFYADSVKGRFTISRHNSKNTLYLQMSSLRAEDTAVYYCARDLVDFGMDVWGQGTTVTVSS

SEQ ID NO: 539 - protein, 11 aa, synthetic construct
ARDLVDFGMDV

SEQ ID NO: 540 - protein, 107 aa, synthetic construct
DIQLTQSPSSLSASVGDRVTITCRASQGISNYLAWYQQKPGKAPKLLIYAASTLQSGVPSRFSGSGSGTDFTLTISSLQPEDFGTYYCQQLNSDSPTFGQGTKVEIK

SEQ ID NO: 541 - protein, 9 aa, synthetic construct
QQLNSDSPT

SEQ ID NO: 542 - protein, 128 aa, synthetic construct
EVQLVQSGAEVKKPGESLKISCKGSGYSFTSYWIGWVRQMPGKGLEWMGIIYPGDTNTRYSPSFQGQVTISADKSISTAYLQWSSLKASDTAMYYCARLRAPSGPWEGAIPGGGMDVWGQGTTVTVSS

SEQ ID NO: 543 - protein, 8 aa, synthetic construct
GYSFTSYW

SEQ ID NO: 544 - protein, 8 aa, synthetic construct
IYPGDTNT

SEQ ID NO: 545 - protein, 21 aa, synthetic construct
ARLRAPSGPWEGAIPGGGMDV

SEQ ID NO: 546 - protein, 108 aa, synthetic construct
EIVMTQSPATLSVSPGERATLSCRASQSVSSNLAWYQQKHGQAPRLLIYGASTRATGIPARFSGSGSGTEFTLTISSLQSEDFAVYYCQQYNNWPPFTFGPGTKVDIK

SEQ ID NO: 547 - protein, 6 aa, synthetic construct
QSVSSN

SEQ ID NO: 548 - protein, 10 aa, synthetic construct
QQYNNWPPFT

SEQ ID NO: 549 - protein, 127 aa, synthetic construct
QVQLVQSGAEVKKPGASVKVSCKASGYTFTDYYMHWVRQAPGQGLEWMGWINPNSGGTNYAQKFQGRVTMTRDTSISTAYMELSRLRSDDTAIYYCARTLFKYYDILIGYGEYFQHWGQGTLVIVSS

SEQ ID NO: 550 - protein, 8 aa, synthetic construct
GYTFTDYY

SEQ ID NO: 551 - protein, 20 aa, synthetic construct
ARTLFKYYDILIGYGEYFQH

SEQ ID NO: 552 - protein, 108 aa, synthetic construct
EIVLTQSPGTLSLSPGERATLSCRASQSVSSSYLAWYQQKPGQAPRLLIYGASSRATAIPDRFSGSGSGTDFTLTISRLEPEDFAVYFCQQYGSSPWTFGQGTKVEIK

SEQ ID NO: 553 - protein, 7 aa, synthetic construct
QSVSSSY

SEQ ID NO: 554 - protein, 120 aa, synthetic construct
EVQLVESGGGLVQPGGSLRLSCAASGFTVSRSYMTWVRQAPGKGLEWVSVIFTGGRTDHADSVKGRFTISRHNSKNTLYLQMNSLRTEDTAVYYCARVAIVGATGAFDIWGQGTMVTVSS

SEQ ID NO: 555 - protein, 8 aa, synthetic construct
GFTVSRSY

SEQ ID NO: 556 - protein, 7 aa, synthetic construct
IFTGGRT

SEQ ID NO: 557 - protein, 14 aa, synthetic construct
ARVAIVGATGAFDI

SEQ ID NO: 558 - protein, 112 aa, synthetic construct
QSVLTQPPSVSGAPGQRVTISCTGSSSNIGAGDDVHWYQQLPGTAPKLLIYGNSNRPSGVPDRFSGSKSGTSASLAITGLQAEDEADYYCQSYDSSLSGSVVFGGGTKLTVL

SEQ ID NO: 559 - protein, 9 aa, synthetic construct
SSNIGAGDD

SEQ ID NO: 560 - protein, 12 aa, synthetic construct
QSYDSSLSGSVV

SEQ ID NO: 561 - protein, 123 aa, synthetic construct
EVQLVESGGGLVQPGRSLRLSCVASGFTFDDSAMHWVRQPPGKGLEWVSGISWNSGIIGYAESVKGRFIISRDNAKNSLFLHMRGLRPEDTAVYYCARDRLELSYYYYGMDVWGQGTTVTVSS

SEQ ID NO: 562 - protein, 8 aa, synthetic construct
GFTFDDSA

SEQ ID NO: 563 - protein, 8 aa, synthetic construct
ISWNSGII

SEQ ID NO: 564 - protein, 16 aa, synthetic construct
ARDRLELSYYYYGMDV

SEQ ID NO: 565 - protein, 110 aa, synthetic construct
QSVLTQPPSASGTPGQRVTISCSGSSSNIGSNTVNWYQQFPGTAPRLLIYSNDQRPSGIPDRFSGSKSGTSASLAISGLQSEDEADYYCSTWDDSLNGAVFGGGTQLTVL

SEQ ID NO: 566 - protein, 11 aa, synthetic construct
STWDDSLNGAV

SEQ ID NO: 567 - protein, 121 aa, synthetic construct
RVTLRESGPALVKPTQTLTLTCTFSGFSLYSSGMSVSWIRQPPGKALEWLARIDWDDDEYYSSFLKTRLTISKDISKNQVVLTMTNLDPVDTATYYCVRPRYSSNFGFEYWGQGTLVTVSS

SEQ ID NO: 568 - protein, 10 aa, synthetic construct
GFSLYSSGMS

SEQ ID NO: 569 - protein, 7 aa, synthetic construct
IDWDDDE

SEQ ID NO: 570 - protein, 13 aa, synthetic construct
VRPRYSSNFGFEY

SEQ ID NO: 571 - protein, 110 aa, synthetic construct
QSVLTQPPSVSAAPGQKVTISCSGSISNIGNNYVSWYQQLPGTAPKLLIYDNDKRPSGIPDRFSGSRSGTSATLGITGLQTGDEADYYCGTWDSSLTAYVFGTGTKVTVL

SEQ ID NO: 572 - protein, 8 aa, synthetic construct
ISNIGNNY

SEQ ID NO: 573 - protein, 11 aa, synthetic construct
GTWDSSLTAYV

SEQ ID NO: 574 - protein, 121 aa, synthetic construct
QVQLVQSGAEVKKPGSSVKVSCKASGDTFSTYAINWVRQAPGQGLEWMGRFIHIFGTANYAQKFQGRVTITADESTSTAYMELRSLRSEDTAVYYCARDGVDYGDYRPDYWGQGTLVTVSS

SEQ ID NO: 575 - protein, 8 aa, synthetic construct
GDTFSTYA

SEQ ID NO: 576 - protein, 8 aa, synthetic construct
FIHIFGTA

SEQ ID NO: 577 - protein, 14 aa, synthetic construct
ARDGVDYGDYRPDY

SEQ ID NO: 578 - protein, 108 aa, synthetic construct
EIVLTQSPGTLSLSPGERATLSCRASQSVSSNYLAWYQQKPGQAPRLLIYGASSRATGIPERFSGSGSGTDFTLTISRLEPEDFAVYYCQQYGSSLYTFGQGTKLEIK

SEQ ID NO: 579 - protein, 9 aa, synthetic construct
QQYGSSLYT

SEQ ID NO: 580 - protein, 119 aa, synthetic construct
QVQLVQSGAEVKKPGSSVKVSCKAPGGTFSTYAINWVRQAPGQGLEWMGRIVPFFGTTNYAQKFQGRVTITADESTSTAYMELSSLTSEDTAVYYCARGPRPFGELLYWGQGTLVTVSS

SEQ ID NO: 581 - protein, 8 aa, synthetic construct
GGTFSTYA

SEQ ID NO: 582 - protein, 8 aa, synthetic construct
IVPFFGTT

SEQ ID NO: 583 - protein, 12 aa, synthetic construct
ARGPRPFGELLY

SEQ ID NO: 584 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQAEDIATYYCQQYDNLLFTFGPGTKVDIK

SEQ ID NO: 585 - protein, 9 aa, synthetic construct
QQYDNLLFT

SEQ ID NO: 586 - protein, 119 aa, synthetic construct
EVQLVETGGGLIQPGGSLRLSCAAPGFIVSSNYMSWVRQAPGKGLEWVSLLYPGGSTYYADSVKGRFTISRDNSKNTLYLQMNSLRADDTAVYYCARVLPIVEDHFDYWGQGTLVTVSS

SEQ ID NO: 587 - protein, 8 aa, synthetic construct
GFIVSSNY

SEQ ID NO: 588 - protein, 7 aa, synthetic construct
LYPGGST

SEQ ID NO: 589 - protein, 13 aa, synthetic construct
ARVLPIVEDHFDY

SEQ ID NO: 590 - protein, 110 aa, synthetic construct
DIQLTQSPSFLSASVGDRVTITCRASQGMSSYLAWYQKKPGKAPKLLIYGATILQSGVPSRFSGSGSGTEFTLTISSLQPEDFATYYCQQLNSDPLYMYTFGQGTKLEIK

SEQ ID NO: 591 - protein, 6 aa, synthetic construct
QGMSSY

SEQ ID NO: 592 - protein, 12 aa, synthetic construct
QQLNSDPLYMYT

SEQ ID NO: 593 - protein, 122 aa, synthetic construct
QVQLVESGGGVVQPGRSLRLSCAASGFVFNSYGMHWVRQAPGKGLEWVAVLWYEGSKNYYADSVKGRFTISRDNSKNTLYLQMNSLRAEDTAVYYCARHGSGSFFGYYLDYWGQGTLVTVSS

SEQ ID NO: 594 - protein, 8 aa, synthetic construct
GFVFNSYG

SEQ ID NO: 595 - protein, 8 aa, synthetic construct
LWYEGSKN

SEQ ID NO: 596 - protein, 15 aa, synthetic construct
ARHGSGSFFGYYLDY

SEQ ID NO: 597 - protein, 107 aa, synthetic construct
DIQMTQSPSSLSASVGDRVTITCRASHNINDFLNWYQQKPGKAPRLLIYAASSLQSGVPSRFSGSGSGTDFTLTISSLQPEDFATYYCQESYTTPPTFGQGTKLEIK

SEQ ID NO: 598 - protein, 6 aa, synthetic construct
HNINDF

SEQ ID NO: 599 - protein, 9 aa, synthetic construct
QESYTTPPT

SEQ ID NO: 600 - protein, 122 aa, synthetic construct
QLQLQESGPGLVKSSETLSLTCTVSASITGSTYYWGWIRQPPGKGLEWIGSIYYSGTTYYKASLKSRVTISVDTSKNQFSLKLTSVTVADTAVYYCVSHLLGNYHKYGMDVWGQGTTVTVSS

SEQ ID NO: 601 - protein, 9 aa, synthetic construct
ASITGSTYY

SEQ ID NO: 602 - protein, 7 aa, synthetic construct
IYYSGTT

SEQ ID NO: 603 - protein, 15 aa, synthetic construct
VSHLLGNYHKYGMDV

SEQ ID NO: 604 - protein, 112 aa, synthetic construct
QSVLTQPPSASGTPGQRVTISCSGSRSNIGINTVNWYQQFPGTALNLLIFSDNQRPSGVPDRFSGSKSGTSASLAISGLQSEDEADYYCAAWDDSLNGPDWVFGGGTKLTVL

SEQ ID NO: 605 - protein, 8 aa, synthetic construct
RSNIGINT

SEQ ID NO: 606 - protein, 13 aa, synthetic construct
AAWDDSLNGPDWV

SEQ ID NO: 607 - protein, 123 aa, synthetic construct
EVQLVESGGALVQPGGSLRLSCAASGFPFTIYAMTWVRQAPGKGLEWVSVITGSGDSTYYADSVKGRFTISRDNSKNTLYLQMNSLRAEDTAVYYCVKTGEGFFYYYYGMDVWGQGTTVTVSS

SEQ ID NO: 608 - protein, 8 aa, synthetic construct
GFPFTIYA

SEQ ID NO: 609 - protein, 8 aa, synthetic construct
ITGSGDST

SEQ ID NO: 610 - protein, 16 aa, synthetic construct
VKTGEGFFYYYYGMDV

SEQ ID NO: 611 - protein, 110 aa, synthetic construct
QSVLTQPPSVSAAPGQKVTISCSGSSSNIGNSYISWYQQLPGTAPKLLIYVNNKRPSGIPDRFSGSKSGTSATLGITGLQTGDEADYYCGTWDSSLSAGVFGSGTKVTVL

SEQ ID NO: 612 - protein, 8 aa, synthetic construct
SSNIGNSY

SEQ ID NO: 613 - protein, 121 aa, synthetic construct
EVQLVESGGGLVQPGGSLRLSCAASGFSFSSNWMSWVRQAPGKGLEWVGNINQDGSEKYYVDSVEGRFTISRDNAKKSLYLQMNSLRAEDTAVYYCARDLGVLWFGDFVDWGQGALVTVSS

SEQ ID NO: 614 - protein, 8 aa, synthetic construct
GFSFSSNW

SEQ ID NO: 615 - protein, 8 aa, synthetic construct
INQDGSEK

SEQ ID NO: 616 - protein, 14 aa, synthetic construct
ARDLGVLWFGDFVD

SEQ ID NO: 617 - protein, 107 aa, synthetic construct
DIQMTQSPSSLSASVGDRVTITCRASQAISNSLAWYQQKPGKAPKLLLYAASTLESGVPSRFSGSGSGTDYTLTISSLQPEDFATYYCLQYYGSPRTFGQGTKVEIK

SEQ ID NO: 618 - protein, 6 aa, synthetic construct
QAISNS

SEQ ID NO: 619 - protein, 9 aa, synthetic construct
LQYYGSPRT

SEQ ID NO: 620 - protein, 117 aa, synthetic construct
EVQLVESGGGLVQPGGSLRLSCAASGITVSSNYMTWVRQAPGKGLEWVSLIYSGGSTFYADSVRGRFTISRDNSKNTLYLQMNSLRAEDTAVYYCARDLVVYGMDVWGQGTTVTVSS

SEQ ID NO: 621 - protein, 11 aa, synthetic construct
ARDLVVYGMDV

SEQ ID NO: 622 - protein, 107 aa, synthetic construct
DIQLTQSPSSLSASVGDRVTITCRASQGISSYLAWYQQKPGKAPKLLIYAASTLQSGVPSRFSGSGSGTDFTLTISSLQPEDFATYYCQHLNSDPHTFGQGTKLEIK

SEQ ID NO: 623 - protein, 9 aa, synthetic construct
QHLNSDPHT

SEQ ID NO: 624 - protein, 123 aa, synthetic construct
EVQLVESGGGLVQPGGSLRLSCSASGFTFSRYAMYWVRQAPGKGLEYVSAISSDGGSTYDADSVKGRFTISRANSKNTLYLQMSSLRAEDTAVYYCVKGLRELLYYYYGMDVWGQGTTVTVSS

SEQ ID NO: 625 - protein, 8 aa, synthetic construct
GFTFSRYA

SEQ ID NO: 626 - protein, 8 aa, synthetic construct
ISSDGGST

SEQ ID NO: 627 - protein, 16 aa, synthetic construct
VKGLRELLYYYYGMDV

SEQ ID NO: 628 - protein, 107 aa, synthetic construct
DIQMTQSPSSLSASVGDRVTITCRAGQSISSFLNWYQQKPGKAPKLLIYAASSLQSGVPSRFSGSGSGTDFTLTISSLQPEDFATYYCQQSYITPFTFGPGTKVDIK

SEQ ID NO: 629 - protein, 9 aa, synthetic construct
QQSYITPFT

SEQ ID NO: 630 - protein, 120 aa, synthetic construct
EVQLVESGGGLVQPGGSLRLSCAASGFTFSSYEMNWVRQAPGKGLEWVSYISSSGSILYYADSVKGRFTASRDNAKNSLYLQMNSLRAEDTGVYYCARREYQLLRPFEFWGQGTLVTVSS

SEQ ID NO: 631 - protein, 8 aa, synthetic construct
GFTFSSYE

SEQ ID NO: 632 - protein, 8 aa, synthetic construct
ISSSGSIL

SEQ ID NO: 633 - protein, 13 aa, synthetic construct
ARREYQLLRPFEF

SEQ ID NO: 634 - protein, 108 aa, synthetic construct
DIQMTQSPSTLSASVGDRVTITCRASQSISSWLAWYQQKSGKAPKLLIYKASSLESGVPSRFSGSGSGTEFTLTISSLQPDDFATYYCQQYDSYSPWTFGQGTKVEIK

SEQ ID NO: 635 - protein, 10 aa, synthetic construct
QQYDSYSPWT

SEQ ID NO: 636 - protein, 116 aa, synthetic construct
EVQLVETGGGLIQPGGSLRLSCAASEFIVSRNYMSWVRQAPGKGLEWVSLIYSGGSTYYADSVKGRFTISRDNSKNILYLQMNSLRAEDTAVYYCARDRGGISDYWGQGTLVTVSS

SEQ ID NO: 637 - protein, 8 aa, synthetic construct
EFIVSRNY

SEQ ID NO: 638 - protein, 10 aa, synthetic construct
ARDRGGISDY

SEQ ID NO: 639 - protein, 107 aa, synthetic construct
DIQMTQSPSSLSASVGDRVTITCQASQDINKYLNWFQQKPGKAPKLLIYDASNLETGVPSRFSGSGSGTDFTFTISSLQPEDIATYYCQQYDNLPRTFGQGTKLEIK

SEQ ID NO: 640 - protein, 122 aa, synthetic construct
QVQLVQSGAEVKKPGASVRVSCKASGYTFTGNYIHWVRQAPGQGLEWMAWINPNSGGKNYAQKFQGRVTLTRDTSVNTAYMELSRLRSDDTAMYYCARDVNYNILTGPGGLWGQGTLVTVSS

SEQ ID NO: 641 - protein, 8 aa, synthetic construct
GYTFTGNY

SEQ ID NO: 642 - protein, 8 aa, synthetic construct
INPNSGGK

SEQ ID NO: 643 - protein, 15 aa, synthetic construct
ARDVNYNILTGPGGL

SEQ ID NO: 644 - protein, 110 aa, synthetic construct
QSALTQPPSASGSPGQSVTISCTGTSSDVGGYNFVSWYQQHPGKAPKLMIYEVSKRPSGVPDRFSGSKSGNTASLTVSGLQAEDEADYYCSSYAGSNNFVFGTGTKVTVL

SEQ ID NO: 645 - protein, 9 aa, synthetic construct
SSDVGGYNF

SEQ ID NO: 646 - protein, 10 aa, synthetic construct
SSYAGSNNFV

SEQ ID NO: 647 - protein, 122 aa, synthetic construct
QVQLVQSGADVKKPGASVKVSCKTSGYIFTGYYIHWVRQAPGQGLEWMGWINPNSGGTNSAQKFQGRVTMTRDTSISTVYMELSRLRSDDTAVYYCAKSNSFSVLDYNFDYWGQGTLVTVSS

SEQ ID NO: 648 - protein, 15 aa, synthetic construct
AKSNSFSVLDYNFDY

SEQ ID NO: 649 - protein, 109 aa, synthetic construct
SSELTQDPAVSVALGQTVRITCQGDSLRSYYASWYQQKPGQAPVLVIYDKNNRPSGIPDRFSGSSSGNTASLTITGAQAEDEADYYCNSRDSSRNHLVVFGGGTKLTVL

SEQ ID NO: 650 - protein, 6 aa, synthetic construct
SLRSYY

SEQ ID NO: 651 - protein, 12 aa, synthetic construct
NSRDSSRNHLVV

SEQ ID NO: 652 - protein, 122 aa, synthetic construct
QVQLVQSGAEVKKPGASVKVSCKASGYIFTDHYMHWVRQAPGQGLEWMGWINPNSGATNYAQNFQGRVTMTRDTSIITVYMELSRLRSDDTAVYYCARDSSPYMIRYTMDVWGQGTTITVSS

SEQ ID NO: 653 - protein, 8 aa, synthetic construct
GYIFTDHY

SEQ ID NO: 654 - protein, 8 aa, synthetic construct
INPNSGAT

SEQ ID NO: 655 - protein, 15 aa, synthetic construct
ARDSSPYMIRYTMDV

SEQ ID NO: 656 - protein, 111 aa, synthetic construct
QSALTQPASVSGSPGQSITISCTGTSSDVGSYNLVSWYQQHPGKAPKLMIYELTKRPSGVSNRFSGSKSGNTASLTISGLQAEDEADYYCSSYRGSVTDVVFGGGTKLTVL

SEQ ID NO: 657 - protein, 11 aa, synthetic construct
SSYRGSVTDVV

SEQ ID NO: 658 - protein, 118 aa, synthetic construct
QVQLQESGPGLVKPSGTLSLTCDVSGGSISSNNWWSWVRQRPGKGLQWIGEIYHNENTNYNPSLKSRVIMSIDKSKNQFSLKLTSVTAADTAVYYCARGDVLDWFDPWGQGTLVTVSS

SEQ ID NO: 659 - protein, 9 aa, synthetic construct
GGSISSNNW

SEQ ID NO: 660 - protein, 7 aa, synthetic construct
IYHNENT

SEQ ID NO: 661 - protein, 11 aa, synthetic construct
ARGDVLDWFDP

SEQ ID NO: 662 - protein, 110 aa, synthetic construct
QSALTQPASVSGSPGQSTTISCTGTSSDVGGYNYVSWYQQYPGKAPKLMIYDVTNRPSGVSHRFSGSKSGNTASLTISGLQAEDEADYYCSSYTSSSTVIFGGGTKLTVL

SEQ ID NO: 663 - protein, 10 aa, synthetic construct
SSYTSSSTVI

SEQ ID NO: 664 - protein, 119 aa, synthetic construct
QITLKESGPTLVKPTQTLTLTCTFSGFSFSTSGVGVGWIRQPPGKTLEWLALIYWDDDKRYSPSLKSRLTITKDTSKNQVVLTMTNMDPVDTATYFCAHHGIPTIFGYWGQGALVTVSS

SEQ ID NO: 665 - protein, 10 aa, synthetic construct
GFSFSTSGVG

SEQ ID NO: 666 - protein, 7 aa, synthetic construct
IYWDDDK

SEQ ID NO: 667 - protein, 11 aa, synthetic construct
AHHGIPTIFGY

SEQ ID NO: 668 - protein, 109 aa, synthetic construct
QSALTQPASVSGSPGQSITISCTGTSSDLGVFNYVSWYQQHPGKAPKLMIYEVTNRPSGVSNRFSGSKSGNTASLTISGLQAEDEADYYCSSYTTSSTVFGGGTKLTVL

SEQ ID NO: 669 - protein, 9 aa, synthetic construct
SSDLGVFNY

SEQ ID NO: 670 - protein, 9 aa, synthetic construct
SSYTTSSTV

SEQ ID NO: 671 - protein, 120 aa, synthetic construct
QVQLVQSGAEVKKPGASVKVSCKASGYTFTGYFIHWVRQAPGQGLEWMGWISPISGGTNYAQKFQGRVTVTRDTSITTAYMELSRLTSDDTAVYHCAREGGTWKGDFDYWGQGTLVTVSS

SEQ ID NO: 672 - protein, 8 aa, synthetic construct
GYTFTGYF

SEQ ID NO: 673 - protein, 8 aa, synthetic construct
ISPISGGT

SEQ ID NO: 674 - protein, 13 aa, synthetic construct
AREGGTWKGDFDY

SEQ ID NO: 675 - protein, 110 aa, synthetic construct
QSALTQPASVSGSPGQSITISCTGTSSDVESYNLVSWYQQHPGKAPKLMIYEGSKRPSGVSNRFSGSKSGNTASLTISGLQAEDEADYYCSSYAGSSTWVFGGGTKLTVL

SEQ ID NO: 676 - protein, 9 aa, synthetic construct
SSDVESYNL

SEQ ID NO: 677 - protein, 122 aa, synthetic construct
QVQLVQSGAEVKKPGASVKVSCKASGYTFTDYFIHWVRQAPGQGLEWMGWINPTSGGTNYAQKFRGRVTMTRDTSISTAYVELSRLRSDDTALYYCARDLPFYSDTKQFDYWGQGTLVTVSS

SEQ ID NO: 678 - protein, 8 aa, synthetic construct
GYTFTDYF

SEQ ID NO: 679 - protein, 8 aa, synthetic construct
INPTSGGT

SEQ ID NO: 680 - protein, 15 aa, synthetic construct
ARDLPFYSDTKQFDY

SEQ ID NO: 681 - protein, 110 aa, synthetic construct
QSALTQPASVSGYPGQSITISCTGTSSDVGGYNYVSWYQQHPGKAPKLMIFDVSNRPSGVSNRFSGSKSGNTASLTFSGLQAEDEADYYCSSYTSSSTLVFGGGTKLTVL

SEQ ID NO: 682 - protein, 10 aa, synthetic construct
SSYTSSSTLV

SEQ ID NO: 683 - protein, 120 aa, synthetic construct
QVQLVQSGAEVKKPGSSVKVSCKASGGTFSNNAISWVRQAPGQGLEWMGKIIPILGIGGDAQKFQGRFTITADKSTSTAYMELSSLRSEDTAVYYCANSGDSRPHAFDIWGQGTMVTVSS

SEQ ID NO: 684 - protein, 8 aa, synthetic construct
GGTFSNNA

SEQ ID NO: 685 - protein, 8 aa, synthetic construct
IIPILGIG

SEQ ID NO: 686 - protein, 13 aa, synthetic construct
ANSGDSRPHAFDI

SEQ ID NO: 687 - protein, 110 aa, synthetic construct
QSALTQPASVSGSPGQSITISCTGTSSDVGSYNLVSWYQQHPGKAPKLMIYEVSKRPSGVSNRFSGSKSGNTASLTISGLQAEDEADYYCSSYAGSRREVFGGGTKLTVL

SEQ ID NO: 688 - protein, 10 aa, synthetic construct
SSYAGSRREV

SEQ ID NO: 689 - protein, 126 aa, synthetic construct
EVQLVESGGGLVQPGGSLRLSCAASGFTLSSYDMHWVRQVIGKGLEWVSTIGTSGDTYYPGSVKGRFTISRENAKNSLYLQMNSLRAGDTAVYYCARGGVPTTIWSGPYYYYMDVWGKGTTVTVSS

SEQ ID NO: 690 - protein, 8 aa, synthetic construct
GFTLSSYD

SEQ ID NO: 691 - protein, 7 aa, synthetic construct
IGTSGDT

SEQ ID NO: 692 - protein, 20 aa, synthetic construct
ARGGVPTTIWSGPYYYYMDV

SEQ ID NO: 693 - protein, 112 aa, synthetic construct
QSVLTQPPSVSAAPGQKVTISCSGSSSNIGNNYVSWYQQLPGTAPKLLIYENNKRPSGIPDRFSGSKSGTSATLGITGLQTGDEADYYCGTWDSSLSAGQGVFGGGTKLTVL

SEQ ID NO: 694 - protein, 13 aa, synthetic construct
GTWDSSLSAGQGV

SEQ ID NO: 695 - protein, 118 aa, synthetic construct
QVQLVQSGPEVKKPGASVKVSCKASGYTFTSYAISWVRQAPGQGLEWMGWISAYNGNTNYAQKLQGRVTMTTDTFTSTAYMELRSLRSDDTAVYYCAVPADHITFDYWGQGTLVTVSS

SEQ ID NO: 696 - protein, 8 aa, synthetic construct
GYTFTSYA

SEQ ID NO: 697 - protein, 8 aa, synthetic construct
ISAYNGNT

SEQ ID NO: 698 - protein, 11 aa, synthetic construct
AVPADHITFDY

SEQ ID NO: 699 - protein, 107 aa, synthetic construct
EIVLTQSPGTLSLSPGERATLSCRASQSVSSYLAWYQQKPGQAPRLLIHGASSRATGIPDRFSGSGSGTDFTLTISRLEPEDFAVYYCQQYGSSFLTFGGGTKVEIK

SEQ ID NO: 700 - protein, 6 aa, synthetic construct
QSVSSY

SEQ ID NO: 701 - protein, 9 aa, synthetic construct
QQYGSSFLT

SEQ ID NO: 702 - protein, 122 aa, synthetic construct
QVQLVQSGAEVKKPGSSVKVSCKASGGSFSNYAISWVRQAPGQGLEWMGGIIPFFGTTNYAQKFQGRLTITADESTSTFYMELSSLGSEDTAVYYCAAGYSSGWYPSWFDPWGQGTLVTVSS

SEQ ID NO: 703 - protein, 8 aa, synthetic construct
GGSFSNYA

SEQ ID NO: 704 - protein, 8 aa, synthetic construct
IIPFFGTT

SEQ ID NO: 705 - protein, 15 aa, synthetic construct
AAGYSSGWYPSWFDP

SEQ ID NO: 706 - protein, 108 aa, synthetic construct
EIVLTQSPGTLSLSPGERATLSCRASQSVNNNYLAWYQQKPGQTPRLLIYGASSRATGIPDRFSGSGSGTDFTLTITRLEPEDFAVYHCQQYGRSPTTFGQGTRLEIK

SEQ ID NO: 707 - protein, 7 aa, synthetic construct
QSVNNNY

SEQ ID NO: 708 - protein, 9 aa, synthetic construct
QQYGRSPTT

SEQ ID NO: 709 - protein, 124 aa, synthetic construct
EVQLVQSGAEVKKPGSTMKISCKISGYIFSDYYMHWVQQAPGKGLEWMGLVDPEDGETRYAEKFQGRVTITADTSTDTAYMELSSLRFEDTAVYYCATVGGSSGPWGETLFDHWGQGSLVTVSS

SEQ ID NO: 710 - protein, 8 aa, synthetic construct
GYIFSDYY

SEQ ID NO: 711 - protein, 8 aa, synthetic construct
VDPEDGET

SEQ ID NO: 712 - protein, 17 aa, synthetic construct
ATVGGSSGPWGETLFDH

SEQ ID NO: 713 - protein, 112 aa, synthetic construct
DIVMTQSPLSLPVTPGEPASISCRSSQSLLHSNGYNYINWYLQKPGQSPQLLMYLGSNRASGVPDRFSGSGSGTDSTLKISRVEAEDAGVYYCMQTRQTPYTFGQGTKLEIK

SEQ ID NO: 714 - protein, 11 aa, synthetic construct
QSLLHSNGYNY

SEQ ID NO: 715 - protein, 9 aa, synthetic construct
MQTRQTPYT

SEQ ID NO: 716 - protein, 126 aa, synthetic construct
EVQLVESGGGLVQPGGSLRLSCVASGFAFSTYEMNWVRQAPGKGLEWVSYISSSGSTIYYADSVKGRLTISRDNVKNSLYLQMNSLRVEDTAIYYCARDAIRNNDILTDPATFDPWGQGTLVTVSS

SEQ ID NO: 717 - protein, 8 aa, synthetic construct
GFAFSTYE

SEQ ID NO: 718 - protein, 8 aa, synthetic construct
ISSSGSTI

SEQ ID NO: 719 - protein, 19 aa, synthetic construct
ARDAIRNNDILTDPATFDP

SEQ ID NO: 720 - protein, 112 aa, synthetic construct
DIVMTQSPLSLPVTPGEPASISCRSSQSLLYSNGYNYLDWYLQKPGQSPQLLISLGSNRASGVPDRFSGSGSGTDFTLKISRVEAEDVGVYYCMQALQTPYTFGQGTKLEIK

SEQ ID NO: 721 - protein, 11 aa, synthetic construct
QSLLYSNGYNY

SEQ ID NO: 722 - protein, 9 aa, synthetic construct
MQALQTPYT

SEQ ID NO: 723 - protein, 130 aa, synthetic construct
QVQLVQSGAEVKKPGASVKVSCKASGYTFTNYYMHWVRQAPGQGLEWMGIINPSGGSTSYAQKFLGRVTMTRDTSTTTVYMELSSLRSEDTAVYFCARVENLPTKWDLLRKGFESGMDVWGQGTTVTVSS

SEQ ID NO: 724 - protein, 8 aa, synthetic construct
GYTFTNYY

SEQ ID NO: 725 - protein, 8 aa, synthetic construct
INPSGGST

SEQ ID NO: 726 - protein, 23 aa, synthetic construct
ARVENLPTKWDLLRKGFESGMDV

SEQ ID NO: 727 - protein, 107 aa, synthetic construct
EIVMTQSPATVSVSPGERATLSCRASQSVSSNLAWYQQKPGQAPRLLIYGASTRATGIPASFSGSGSGTEFTLTISSLQSEDFAVYYCQQYNNWPITFGPGTKVDIK

SEQ ID NO: 728 - protein, 9 aa, synthetic construct
QQYNNWPIT

SEQ ID NO: 729 - protein, 128 aa, synthetic construct
QLQLQESGPGLVKPSETLSLTCTVSGGSISSSGHYWGWIRQSPGKGLDWIGSIYYSGSTYYNPSLKSRVTISVDTSKNQFSLKLNSVTAADTAVYFCARLEAYCSGDTCHYFYGLDVWGQGTTVTVSS

SEQ ID NO: 730 - protein, 10 aa, synthetic construct
GGSISSSGHY

SEQ ID NO: 731 - protein, 20 aa, synthetic construct
ARLEAYCSGDTCHYFYGLDV

SEQ ID NO: 732 - protein, 107 aa, synthetic construct
DIQMTQSPSSLSASVGDRVTITCRASQSINTYLNWYQQKPGKAPNLLIDGASSLQSGVPSRFSGSGSGTDFILTISSLRPEDFATYYCQQSYSIPLTFGGGTRVEIK

SEQ ID NO: 733 - protein, 6 aa, synthetic construct
QSINTY

SEQ ID NO: 734 - protein, 9 aa, synthetic construct
QQSYSIPLT

SEQ ID NO: 735 - protein, 117 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDIGITTPYWGQGTLVTVSS

SEQ ID NO: 736 - protein, 10 aa, synthetic construct
AKDIGITTPY

SEQ ID NO: 737 - protein, 107 aa, synthetic construct
AIQMTQSPSSLSASVGDRVTITCRASQDIRNDLGWYQQKPGKAPKLLIYSASSLQTGVPSRFSGSGSGTDFTLTISSLHPEDFATYYCLQDYNYVWTFGQGTKVEIK

SEQ ID NO: 738 - protein, 6 aa, synthetic construct
QDIRND

SEQ ID NO: 739 - protein, 9 aa, synthetic construct
LQDYNYVWT

SEQ ID NO: 740 - protein, 124 aa, synthetic construct
EVQLVESGGGLIQPGRSLRLSCAASGFTFDDYAMHWVRQAPGKGLEWVSGISWNSVSIDYADSVKGRFTISRDNAKNSLYLQMTSLTVEDTALYYCAKAFGYETLTGSDYFAYWGPGTLVTVSS

SEQ ID NO: 741 - protein, 8 aa, synthetic construct
ISWNSVSI

SEQ ID NO: 742 - protein, 17 aa, synthetic construct
AKAFGYETLTGSDYFAY

SEQ ID NO: 743 - protein, 109 aa, synthetic construct
EIVLTQSPGTLSLSPGERATLSCRASQTVISTYLAWYQQKPGQSPRLLIYGASSRATGIPDRFSGSGSGTDFTLTISRLEPEDFAVYYCQQYGSSPQLTFGGGTKVEIK

SEQ ID NO: 744 - protein, 7 aa, synthetic construct
QTVISTY

SEQ ID NO: 745 - protein, 10 aa, synthetic construct
QQYGSSPQLT

SEQ ID NO: 746 - protein, 128 aa, synthetic construct
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARAPGNFDWLFRSDYYYGMDVWGQGTTVTVSS

SEQ ID NO: 747 - protein, 8 aa, synthetic construct
GFTFSSYW

SEQ ID NO: 748 - protein, 8 aa, synthetic construct
IKQDGSEK

SEQ ID NO: 749 - protein, 21 aa, synthetic construct
ARAPGNFDWLFRSDYYYGMDV

SEQ ID NO: 750 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPVTFGQGTRLEIK

SEQ ID NO: 751 - protein, 9 aa, synthetic construct
QQSYSTPVT

SEQ ID NO: 752 - protein, 119 aa, synthetic construct
QITLKESGPTLVKPTQTLTLTCTFSGFSLSASGVGVGWIRQPPGKALEWLALIYWDDDKRYSPSLKSRLTITKDTSKNQVVLTMTNMDPVDTATYYCAQHKVATIFDYWGQGTLVTVSS

SEQ ID NO: 753 - protein, 10 aa, synthetic construct
GFSLSASGVG

SEQ ID NO: 754 - protein, 11 aa, synthetic construct
AQHKVATIFDY

SEQ ID NO: 755 - protein, 109 aa, synthetic construct
QSALTQPPSASGSPGQSVTISCTATSSDVGNYNYVSWYQQHPGKAPKLMIYEVTKRPSGVPDRFSGSKSGNTASLTVSGLQAEDEADYYCSSYAGSWGVFGGGTKLTVL

SEQ ID NO: 756 - protein, 9 aa, synthetic construct
SSDVGNYNY

SEQ ID NO: 757 - protein, 9 aa, synthetic construct
SSYAGSWGV

SEQ ID NO: 758 - protein, 119 aa, synthetic construct
QVQLVESGGGVVQPGRSLRLSCAASGFIFSSYGMHWVRQAPGKGLEWVAVIWYDGSNKFYADSVKGRFTISRDNSKNTLYLQVNSLRAEDTAVYYCARAVQGDWYFDHWGQGTLVTVSS

SEQ ID NO: 759 - protein, 8 aa, synthetic construct
GFIFSSYG

SEQ ID NO: 760 - protein, 12 aa, synthetic construct
ARAVQGDWYFDH

SEQ ID NO: 761 - protein, 108 aa, synthetic construct
SYELTQPPSVSVSPGQTARITCSGDVLAKLYAYWYQHKPGQAPVLVIYKDSERPSGIPERFSGSSSGTTVTLTISGVQAEDEADYYCQSADSSGSLWVFGGGTKLTVL

SEQ ID NO: 762 - protein, 6 aa, synthetic construct
VLAKLY

SEQ ID NO: 763 - protein, 11 aa, synthetic construct
QSADSSGSLWV

SEQ ID NO: 764 - protein, 127 aa, synthetic construct
QVQLVESGGGVVQPGRSLRLSCAASGFTFSSYGIHWVRQAPGKGLEWVAIIWYDGSEKYYADSVKGRFTISRDNSKNTLFLQMNSLEAEDTAVYYCARGPDLLTGYYNYFYYGMDVWGQGTTVTVSS

SEQ ID NO: 765 - protein, 8 aa, synthetic construct
IWYDGSEK

SEQ ID NO: 766 - protein, 20 aa, synthetic construct
ARGPDLLTGYYNYFYYGMDV

SEQ ID NO: 767 - protein, 111 aa, synthetic construct
QSVLTQPPSVSGAPGQRVTISCTGSSSNIGIGYDVHWYQQLPGTVPKLLIYGNTNRPSGVPDRFSGSKSGTSASLAITGLQAEDEADYYCQSYDSSLSSYVFGTGTKVTVL

SEQ ID NO: 768 - protein, 9 aa, synthetic construct
SSNIGIGYD

SEQ ID NO: 769 - protein, 11 aa, synthetic construct
QSYDSSLSSYV

SEQ ID NO: 770 - protein, 119 aa, synthetic construct
QITLKESGPTLVKPTQTLTLTCTFSGFSLNTGGVGVGWIRQPPGNALEWLALIYWDDDKRYNPSLKTRLTISKDTSKNQVVLTMTNMDPVDTATYFCAHLTIAVIVDFWGQGTLVTVSS

SEQ ID NO: 771 - protein, 10 aa, synthetic construct
GFSLNTGGVG

SEQ ID NO: 772 - protein, 11 aa, synthetic construct
AHLTIAVIVDF

SEQ ID NO: 773 - protein, 109 aa, synthetic construct
QSALTQPASVSGSPGQSITISCTATSSDVGTYNYVSWFQHHPGKAPKLMIYEVTNRPSGVSNRFSGSKSGNTASLTISGLQAEDEADYYCSSYTTTSIVFGGGTKLTVL

SEQ ID NO: 774 - protein, 9 aa, synthetic construct
SSYTTTSIV

SEQ ID NO: 775 - protein, 119 aa, synthetic construct
QITLKESGPTLVKPTQTLTLTCTFSGFSLSTSAVGVGWIRQPPGKALEWLALIYWDDDKRYSPSLKSRLTITKDTSKNQLVLTMTNVDPVDTATYYCAHHTIQLIFDYWGQGTLVTVSS

SEQ ID NO: 776 - protein, 10 aa, synthetic construct
GFSLSTSAVG

SEQ ID NO: 777 - protein, 11 aa, synthetic construct
AHHTIQLIFDY

SEQ ID NO: 778 - protein, 111 aa, synthetic construct
QSALTQPASVSGSPGQSITISCTATSSDVGNYNYVSWYQQHPGKAPKLMIYEVSNRPSGVSNRFSGSKSGNTASLTISGLQAEDEADYYCSSYTSSSTPLVFGGGTKLTVL

SEQ ID NO: 779 - protein, 11 aa, synthetic construct
SSYTSSSTPLV

SEQ ID NO: 780 - protein, 130 aa, synthetic construct
QVQLVQSGAEVKKPGASVKVSCKVSGYTLTELSIHWVRQAPGKGLEWMGGFNPEDAETIYAQKFQGRVTMTEDTSTDTAYMELRALRFEDTAVYYCATAFAVVSVGTGLNYNYYYGMDVWGQGTTVTVSS

SEQ ID NO: 781 - protein, 8 aa, synthetic construct
GYTLTELS

SEQ ID NO: 782 - protein, 8 aa, synthetic construct
FNPEDAET

SEQ ID NO: 783 - protein, 23 aa, synthetic construct
ATAFAVVSVGTGLNYNYYYGMDV

SEQ ID NO: 784 - protein, 112 aa, synthetic construct
DIVMTQTPLSSPVTLGQPASISCRSSQSLVHSDGNTYLSWLQQRPGQPPRLLIYKISNRFSGVPDRFSGSGAGTDFTLKISMVEAEDVGVYYCTQATQFPYTFGQGTNLEIK

SEQ ID NO: 785 - protein, 11 aa, synthetic construct
QSLVHSDGNTY

SEQ ID NO: 786 - protein, 9 aa, synthetic construct
TQATQFPYT

SEQ ID NO: 787 - protein, 123 aa, synthetic construct
QVQLVQSGAEVKKPGASVKVSCKASGYTFTGYYMHWVRQAPGQGLEWMGRINPYSGGTNYAQKFQGRVTMTRDTSISTAYMELSRLRSDDTAVYYCASGFEPHDYGDPNLPPWGQGTLVTVSS

SEQ ID NO: 788 - protein, 8 aa, synthetic construct
GYTFTGYY

SEQ ID NO: 789 - protein, 8 aa, synthetic construct
INPYSGGT

SEQ ID NO: 790 - protein, 16 aa, synthetic construct
ASGFEPHDYGDPNLPP

SEQ ID NO: 791 - protein, 108 aa, synthetic construct
DIQMTQSPSSLSASVGDRVTITCRASQSISNYLNWYQQKPGKAPKLLIYAASSLQSGVPSRFSGSGSGTDFTLTISSLQPEDFATYYCQHSYSTPPVTFGGGTKVEIK

SEQ ID NO: 792 - protein, 10 aa, synthetic construct
QHSYSTPPVT

SEQ ID NO: 793 - protein, 123 aa, synthetic construct
QVQLQESGPGLVKPSETLSLTCTVSGGSISGYYWSWIRQPPGKGLDWIGYIYYRGNTNYNPSLKSRVTISVDTSNNQFSLKLSSVTAADTAVYYCARYGEGYGDASGYHFDYWGQGTLVTVSS

SEQ ID NO: 794 - protein, 7 aa, synthetic construct
IYYRGNT

SEQ ID NO: 795 - protein, 17 aa, synthetic construct
ARYGEGYGDASGYHFDY

SEQ ID NO: 796 - protein, 110 aa, synthetic construct
QSALTQPASVSGSPGQSITISCTGTSSDVGSYNLVSWYQQHPGKAPKLMIYEDSKRPSGVANPFSGSKSGNTASLTISGLQAEDEADYYCSSYAGSSTWVFGGGTKLTVL

SEQ ID NO: 797 - protein, 121 aa, synthetic construct
QVQLQESGPGLVKPSETLSLTCTVSGGSISSHYWSWIRQPPGKGLEWIGYIYYSGSSNYNPSLKSRVTISVDTSKNQFSLKLNSVTAADTAVYYCARHYDILTGFDWFDPWGQGTLVTVSS

SEQ ID NO: 798 - protein, 8 aa, synthetic construct
GGSISSHY

SEQ ID NO: 799 - protein, 7 aa, synthetic construct
IYYSGSS

SEQ ID NO: 800 - protein, 15 aa, synthetic construct
ARHYDILTGFDWFDP

SEQ ID NO: 801 - protein, 112 aa, synthetic construct
QSVLTQPPSVSGAPGQRVTISCTGSSSNIGTHYDVHWYQQLPGTAPKLLIYGNSNRPSGVPDRFSGSKSGTSASLAITGLQAEDEADYYCQSFDNSLTAPYVFGTGTKVTVL

SEQ ID NO: 802 - protein, 9 aa, synthetic construct
SSNIGTHYD

SEQ ID NO: 803 - protein, 12 aa, synthetic construct
QSFDNSLTAPYV

SEQ ID NO: 804 - protein, 120 aa, synthetic construct
EVQLVESGGGLVQPGGSLRLSCAASGFTVSRSYMNWVRQAPGKGLEWVSVVFTGGTTKHTDSVKGRFTISRDNSMNTLYLQMNSLRPEDTAVYYCARVAIVETTGAFDIWGQGTMVTVSS

SEQ ID NO: 805 - protein, 7 aa, synthetic construct
VFTGGTT

SEQ ID NO: 806 - protein, 14 aa, synthetic construct
ARVAIVETTGAFDI

SEQ ID NO: 807 - protein, 112 aa, synthetic construct
QSVLTQPPSVSGAPGQRVTISCTGTSSNIGADFDVHWYQQFPGTAPKLLIYGHTNRPSGIPDRFSGSKSGTSASLAITGLQAEDEADYYCQSYDSSLSGSVVFGGGTKLTVL

SEQ ID NO: 808 - protein, 9 aa, synthetic construct
SSNIGADFD

SEQ ID NO: 809 - protein, 119 aa, synthetic construct
QLQLQESGPGLVKPSETLSLICTGSGDSISSSHYYWGWIRQPPGKGLEWIGSTYFSGSTYYNPSLKSRVSISVDTSKNQVSLKLISVTAADTAVYYCAREMGATLGDYWGPGTLVTVSS

SEQ ID NO: 810 - protein, 10 aa, synthetic construct
GDSISSSHYY

SEQ ID NO: 811 - protein, 7 aa, synthetic construct
TYFSGST

SEQ ID NO: 812 - protein, 11 aa, synthetic construct
AREMGATLGDY

SEQ ID NO: 813 - protein, 111 aa, synthetic construct
QSVLTQPPSASGTPGQRVTISCSGSSSNIGNNYVYWYQQFPGTTPRLLIYRNNERPSGVPDRFSGSKSGTSASLVISGLRSEDEADYYCAAWDDSLSAHYVFGTGTKVTVL

SEQ ID NO: 814 - protein, 12 aa, synthetic construct
AAWDDSLSAHYV

SEQ ID NO: 815 - protein, 126 aa, synthetic construct
EVQLVESGGGLVQPGGSLRLSCAASGFPFSTYEMNWVRQAPGKGLEWVSYISSSGNTIYYADSVKGRFTISRDNAKNSLYLQMNTLRAEDTAVYYCARDAVRDHDILTDPATFDPWGQGTLVTVSS

SEQ ID NO: 816 - protein, 8 aa, synthetic construct
GFPFSTYE

SEQ ID NO: 817 - protein, 8 aa, synthetic construct
ISSSGNTI

SEQ ID NO: 818 - protein, 19 aa, synthetic construct
ARDAVRDHDILTDPATFDP

SEQ ID NO: 819 - protein, 112 aa, synthetic construct
DIVMTQSPLSLPVTPGEPASISCRSSQTLLHSNGYNYLDWYLQKPGQSPQLLIHLGSNRASGVPDRFSGSGSVTDFTLKISRVEAEDVGVYYCMQALQTPYTFGQGTKLEIK

SEQ ID NO: 820 - protein, 11 aa, synthetic construct
QTLLHSNGYNY

SEQ ID NO: 821 - protein, 119 aa, synthetic construct
EVQLVESGGGLVQPGGSLRLSCAASGFTFSIYEMNWVRQAPGKGLEWVSYITSSGFTIYYADSVKGRFTISRDNAKNSLYLQMHSLRAEDTAVYYCATSSSSGYYFDYWGQGTLVTVSS

SEQ ID NO: 822 - protein, 8 aa, synthetic construct
ITSSGFTI

SEQ ID NO: 823 - protein, 12 aa, synthetic construct
ATSSSSGYYFDY

SEQ ID NO: 824 - protein, 107 aa, synthetic construct
AIQMTQSPSSLSASVGDRVTITCRASQDIRNDLGWYQQKPGKAPKLLIYSASSLQTGVPSRFSGSGSGTDFTLTISSLHPEDFATYYCLQDYDYTWTFGQGTKVEIK

SEQ ID NO: 825 - protein, 9 aa, synthetic construct
LQDYDYTWT

SEQ ID NO: 826 - protein, 107 aa, synthetic construct
AIQMTQSPSSLSASVGDRVTITCRASQDIRNDLGWYQQKPGKAPKLLIYSASSLQTGVPSRFSGSGSGTDFTLTISSLHPEDFATYYCLQDYWYTWTFGQGTKVEIK

SEQ ID NO: 827 - protein, 9 aa, synthetic construct
LQDYWYTWT

SEQ ID NO: 828 - protein, 126 aa, synthetic construct
EVQLVESGGGLVQPGGSLRLSCAASGFTFSNHWMDWVRQAPGKGLVWVSRINSDGSSTDYADSVRGRFTMSRDNAKNTLYLQMSSLRAEDTAVYYCAREGMIFGAVIIPGWYFDLWGRGALVTVSS

SEQ ID NO: 829 - protein, 8 aa, synthetic construct
GFTFSNHW

SEQ ID NO: 830 - protein, 8 aa, synthetic construct
INSDGSST

SEQ ID NO: 831 - protein, 19 aa, synthetic construct
AREGMIFGAVIIPGWYFDL

SEQ ID NO: 832 - protein, 104 aa, synthetic construct
EIVLTQSPGTLSLSPGERATLSCRASQSVSSSQLAWYQQKPGQAPRLLIYGASSRATGIPDRFSGSGSGTDFTLTISRLEPEDFAVYYCQQYDSFGQGTKLEIK

SEQ ID NO: 833 - protein, 7 aa, synthetic construct
QSVSSSQ

SEQ ID NO: 834 - protein, 5 aa, synthetic construct
QQYDS